(19) United States
(12) Patent Application Publication
(10) Pub. No.: US 2025/0114346 A1
(43) Pub. Date: Apr. 10, 2025

(54) COMBINATION THERAPY USING A KRAS G12D INHIBITOR AND PD-1 INHIBITOR OR PD-L1 INHIBITOR

(71) Applicant: Incyte Corporation, Wilmington, DE (US)

(72) Inventors: Matthew Farren, Wilmington, DE (US); Alexandra Gallion, Middletown, DE (US); Sunkyu Kim, Hockessin, DE (US); Valerie Roman, Hockessin, DE (US); Amanda Smith, Wilmington, DE (US); Renee Wallower, Swedesboro, NJ (US); Hui Wang, Hockessin, DE (US)

(21) Appl. No.: 18/909,683

(22) Filed: Oct. 8, 2024

Related U.S. Application Data
(60) Provisional application No. 63/588,924, filed on Oct. 9, 2023.
(60) Provisional application No. 63/680,218, filed on Aug. 7, 2024.

(51) Int. Cl. A61K 31/4745; A61K 31/519; A61K 39/395; A61P 35/00

(52) U.S. Cl. CPC A61K 31/4745; A61K 31/519; A61K 39/3955; A61P 35/00

(57) ABSTRACT

Provided herein are methods of treating cancer by administering a combination therapy comprising a KRAS G12D inhibitor and a PD-1 inhibitor or a PD-L1 inhibitor.

### RELATED APPLICATIONS

This application is related to U.S. Provisional Application No. 63/588,924, filed on Oct. 9, 2023, and U.S. Provisional Application No. 63/680,218, filed on Aug. 7, 2024, the entire content of each is incorporated herein.

### BACKGROUND

KRAS mutations are among the most common genetic alterations in cancer (D. A. Erlanson et. al., Curr. Opin. Chem. Biol., 2021, 62, 101-108). KRAS is a membrane-bound GTPase that, when activated through upstream receptor tyrosine kinases, promotes cell survival and proliferation (D. Uprety et al., Cancer Treat. Rev., 2020, 89, 102070). KRAS proteins exist in a GTP-bound ‘on’ state and GDP-bound ‘off’ state. When GTP-bound, signals are transduced through activation of the mitogen activated protein kinase pathway and the PI3K pathway, in addition to others. KRAS mutations are found in approximately 23% of solid tumors. The G12D isoform is the most common, accounting for approximately 29% of KRAS mutations in cancer (J. K. Lee, et al., NPJ Precis. Oncol., 2022, 6, 91). KRAS G12D mutations are found in approximately 40% of pancreatic cancers (pancreatic ductal adenocarcinoma), 15% of colorectal carcinomas, and 5% of non-small cell lung adenocarcinomas, representing major unmet medical needs. The KRAS G12D mutation impairs GTP hydrolysis, resulting in a hyperactivated KRAS isoform that drives high levels of oncogenic ERK and PI3K signaling (M. Malumbres, et al., Nat. Rev. Cancer., 2003, 3, 459-65).

Inhibiting KRAS G12D by binding to the KRAS G12D Switch-II pocket, which leads to conformational changes disfavoring GTP binding and RAF association is hypothesized to abrogate KRAS signaling and halt tumor growth in KRAS G12D mutant tumors. In addition to direct antitumor effects, inhibition of mutant KRAS signaling with small molecule inhibitors induces immunomodulatory changes in the tumor microenvironment in preclinical models. These immunomodulatory changes include increased antigen presentation by tumor cells, increased frequencies of tumor infiltrating T cells, and decreased frequencies of myeloid derived suppressor cells (S. B. Kemp, et al., Cancer Discov. 2023, 13(2), 298-311). Further, combining mutant KRAS inhibitors with immune checkpoint blockade, specifically PD-1/PD-L1 blockade, in mutant KRAS tumor models results in enhanced antitumor activity and durable responses in published preclinical studies (D. M. Briere, et al., Mol. Cancer Ther., 2021, 20(6), 975-85).

### SUMMARY

Provided herein is a method of treating cancer in a subject in need thereof comprising administering to the subject a KRAS G12D inhibitor, or a pharmaceutically acceptable salt thereof, and a PD-1 inhibitor or a PD-L1 inhibitor, or pharmaceutically acceptable salt thereof. Targeting KRAS G12D mutant tumors with a selective and reversible inhibitor in combination with PD-1/PD-L1 disrupting agents may be a promising cancer treatment for patients with KRAS G12D mutations.

### BRIEF DESCRIPTION OF THE DRAWINGS

FIG. 1 shows antitumor activity of Compound 1±retifanlimab in the CT-26 Clone 299 Model.

FIG. 2 shows tumor growth delay following cessation of treatment with Compound 1±retifanlimab in the CT-26 Clone 299 model.

FIG. 3 shows body weight changes of CT-26 Clone 299 tumor-bearing mice administered Compound 1±retifanlimab.

FIG. 4 shows inhibition of pERK in CT-26 clone 299 tumors by Compound 1±retifanlimab after 5 days of treatment.

FIG. 5 shows antitumor activity of Compound 1±Compound 2 in the CT-26 Clone 299 model.

FIG. 6 shows tumor growth delay following cessation of treatment with Compound 1±Compound 2 in the CT-26 Clone 299 model.

FIG. 7 shows body weight changes of CT-26 Clone 299 tumor-bearing mice administered Compound 1±Compound 2.

FIG. 8 shows inhibition of pERK in CT-26 Clone 299 tumors by Compound 1±Compound 2 after 5 days of treatment.

FIG. 9 shows antitumor activity of Compound 1±anti-mouse-PD-1 antibody RMP1-14 in the KPCY-013 (2838c3) Model.

FIG. 10 shows antitumor activity of Compound 1±anti-mouse-PD-L1 antibody 10F.9G2 in the KPCY-013 (2838c3) Model.

FIG. 11 shows antitumor activity of Compound 3±retifanlimab in the CT-26 Clone 299 Model.

FIG. 12 shows antitumor activity of Compound 3±Compound 2 in the CT-26 Clone 299 Model.

### DETAILED DESCRIPTION

Ras proteins are part of the family of small GTPases that are activated by growth factors and various extracellular stimuli. The Ras family regulates intracellular signaling pathways responsible for growth, migration, survival and differentiation of cells. Activation of Ras proteins at the cell membrane results in the binding of key effectors and initiation of a cascade of intracellular signaling pathways within the cell, including the RAF and PI3K kinase pathways. Somatic mutations in RAS may result in uncontrolled cell growth and malignant transformation while the activation of RAS proteins is tightly regulated in normal cells (D. Simanshu, et al., Cell, 2017, 170(1), 17-33). The Ras family is comprised of three members: KRAS, NRAS and HRAS. RAS mutant cancers account for about 25% of human cancers. KRAS is the most frequently mutated isoform accounting for 85% of all RAS mutations whereas NRAS and HRAS are found mutated in 12% and 3% of all Ras mutant cancers respectively (D. Simanshu, et al., Cell, 2017, 170(1), 17-33). KRAS mutations are prevalent amongst the top three most deadly cancer types: pancreatic (97%), colorectal (44%), and lung (30%) (A. D. Cox, et al. Nat. Rev. Drug. Discov., 2014, 13(11), 828-51). The majority of RAS mutations occur at amino acid residue 12, 13, and 61. The frequency of specific mutations varies between RAS gene isoforms and while G12 and Q61 mutations are predominant in KRAS and NRAS respectively, G12, G13 and Q61 mutations are most frequent in HRAS. Furthermore, the spectrum of mutations in a RAS isoform differs between cancer types. HRAS. Furthermore, the spectrum of mutations in a RAS isoform differs between cancer types. For example, KRAS G12D mutations predominate in pancreatic cancers (40%), followed by colorectal adenocarcinomas (15%) and lung cancers (5%)(Lee J K, et al. NPJ Precis. Oncol., 2022, 6, 459-465). Genomic studies across hundreds of cancer cell lines have demonstrated that cancer cells harboring KRAS mutations are highly dependent on KRAS function for cell growth and survival (R. McDonald, et al., Cell, 2017, 170(3), 577-92). The role of mutant KRAS as an oncogenic driver is further supported by extensive in vivo experimental evidence showing mutant KRAS is required for early tumor onset and maintenance in animal models (A. D. Cox, et al. Nat. Rev. Drug. Discov., 2014, 13(11), 828-51).

The immune system plays an important role in controlling and eradicating diseases such as cancer. However, cancer cells often develop strategies to evade or to suppress the immune system in order to favor their growth. One such mechanism is altering the expression of co-stimulatory and co-inhibitory molecules expressed on immune cells (M. A. Postow et al., J. Clin. Oncol., 2015, 33(17), 1974-82). Blocking the signaling of an inhibitory immune checkpoint, such as PD-1, has proven to be a promising and effective treatment modality.

Programmed Death-1 (“PD-1,” also known as “CD279”) is an approximately 31 kD type I membrane protein member of the extended CD28/CTLA-4 family of T-cell regulators that broadly negatively regulates immune responses (Y. Ishida, et al., EMBO J., 1992, 11, 3887-95). PD-1 is expressed on activated T-cells, B-cells, and monocytes (T. Yamazaki, et al., J. Immunol., 2002,169(10), 5538-45) and at low levels in natural killer (NK) T-cells (N. Martin-Orozco, et al., Semin. Cancer Biol., 2007, 17(4), 288-98).

Several lines of evidence from preclinical animal studies indicate that PD-1 and its ligands negatively regulate immune responses. PD-1-deficient mice have been shown to develop lupus-like glomerulonephritis and dilated cardiomyopathy (H. Nishimura, et al., Science, 2001, 291(5502), 319-22). Using an LCMV model of chronic infection, it has been shown that PD-1/PD-L1 interaction inhibits activation, expansion and acquisition of effector functions of virus-specific CD8 T cells (D. L. Barber, et al., Nature, 2006, 439, 682-87). Together, these data support the development of a therapeutic approach to block the PD-1-mediated inhibitory signaling cascade in order to augment or “rescue” T cell response. Accordingly, there is a need for new methods of blocking PD-1/PD-L1 protein/protein interaction, and thereby treating cancer in a subject.

The present disclosure is related to methods of treating cancer in a subject in need thereof comprising administering to the subject a KRAS G12D inhibitor, or a pharmaceutically acceptable salt thereof, and a PD-1 inhibitor or a PD-L1 inhibitor, or pharmaceutically acceptable salt thereof.

Certain terms used herein are described below. Compounds of the present disclosure are described using standard nomenclature. Unless defined otherwise, all technical and scientific terms used herein have the same meaning as is commonly understood by one of skill in the art to which this invention belongs.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

The term “about” when used in connection with a numerical value, means that a collection or range of values is included. For example, “about X” includes a range of values that are 10%, +5%, 2%, 1%, 0.5%, 0.2%, or 0.1% of X, where X is a numerical value. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

As used herein, “pharmaceutical combination” or “combination” refers to formulations of the separate compounds with or without instructions for combined use or to combination products. The combination compounds may thus be entirely separate pharmaceutical dosage forms or in pharmaceutical compositions that are also sold independently of each other and where instructions for their combined use are provided in the package equipment, e.g., leaflet or the like, or in other information, e.g., provided to physicians and medical staff (e.g., oral communications, communications in writing or the like), for simultaneous or sequential use for being jointly active.

The term “treat,” “treated,” “treating,” or “treatment” includes the diminishment or alleviation of at least one symptom associated or caused by the state, disorder or disease being treated. In certain embodiments, the treatment comprises bringing into contact with KRAS or PD-1 an effective amount of a compound disclosed herein for conditions related to cancer.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the term “free base equivalent” refers to the amount of active agent, or a pharmaceutically acceptable salt of the active agent (e.g., Compound 1) that is equivalent to the free-base of the active agent dose. Stated alternatively, the term “free base equivalent” means either an amount of Compound 1 free base, or the equivalent amount of Compound 1 free base that is provided by a salt of said compound.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein a parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts described herein include the conventional non-toxic salts of the parent compound formed, for example, from non-toxic inorganic or organic acids. The pharmaceutically acceptable salts discussed herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in A. R. Gennaro (Ed.), Remington's Pharmaceutical Sciences, 17^{th} Ed., (Mack Publishing Company, Easton, 1985), p. 1418, S. M. Berge et al., J. Pharm. Sci., 1977, 66(1), 1-19, S. Gaisford in A. Adejare (Ed.), Remington, The Science and Practice of Pharmacy, 23^{rd} Ed., (Elsevier, 2020), Chapter 17, pp. 307-14; S. M. Berge et al., J. Pharm. Sci., 1977, 66(1), 1-19, T. S. Wiedmann, et al., Asian J. Pharm. Sci., 2016; 11, 722-34. D. Gupta et al., Molecules, 2018, 23(7), 1719; P. H. Stahl et al., Handbook of Pharmaceutical Salts: Properties, Selection, and Use, (Wiley, 2002) and in P. H. Stahl et al., Handbook of Pharmaceutical Salts: Properties, Selection, and Use, 2^{nd} Ed. (Wiley, 2011).

As used herein, the term “composition” or “pharmaceutical composition” refers to a mixture of at least one compound with a pharmaceutically acceptable carrier. The pharmaceutical composition facilitates administration of the composition to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound disclosed herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of a compound disclosed herein, and are physiologically acceptable to the patient. Supplementary active compounds may also be incorporated into the compositions. The “pharmaceutically acceptable carrier” may further include a pharmaceutically acceptable salt of the compound(s) disclosed herein. Other additional ingredients that may be included in the pharmaceutical compositions are known in the art and described, for example, in P. Beringer, et al., (Eds.), Remington: The Science and Practice of Pharmacy, 21^{st} Ed.; (Lippincott Williams & Wilkins: Philadelphia, Pa., 2005); A. Adejare (Ed.), Remington, The Science and Practice of Pharmacy, 23^{rd} Ed., (Elsevier, 2020); R. C. Rowe et al., Eds., Handbook of Pharmaceutical Excipients, 6^{th} Ed.; (Pharmaceutical Press, 2009); P. J. Shesky et al., Eds., Handbook of Pharmaceutical Excipients, 9^{th} Ed.; (The Pharmaceutical Press, 2020); M. Ash, et al., (Eds.), Handbook of Pharmaceutical Additives, 3^{rd} Ed.; (Gower Publishing Company: 2007); and M. Gibson (Ed.), Pharmaceutical Preformulation and Formulation, 2^{nd} Ed. (CRC Press LLC, 2009).

The term “single formulation” as used herein refers to a single carrier or vehicle formulated to deliver effective amounts of both therapeutic agents to a patient. The single vehicle is designed to deliver an effective amount of each of the agents, along with any pharmaceutically acceptable carriers or excipients. In some embodiments, the vehicle is a tablet, capsule, pill, or a patch. In other embodiments, the vehicle is a solution or a suspension.

The term “combination therapy” refers to the administration of two or more therapeutic compounds to treat a therapeutic condition or disorder described in the present disclosure. Such administration encompasses co-administration of these therapeutic compounds in a substantially simultaneous manner, such as in a single capsule having a fixed ratio of active ingredients or in multiple, or in separate containers (e.g., capsules) for each active ingredient. In addition, such administration also encompasses use of each type of therapeutic compound in a sequential manner, either at approximately the same time or at different times. In either case, the treatment regimen will provide beneficial effects of the drug combination in treating the conditions or disorders described herein.

The combination of agents described herein may display a synergistic effect. The term “synergistic effect” as used herein, refers to action of two agents such as, for example, a KRAS inhibitor (e.g., a KRAS inhibitor of formula I) and a PD-1 or PD-L1 inhibitor, producing an effect, for example, slowing the symptomatic progression of cancer or symptoms thereof, which is greater than the simple addition of the effects of each drug administered by themselves. A synergistic effect can be calculated, e.g., using suitable methods such as the Sigmoid-Emax equation (N. H. G. Holford, et al., Clin. Pharmacokinet., 1981, 6: 429-53), the equation of Loewe additivity (S. Loewe, et al., Arch. Exp. Pathol Pharmacol., 1926, 114, 313-26) the median-effect equation (T. C. Chou, et al., Adv. Enzyme Regul., 1984, 22: 27-55), or based on the Bliss definition of drugs independence (E. Demidenko, et al., PLoS ONE, 2019, 14(11): e0224137). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

As used herein, the term “synergy” refers to the effect achieved when the active ingredients, i.e., KRAS inhibitor and PD-1 inhibitor or PD-L1 inhibitor, used together is greater than the sum of the effects that results from using the compounds separately.

In an embodiment, provided herein is a combination therapy comprising an effective amount of a KRAS inhibitor and PD-1 inhibitor or PD-L1 inhibitor. An “effective amount” of a combination of agents (i.e., KRAS inhibitor and PD-1 inhibitor or PD-L1 inhibitor) is an amount sufficient to provide an observable improvement over the baseline clinically observable signs and symptoms of the disorders treated with the combination.

Provided herein is a combination of therapeutic agents and administration of the combination of agents to treat cancer, and related indications. As used herein, the term “cancer” includes related indications, such as anemia. As used herein, a “combination of agents” and similar terms refer to a combination of two types of agents: a KRAS inhibitor, or a pharmaceutically acceptable salt thereof, and a PD-1 inhibitor, or a pharmaceutically acceptable salt thereof, or a PD-L1 inhibitor, or a pharmaceutically acceptable salt thereof. Use of racemic mixtures of the individual agents is also provided. Pharmacologically active metabolites include those that are inactive but converted into pharmacologically active forms in the body after administration.

As used herein, the term “alkyl,” by itself or as part of another substituent means, unless otherwise stated, a straight or branched chain hydrocarbon having the number of carbon atoms designated (i.e., C₁-C₆-alkyl means an alkyl having one to six carbon atoms) and includes straight and branched chains. In an embodiment, C₁-C₃, C₁-C₄, C₁-C₆alkyl groups are provided herein. Examples include methyl, ethyl, propyl, isopropyl, butyl, isobutyl, tert-butyl, pentyl, neopentyl, and hexyl.

The term “alkylene,” employed alone or in combination with other terms, refers to a divalent alkyl linking group. An alkylene group formally corresponds to an alkane with two C—H bond replaced by points of attachment of the alkylene group to the remainder of the compound. The term “Cₙ₋ₘ alkylene” refers to an alkylene group having n to m carbon atoms. Examples of alkylene groups include, but are not limited to, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

As used herein, the term “alkoxy,” refers to the group —O-alkyl, wherein alkyl is as defined herein. Alkoxy includes, by way of example, methoxy, ethoxy, n-propoxy, isopropoxy, n-butoxy, sec-butoxy, t-butoxy and the like. In an embodiment, C₁-C₃, C₁-C₄, C₁-C₆ alkoxy groups are provided herein.

The term “amino,” employed alone or in combination with other terms, refers to a group of formula —NH₂, wherein the hydrogen atoms may be substituted with a substituent described herein. For example, “alkylamino” can refer to —NH(alkyl) and —N(alkyl)₂.

As used herein, the term “halo” or “halogen” alone or as part of another substituent means, unless otherwise stated, a fluorine, chlorine, bromine, or iodine atom.

The term “haloalkyl” as used herein refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1}halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CC₃, CHCl₂, C₂Cl₅ and the like. In some embodiments, the haloalkyl group is a fluoroalkyl group.

The term “haloalkoxy,” employed alone or in combination with other terms, refers to a group of formula —O-haloalkyl, wherein the haloalkyl group is as defined above. The term “Cₙ₋ₘ haloalkoxy” refers to a haloalkoxy group, the haloalkyl group of which has n to m carbons. Example haloalkoxy groups include trifluoromethoxy and the like. In some embodiments, the haloalkoxy group has 1 to 6, 1 to 4, or 1 to 3 carbon atoms.

As used herein, the term “cycloalkyl” means a non-aromatic carbocyclic system that is partially or fully saturated having 1, 2 or 3 rings wherein such rings may be fused. The term “fused” means that a second ring is present (i.e., attached or formed) by having two adjacent atoms in common (i.e., shared) with the first ring. Cycloalkyl also includes bicyclic structures that may be bridged or spirocyclic in nature with each individual ring within the bicycle varying from 3-10, 3-8, 3-7, 3-6, and 5-10 atoms. The term “cycloalkyl” includes, but is not limited to, cyclopropyl, cyclobutyl, cyclopentyl, cyclohexyl, bicyclo[3.1.0]hexyl, spiro[3.3]heptanyl, bicyclo[2.2.2]octanyl and bicyclo[1.1.1]pentyl. In an embodiment, 3-10 membered cycloalkyl groups are provided herein.

As used herein, the term “heterocycloalkyl” means a non-aromatic carbocyclic system containing 1, 2, 3 or 4 heteroatoms selected independently from N, O, and S and having 1, 2 or 3 rings wherein such rings may be fused, wherein fused is defined above. Heterocycloalkyl also includes bicyclic structures that may be bridged or spirocyclic in nature with each individual ring within the bicycle varying from 3-8, 5-10, 4-6, or 3-10 atoms, and containing 0, 1, or 2 N, O, or S atoms. The term “heterocycloalkyl” includes cyclic esters (i.e., lactones) and cyclic amides (i.e., lactams) and also specifically includes, but is not limited to, epoxidyl, oxetanyl, tetrahydrofuranyl, tetrahydropyranyl (i.e., oxanyl), pyranyl, dioxanyl, aziridinyl, azetidinyl, pyrrolidinyl, 2,5-dihydro-1H-pyrrolyl, oxazolidinyl, thiazolidinyl, piperidinyl, morpholinyl, piperazinyl, thiomorpholinyl, 1,3-oxazinanyl, 1,3-thiazinanyl, 2-aza-bicyclo[2.1.1]hexanyl, 5-azabicyclo[2.1.1]hexanyl, 6-azabicyclo[3.1.1]heptanyl, 2-azabicyclo-[2.2.1]heptanyl, 3-aza-bicyclo[3.1.1]heptanyl, 2-azabicyclo[3.1.1]heptanyl, 3-azabicyclo-[3.1.0]hexanyl, 2-aza-bicyclo[3.1.0]hexanyl, 3-azabicyclo[3.2.1]octanyl, 8-azabicyclo[3.2.1]-octanyl, 3-oxa-7-aza-bicyclo[3.3.1]nonanyl, 3-oxa-9-azabicyclo[3.3.1]nonanyl, 2-oxa-5-aza-bicyclo[2.2.1]heptanyl, 6-oxa-3-azabicyclo[3.1.1]heptanyl, 2-azaspiro[3.3]heptanyl, 2-oxa-6-azaspiro[3.3]heptanyl, 2-oxaspiro[3.3]heptanyl, 2-oxaspiro[3.5]nonanyl, 3-oxaspiro[5.3]-nonanyl, and 8-oxabicyclo-[3.2.1]octanyl. In an embodiment, 3-10 membered heterocycloalkyl groups are provided herein. In another embodiment, 5-10 membered heterocycloalkyl groups are provided herein. In still another embodiment, 4-6 membered heterocycloalkyl groups are provided herein.

The term “aromatic” refers to a carbocycle or heterocycle having one or more polyunsaturated rings having aromatic character (i.e., having (4n+2) delocalized □ (pi) electrons where n is an integer).

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. Aryl groups include, e.g., phenyl, naphthyl, and the like. In some embodiments, aryl groups have from 6 to about 10 carbon atoms. In some embodiments, aryl groups have 6 carbon atoms. In some embodiments, aryl groups have 10 carbon atoms. In some embodiments, the aryl group is phenyl. In some embodiments, the aryl group is naphthyl.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. The term “heteroaryl” includes, but is not limited to, furanyl, thiophenyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo[1,2-a]-pyridinyl, pyrazolo[1,5-a]pyridinyl, 5,6,7,8-tetrahydroisoquinolinyl, 5,6,7,8-tetra-hydroquinolinyl, 6,7-dihydro-5H-cyclopenta[b]pyridinyl, 6,7-dihydro-5H-cyclopenta[c]-pyridinyl, 1,4,5,6-tetrahydrocyclopenta[c]pyrazolyl, 2,4,5,6-tetrahydrocyclopenta[c]-pyrazolyl, 5,6-dihydro-4H-pyrrolo[1,2-b]pyrazolyl, 6,7-dihydro-5H-pyrrolo[1,2-b]-[1,2,4]triazolyl, 5,6,7,8-tetrahydro-[1,2,4]triazolo[1,5-a]pyridinyl, 4,5,6,7-tetrahydro-pyrazolo[1,5-a]pyridinyl, 4,5,6,7-tetrahydro-1H-indazolyl and 4,5,6,7-tetrahydro-2H-indazolyl. In an embodiment, 5-10 membered heteroaryl groups are provided herein.

It is to be understood that if a cycloalkyl, heterocycloalkyl, aryl, or heteroaryl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3- or 4-pyridinyl, the term “thienyl” means 2- or 3-thioenyl, and so forth.

As used herein, the term “substituted” means that an atom or group of atoms has replaced hydrogen as the substituent attached to another group.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

### KRAS G12D Inhibitors

The present disclosure relates to a combination therapy comprising a KRAS G12D inhibitor and a PD-1 inhibitor or a PD-L1 inhibitor. This combination therapy can be used to treat various disorders associated with abnormal activity of KRAS or PD-1/PD-L1.

In an embodiment, the KRAS G12D inhibitor is a compound of Formula I:

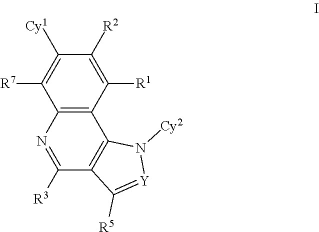

or a pharmaceutically acceptable salt thereof, wherein:
Y is N or CR⁶;
R¹ is selected from H, C₁₋₃ alkyl, C₁₋₃ haloalkyl, cyclopropyl, halo, D, CN, and OR^{a1}; wherein said C₁₋₃ alkyl and cyclopropyl are each optionally substituted with 1 or 2 substituents independently selected from R^{g};
R² is selected from H, C₁₋₃ alkyl, C₁₋₃ haloalkyl, 4-6 membered heterocycloalkyl, phenyl, 5-6 membered heteroaryl, 4-6 membered heterocycloalkyl-C₁₋₃ alkylene, phenyl-C₁₋₃ alkylene, 5-6 membered heteroaryl-C₁₋₃ alkylene, halo, D, CN, and OR^{a2}; wherein said C₁₋₃ alkyl, 4-6 membered heterocycloalkyl, phenyl, 5-6 membered heteroaryl, 4-6 membered heterocycloalkyl-C₁₋₃ alkylene, phenyl-C₁₋₃ alkylene, 5-6 membered heteroaryl-C₁₋₃ alkylene are each optionally substituted with 1 or 2 substituents independently selected from R^{g};
Cy¹ is selected from C₃₋₁₀cycloalkyl, 4-10 membered heterocycloalkyl, C₆₋₁₀ aryl and 6-10 membered heteroaryl; wherein the 4-10 membered heterocycloalkyl and 6-10 membered heteroaryl each has at least one ring-forming carbon atom and 1, 2, 3, or 4 ring-forming heteroatoms independently selected from N, O, and S; wherein a ring-forming carbon atom of 6-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 6-10 membered heteroaryl are each optionally substituted with 1, 2, 3, or 4 substituents independently selected from R¹⁰;
R³ is selected from H, C₁₋₃ alkyl, C₁₋₃ haloalkyl, C₃₋₆ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, D, CN, OR^{f3}, C(O)NR^{c3}R^{d3}, NR^{c3}R^{d3}, and NR^{c3}C(O)R^{b3}; wherein said C₁₋₃ alkyl, C₃₋₆ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 substituents independently selected from R³⁰;
R⁵ is selected from H, C₁₋₃ alkyl, C₁₋₃ haloalkyl, cyclopropyl, halo, D, CN, and OR^{a5}; wherein said C₁₋₃ alkyl and cyclopropyl are each optionally substituted with 1 or 2 substituents independently selected from R^{g};
R⁶ is selected from H, C₁₋₃ alkyl, C₁₋₃ haloalkyl, C₃₋₆ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, D, CN, OR^{a6}, and C(O)NR^{c6}R^{d6}; wherein said C₁₋₃ alkyl, C₃₋₆cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1 or 2 substituents independently selected from R⁶⁰;
R⁷ is selected from H, C₁₋₃ alkyl, C₁₋₃ haloalkyl, cyclopropyl, halo, D, CN, and OR^{a7}; wherein said C₁₋₃ alkyl and cyclopropyl are each optionally substituted with 1 or 2 substituents independently selected from R^{g};
Cy² is selected from

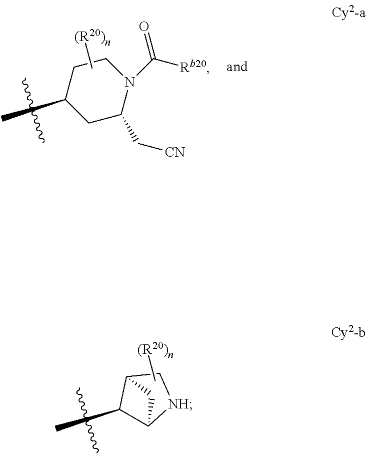

wherein n is 0, 1, or 2;
each R¹⁰ is independently selected from C₁₋₃ alkyl, C₁₋₃ haloalkyl, halo, D, CN, OR^{a10}, C(O)R^{b10}, C(O)NR^{c10}R^{d10}, C(O)OR^{a10}, NR^{c10}R^{d10}, and S(O)₂R^{b10};
each R²⁰ is independently selected from C₁₋₃ alkyl, C₁₋₃ haloalkyl, halo, D, CN, and OR^{a20};
each R³⁰ is independently selected from C₁₋₃ alkyl, C₁₋₃ haloalkyl, C₃₋₆ cycloalkyl, 4-6 membered heterocycloalkyl, phenyl, 5-6 membered heteroaryl, halo, D, CN, OR^{a30}, C(O)R^{b30}, C(O)NR^{c30}R^{d30}, C(O)OR^{a30}, NR^{c30}R^{d30}, and S(O)₂R^{b30}; wherein said C₁₋₃ alkyl, C₃₋₆ cycloalkyl, 4-6 membered heterocycloalkyl, phenyl, and 5-6 membered heteroaryl are each optionally substituted with 1 or 2 substituents independently selected from R³¹;
each R³¹ is independently selected from C₁₋₃ alkyl, C₁₋₃ haloalkyl, halo, D, CN, OR^{a31}, C(O)R^{b31}, C(O)NR^{c31}R^{d31}, C(O)OR^{a31}, NR^{c31}R^{d31}, and S(O)₂R^{b31};
each R³³ is independently selected from C₁₋₃ alkyl, C₁₋₃ haloalkyl, C₃₋₆ cycloalkyl, 4-membered heterocycloalkyl, 6-membered heterocycloalkyl, phenyl, 5-6 membered heteroaryl, halo, D, CN, OR^{a30}, C(O)NR^{c30}R^{d30}, and NR^{c30}R^{d30}; wherein said C₁₋₃ alkyl, C₃₋₆ cycloalkyl, 4-membered heterocycloalkyl, 6-membered heterocycloalkyl, phenyl, and 5-6 membered heteroaryl are each optionally substituted with 1 or 2 substituents independently selected from R³¹;
each R⁶⁰ is independently selected from C₁₋₃ alkyl, C₁₋₃ haloalkyl, 4-6 membered heterocycloalkyl, 5-6 membered heteroaryl, halo, D, CN, OR^{a60}, C(O)R^{b60}, C(O)NR^{c60}R^{d60}, NR^{c60}C(O)R^{b60}, C(O)OR^{a60}, NR^{c60}C(O)OR^{a60}, NR^{c60}R^{d60}, NR^{c60}S(O)₂R^{b60}, and S(O)₂R^{b60}; wherein said C₁₋₃ alkyl, 4-6 membered heterocycloalkyl, and 5-6 membered heteroaryl are each optionally substituted with 1 or 2 substituents independently selected from R⁶¹;
each R⁶¹ is independently selected from C₁₋₃ alkyl, C₁₋₃ haloalkyl, halo, D, CN, OR^{a61}, and NR^{c61}R^{d61};
R^{a1} is selected from H, C₁₋₃ alkyl, and C₁₋₃ haloalkyl;
each R^{a2} is independently selected from H, C₁₋₃ alkyl, and C₁₋₃ haloalkyl;
each R^{b3}, R^{c3} and R^{d3} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R³⁰;
or R^{c3} and R^{d3} attached to the same N atom, together with the N atom to which they are attached, form a 4-, 5-, or 6-membered heterocycloalkyl group optionally substituted with 1, 2, or 3 substituents independently selected from R³⁰;
R^{j3} is selected from C₁₋₃ alkyl, C₁₋₃ haloalkyl, C₃₋₆ cycloalkyl, 4-6 membered heterocycloalkyl, phenyl, and 5-6 membered heteroaryl; wherein said C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R³⁰;
or R^{c3} and R^{d3} attached to the same N atom, together with the N atom to which they are attached, form a 4-, 5-, or 6-membered heterocycloalkyl group optionally substituted with 1, 2, or 3 substituents independently selected from R³⁰;
R^{f3} is selected from C₁₋₃ haloalkyl, C₃₋₆ cycloalkyl, 4-6 membered heterocycloalkyl, phenyl and 5-6 membered heteroaryl; wherein said C₁₋₃ haloalkyl, C₃₋₆ cycloalkyl, 4-6 membered heterocycloalkyl, phenyl, and 5-6 membered heteroaryl are each optionally substituted with 1, 2, or 3 substituents independently selected from R³⁰; or
R^{f3} is selected from

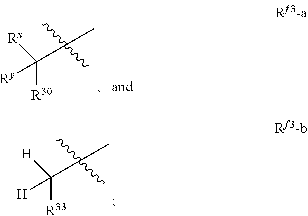

wherein R^{x} is H or C₁₋₂ alkyl and R^{y} is C₁₋₂ alkyl;
or R^{x} and R^{y}, together with the C atom to which they are attached, form a 3-, or 4-membered cycloalkyl group;
R^{a5} is selected from H, C₁₋₃ alkyl, and C₁₋₃ haloalkyl;
each R^{a6}, R^{c6} and R^{d6} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said C₁₋₃ alkyl, C₃₋₆ cycloalkyl, 4-6 membered heterocycloalkyl, phenyl and 5-6 membered heteroaryl are each optionally substituted with 1 or 2 substituents independently selected from R⁶⁰;
R^{a7} is selected from H, C₁₋₃ alkyl, and C₁₋₃ haloalkyl;
each R^{a10}, R^{b10}, R^{c10} and R^{d10} is independently selected from H, C₁₋₃ alkyl, and C₁₋₃ haloalkyl;
each R^{a20} is independently selected from H, C₁₋₃ alkyl, and C₁₋₃ haloalkyl;
R^{b20} is selected from NH₂, C₁₋₃ alkyl, and C₁₋₃ haloalkyl;
each R^{a30}, R^{b30}, R^{c30} and R^{d30} is independently selected from H, C₁₋₃ alkyl, and C₁₋₃ haloalkyl;
each R^{a31}, R^{b31}, R^{c31} and R^{d31} is independently selected from H, C₁₋₃ alkyl, and C₁₋₃ haloalkyl;
each R^{a60}, R^{b60}, R^{c60} and R^{d60} is independently selected from H, C₁₋₃ alkyl, C₁₋₃ haloalkyl, C₃₋₆ cycloalkyl, 4-6 membered heterocycloalkyl, and 5-6 membered heteroaryl; wherein said C₁₋₃ alkyl, C₃₋₆cycloalkyl, 4-6 membered heterocycloalkyl, and 5-6 membered heteroaryl are each optionally substituted with 1 or 2 substituents independently selected from R⁶¹;
or any R^{c60} and R^{d60} attached to the same N atom, together with the N atom to which they are attached, form a 4-, 5-, or 6-membered heterocycloalkyl group optionally substituted with 1 or 2 substituents independently selected from R⁶¹;
each R^{a61}, R^{c61}, and R^{d61}, is independently selected from H, C₁₋₃ alkyl, and C₁₋₃ haloalkyl; and
each R^{g} is independently selected from D, OH, CN, halo, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino.

In an embodiment of Formula I, or a pharmaceutically acceptable salt thereof,
Y is CR⁶;
R¹ is selected from H, C₁₋₃ alkyl, and C₁₋₃ haloalkyl;
R² is selected from H, C₁₋₃ alkyl, C₁₋₃ haloalkyl, halo, D, CN, and OR^{a2}; wherein said C₁₋₃ alkyl is optionally substituted with 1 or 2 substituents independently selected from R^{g};
Cy¹ is selected from C₃₋₁₀cycloalkyl, 4-10 membered heterocycloalkyl, C₆₋₁₀ aryl and 6-10 membered heteroaryl; wherein the 4-10 membered heterocycloalkyl and 6-10 membered heteroaryl each has at least one ring-forming carbon atom and 1, 2, 3, or 4 ring-forming heteroatoms independently selected from N, O, and S; wherein a ring-forming carbon atom of 6-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 6-10 membered heteroaryl are each optionally substituted with 1, 2, 3, or 4 substituents independently selected from R¹⁰;
R³ is selected from H, C₁₋₃ alkyl, C₁₋₃ haloalkyl, C₃₋₆ cycloalkyl, 4-6 membered heterocycloalkyl, phenyl, 5-6 membered heteroaryl, halo, D, CN, C(O)NR^{c3}R^{d3}, and NR^{c3}C(O)R^{b3}; wherein said C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R³⁰;
R⁵ is selected from H, C₁₋₃ alkyl, C₁₋₃ haloalkyl, and halo;
R⁶ is selected from H, C₁₋₃ haloalkyl, C₃₋₆ cycloalkyl, 4-8 membered heterocycloalkyl, phenyl, 5-6 membered heteroaryl, halo, D, CN, OR^{a6}, and C(O)NR^{c6}R^{d6}; wherein said C₃₋₆ cycloalkyl, 4-8 membered heterocycloalkyl, phenyl, and 5-6 membered heteroaryl are each optionally substituted with 1 or 2 substituents independently selected from R⁶⁰;
R⁷ is selected from H, C₁₋₃ alkyl, C₁₋₃ haloalkyl, halo, and CN;
Cy² is selected from

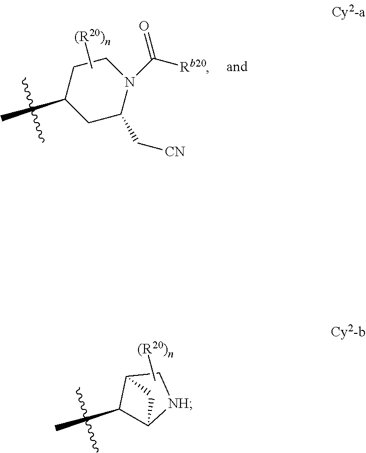

wherein n is 0, 1, or 2;
each R¹⁰ is independently selected from C₁₋₃ alkyl, C₁₋₃ haloalkyl, halo, D, CN, OR^{a10}, C(O)R^{b10}, C(O)NR^{c10}R^{d10}, C(O)OR^{a10}, NR^{c10}R^{d10}, and S(O)₂R^{b10};
each R²⁰ is independently selected from C₁₋₃ alkyl, C₁₋₃ haloalkyl, halo, D, CN, and OR^{a20};
each R³⁰ is independently selected from C₁₋₃ alkyl, C₁₋₃ haloalkyl, C₃₋₆ cycloalkyl, 4-6 membered heterocycloalkyl, phenyl, 5-6 membered heteroaryl, halo, D, CN, OR^{a30}, C(O)R^{b30}, C(O)NR^{c30}R^{d30}, C(O)OR^{a30}, NR^{c30}R^{d30}, and S(O)₂R³⁰; wherein said C₁₋₃ alkyl, C₃₋₆ cycloalkyl, 4-6 membered heterocycloalkyl, phenyl, and 5-6 membered heteroaryl are each optionally substituted with 1 or 2 substituents independently selected from R³¹;
each R³¹ is independently selected from C₁₋₃ alkyl, C₁₋₃ haloalkyl, halo, D, CN, OR^{a31}, C(O)R^{b31}, C(O)NR^{c31}R^{d31}, C(O)OR^{a31}, NR^{c31}R^{d31}, and S(O)₂R^{b31};
each R⁶⁰ is independently selected from C₁₋₃ alkyl, C₁₋₃ haloalkyl, C₁₋₃ haloalkoxy, 4-6 membered heterocycloalkyl, 5-6 membered heteroaryl, halo, D, CN, OR^{a60}, C(O)R^{b60}, C(O)NR^{c60}R^{d60}, NR^{c60}C(O)R^{b60}, C(O)OR^{a60}, NR^{c60}C(O)OR^{a60}, NR^{c60}R^{d60}, NR^{c60}S(O)₂R^{b60}, and S(O)₂R^{b60}; wherein said C₁₋₃ alkyl, 4-6 membered heterocycloalkyl, and 5-6 membered heteroaryl are each optionally substituted with 1 or 2 substituents independently selected from R⁶¹;
each R⁶¹ is independently selected from C₁₋₃ alkyl, C₁₋₃ haloalkyl, halo, D, CN, OR^{a61}, and NR^{c61}R^{d61};
each R^{a2} is independently selected from H, C₁₋₃ alkyl, and C₁₋₃ haloalkyl;
each R^{b3}, R^{c3} and R^{d3} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said, C₁₋₃ alkyl, C₃₋₆cycloalkyl, 4-6 membered heterocycloalkyl, phenyl and 5-6 membered heteroaryl are each optionally substituted with 1, 2, or 3 substituents independently selected from R³⁰;
or R^{c3} and R^{d3} attached to the same N atom, together with the N atom to which they are attached, form a 4-, 5-, or 6-membered heterocycloalkyl group optionally substituted with 1, 2, or 3 substituents independently selected from R³⁰;
each R^{a6}, R^{c6} and R^{d6} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said C₁₋₃ alkyl, C₃₋₆ cycloalkyl, 4-6 membered heterocycloalkyl, phenyl and 5-6 membered heteroaryl are each optionally substituted with 1 or 2 substituents independently selected from R⁶⁰;
each R^{a10}, R^{b10}, R^{c10} and R^{d10} is independently selected from H, C₁₋₃ alkyl, and C₁₋₃ haloalkyl;
each R^{a20} is independently selected from H, C₁₋₃ alkyl, and C₁₋₃ haloalkyl; R^{b20} is selected from NH₂, C₁₋₃ alkyl, and C₁₋₃ haloalkyl;
each R^{a30}, R^{b30}, R^{c30} and R^{d30} is independently selected from H, C₁₋₃ alkyl, and C₁₋₃ haloalkyl;
each R^{a31}, R^{b31}, R^{c31} and R^{d31} is independently selected from H, C₁₋₃ alkyl, and C₁₋₃ haloalkyl;
each R^{a60}, R^{b60}, R^{c60} and R^{d60} is independently selected from H, C₁₋₃ alkyl, C₁₋₃ haloalkyl, C₃₋₆ cycloalkyl, 4-6 membered heterocycloalkyl, and 5-6 membered heteroaryl; wherein said C₁₋₃ alkyl, C₃₋₆cycloalkyl, 4-6 membered heterocycloalkyl, and 5-6 membered heteroaryl are each optionally substituted with 1 or 2 substituents independently selected from R⁶¹;
or any R^{c60} and R^{d60} attached to the same N atom, together with the N atom to which they are attached, form a 4-, 5-, or 6-membered heterocycloalkyl group optionally substituted with 1 or 2 substituents independently selected from R⁶¹; and
each R^{a61}, R^{c61}, and R^{d61}, is independently selected from H, C₁₋₃ alkyl, and C₁₋₃ haloalkyl; and
each R⁹ is independently selected from D, CN, halo, C₁₋₃ alkyl, and C₁₋₃ haloalkyl.

In another embodiment of Formula I, or a pharmaceutically acceptable salt thereof,
Y is CR⁶;
R¹ is H;
R² is selected from C₁₋₃ alkyl, C₁₋₃ haloalkyl, halo, CN, and —CH₂CH₂CN;
Cy¹ is selected from C₃₋₁₀ cycloalkyl, C₆₋₁₀ aryl and 6-10 membered heteroaryl; wherein the 6-10 membered heteroaryl has at least one ring-forming carbon atom and 1, ring-forming heteroatoms independently selected from N and S; and wherein the C₃₋₁₀ cycloalkyl, C₆₋₁₀ aryl and 6-10 membered heteroaryl are each optionally substituted with 1 or 2 substituents independently selected from R¹⁰;
R³ is selected from H, C₁₋₃ alkyl, C₁₋₃ haloalkyl, 4-6 membered heterocycloalkyl, phenyl, and 5-6 membered heteroaryl; wherein said C₁₋₃ alkyl, 4-6 membered heterocycloalkyl, phenyl, and 5-6 membered heteroaryl are each optionally substituted with 1, 2, or 3 substituents independently selected from R³⁰;
R⁵ is selected from H and halo;
R⁶ is selected from H, C₁₋₃ haloalkyl, 4-8 membered heterocycloalkyl, and 5-6 membered heteroaryl; wherein said 4-8 membered heterocycloalkyl and 5-6 membered heteroaryl are each optionally substituted with 1 or 2 substituents independently selected from R⁶⁰; or
R⁷ is halo;
Cy² is

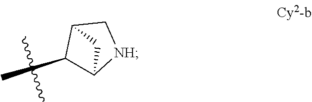

each R¹⁰ is independently selected from C₁₋₃ alkyl, C₁₋₃ haloalkyl, halo, D, CN, and OR^{a10};
each R³⁰ is independently selected from C₁₋₃ alkyl, C₁₋₃ haloalkyl, 4-6 membered heterocycloalkyl, halo, D, CN, OR^{a30}, C(O)NR^{c30}R^{d30}, and NR^{c30}R^{d30}; wherein said C₁₋₃ alkyl and 4-6 membered heterocycloalkyl are each optionally substituted with 1 or 2 substituents independently selected from R³¹;
each R³¹ is independently selected from C₁₋₃ alkyl, C₁₋₃ haloalkyl, halo, CN, OR^{a31}, and NR^{c31}R^{d31};
each R⁶⁰ is independently selected from C₁₋₃ alkyl, C₁₋₃ haloalkyl, C₁₋₃ haloalkoxy, 4-6 membered heterocycloalkyl, 5-6 membered heteroaryl, halo, D, CN, OR^{a60}, C(O)R^{b60}, C(O)NR^{c60}R^{d60}, NR^{c60}C(O)R^{b60}, C(O)OR^{a60}, NR^{c60}C(O)OR^{a60}, NR^{c60}R^{d60}, NR^{c60}S(O)₂R^{b60}, and S(O)₂R^{b60}; wherein said C₁₋₃ alkyl, 4-6 membered heterocycloalkyl, and 5-6 membered heteroaryl are each optionally substituted with 1 or 2 substituents independently selected from R⁶¹;
each R⁶¹ is independently selected from C₁₋₃ alkyl, C₁₋₃ haloalkyl, halo, and CN;
each R^{a10} is independently selected from H and C₁₋₃ alkyl;
each R^{a30}, R^{c30} and R^{d30} is independently selected from H and C₁₋₃ alkyl;
each R^{a31}, R^{c31} and R^{d31} is independently selected from H and C₁₋₃ alkyl;
each R^{a60}, R^{b60}, R^{c60} and R^{d60} is independently selected from H, C₁₋₃ alkyl, C₁₋₃ haloalkyl, C₃₋₆ cycloalkyl, 4-6 membered heterocycloalkyl, and 5-6 membered heteroaryl; wherein said C₁₋₃ alkyl, C₃₋₆cycloalkyl, 4-6 membered heterocycloalkyl, and 5-6 membered heteroaryl are each optionally substituted with 1 or 2 substituents independently selected from R⁶¹;
or any R^{c60} and R^{d60} attached to the same N atom, together with the N atom to which they are attached, form a 4-, 5-, or 6-membered heterocycloalkyl group optionally substituted with 1 or 2 substituents independently selected from R⁶¹.

In yet another embodiment of Formula I, or a pharmaceutically acceptable salt thereof,
Y is CR⁶;
R¹ is H;
R² is —CH₂CH₂CN;
Cy¹ is phenyl; wherein the phenyl is optionally substituted with 1 or 2 substituents independently selected from R¹⁰;
R³ is selected from H, C₁₋₃ alkyl, phenyl, and 5-6 membered heteroaryl; wherein said C₁₋₃ alkyl, phenyl, and 5-6 membered heteroaryl are each optionally substituted with 1, 2 or 3 substituents independently selected from R³⁰;
R⁵ is selected from H and halo;
R⁶ is selected from 4-8 membered heterocycloalkyl; wherein said 4-8 membered heterocycloalkyl is optionally substituted with 1 or 2 substituents independently selected from R⁶⁰; or
R⁶ is selected from C₁₋₃ alkyl; wherein said C₁₋₃ alkyl is substituted with 1 or 2 substituents independently selected from R⁶⁰;
R⁷ is halo;
Cy² is

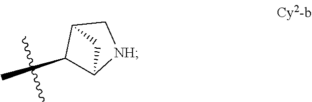

each R¹⁰ is independently selected from C₁₋₃ alkyl and halo;
each R³⁰ is independently selected from C₁₋₃ alkyl, halo, D, OH, and C(O)NR^{c30}R^{d30};
wherein said C₁₋₃ alkyl is optionally substituted with 1 substituent independently selected from R³¹;
each R³¹ is OR^{a31};
each R⁶⁰ is independently selected from C₁₋₃ alkyl, C₁₋₃ haloalkoxy, 4-6 membered heterocycloalkyl, 5-6 membered heteroaryl, halo, C(O)R^{b60}, C(O)NR^{c60}R^{d60}, NR^{c60}C(O)R^{b60}, C(O)OR^{a60}, NR^{c60}C(O)OR^{a60}, and NR^{c60}S(O)₂R^{b60}; wherein said C₁₋₃ alkyl, 4-6 membered heterocycloalkyl, and 5-6 membered heteroaryl are each optionally substituted with 1 or 2 substituents independently selected from R⁶¹;
each R⁶¹ is independently selected from C₁₋₃ alkyl, and halo;
each R^{c30} and R^{d30} is independently selected from H and C₁₋₃ alkyl;
each R^{a31} is independently selected from H and C₁₋₃ alkyl; and
each R^{a60}, R^{b60}, R^{c60} and R^{d60} is independently selected from H, C₁₋₃ alkyl, C₁₋₃ haloalkyl, C₃₋₆ cycloalkyl, 4-6 membered heterocycloalkyl, and 5-6 membered heteroaryl; wherein said C₁₋₃ alkyl, C₃₋₆cycloalkyl, 4-6 membered heterocycloalkyl, and 5-6 membered heteroaryl are each optionally substituted with 1 or 2 substituents independently selected from R⁶¹;
or any R^{c60} and R^{d60} attached to the same N atom, together with the N atom to which they are attached, form a 4-, 5-, or 6-membered heterocycloalkyl group optionally substituted with 1 or 2 substituents independently selected from R⁶¹.

In still another embodiment of Formula I, or a pharmaceutically acceptable salt thereof,
Y is CR⁶;
R¹ is H;
R² is —CH₂CH₂CN;
Cy¹ is phenyl; wherein the phenyl is optionally substituted with 1 or 2 substituents independently selected from R¹⁰;
R³ is selected from H, methyl, ethyl, phenyl, 1,2,4-triazolyl, pyrazyl, and pyridyl; wherein said methyl, phenyl, 1,2,4-triazolyl, pyrazyl, and pyridyl are each optionally substituted with 1, 2 or 3 substituents independently selected from R³⁰;
R⁵ is selected from H and chloro;
R⁶ is selected from pyrrolidinyl, 2-azabicyclo[3.1.0]hexanyl, 2-azabicyclo[2.2.1]heptanyl, and 5-oxo-1,2,3,5-tetrahydroindolizin-3-yl; wherein said pyrrolidinyl, 2-azabicyclo[3.1.0]hexanyl, 2-azabicyclo[2.2.1]heptanyl, and 5-oxo-1,2,3,5-tetrahydroindolizin-3-yl are optionally substituted with 1 or 2 substituents independently selected from R⁶⁰;
R⁷ is fluoro;
Cy² is

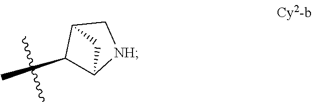

each R¹⁰ is independently selected from methyl, fluoro, and chloro;
each R³⁰ is independently selected from methyl, fluoro, OH, D, and C(O)NR^{c30}R^{d30}; wherein said methyl is optionally substituted with 1 substituent that is R³¹;
each R³¹ is OR^{a31};
each R⁶⁰ is independently selected from methyl, fluoro, C₁₋₂ haloalkoxy, 3-oxomorpholinyl, 2-oxopyrazin-1(2H)-yl), C(O)R^{b60}, C(O)NR^{c60}R^{d60}, NR^{c60}C(O)R^{b60}, C(O)OR^{a60}, NR^{c60}C(O)OR^{a60}, and NR^{c60}S(O)₂R^{b60}; wherein said 3-oxomorpholinyl, and 2-oxopyrazin-1(2H)-yl) are each optionally substituted with 1 or 2 substituents independently selected from R⁶¹;
each R⁶¹ is independently selected from methyl and fluoro;
each R^{c30} and R^{d30} is independently selected from H and methyl;
each R^{a31} is independently selected from H and methyl; and
each R^{a60}, R^{b60}, R^{c60} and R^{d60} is independently selected from H, C₁₋₂ alkyl, C₁ haloalkyl, cyclopropyl, tetrahydrofuranyl, and thiazolyl; wherein said C₁₋₂ alkyl, cyclopropyl, tetrahydrofuranyl, and thiazolyl are each optionally substituted with 1 or 2 substituents independently selected from R⁶¹;
or any R^{c60} and R^{d60} attached to the same N atom, together with the N atom to which they are attached, form an azetidinyl group optionally substituted with 1 or 2 substituents independently selected from R⁶¹.

In an embodiment, the compound of Formula I is a compound of Formula II:

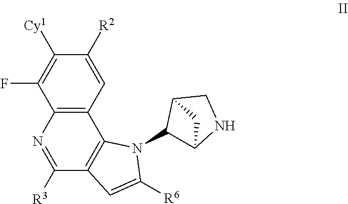

or a pharmaceutically acceptable salt thereof.

In another embodiment, the compound of Formula I is a compound of Formula III:

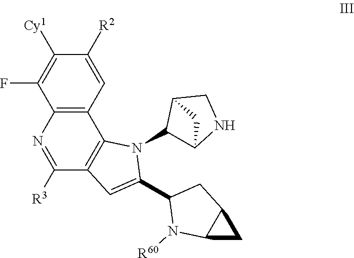

or a pharmaceutically acceptable salt thereof.

In an embodiment of Formula I, or a pharmaceutically acceptable salt thereof, Y is CR⁶. In another embodiment of Formula I, or a pharmaceutically acceptable salt thereof, R¹ is H. In yet another embodiment of Formula I, or a pharmaceutically acceptable salt thereof, Cy¹ is phenyl optionally substituted with 1 or 2 substituents independently selected from halo. In still another embodiment of Formula I, or a pharmaceutically acceptable salt thereof, R³ is methyl. In an embodiment of Formula I, or a pharmaceutically acceptable salt thereof, R⁵ is H. In an embodiment of Formula I, or a pharmaceutically acceptable salt thereof, R⁶ is 2-azabicyclo[3.1.0]hexanyl substituted with R⁶⁰. In another embodiment of Formula I, or a pharmaceutically acceptable salt thereof, R⁷ is fluoro. In yet another embodiment of Formula I, or a pharmaceutically acceptable salt thereof, Cy² is Cy²-b. In still another embodiment, R⁶⁰ is C(O)cyclopropyl.

In an embodiment, the KRAS G12D inhibitor is selected from
3-(1-((1R,4R,5S)-2-azabicyclo[2.1.1]hexan-5-yl)-7-(7-chloro-3-hydroxynaphthalen-1-yl)-6-fluoro-2-methyl-4-(1H-1,2,4-triazol-1-yl)-1H-pyrrolo[3,2-c]quinolin-8-yl)propanenitrile;
3-(1-((1R,4R,5S)-2-azabicyclo[2.1.1]hexan-5-yl)-7-(5,7-difluoro-1H-indol-3-yl)-6-fluoro-2-methyl-4-((S)-1-((S)-1-methylpyrrolidin-2-yl)ethoxy)-1H-pyrrolo[3,2-c]quinolin-8-yl)propanenitrile;
3-(1-((1R,4R,5S)-2-azabicyclo[2.1.1]hexan-5-yl)-6-fluoro-7-(6-fluoro-5-methyl-1H-indol-3-yl)-2-methyl-4-((S)-1-((S)-1-methylpyrrolidin-2-yl)ethoxy)-1H-pyrrolo[3,2-c]quinolin-8-yl)propanenitrile;
3-(2-(3-(azetidin-1-yl)-3-oxopropyl)-1-((1R,4R,5S)-2-azabicyclo[2.1.1]hexan-5-yl)-7-(2,3-dichlorophenyl)-6-fluoro-4-((S)-1-((S)-1-methylpyrrolidin-2-yl)ethoxy)-1H-pyrrolo[3,2-c]quinolin-8-yl)propanenitrile;
3-((1-((1R,4R,5S)-2-azabicyclo[2.1.1]hexan-5-yl)-6-fluoro-7-(3-hydroxynaphthalen-1-yl)-8-methyl-4-((S)-1-((S)-1-methylpyrrolidin-2-yl)ethoxy)-1H-pyrrolo[3,2-c]quinolin-2-yl)methyl)oxazolidin-2-one;
8-(1-((1R,4R,5S)-2-azabicyclo[2.1.1]hexan-5-yl)-6-fluoro-2,8-dimethyl-4-((S)-1-((S)-1-methylpyrrolidin-2-yl)ethoxy)-1H-pyrrolo[3,2-c]quinolin-7-yl)-1-naphthonitrile;
1-((2S,4S)-1-acetyl-2-(cyanomethyl)piperidin-4-yl)-7-(8-cyanonaphthalen-1-yl)-6-fluoro-4-((S)-1-((S)-1-methylpyrrolidin-2-yl)ethoxy)-1H-pyrazolo[4,3-c]quinoline-8-carbonitrile;
8-(1-((1R,4R,5S)-2-azabicyclo[2.1.1]hexan-5-yl)-6-fluoro-8-methyl-4-((S)-1-((S)-1-methylpyrrolidin-2-yl)ethoxy)-2-((3-oxomorpholino)methyl)-1H-pyrrolo[3,2-c]quinolin-7-yl)-1-naphthonitrile;
3-(7-(benzo[b]thiophen-3-yl)-1-((1R,4R,5S)-2-azabicyclo[2.1.1]hexan-5-yl)-6-fluoro-4-((S)-1-((S)-1-methylpyrrolidin-2-yl)ethoxy)-2-((2-oxopyrrolidin-1-yl)methyl)-1H-pyrrolo[3,2-c]quinolin-8-yl)propanenitrile;
3-(1-((1R,4R,5S)-2-azabicyclo[2.1.1]hexan-5-yl)-4-(((S)-1-(dimethylamino)propan-2-yl)oxy)-6-fluoro-7-(7-fluoronaphthalen-1-yl)-2-((2-oxopyrrolidin-1-yl)methyl)-1H-pyrrolo[3,2-c]quinolin-8-yl)propanenitrile;
8-(1-((1R,4R,5S)-2-azabicyclo[2.1.1]hexan-5-yl)-8-(2-cyanoethyl)-6-fluoro-2-methyl-4-((S)-1-((S)-1-methylpyrrolidin-2-yl)ethoxy)-1H-pyrrolo[3,2-c]quinolin-7-yl)-1-naphthonitrile;
3-(1-((1R,4R,5S)-2-azabicyclo[2.1.1]hexan-5-yl)-7-(2,3-dichloro-5-hydroxyphenyl)-6-fluoro-2-methyl-4-((S)-1-((S)-1-methylpyrrolidin-2-yl)ethoxy)-1H-pyrrolo[3,2-c]quinolin-8-yl)propanenitrile;
3-(1-((1R,4R,5S)-2-azabicyclo[2.1.1]hexan-5-yl)-8-(2-cyanoethyl)-6-fluoro-4-((3-fluoro-1-methylazetidin-3-yl)methoxy)-7-(3-hydroxynaphthalen-1-yl)-1H-pyrrolo[3,2-c]quinolin-2-yl)-N,N-dimethylpropanamide;
3-(1-((1R,4R,5S)-2-azabicyclo[2.1.1]hexan-5-yl)-6-fluoro-7-(3-hydroxynaphthalen-1-yl)-2-methyl-4-(5-methylpyrazin-2-yl)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoronaphthalen-1-yl)-4-methyl-2-((4-methyl-2-oxopiperazin-1-yl)methyl)-1H-pyrrolo[3,2-c]quinolin-8-yl)propanenitrile;
3-(1-((1R,4R,5S)-2-azabicyclo[2.1.1]hexan-5-yl)-7-(2,3-dichloro-5-hydroxyphenyl)-4-ethoxy-6-fluoro-2-((4-isopropyl-2-oxopiperazin-1-yl)methyl)-1H-pyrrolo[3,2-c]quinolin-8-yl)propanenitrile;
3-(1-((1R,4R,5S)-2-azabicyclo[2.1.1]hexan-5-yl)-4-(3-(dimethylamino)-3-methylazetidin-1-yl)-6-fluoro-7-(7-fluoronaphthalen-1-yl)-2-((3-oxomorpholino)methyl)-1H-pyrrolo[3,2-c]quinolin-8-yl)propanenitrile;
3-(1-((1R,4R,5S)-2-azabicyclo[2.1.1]hexan-5-yl)-4-ethoxy-6-fluoro-7-(3-hydroxynaphthalen-1-yl)-2-(1-(3-oxomorpholino)ethyl)-1H-pyrrolo[3,2-c]quinolin-8-yl)propanenitrile;
3-(1-((endo)-2-azabicyclo[2.1.1]hexan-5-yl)-6-fluoro-7-(3-hydroxynaphthalen-1-yl)-4-((S)-1-((S)-1-methylpyrrolidin-2-yl)ethoxy)-2-(pyridin-3-yl)-1H-pyrrolo[3,2-c]quinolin-8-yl)propanenitrile;
3-(2-(3-(azetidin-1-yl)-3-oxopropyl)-1-((1R,4R,5S)-2-azabicyclo[2.1.1]hexan-5-yl)-7-(7,8-difluoronaphthalen-1-yl)-6-fluoro-4-((S)-1-((S)-1-methylpyrrolidin-2-yl)ethoxy)-1H-pyrrolo[3,2-c]quinolin-8-yl)propanenitrile;
3-(2-(3-(azetidin-1-yl)-3-oxopropyl)-1-((1R,4R,5S)-2-azabicyclo[2.1.1]hexan-5-yl)-7-(6,7-difluoronaphthalen-1-yl)-6-fluoro-4-((S)-1-((S)-1-methylpyrrolidin-2-yl)ethoxy)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoro-3-hydroxynaphthalen-1-yl)-2-methyl-4-((S)-1-((S)-1-methylpyrrolidin-2-yl)ethoxy)-1H-pyrrolo[3,2-c]quinolin-8-yl)propanenitrile;
1-(1-((2S,4S)-1-acetyl-2-(cyanomethyl)piperidin-4-yl)-8-chloro-6-fluoro-4-((S)-1-((S)-1-methylpyrrolidin-2-yl)ethoxy)-1H-pyrazolo[4,3-c]quinolin-7-yl)isoquinoline-8-carbonitrile;
8-(1-((2S,4S)-1-acetyl-2-(cyanomethyl)piperidin-4-yl)-8-chloro-6-fluoro-4-((S)-1-((S)-1-methylpyrrolidin-2-yl)ethoxy)-1H-pyrrolo[3,2-c]quinolin-7-yl)-1-naphthonitrile;
8-(1-((2S,4S)-1-acetyl-2-(cyanomethyl)piperidin-4-yl)-8-chloro-6-fluoro-4-((S)-1-((S)-1-methylpyrrolidin-2-yl)ethoxy)-1H-pyrazolo[4,3-c]quinolin-7-yl)-1-naphthonitrile;
3-(1-((1R,4R,5S)-2-azabicyclo[2.1.1]hexan-5-yl)-6-fluoro-7-(7-fluoro-3-hydroxynaphthalen-1-yl)-2-methyl-4-(1H-1,2,4-triazol-1-yl)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoronaphthalen-1-yl)-2-methyl-4-((S)-1-((S)-1-methylpyrrolidin-2-yl)ethoxy)-1H-pyrrolo[3,2-c]quinolin-8-yl)propanenitrile;
(2R)-2-(1-((1R,4R,5S)-2-azabicyclo[2.1.1]hexan-5-yl)-8-(2-cyanoethyl)-7-(2,3-dichlorophenyl)-6-fluoro-4-(1H-1,2,4-triazol-1-yl)-1H-pyrrolo[3,2-c]quinolin-2-yl)-N,N-dimethylpyrrolidine-1-carboxamide;
methyl (2R)-2-(1-((1R,4R,5S)-2-azabicyclo[2.1.1]hexan-5-yl)-7-(2-chloro-3-methylphenyl)-8-(2-cyanoethyl)-6-fluoro-4-(1H-1,2,4-triazol-1-yl)-1H-pyrrolo[3,2-c]quinolin-2-yl)pyrrolidine-1-carboxylate;
methyl (1 S,3R,5S)-3-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)-2-azabicyclo[3.1.0]hexane-2-carboxylate;
3-(1-((1R,4R,5S)-2-azabicyclo[2.1.1]hexan-5-yl)-7-(2,3-dichlorophenyl)-6-fluoro-4-methyl-2-(5-oxo-1,2,3,5-tetrahydroindolizin-3-yl)-1H-pyrrolo[3,2-c]quinolin-8-yl)propanenitrile;
methyl (2R)-2-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)pyrrolidine-1-carboxylate;
methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(methylcarbamoyl)pyridin-3-yl)-1H-pyrrolo[3,2-c]quinolin-2-yl)pyrrolidine-1-carboxylate;
3-(1-((1R,4R,5S)-2-azabicyclo[2.1.1]hexan-5-yl)-7-(2-chloro-3-fluorophenyl)-2-((R)-1-(cyclopropanecarbonyl)pyrrolidin-2-yl)-6-fluoro-4-methyl-1H-pyrrolo[3,2-c]quinolin-8-yl)propanenitrile;
8-(2-((R)-1-acetylpyrrolidin-2-yl)-1-((1R,4R,5S)-2-azabicyclo[2.1.1]hexan-5-yl)-6-fluoro-8-methyl-4-(2-methylpyridin-4-yl)-1H-pyrrolo[3,2-c]quinolin-7-yl)-1,2,3,4-tetrahydronaphthalene-1-carbonitrile;
5-(1-((1R,4R,5S)-2-azabicyclo[2.1.1]hexan-5-yl)-7-(3-chloro-2-methylphenyl)-8-(2-cyanoethyl)-6-fluoro-2-((R)-1-(2-oxopyrazin-1 (2H)-yl)ethyl)-1H-pyrrolo[3,2-c]quinolin-4-yl)-N-methylpicolinamide;
3-(1-((1R,4R,5S)-2-azabicyclo[2.1.1]hexan-5-yl)-6-fluoro-7-(7-fluoronaphthalen-1-yl)-4-(6-(2-hydroxypropan-2-yl)pyridin-3-yl)-2-((R)-1-(2-oxopyrazin-1 (2H)-yl)ethyl)-1H-pyrrolo[3,2-c]quinolin-8-yl)propanenitrile;
3-(1-((1R,4R,5S)-2-azabicyclo[2.1.1]hexan-5-yl)-7-(3-chloro-2-methylphenyl)-6-fluoro-4-(5-methylpyrazin-2-yl)-2-((R)-1-(2-oxopyrazin-1(2H)-yl)ethyl)-1H-pyrrolo[3,2-c]quinolin-8-yl)propanenitrile;
methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(5-fluoro-6-(methylcarbamoyl)pyridin-3-yl)-1H-pyrrolo[3,2-c]quinolin-2-yl)pyrrolidine-1-carboxylate;
methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;
ethyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;
3-(1-((1R,4R,5S)-2-azabicyclo[2.1.1]hexan-5-yl)-7-(2,3-dichlorophenyl)-2-((R)-1-(3,3-difluoroazetidine-1-carbonyl)pyrrolidin-2-yl)-6-fluoro-4-(methyl-d3)-1H-pyrrolo[3,2-c]quinolin-8-yl)propanenitrile;
3-(1-((1R,4R,5S)-2-azabicyclo[2.1.1]hexan-5-yl)-7-(2,3-dichlorophenyl)-2-((R)-1-(3,3-difluoroazetidine-1-carbonyl)pyrrolidin-2-yl)-6-fluoro-1H-pyrrolo[3,2-c]quinolin-8-yl)propanenitrile;
3-(1-((1R,4R,5S)-2-azabicyclo[2.1.1]hexan-5-yl)-7-(3-chloro-2-methylphenyl)-6-fluoro-4-(5-methylpyrazin-2-yl)-2-((R)-1-(3-oxomorpholino)ethyl)-1H-pyrrolo[3,2-c]quinolin-8-yl)propanenitrile;
5-(1-((1R,4R,5S)-2-azabicyclo[2.1.1]hexan-5-yl)-8-(2-cyanoethyl)-6-fluoro-7-(7-fluoronaphthalen-1-yl)-2-((R)-1-(3-oxomorpholino)ethyl)-1H-pyrrolo[3,2-c]quinolin-4-yl)-N-methylpicolinamide;
3-(1-((1R,4R,5S)-2-azabicyclo[2.1.1]hexan-5-yl)-6-fluoro-7-(7-fluoronaphthalen-1-yl)-4-(6-(2-hydroxypropan-2-yl)pyridin-3-yl)-2-((R)-1-(3-oxomorpholino)ethyl)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoronaphthalen-1-yl)-4-(5-methylpyrazin-2-yl)-2-((R)-1-(3-oxomorpholino)ethyl)-1H-pyrrolo[3,2-c]quinolin-8-yl)propanenitrile;
methyl (1R,3R,5R)-3-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)-2-azabicyclo[3.1.0]hexane-2-carboxylate;
3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-7-(2,3-dichlorophenyl)-6-fluoro-4-(6-(2-hydroxypropan-2-yl)pyridin-3-yl)-2-((R)-1-(2-oxopyrazin-1(2H)-yl)ethyl)-1H-pyrrolo[3,2-c]quinolin-8-yl)propanenitrile;
methyl (2R,4S)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)-4-fluoropyrrolidine-1-carboxylate;
methyl (2R,5R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)-5-methylpyrrolidine-1-carboxylate;
methyl (2R)-2-(1-((1R,4R,5S)-2-azabicyclo[2.1.1]hexan-5-yl)-3-chloro-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;
4-(1-((1R,4R,5S)-2-azabicyclo[2.1.1]hexan-5-yl)-8-(2-cyanoethyl)-7-(2,3-dichlorophenyl)-6-fluoro-2-((R)-1-(2-oxopyrazin-1(2H)-yl)ethyl)-1H-pyrrolo[3,2-c]quinolin-4-yl)-2-fluoro-N-methylbenzamide;
methyl ((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)carbamate;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-2,2-difluoroacetamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-2,2-difluoroacetamide;
(2S)—N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)tetrahydrofuran-2-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)cyclopropanesulfonamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)thiazole-4-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-N-methylcyclopropanecarboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-methylcyclopropane-1-carboxamide;
3-(1-((1R,4R,5S)-2-azabicyclo[2.1.1]hexan-5-yl)-7-(2,3-dichlorophenyl)-6-fluoro-4-(1-hydroxyethyl)-2-((1R,3R,5R)-2-(1-methylcyclopropane-1-carbonyl)-2-azabicyclo[3.1.0]hexan-3-yl)-1H-pyrrolo[3,2-c]quinolin-8-yl)propanenitrile;
3-(1-((1R,4R,5S)-2-azabicyclo[2.1.1]hexan-5-yl)-7-(2,3-dichlorophenyl)-6-fluoro-2-((1R,3R,5R)-2-(1-fluorocyclopropane-1-carbonyl)-2-azabicyclo[3.1.0]hexan-3-yl)-4-(1-hydroxyethyl)-1H-pyrrolo[3,2-c]quinolin-8-yl)propanenitrile;
3-(1-((1R,4R,5S)-2-azabicyclo[2.1.1]hexan-5-yl)-7-(2,3-dichlorophenyl)-6-fluoro-2-((1R,3R,5R)-2-(1-fluorocyclopropane-1-carbonyl)-2-azabicyclo[3.1.0]hexan-3-yl)-4-methyl-1H-pyrrolo[3,2-c]quinolin-8-yl)propanenitrile;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-fluorocyclopropane-1-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-fluorocyclobutane-1-carboxamide;
3-(1-((1R,4R,5S)-2-azabicyclo[2.1.1]hexan-5-yl)-7-(3-chloro-2-methylphenyl)-2-(1-(2,6-dimethyl-3-oxo-2,3-dihydropyridazin-4-yl)ethyl)-6-fluoro-4-methyl-1H-pyrrolo[3,2-c]quinolin-8-yl)propanenitrile;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)pyrimidine-4-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)pyridazine-3-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-3,3-difluoroazetidine-1-carboxamide;
3-(1-((1R,4R,5S)-2-azabicyclo[2.1.1]hexan-5-yl)-7-(2,3-dichlorophenyl)-6-fluoro-4-methyl-2-((R)-1-((1-methyl-1H-pyrazol-4-yl)amino)ethyl)-1H-pyrrolo[3,2-c]quinolin-8-yl)propanenitrile;
5-(1-((1R,4R,5S)-2-azabicyclo[2.1.1]hexan-5-yl)-8-(2-cyanoethyl)-7-(2,3-dichlorophenyl)-6-fluoro-2-((R)-1-(1-fluorocyclopropane-1-carbonyl)pyrrolidin-2-yl)-1H-pyrrolo[3,2-c]quinolin-4-yl)-N,N-dimethylpicolinamide; and
methyl (2R)-2-(1-((1R,4R,5S)-2-azabicyclo[2.1.1]hexan-5-yl)-8-(2-cyanoethyl)-7-(2,3-dichlorophenyl)-4-(4-((dimethylamino)methyl)-2,3-difluorophenyl)-6-fluoro-1H-pyrrolo[3,2-c]quinolin-2-yl)pyrrolidine-1-carboxylate;
   and pharmaceutically acceptable salts thereof.

In another embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

In yet another embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1*”).

In still another embodiment, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1”):

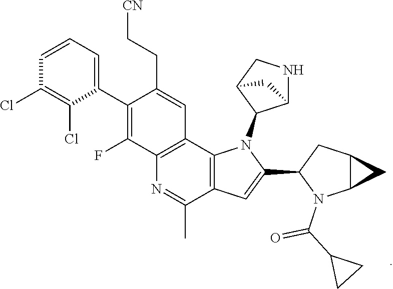

In an embodiment, the KRAS G12D inhibitor is 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1a”)

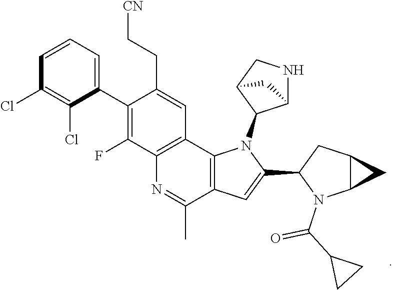

In another aspect, the KRAS G12D inhibitor is a compound of Formula IV

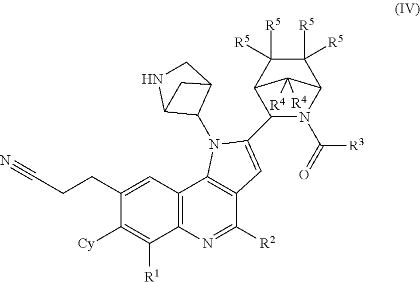

or a pharmaceutically acceptable salt thereof, wherein:
Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
R¹ is halogen;
R² is selected from H, D, C₁₋₃ alkyl, C₂₋₃ alkenyl, C₂₋₃ alkynyl, C₁₋₃ haloalkyl, C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, CN, OR^{a2}, C(O)R^{b2}, C(O)NR^{c2}R^{d2}, NR^{c2}R^{d2}, and NR^{c2}C(O)R^{b2}; wherein the C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene forming R² are each optionally substituted with 1, 2, or 3 substituents independently selected from R^{2A} wherein the ring-forming atoms of the 4-6 membered heterocycloalkyl, 5-6 membered heteroaryl, C₃₋₆ cycloalkyl-C₁₋₃ alkylene, and 4-6 membered heterocycloalkyl-C₁₋₃ alkylene forming R² consist of at least one carbon atom and 1, 2, 3, or 4 heteroatoms selected from O, N, and S; wherein a ring-forming carbon atom of the 4-6 membered heterocycloalkyl, 5-6 membered heteroaryl, C₃₋₆ cycloalkyl-C₁₋₃ alkylene, and 4-6 membered heterocycloalkyl-C₁₋₃ alkylene forming R² is optionally substituted by oxo to form a carbonyl group; and wherein the C₁₋₃ alkyl, C₂₋₃ alkenyl, and C₂₋₃ alkynyl forming R² are each optionally substituted with 1, 2, or 3 substituents independently selected from R^{2B};
each R^{a2} is independently selected from C₁₋₆ alkyl, C₂₋₆ alkenyl, C₂₋₆ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a2} are each optionally substituted with 1, 2, or 3 substituents independently selected from R^{2A}; wherein the ring-forming atoms of the 4-6 membered heterocycloalkyl and 5-6 membered heteroaryl forming R^{a2} consist of at least one carbon atom and 1, 2, 3, or 4 heteroatoms selected from O, N, and S; wherein a ring-forming carbon atom of the 4-6 membered heterocycloalkyl and 5-6 membered heteroaryl forming R^{a2} is optionally substituted by oxo to form a carbonyl group; and wherein the C₁₋₆ alkyl, C₂₋₆ alkenyl, and C₂₋₆ alkynyl forming R^{a2} are each optionally substituted with 1, 2, or 3 substituents independently selected from R^{2B};
each R^{b2}, R^{c2}, and R^{d2} is independently selected from H, C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{b2}, R^{c2}, and R^{d2} are each optionally substituted with 1, 2, or 3 substituents independently selected from R^{2A}; the ring-forming atoms of the 4-6 membered heterocycloalkyl and 5-6 membered heteroaryl forming R^{b2}, R^{c2}, and R^{d2} consist of at least one carbon atom and 1, 2, 3, or 4 heteroatoms selected from O, N, and S; wherein a ring-forming carbon atom of the 4-6 membered heterocycloalkyl and 5-6 membered heteroaryl forming R^{b2}, R^{c2}, and R^{d2} is optionally substituted by oxo to form a carbonyl group; and wherein the C₁₋₃ alkyl, C₂₋₃ alkenyl, and C₂₋₃ alkynyl forming R^{b2}, R^{c2}, and R^{d2} are each optionally substituted with 1, 2, or 3 substituents independently selected from R^{2B}; or
any R^{c2} and R^{d2} attached to the same N atom, together with the N atom to which they are attached, form a 4-, 5-, or 6-membered heterocycloalkyl group optionally substituted with 1, 2, or 3 substituents independently selected from R^{2B};
each R^{e2} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl and 5-6 membered heteroaryl; wherein the C₃₋₆ cycloalkyl, 4-6 membered heterocycloalkyl, phenyl and 5-6 membered heteroaryl forming R^{e2} are each optionally substituted with 1, 2, or 3 substituents independently selected from R^{2A}; wherein the ring-forming atoms of the 4-6 membered heterocycloalkyl and 5-6 membered heteroaryl forming R^{e2} consist of at least one carbon atom and 1, 2, 3, or 4 heteroatoms selected from O, N, and S; wherein a ring-forming carbon atom of the 4-6 membered heterocycloalkyl and 5-6 membered heteroaryl forming R^{e2} is optionally substituted by oxo to form a carbonyl group; and wherein the C₁₋₃ alkyl, C₂₋₃ alkenyl, and C₂₋₃ alkynyl, forming R^{e2} are each optionally substituted with 1, 2, or 3 substituents independently selected from R^{2B}; or
R^{c2} and R^{e2} attached to the same N atom, together with the N atom to which they are attached, form a 4-, 5-, or 6-membered heterocycloalkyl group optionally substituted with 1, 2, or 3 substituents independently selected from R^{2B};
each R^{2A} is independently selected from C₁₋₃ alkyl, C₂₋₃ alkenyl, C₂₋₃ alkynyl, C₁₋₃ haloalkyl, and R^{2B}, wherein the C₁₋₃ alkyl, C₂₋₃ alkenyl, and C₂₋₃ alkynyl, forming R^{2A} are each optionally substituted with 1, 2 or 3 substituents independently selected from R^{2B};
each R^{2B} is independently selected from C₃₋₆ cycloalkyl, 4-10 membered heterocycloalkyl, phenyl, 5-6 membered heteroaryl, halo, D, CN, OR^{a2}B, C(O)R^{b2B}, C(O)NR^{c2B}R^{d2B}, C(O)OR^{a2B}, NR^{c2B}R^{d2B}, and S(O)₂R^{b2B}; wherein the C₁₋₃ alkyl, C₃₋₆cycloalkyl, 4-6 membered heterocycloalkyl, phenyl, and 5-6 membered heteroaryl are each optionally substituted with 1, 2, or 3 substituents independently selected from R^{2C};
each R^{2C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, 5-6 membered heteroaryl, halo, D, CN, OR^{a2C}, C(O)R^{b2C}, C(O)NR^{c2C}R^{d2C}, C(O)OR^{a2C}, NR^{c2C}R^{d2C}, and S(O)₂R^{b2C};
each R^{a2B}, R^{b2B}, R^{c2B} and R^{d2B} is independently selected from H, C₁₋₃ alkyl, and C₁₋₃ haloalkyl;
each R^{a2C}, R^{b2C}, R^{c2C} and R^{d2C} is independently selected from H, C₁₋₃ alkyl, and C₁₋₃ haloalkyl;
R³ is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, OR^{3A}, and NR^{3B}R^{3C}; wherein the C₃₋₁₀ cycloalkyl, 4-10 membered heterocycloalkyl, C₆₋₁₀ aryl, and 5-10 membered heteroaryl C₁₋₃ alkyl forming R³ are each optionally substituted with 1, 2, or 3 substituents independently selected from R^{3D}; wherein the ring-forming atoms of the 4-10 membered heterocycloalkyl and 5-10 membered heteroaryl forming R³ consist of at least one carbon atom and 1, 2, 3, or 4 heteroatoms selected from O, N, and S; wherein a ring-forming carbon atom of the 4-10 membered heterocycloalkyl or 5-10 membered heteroaryl forming R³ is optionally substituted by oxo to form a carbonyl group; and wherein the C₁₋₃ alkyl, C₂₋₃ alkenyl, and C₂₋₃ alkynyl forming R³ are each optionally substituted with 1, 2, or 3 substituents independently selected from R^{3E};
R^{3A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, and 5-10 membered heteroaryl; wherein the C₃₋₁₀ cycloalkyl, 4-10 membered heterocycloalkyl, C₆₋₁₀ aryl, and 5-10 membered heteroaryl C₁₋₃ alkyl forming R^{3A} are each optionally substituted with 1, 2, or 3 substituents independently selected from R^{3D}; wherein the ring-forming atoms of the 4-10 membered heterocycloalkyl and 5-10 membered heteroaryl forming R^{3A} consist of at least one carbon atom and 1, 2, 3, or 4 heteroatoms selected from O, N, and S; wherein a ring-forming carbon atom of the 4-10 membered heterocycloalkyl or 5-10 membered heteroaryl forming R^{3A} is optionally substituted by oxo to form a carbonyl group; and wherein the C₁₋₃ alkyl, C₂₋₃ alkenyl, and C₂₋₃ alkynyl forming R^{3A} are each optionally substituted with 1, 2, or 3 substituents independently selected from R^{3E};
R^{3B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, and 5-10 membered heteroaryl; wherein the C₃₋₁₀ cycloalkyl, 4-10 membered heterocycloalkyl, C₆₋₁₀ aryl, and 5-10 membered heteroaryl forming R^{3B} are each optionally substituted with 1, 2, or 3 substituents independently selected from R^{3D}; wherein the ring-forming atoms of the 4-10 membered heterocycloalkyl and 5-10 membered heteroaryl forming R^{3B} consist of at least one carbon atom and 1, 2, 3, or 4 heteroatoms selected from O, N, and S; wherein a ring-forming carbon atom of the 4-10 membered heterocycloalkyl and 5-10 membered heteroaryl forming R^{3B} is optionally substituted by oxo to form a carbonyl group; and wherein the C₁₋₃ alkyl, C₂₋₃ alkenyl, and C₂₋₃ alkynyl forming R^{3B} are each optionally substituted with 1, 2, or 3 substituents independently selected from R^{3E};
R^{3B} and R^{3C}, together with the N atom to which they are both attached, optionally form a 4-, 5-, 6-, or 7-membered heterocycloalkyl group that is optionally substituted with 1, 2, or 3 substituents independently selected from independently selected from R^{3D}; R^{3C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{3C} are each optionally substituted with 1, 2, or 3 substituents independently selected from R^{3E};
each R^{3D} is independently selected from C₁₋₃ alkyl, C₁₋₃ haloalkyl, C₂₋₃ alkenyl, C₂₋₃ alkynyl, and R^{3E}; wherein each of the C₁₋₃ alkyl, C₂₋₃ alkenyl, and C₂₋₃ alkynyl forming R^{3D} is optionally substituted with 1, 2, or 3 substituents independently selected from R^{3E};
each R^{3E} is independently selected from D, halo, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(=NR^{e3})NR^{c3}R^{d3}, NR^{c3}C(=NR^{e3})NR^{c3}R^{d3}, S(O)R^{b3}, S(O)NR^{c3}R^{d3}, S(O)₂R^{b3}, NR^{c3}S(O)₂R^{b3}, and S(O)₂NR^{c3}R^{d3};
R^{a3}, R^{b3}, R^{c}s, and R^{d3} are each independently selected from H, C₁₋₃ alkyl, C₂₋₃ alkenyl, C₂₋₃ alkynyl, C₆₋₁₀ aryl, C₃₋₇ cycloalkyl, 5-10 membered heteroaryl, 4-10 membered heterocycloalkyl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a3}, R^{b3}, R^{c3}, and R^{d3} are each optionally substituted with 1, 2, 3, 4, or 5 substituents independently selected from C₁₋₃ alkyl, C₁₋₃ haloalkyl, C₂₋₃ alkenyl, C₂₋₃ alkynyl, halo, CN, OR^{a3A}, SR^{a3A}, C(O)R^{b3A}, C(O)NR^{c3A}R^{d3A}, C(O)OR^{a3A}, OC(O)R^{b3A}, OC(O)NR^{c3A}R^{d3A}, NR^{c3A}R^{d3A}, NR^{c3A}C(O)R^{b3A}, NR^{c3A}C(O)NR^{c3A}R^{d3A}, NR^{c3A}C(O)OR^{a3A}, C(═NR^{e3A})NR^{c3A}R^{d3A}, NR^{c3A}C(═NR^{e3A})NR^{c3A}R^{d3A}, S(O)R^{b3A}, S(O)NR^{c3A}R^{d3A}, S(O)₂R^{b3A}, NR^{c3A}S(O)₂R^{b3A}, and S(O)₂NR^{c3A}R^{d3A}; wherein the ring-forming atoms of the 4-10 membered heterocycloalkyl, 5-10 membered heteroaryl, 5-10 membered heteroaryl-C₁₋₃ alkyl, and 4-10 membered heterocycloalkyl-C₁₋₃ alkyl forming R^{a3}, R^{b3}, R^{c3}, and R^{d3} consist of at least one carbon atom and 1, 2, 3, or 4 heteroatoms selected from O, N, and S; wherein a ring-forming carbon atom of the 4-10 membered heterocycloalkyl, 5-10 membered heteroaryl, 5-10 membered heteroaryl-C₁₋₃ alkyl, and 4-10 membered heterocycloalkyl-C₁₋₃ alkyl forming R^{a3}, R^{b3}, R^{c3}, and R^{d3} is optionally substituted by oxo to form a carbonyl group; or R^{c3} and R^{d3} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{a3A}, SR^{a3A}, C(O)R^{b3A}, C(O)NR^{c3A}R^{d3A}, C(O)OR^{a3A}, OC(O)R^{b3A}, OC(O)NR^{c3A}R^{d3A}, NR^{c3A}R^{d3A}, NR^{c3A}C(O)R^{b3A}, NR^{c3A}C(O)NR^{c3A}R^{d3A}, NR^{c3A}C(O)OR^{a3A}, C(═NR^{e3A})NR^{c3A}R^{d3A}, NR^{c3A}C(═NR^{e3A})NR^{c3A}R^{d3A}, S(O)R^{b3A}, S(O)NR^{c3A}R^{d3A}, S(O)₂R^{b3A}, NR^{c3A}S(O)₂R^{b3A}, and S(O)₂NR^{c3A}R^{d3A};
R^{a3A}, R^{b3A}, R^{c3A}, and R^{d3A} are each independently selected from H, C₁₋₃ alkyl, C₁₋₃ haloalkyl, C₂₋₃ alkenyl, C₂₋₃ alkynyl, aryl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₁₋₆ alkyl, C₁₋₆ haloalkyl, C₂₋₆ alkenyl, C₂₋₆ alkynyl C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a3A}, R^{b3A}, R^{c3A}, and R^{d3A} are each optionally substituted with 1, 2, or 3 substituents independently selected from OH, CN, amino, NH(C₁₋₆ alkyl), N(C₁₋₆ alkyl)₂, halo, C₁₋₆alkyl, C₁₋₆ alkoxy, C₁₋₆ haloalkyl, and C₁₋₆ haloalkoxy; wherein the ring-forming atoms of the 4-10 membered heterocycloalkyl, 5-10 membered heteroaryl, 5-10 membered heteroaryl-C₁₋₃ alkyl, and 4-10 membered heterocycloalkyl-C₁₋₃ alkyl forming R^{a3A}, R^{b3A}, R^{c3A}, and R^{d3A} consist of at least one carbon atom, and 1, 2, 3, or 4 heteroatoms selected from O, N, and S; and wherein a ring-forming carbon atom of the 4-10 membered heterocycloalkyl, 5-10 membered heteroaryl, 5-10 membered heteroaryl-C₁₋₃ alkyl, and 4-10 membered heterocycloalkyl-C₁₋₃ alkyl forming R^{a3A}, R^{b3A}, R^{c3A}, and R^{d3A} is optionally substituted by oxo to form a carbonyl group; or
R^{c3A} and R^{d3A} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₆ alkyl, C₁₋₆ alkoxy, C₁₋₆ haloalkyl, and C₁₋₆ haloalkoxy;R^{e3}, and R^{e3A} are each, independently, H, CN or NO₂;
each R⁴ is independently selected from H, D, C₁₋₃ alkyl, C₁₋₃ haloalkyl, C₂₋₃ alkenyl, C₂₋₃ alkynyl, halo, and OR^{a4};
each R^{a4} is independently selected from H, C₁₋₃ alkyl, and C₁₋₃ haloalkyl;
one R⁵ is R^{5A}; and each other R⁵ is independently selected from H, D, halo, C₁₋₃ alkyl, OR^{a5}, C₁₋₃ haloalkyl, C₂₋₃ alkenyl, and C₂₋₃ alkynyl; or, optionally, two other R⁵ attached to the same carbon atom, together with the carbon atom to which they are both attached, form a spiro C₃₋₆ cycloalkyl ring that is optionally substituted with 1, 2, 3, or 4 substituents each selected from D, C₁₋₃ alkyl, and halo; or, optionally, two other R⁵ attached to adjacent carbon atoms, together with the carbon atoms to which they are each attached, form a fused C₃₋₆ cycloalkyl ring that is optionally substituted with 1, 2, 3, or 4 substituents each selected from D, C₁₋₃ alkyl, and halo;
R^{5A} is H, D, C₁₋₃ alkyl, C₁₋₃ haloalkyl, C₂₋₃ alkenyl, C₂₋₃ alkynyl, halo, OR^{a5A}, CN, or Cy²; wherein the C₁₋₃ alkyl forming R^{5A} is optionally substituted with 1, 2, 3 or 4 substituents each selected from R^{5B} and also optionally substituted with Cy², or, optionally, R^{5A} and R⁵ attached to the same carbon atom, together with the carbon atom to which they are both attached, form a spiro C₃₋₆ cycloalkyl ring that is optionally substituted with 1, 2, 3, or 4 substituents each selected from D, C₁₋₃ alkyl, and halo; or, optionally, R^{5A} and R⁵ attached to adjacent carbon atoms, together with the carbon atoms to which they are each attached, form a fused C₃₋₆ cycloalkyl ring that is optionally substituted with 1, 2, 3, or 4 substituents each selected from D, C₁₋₃ alkyl, and halo;
each R^{5B} is independently selected from 0 and halo;
each R^{a5} is independently selected from H, C₁₋₃ alkyl, and C₁₋₃ haloalkyl; R^{a5A} is selected from H, C₁₋₃ alkyl, C₁₋₃ haloalkyl, and Cy², wherein the C₁₋₃ alkyl forming R^{a5A} is optionally substituted with 1, 2, 3 or 4 substituents each selected from R^{5B} and also optionally substituted with Cy²;
Cy² is selected from C₃₋₇ cycloalkyl, 4-10 membered heterocycloalkyl, C₆₋₁₀ aryl, and 5-10 membered heteroaryl; wherein the C₃₋₇ cycloalkyl, 4-10 membered heterocycloalkyl, C₆₋₁₀ aryl, 5-10 membered heteroaryl, C₆₋₁₀ aryl, and 5-10 membered heteroaryl forming Cy² is optionally substituted with 1, 2, 3, or 4 substituents independently selected from R^{Cy2}; wherein the ring-forming atoms of the 4-10 membered heterocycloalkyl and 5-10 membered heteroaryl forming Cy² consist of at least one carbon atom and 1, 2, 3, or 4 heteroatoms independently selected from N, O, and S; and wherein a ring-forming carbon atom of the 4-10 membered heterocycloalkyl and 5-10 membered heteroaryl forming Cy² is optionally substituted by oxo to form a carbonyl group;
each R^{Cy2} is independently selected from D, C₁₋₃ alkyl, C₁₋₃ haloalkyl, C₂₋₃ alkenyl, C₂₋₃ alkynyl, C₃₋₆ cycloalkyl, 4-10 membered heterocycloalkyl, C₆₋₁₀ aryl, 5-10 membered heteroaryl, halo, CN, OR^{aCy21}, SR^{aCy21}, C(O)R^{bCy21}, C(O)NR^{cCy21}R^{dCy21}, C(O)OR^{aCy21}, OC(O)R^{bCy21}, OC(O)NR^{cCy21}R^{dCy21}, NR^{cCy21}R^{dCy21}, NR^{cCy21}C(O)R^{bCy21}, NR^{cCy21}C(O)NR^{cCy21}R^{dCy21}, NR^{cCy21}C(O)OR^{aCy21}, C(═NR^{cCy21})NR^{cCy21}R^{dCy21}, NR^{cCy21}C(═NR^{cCy21})NR^{cCy21}R^{dCy21}, S(O)R^{bCy21}, S(O)NR^{cCy21}R^{dCy21}, S(O)₂R^{bCy21}, NR^{cCy21}S(O)₂R^{bCy21}, and S(O)₂NR^{CCy21}R^{dCy21}; wherein the C₃₋₆ cycloalkyl, 4-10 membered heterocycloalkyl, C₆₋₁₀ aryl, and 5-10 membered heteroaryl forming R^{Cy2} are each optionally substituted by 1, 2, 3, or 4 substituents independently selected from R^{Cy2A}; wherein the ring-forming atoms of the 4-10 membered heterocycloalkyl and 5-10 membered heteroaryl forming R^{Cy2} consist of at least one carbon atom and 1, 2, 3, or 4 heteroatoms independently selected from N, O, and S; wherein a ring-forming carbon atom of the 4-10 membered heterocycloalkyl and 5-10 membered heteroaryl forming R^{Cy2} is optionally substituted by oxo to form a carbonyl group; and wherein the C₁₋₃ alkyl, C₂₋₃ alkenyl, and C₂₋₃ alkynyl forming R^{Cy2} are each optionally substituted by 1, 2, or 3 substituents independently selected from R^{Cy2B};
each R^{Cy2A} is independently selected from C₁₋₃ alkyl, C₁₋₃ haloalkyl, C₂₋₃ alkenyl, C₂₋₃ alkynyl, and R^{Cy2B}; wherein the C₁₋₃ alkyl, C₂₋₃ alkenyl, and C₂₋₃ alkynyl forming R^{Cy2A} are each optionally substituted by 1, 2, or 3 substituents independently selected from R^{Cy2B},
each R^{Cy2B} is independently selected from D, halo, CN, OR^{aCy21}, SR^{aCy21}, C(O)R^{bCy21}, C(O)NR^{cCy21}R^{dCy21}, C(O)OR^{aCy21}, OC(O)R^{bCy21}, OC(O)NR^{cCy21}R^{dCy21}, NR^{cCy21}R^{dCy21}, NR^{cCy21}C(O)R^{bCy21}, NR^{cCy21}C(O)NR^{cCy21}R^{dCy21}, NR^{cCy21}C(O)OR^{aCy21}, C(═NR^{cCy21})NR^{cCy21}R^{dCy21}, NR^{cCy21}C(═NR^{cCy21})NR^{cCy21}R^{dCy21}, S(O)R^{bCy21}, S(O)NR^{cCy21}R^{dCy21}, S(O)₂R^{bCy21}, NR^{cCy21}S(O)₂R^{bCy21}, and S(O)₂NR^{cCy21}R^{dCy21}
R^{aCy21}, R^{bCy21}, R^{cCy21}, and R^{dCy21} are each independently selected from H, C₁₋₃ alkyl, C₁₋₃ haloalkyl, C₂₋₃ alkenyl, C₂₋₃ alkynyl, C₆₋₁₀ aryl, C₃₋₇cycloalkyl, 5-10 membered heteroaryl, 4-10 membered heterocycloalkyl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{aCy21}, R^{bCy21}, R^{cCy21}, and R^{dCy21} are each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{aCy22}, SR^{aCy22}, C(O)R^{bCy22}, C(O)NR^{cCy22}R^{aCy22}, C(O)OR^{aCy22}, OC(O)R^{bCy22}, OC(O)NR^{cCy22}R^{aCy22}, NR^{cCy22}R^{aCy22}, NR^{cCy22}C(O)R^{bCy22}, NR^{cCy22}C(O)NR^{cCy22}R^{aCy22}, NR^{cCy22}C(O)OR^{aCy22}, C(═NR^{cCy22})NR^{cCy22}R^{aCy22}, NR^{cCy22}C(═NR^{cCy22})NR^{cCy22}R^{aCy22}, S(O)R^{bCy22}, S(O)NR^{cCy22}R^{aCy22}, S(O)₂R^{bCy22}, NR^{cCy22}S(O)₂R^{bCy22}, and S(O)₂NR^{cCy22}R^{aCy22}; wherein the ring-forming atoms each of the 5-10 membered heteroaryl, 4-10 membered heterocycloalkyl, 5-10 membered heteroaryl-C₁₋₃ alkyl, and 4-10 membered heterocycloalkyl-C₁₋₃ alkyl forming R^{aCy21}, R^{bCy21}, R^{cCy21}, and R^{dCy21} consist of at least one carbon atom and 1, 2, 3, or 4 heteroatoms independently selected from N, O, and S; and wherein a ring-forming carbon atom of the 5-10 membered heteroaryl, 4-10 membered heterocycloalkyl, 5-10 membered heteroaryl-C₁₋₃ alkyl, and 4-10 membered heterocycloalkyl-C₁₋₃alkyl forming R^{aCy21}, R^{bCy21}, R^{cCy21}, and R^{dCy21} is optionally substituted by oxo to form a carbonyl group;
or R^{cCy21} and R^{dCy21} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{aCy22}, SR^{aCy22}, C(O)R^{bCy22}, C(O)NR^{cCy22}R^{dCy22}, C(O)OR^{aCy22}, OC(O)R^{bCy22}, OC(O)NR^{cCy22}R^{dCy22}, NR^{cCy22}R^{aCy22}, NR^{cCy22}C(O)R^{bCy22}, NR^{cCy22}C(O)NR^{cCy22}R^{dCy22}, NR^{cCy22}C(O)OR^{aCy22}, C(═NR^{cCy22})NR^{cCy22}R^{aCy22}, NR^{cCy22}C(═NR^{cCy22})NR^{cCy22}R^{aCy22}, S(O)R^{bCy22}, S(O)NR^{cCy22}R^{aCy22}, S(O)₂R^{bCy22}, NR^{cCy22}S(O)₂R^{bCy22}, and S(O)₂NR^{cCy22}R^{aCy22}; R^{aCy22}, R^{bCy22}, R^{cCy22}, and R^{dCy22} are each independently selected from H, C₁₋₃ alkyl, C₁₋₃ haloalkyl, C₂₋₃ alkenyl, C₂₋₃ alkynyl, aryl, C₆₋₁₀ aryl-C₁₋₃alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₁₋₃ alkyl, C₂₋₃ alkenyl, C₂₋₃ alkynyl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{aCy22}, R^{bCy22}, R^{cCy22}, and R^{dCy22} are each optionally substituted with 1, 2, or 3 substituents independently selected from OH, CN, amino, NH(C₁₋₃ alkyl), N(C₁₋₃ alkyl)₂, halo, C₁₋₃alkyl, C₁₋₃ alkoxy, C₁₋₃ haloalkyl, and C₁₋₃ haloalkoxy; wherein the ring-forming atoms each of the 5-membered heteroaryl, 4-10 membered heterocycloalkyl, 5-10 membered heteroaryl-C₁₋₃ alkyl, and 4-10 membered heterocycloalkyl-C₁₋₃ alkyl forming R^{aCy22}, R^{bCy22}, R^{cCy22}, and R^{dCy22} consist of at least one carbon atom and 1, 2, 3, or 4 heteroatoms independently selected from N, O, and S; and wherein a ring-forming carbon atom of the 5-10 membered heteroaryl, 4-10 membered heterocycloalkyl, 5-10 membered heteroaryl-C₁₋₃ alkyl, and 4-10 membered heterocycloalkyl-C₁₋₃alkyl forming R^{aCy22}, R^{bCy22}, R^{cCy22}, and R^{dCy22} is optionally substituted by oxo to form a carbonyl group; or
R^{cCy22} and R^{dCy22} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₃ alkyl, C₁₋₃ alkoxy, C₁₋₃ haloalkyl, and C₁₋₃ haloalkoxy; and
R^{eCy21} and R^{eCy22} are each, independently, H, CN or NO₂.

In an embodiment of Formula IV,
Cy¹ is phenyl optionally substituted with 1 or 2 substituents each selected from D, C₁₋₃ alkyl, C₁₋₃ haloalkyl, halo, OH, and C₁₋₃ alkoxy;
R¹ is halo;
R² is C₁₋₃ alkyl optionally substituted with OH;
R³ is C₃₋₁₀ cycloalkyl optionally substituted with halo;
each R⁴ is H;
one R⁵ is R^{5A}; and each other R⁵ is independently selected from H, D, halo, C₁₋₃ alkyl, OC₁₋₃ alkyl, C₁₋₃ haloalkyl; or, optionally, two other R⁵ attached to adjacent carbon atoms, together with the carbon atoms to which they are each attached, form a fused C₃₋₆ cycloalkyl ring that is optionally substituted with 1 or 2 substituents each selected from D, C₁₋₃ alkyl, and halo; and
R^{5A} is H, halo, or OR^{a5A};
R^{a5A} is selected from C₁₋₃ alkyl, C₁₋₃ haloalkyl, and Cy², wherein the C₁₋₃ alkyl forming
R^{a5A} is optionally substituted with 1, 2, or 3 D, and also optionally substituted with Cy²; and
Cy² is selected from C₆₋₁₀ aryl and 5-10 membered heteroaryl.

In another embodiment of Formula IV,
Cy¹ is phenyl optionally substituted with 1 or 2 substituents each selected from D, C₁₋₃ alkyl, C₁₋₃ haloalkyl, halo, OH, and C₁₋₃ alkoxy;
R¹ is halo;
R² is C₁₋₃ alkyl optionally substituted with OH;
R³ is OR^{3A} or C₃₋₁₀ cycloalkyl optionally substituted with halo;
R^{3A} is C₁₋₃ alkyl;
each R⁴ is H;
one R⁵ is R^{5A}; and each other R⁵ is independently selected from H, D, halo, C₁₋₃ alkyl, OC₁₋₃ alkyl, C₁₋₃ haloalkyl; or, optionally, two other R⁵ attached to adjacent carbon atoms, together with the carbon atoms to which they are each attached, form a fused C₃₋₆ cycloalkyl ring that is optionally substituted with 1 or 2 substituents each selected from D, C₁₋₃ alkyl, and halo;
R^{5A} is H, halo, or OR^{a5A};
R^{a5A} is selected from C₁₋₃ alkyl, C₁₋₃ haloalkyl, and Cy², wherein the C₁₋₃ alkyl forming R^{a5A} is optionally substituted with 1, 2, or 3 D, and also optionally substituted with Cy²; and
Cy² is selected from C₆₋₁₀ aryl and 5-10 membered heteroaryl.

In another embodiment of Formula IV,
Cy¹ is phenyl optionally substituted with 1 or 2 substituents each selected from C₁₋₃ alkyl, C₁₋₃ haloalkyl, halo, OH, and C₁₋₃ alkoxy;
R¹ is halo;
R² is C₁₋₃ alkyl optionally substituted with OH;
R³ is OR^{3A} or C₃₋₁₀ cycloalkyl optionally substituted with halo;
R^{3A} is C₁₋₃ alkyl;
each R⁴ is H;
one R⁵ is R^{5A}; and each other R⁵ is independently selected from H, halo, C₁₋₃ alkyl, OC₁₋₃ alkyl, C₁₋₃ haloalkyl;
R^{5A} is H, halo, or OR^{a5A}; and
R^{a5A} is selected from C₁₋₃ alkyl and C₁₋₃ haloalkyl, wherein the C₁₋₃ alkyl forming R^{a5A} is optionally substituted with 1, 2, or 3 D.

In an embodiment, the compound of Formula IV is a compound of Formula IV-A or Formula IV-B

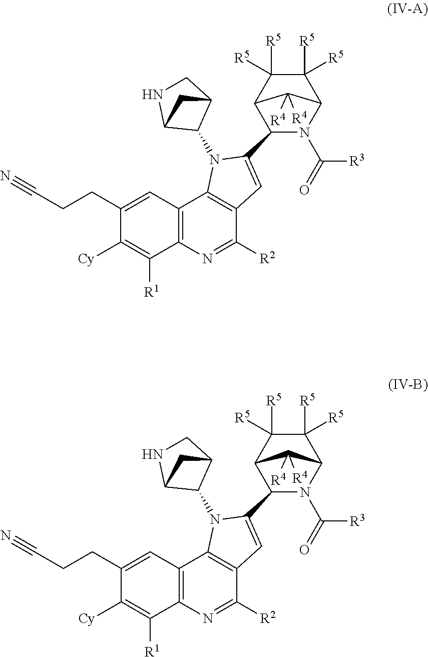

or a pharmaceutically acceptable salt thereof.

In another embodiment, the compound of Formula IV is selected from:
3-(1-(2-azabicyclo[2.1.1]hexan-5-yl)-2-(2-(cyclopropanecarbonyl)-5-methoxy-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-fluoro-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-6-azatricyclo[3.2.1.02,4]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-(methoxy-d3)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-(pyridin-3-yloxy)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(2-(5-(benzyloxy)-2-(cyclopropanecarbonyl)-2-azabicyclo[2.2.1]heptan-3-yl)-1-(2-azabicyclo[2.1.1]hexan-5-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-(difluoromethoxy)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-7-(2,3-dichlorophenyl)-6-fluoro-2-(5-fluoro-2-(1-fluorocyclopropane-1-carbonyl)-2-azabicyclo[2.2.1]heptan-3-yl)-4-((R)-1-hydroxyethyl)-1H-pyrrolo[3,2-c]quinolin-8-yl)propanenitrile;
3-(1-(2-azabicyclo[2.1.1]hexan-5-yl)-2-(2-(cyclopropanecarbonyl)-5-(difluoromethyl)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
5-(1-(2-azabicyclo[2.1.1]hexan-5-yl)-8-(2-cyanoethyl)-2-(2-(cyclopropanecarbonyl)-2-azabicyclo[2.2.1]heptan-3-yl)-7-(2,3-dichlorophenyl)-6-fluoro-1H-pyrrolo[3,2-c]quinolin-4-yl)-N,N-dimethylpicolinamide;
3-(1-(2-azabicyclo[2.1.1]hexan-5-yl)-2-(2-(cyclopropanecarbonyl)-2-azabicyclo[2.2.1]heptan-3-yl)-7-(2,3-dichlorophenyl)-6-fluoro-4-(6-(2-hydroxypropan-2-yl)pyridin-3-yl)-1H-pyrrolo[3,2-c]quinolin-8-yl)propanenitrile;
4-(1-(2-azabicyclo[2.1.1]hexan-5-yl)-8-(2-cyanoethyl)-2-(2-(cyclopropanecarbonyl)-2-azabicyclo[2.2.1]heptan-3-yl)-7-(2,3-dichlorophenyl)-6-fluoro-1H-pyrrolo[3,2-c]quinolin-4-yl)-2-fluoro-N-methylbenzamide;
3-(1-(2-azabicyclo[2.1.1]hexan-5-yl)-2-(2-(cyclopropanecarbonyl)-5-methyl-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-hydroxy-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-(pyridin-2-yloxy)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-(pyridin-4-yloxy)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-7-(2,3-dichlorophenyl)-2-(5-(difluoromethoxy)-2-(1-fluorocyclopropane-1-carbonyl)-2-azabicyclo[2.2.1]heptan-3-yl)-6-fluoro-4-methyl-1H-pyrrolo[3,2-c]quinolin-8-yl)propanenitrile;
3-(1-(2-azabicyclo[2.1.1]hexan-5-yl)-7-(2,3-dichlorophenyl)-6-fluoro-2-(5-fluoro-2-(1-fluorocyclopropane-1-carbonyl)-2-azabicyclo[2.2.1]heptan-3-yl)-4-(1-hydroxyethyl)-1H-pyrrolo[3,2-c]quinolin-8-yl)propanenitrile;
3-(1-(2-azabicyclo[2.1.1]hexan-5-yl)-2-(5-chloro-2-(1-fluorocyclopropane-1-carbonyl)-2-azabicyclo[2.2.1]heptan-3-yl)-7-(2,3-dichlorophenyl)-6-fluoro-4-(1-hydroxyethyl)-1H-pyrrolo[3,2-c]quinolin-8-yl)propanenitrile;
3-(1-(2-azabicyclo[2.1.1]hexan-5-yl)-2-(2-(cyclopropanecarbonyl)-5-(trifluoromethoxy)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-7-(2,3-dichlorophenyl)-2-(5-(difluoromethoxy)-2-(1-fluorocyclopropane-1-carbonyl)-2-azabicyclo[2.2.1]heptan-3-yl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-cyclopropoxy-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-7-(2,3-dichlorophenyl)-2-(5-(difluoromethoxy)-2-(1-fluorocyclopropane-1-carbonyl)-2-azabicyclo[2.2.1]heptan-3-yl)-6-fluoro-4-(1-hydroxyethyl)-1H-pyrrolo[3,2-c]quinolin-8-yl)propanenitrile;
3-(1-(2-azabicyclo[2.1.1]hexan-5-yl)-2-(5-cyclopropoxy-2-(1-fluorocyclopropane-1-carbonyl)-2-azabicyclo[2.2.1]heptan-3-yl)-7-(2,3-dichlorophenyl)-6-fluoro-4-(1-hydroxyethyl)-1H-pyrrolo[3,2-c]quinolin-8-yl)propanenitrile;
methyl 3-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(trifluoromethoxy)-2-azabicyclo[2.2.1]heptane-2-carboxylate;
3-(1-(2-azabicyclo[2.1.1]hexan-5-yl)-7-(2,3-dichlorophenyl)-6-fluoro-2-(2-(1-fluorocyclopropane-1-carbonyl)-5-(trifluoromethoxy)-2-azabicyclo[2.2.1]heptan-3-yl)-4-methyl-1H-pyrrolo[3,2-c]quinolin-8-yl)propanenitrile;
methyl 3-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethyl)-2-azabicyclo[2.2.1]heptane-2-carboxylate; and
3-(1-(2-azabicyclo[2.1.1]hexan-5-yl)-7-(2,3-dichlorophenyl)-2-(5-(difluoromethyl)-2-(1-fluorocyclopropane-1-carbonyl)-2-azabicyclo[2.2.1]heptan-3-yl)-6-fluoro-4-methyl-1H-pyrrolo[3,2-c]quinolin-8-yl)propanenitrile;
   and pharmaceutically acceptable salts thereof.

In another embodiment, the KRAS G12D inhibitor is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 3”), or a pharmaceutically acceptable salt thereof.

In still another embodiment, the KRAS G12D inhibitor is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate

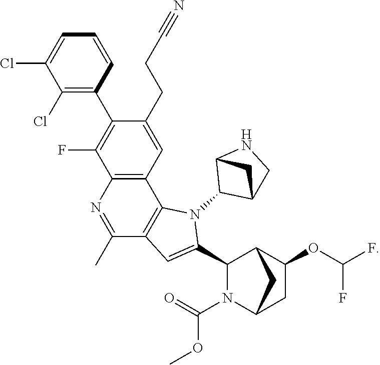

In an embodiment, the KRAS G12D inhibitor is methyl (1R,3R,4R,5S)-3-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate

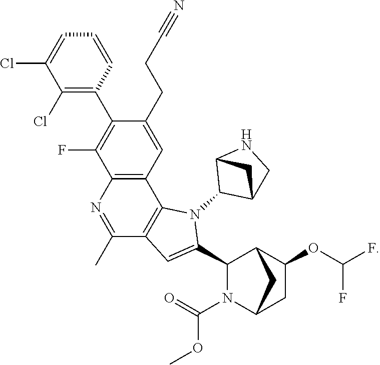

In another embodiment, the KRAS G12D inhibitor is selected from MRTX1133, RMC-9805, HRS-4642, ASP-3082, BI-2852, MRTX-EX185, 3144, QTX3046, VRTX153, JAB-22000, TH-Z827, TH-Z801, TH-Z814, TH-Z816, TH-Z835, TH-Z827, TH-Z837, KD-8, NS-1, and CAS No.: 2765254-39-3.

In some embodiments, the inhibitor of KRAS G12D inhibitor is selected from a compound as disclosed in WO2018/145020, WO2022/015375, WO2021/091967, WO2022/060836, US2023/0293464A1, US2023/0219951A1, or US2023/0285498A1, the contents of which are incorporated by reference in their entirety.

In some embodiments, the inhibitor of KRAS G12D inhibitor is selected from a compound as disclosed in WO2016161361; WO2020212895; WO2021041671; WO2021081212; WO2021106231; WO2021107160; WO2021126799; WO2021215544; WO2021248079; WO2021248082 WO2021248095; WO2022002102; WO2022015375; WO2022031678; WO2022042630; WO2022066646; WO2022098625; WO2022105855; WO2022105857; WO2022105859; WO2022173033; WO2022177917; WO2022184178; WO2022188729; WO2022192794; WO2022194066; WO2022194191; WO2022194192; WO2022198905; WO2022199170; WO2022199586; WO2022206723; WO2022206724; WO2022212947; WO2022214102; WO2022217042; WO2022221739; WO2022223020; WO2022227987; WO2022228543; WO2022232331; WO2022232332; WO2022234639; WO2022234851; WO2022240971; WO2022261154; WO2022262686; WO2022262838; WO2022266069; WO2022268051; WO2023001123; WO2023001141; WO2023018810; WO2023018812; WO2023020347; WO2023025116; WO2023030495; WO2023051586; WO2023056951; WO2023059594; WO2023059596; WO2023059597; WO2023059598; WO2023059600; WO2023061294; WO2023061463; WO2023072188; WO2023085657; WO2023098425; WO2023098426; WO2023098832; WO2023101928; WO2023103523; WO2023103906; WO2023104018; WO2023113739; WO2023122662; WO2023125627; WO2023125989; WO2023133183; WO2023134465; WO2023143312; WO2023159086; WO2023159087; WO2023179629; WO2023179703; WO2023274324; WO2023274383; WO2023278600; WO2023280026; WO2023280280; WO2023283933; WO2023284537; WO2023284881; U.S. Ser. No. 11/453,683; US20180086752; US20180201610; US20220323614; US20220402971; US20230077225; US20230083431; US20230174518; US20230242544; and US20230279025, the contents of which are incorporated by reference in their entirety.

In yet another embodiment, the KRAS G12D inhibitor is a proteolysis targeting chimera (PROTAC). PROTACs are heterobifunctional compounds comprised of a ligand for a target protein (e.g., KRAS with a G12D mutation) and a ligand for an E3 ligase joined by a linker.

In some embodiments, the inhibitor of KRAS G12D proteolysis targeting chimera (PROTAC) is selected from compounds as disclosed in WO2022148421; WO2022148422; WO2022173032; WO2023077441; WO2023081476; WO2023119677; WO2023120742; WO2023138524; and WO2023171781; the contents of which are incorporated by reference in their entirety.

In one embodiment, the disclosed compounds may exist as tautomers. All tautomers are included within the scope of the compounds presented herein.

Compounds provided herein can exist in the form of atropisomers (i.e., conformational diastereoisomers) that can be stable at ambient temperature and separable, e.g., by chromatography. For example, the compounds of Formula I can exist in the form of atropisomers that are interchangeable by rotation around the bond connecting Cy¹ (or any of the embodiments thereof) to the remainder of the molecule. Reference to the compounds described herein or any of the embodiments is understood to include all such atropisomeric forms of the compounds. Without being limited by any theory, it is understood that, for a given compound, one atropisomer may be more potent as an inhibitor of KRAS (including G12D mutated form of KRAS) than another atropisomer. For example, compounds of formula I as described herein in which Cy¹ is 2,3-dichlorophenyl can exist in the form of atropisomers in which the conformation of the dichlorophenyl relative to the remainder of the molecule is as shown by the partial formulae Formula IV-A or Formula IV-B below. The asymmetry of atropisomers is assigned as either Rₐ or Sₐ, as determined by conventional methods of characterizing points of asymmetry. Without being limited by any theory, it is understood that, for a given compound, the atropisomer represented by Formula IV-A is generally more potent as an inhibitor of KRAS (including G12D mutated forms of KRAS) than the atropisomer represented by Formula IV-B.

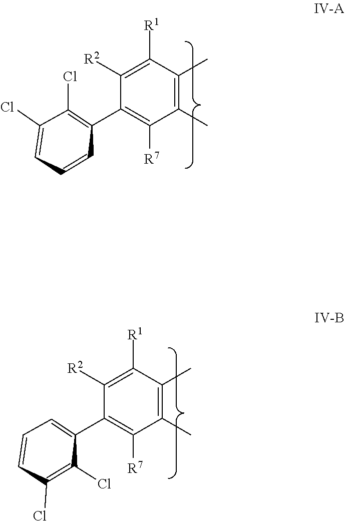

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In another embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements). In yet another embodiment, the compounds described herein include a ²H (i.e., deuterium) isotope.

In still another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

The specific compounds described herein, and other compounds encompassed by one or more of the formulas described herein having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Carey and Sundberg, Advanced Organic Chemistry 4th Ed., Vols. A and B (Plenum 2000, 2001), Advances in Heterocyclic Chemistry, Vols. 1-114 (Elsevier, 1963-2023); Journal of Heterocyclic Chemistry Vols. 1-60 (Journal of Heterocyclic Chemistry, 1964-2023); E. M. Carreira, et al. (Eds.) Science of Synthesis, Vols. 1-48 (2001-2010) and Knowledge Updates KU2010/1-4; 2011/1-4; 2012/1-4, 2013/1-4; 2014/1-4, 2015/1-2; 2016/1-3, 2017/1-3; 2018/1-4, 2019/1-3; 2020/1-3, 2021/1-3, 2022/1-3, 2023/1 (Thieme, 2001-2023); Houben-Weyl, Methoden der Organischen Chemie, 4^{th} Ed. Vols. 1-67 (Thieme, 1952-1987); Houben-Weyl, Methoden der Organischen Chemie, E-Series. Vols. 1-23 (Thieme, 1982-2003); A. R. Katritzky, et al. (Eds.), Comprehensive Organic Functional Group Transformations, Vols. 1-6 (Pergamon Press, 1995); A. R. Katritzky et al. (Eds.), Comprehensive Organic Functional Group Transformations II, Vols. 1-6 (Elsevier, 2^{nd} Edition, 2005); A. R. Katritzky et al. (Eds.); Comprehensive Heterocyclic Chemistry, Vols. 1-8 (Pergamon Press, 1984); A. R. Katritzky, et al. (Eds.); Comprehensive Heterocyclic Chemistry II, Vols. 1-10 (Pergamon Press, 1996); A. R. Katritzky, et al. (Eds.); Comprehensive Heterocyclic Chemistry III, Vols. 1-14 (Elsevier Science, 2008); D. St. C. Black, et al. (Eds.); Comprehensive Heterocyclic Chemistry IV, Vols. 1-14 (Elsevier Science, 2022); M. B. Smith et al., March's Advanced Organic Chemistry: Reactions, Mechanisms, and Structure, 6^{th} Ed. (Wiley, 2007); M. B. Smith et al., March's Advanced Organic Chemistry: Reactions, Mechanisms, and Structure, 8^{th} Ed. (Wiley, 2020); B. M. Trost et al. (Ed.), Comprehensive Organic Synthesis, Vols. 1-9 (Pergamon Press, 1991); and Patai's Chemistry of Functional Groups, 100 Vols. (Wiley 1964-2022) (all of which are incorporated by reference for such disclosure). General methods for the preparation of compounds as described herein are modified by the use of appropriate reagents and conditions, for the introduction of the various moieties found in the Formulas as provided herein.

Compounds described herein are synthesized using any suitable procedures starting from compounds that are available from commercial sources, or are prepared using procedures described herein.

### PD-1 Inhibitors and PD-L1 Inhibitors

The combination therapy provided herein can comprise a KRAS G12D inhibitor and any one of a number of PD-1 inhibitors or PD-L1 inhibitors.

The amino acid sequence of the human PD-1 protein (Genbank Accession No. NP_005009) is: MQIPQAPWPVVWAVLQLGWRPGWFLDSPDRPWNPPTFSPALLVVTEGDNATFTCSFSNT SESFVLNWYRMSPSNQTDKLAAFPEDRSQPGQDCRFRVTQLPNGRDFHMSVVRARRNDS GTYLCGAISLAPKAQIKESLRAELRVTERRAEVPTAHPSPSPRPAGQFQTLVVGVVGGLLGS LVLLVWVLAVICSRAARGTIGARRTGQPLKEDPSAVPVFSVDYGELDFQWREKTPEPPVPC VPEQTEYATIVFPSGMGTSSPARRGSADGPRSAQPLRPEDGHCSWPL (SEQ ID NO:1).

PD-1 has two ligands, PD-L1 and PD-L2 (R. V. Parry et al., Mol. Cell Biol., 2005, 25(21), 9543-53), and they differ in their expression patterns. PD-L1 protein is upregulated on macrophages and dendritic cells in response to lipopolysaccharide and GM-CSF treatment, and on T cells and B cells upon T cell receptor and B cell receptor signaling. PD-L1 is also highly expressed on almost all tumor cells, and the expression is further increased after IFN-γ treatment (R. V. Blank, et al., Cancer Res., 2004, 64(3):1140-45). In fact, tumor PD-L1 expression status has been shown to be prognostic in multiple tumor types (R. Sabatier et al., Oncotarget, 2015, 6(7): 5449-64). PD-L2 expression, in contrast, is more restricted and is expressed mainly by dendritic cells (Y. Komiyama, et al., J. Immunol., 2006, 177(1), 566-73). Ligation of PD-1 with its ligands PD-L1 and PD-L2 on T cells delivers a signal that inhibits IL-2 and IFN-γ production, as well as cell proliferation induced upon T cell receptor activation (L. L. Carter, et al., Eur. J. Immunol., 2002, 32(3), 634-43). The mechanism involves recruitment of SHP-2 or SHP-1 phosphatases to inhibit T cell receptor signaling such as Syk and Lck phosphorylation (A. H. Sharpe, et al., Nat Immunol., 2007, 8(1), 239-45). Activation of the PD-1 signaling axis also attenuates PKC-θ activation loop phosphorylation, which is necessary for the activation of NF-κB and AP1 pathways, and for cytokine production such as IL-2, IFN-γ and TNF.

In some embodiments, the inhibitor of PD-1/PD-L1 that can be used in the combination therapy is a compound selected from nivolumab (OPDIVO®, BMS-936558, MDX1106, or MK-34775), pembrolizumab (KEYTRUDA®, MK-3475, SCH-900475, lambrolizumab, CAS Reg. No. 1374853-91-4), atezolizumab (Tecentriq®, CAS Reg. No. 1380723-44-3), durvalumab, avelumab (Bavencio®), cemiplimab, AMP-224, AMP-514/MEDI-0680, atezolizumab, avelumab, BGB-A317, BMS936559, durvalumab, JTX-4014, SHR-1210, pidilizumab (CT-011), REGN2810, BGB-108, BGB-A317, SHR-1210 (HR-301210, SHR1210, or SHR-1210), BMS-936559, MPDL3280A, MED14736, MSB0010718C, MDX1105-01, and one or more of the PD-1/PD-L1 blocking agents described in U.S. Pat. Nos. 7,488,802, 7,943,743, 8,008,449, 8,168,757, 8,217,149, or Pub. Nos. WO 03042402, WO 2008/156712, WO 2010/089411, WO 2010/036959, WO 2011/066342, WO 2011/159877, WO 2011/082400, WO 2011/161699, WO 2017/070089, WO 2017/087777, WO 2017/106634, WO 2017/112730, WO 2017/192961, WO 2017/205464, WO 2017/222976, WO 2018/013789, WO 2018/04478, WO 2018/119236, WO 2018/119266, WO 2018/119221, WO 2018/119286, WO 2018/119263, WO 2018/119224, WO 2019/191707, and WO 2019/217821, and any combinations thereof. The disclosure of each of the preceding patents, applications, and publications is incorporated herein by reference in its entirety.

In some embodiments, the inhibitor of PD-1/PD-L1 is selected from a compound as disclosed in WO 2018/119266, such as, e.g.,
(S)-1-((7-chloro-2-(2′-chloro-3′-(5-(((2-hydroxyethyl)amino)methyl)picolinamido)-2-methyl-[1,1′-biphenyl]-3-yl)benzo[d]oxazol-5-yl)methyl)piperidine-2-carboxylic acid, or a pharmaceutically acceptable salt thereof;
(S)-1-((7-chloro-2-(3′-(7-chloro-5-(((S)-3-hydroxypyrrolidin-1-yl)methyl)benzo[d]oxazol-2-yl)-2,2′-dimethylbiphenyl-3-yl)benzo[d]oxazol-5-yl)methyl)pyrrolidine-3-carboxylic acid, or a pharmaceutically acceptable salt thereof;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof;
(S)-1-((2-(2′-chloro-3′-(1,5-dimethyl-4,5,6,7-tetrahydro-1H-imidazo[4,5-c]pyridine-2-carboxamido)-2-methylbiphenyl-3-yl)-7-cyanobenzo[d]oxazol-5-yl)methyl)pyrrolidine-3-carboxylic acid, or a pharmaceutically acceptable salt thereof;
(R)-1-((7-cyano-2-(2,2′-dimethyl-3′-(4,5,6,7-tetrahydrothiazolo[5,4-c]pyridin-2-yl)biphenyl-3-yl)benzo[d]oxazol-5-yl)methyl)pyrrolidine-3-carboxylic acid, or a pharmaceutically acceptable salt thereof;
(R)-1-((7-cyano-2-(3′-(5-(2-(dimethylamino)acetyl)-5,6-dihydro-4H-pyrrolo[3,4-d]thiazol-2-yl)-2,2′-dimethylbiphenyl-3-yl)benzo[d]oxazol-5-yl)methyl)pyrrolidine-3-carboxylic acid, or a pharmaceutically acceptable salt thereof; and
1-((7-cyano-2-(3′-(5-(2-(dimethylamino)acetyl)-5,6-dihydro-4H-pyrrolo[3,4-d]thiazol-2-yl)-2,2′-dimethylbiphenyl-3-yl)benzo[d]oxazol-5-yl)methyl)piperidine-4-carboxylic acid, or a pharmaceutically acceptable salt thereof.

In some embodiments, the inhibitor of PD-1/PD-L1 is 1-1-((7-cyano-2-(3′-(3-((I-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof. The synthesis and characterization of I-1-((7-cyano-2-(3′-(3-((I-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid is disclosed in WO 2018/119266, which is hereby incorporated by reference in its entirety.

In some embodiments, the inhibitor of PD-1/PD-L1 is selected from:
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid hydrobromic acid salt;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid oxalic acid salt;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid hydrochloric acid salt;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid L-tartaric acid salt;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid malonic acid salt; and
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid phosphoric acid salt.

In some embodiments, the inhibitor of PD-1/PD-L1 is selected from a compound disclosed in WO 2018/119224 such as, e.g.,
(S)-1-((2-(2′-chloro-3′-(1,5-dimethyl-4,5,6,7-tetrahydro-1H-imidazo[4,5-c]pyridine-2-carboxamido)-2-methylbiphenyl-3-yl)-7-cyanobenzo[d]oxazol-5-yl)methyl)pyrrolidine-3-carboxylic acid, or a pharmaceutically acceptable salt thereof;
(R)-1-((2-(2′-chloro-3′-(6-isopropyl-4,5,6,7-tetrahydro-2H-pyrazolo[3,4-c]pyridin-2-yl)-2-methylbiphenyl-3-yl)-7-cyanobenzo[d]oxazol-5-yl)methyl)pyrrolidine-3-carboxylic acid, or a pharmaceutically acceptable salt thereof;
(S)—N-(2-chloro-3′-(5-(2-hydroxypropyl)-1-methyl-4,5,6,7-tetrahydro-1H-imidazo[4,5-c]pyridine-2-carboxamido)-2′-methylbiphenyl-3-yl)-5-isopropyl-1-methyl-4,5,6,7-tetrahydro-1H-imidazo[4,5-c]pyridine-2-carboxamide, or a pharmaceutically acceptable salt thereof;
cis-4-((2-((2,2′-dichloro-3′-(1-methyl-5-(tetrahydro-2H-pyran-4-yl)-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)methyl)cyclohexane-1-carboxylic acid, or a pharmaceutically acceptable salt thereof;
trans-4-(2-(2-((2,2′-dichloro-3′-(5-(2-hydroxyethyl)-1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)cyclohexane-1-carboxylic acid, or a pharmaceutically acceptable salt thereof;
trans-4-(2-(2-((2-chloro-2′-methyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)cyclohexane-1-carboxylic acid, or a pharmaceutically acceptable salt thereof; and
cis-4-((2-(2-chloro-3′-(5-(2-(ethyl(methyl)amino)acetyl)-5,6-dihydro-4H-pyrrolo[3,4-d]thiazol-2-yl)-2′-methylbiphenyl-3-ylcarbamoyl)-1-methyl-6,7-dihydro-1H-imidazo[4,5-c]pyridin-5(4H)-yl)methyl)cyclohexane-1-carboxylic acid, or a pharmaceutically acceptable salt thereof.

In some embodiments, the inhibitor of PD-1/PD-L1 is selected from a compound disclosed in WO 2019/191707 such as, e.g.,
(R)-1-((7-cyano-2-(3′-(7-((3-hydroxypyrrolidin-1-yl)methyl)-2-methylpyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof;
(R)-1-((7-cyano-2-(3′-(7-(((S)-1-hydroxypropan-2-ylamino)methyl)-2-methylpyrido[3,2-d]pyrimidin-4-ylamino)-2,2′-dimethylbiphenyl-3-yl)benzo[d]oxazol-5-yl)methyl)pyrrolidine-3-carboxylic acid, or a pharmaceutically acceptable salt thereof;
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof;
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)-N,N-dimethylpiperidine-4-carboxamide, or a pharmaceutically acceptable salt thereof;
(R)-1-((7-cyano-2-(3′-(2-cyclopropyl-7-(((R)-3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)pyrrolidine-3-carboxylic acid, or a pharmaceutically acceptable salt thereof; and
(R)-1-((7-cyano-2-(3′-(3-(((R)-3-hydroxypyrrolidin-1-yl)methyl)-6-methyl-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof.

In some embodiments, the inhibitor of PD-1/PD-L1 is selected from a compound disclosed in WO 2019/217821 such as, e.g.,
4-(2-(2-((2,2′-dichloro-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof;
4-(2-(2-((3′-(5-((1H-pyrazol-3-yl)methyl)-1-methyl-4,5,6,7-tetrahydro-1H-imidazo[4,5-c]pyridine-2-carboxamido)-2,2′-dichloro-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof;
I-4-(2-(2-((2,2′-dichloro-3′-(5-(2-hydroxypropyl)-1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof;
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid), or a pharmaceutically acceptable salt thereof;
4-(2-(2-((2-chloro-2′-methyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof;
4-(2-(2-((2,2′-dimethyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof; and
4-(2-(2-((3′-(5-(trans-4-carboxy-4-methylcyclohexyl)-1-methyl-4,5,6,7-tetrahydro-1H-imidazo[4,5-c]pyridine-2-carboxamido)-2,2′-dichloro-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof.

In some embodiments, the inhibitor of PD-1/PD-L1 is an antibody or antigen-binding fragment thereof that binds to human PD-1. In some embodiments, the antibody or antigen-binding fragment thereof that binds to human PD-1 is a humanized antibody.

In some embodiments, the inhibitor of PD-1/PD-L1 is retifanlimab (i.e., MGA-012).

Retifanlimab is a humanized IgG4 monoclonal antibody that binds to human PD-1. See hPD-1 mAb 7(1.2) in U.S. Pat. No. 10,577,422, which is incorporated herein by reference in its entirety. The amino acid sequences of the mature retifanlimab heavy and light chains are shown below. Complementarity-determining regions (CDRs) 1, 2, and 3 of the variable heavy (VH) domain and the variable light (VL) domain are shown in that order from N to the C-terminus of the mature VL and VH sequences and are both underlined and bolded. An antibody consisting of the mature heavy chain (SEQ ID NO:2) and the mature light chain (SEQ ID NO:3) listed below is termed retifanlimab.

### Mature Retifanlimab Heavy Chain (HC)

(SEQ ID NO: 2)
QVQLVQSGAEVKKPGASVKVSCKASGYSFTSYWMNWVRQAPGQGLEWIGV
IHPSDSETWLDQKFKDRVTITVDKSTSTAYMELSSLRSEDTAVYYCAREH
YGTSPFAYWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLG

### Mature Retifanlimab Light Chain (LC)

(SEQ ID NO: 3)
EIVLTQSPATLSLSPGERATLSCRASESVDNYGMSFMNWFQQKPGQPPKL
LIHAASNQGSGVPSRFSGSGSGTDFTLTISSLEPEDFAVYFCQQSKEVPY
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

The variable heavy (VH) domain of retifanlimab has the following amino acid sequence

(SEQ ID NO: 4)
QVQLVQSGAEVKKPGASVKVSCKASGYSFTSYWMNWVRQAPGQGLEWIGV
IHPSDSETWLDQKFKDRVTITVDKSTSTAYMELSSLRSEDTAVYYCAREH
YGTSPFAYWGQGTLVTVSS

The variable light (VL) domain of retifanlimab has the following amino acid sequence

(SEQ ID NO: 5)
EIVLTQSPATLSLSPGERATLSCRASESVDNYGMSFMNWFQQKPGQPPKL
LIHAASNQGSGVPSRFSGSGSGTDFTLTISSLEPEDFAVYFCQQSKEVPY
TFGGGTKVEIK

The amino acid sequences of the VH CDRs of retifanlimab are listed below:
VH CDR1: SYWMN (SEQ ID NO:6);
VH CDR2: VIHPSDSETWLDQKFKD (SEQ ID NO:7);
VH CDR3: EHYGTSPFAY (SEQ ID NO:8)

The amino acid sequences of VL CDRs of retifanlimab are listed below:
VL CDR1: RASESVDNYGMSFMNW (SEQ ID NO:9);
VL CDR2: AASNQGS (SEQ ID NO:10); and
VL CDR3: QQSKEVPYT (SEQ ID NO:11).

In an embodiment, the PD-1 inhibitor is a PD-1 immune checkpoint inhibitor. In another embodiment, the PD-1 inhibitor is an anti-PD-1 antibody. In yet another embodiment, the PD-1 inhibitor is selected from retifanlimab, nivolumab, pembrolizumab, cemiplimab, and dostarlimab. In still another embodiment, the PD-1 inhibitor is retifanlimab.

In an embodiment, the PD-1 inhibitor is a small molecule inhibitor.

In an embodiment, the PD-L1 inhibitor is a small molecule inhibitor. In another embodiment, the PD-L1 inhibitor is

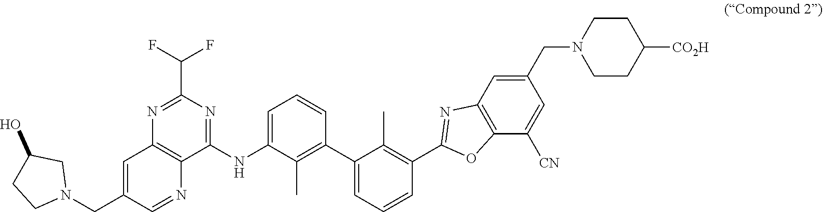 [SMILES: Cc1c(Nc2nc(C(F)F)nc3cc(CN4CC[C@@H](O)C4)cnc23)cccc1-c1cccc(-c2nc3cc(CN4CCC(C(=O)O)CC4)cc(C#N)c3o2)c1C]

or a pharmaceutically acceptable salt thereof.

### Methods of Treatment

In an aspect, provided herein is a method of treating cancer in a subject in need thereof comprising administering to the subject a KRAS G12D inhibitor, or a pharmaceutically acceptable salt thereof, and a PD-1 inhibitor or a PD-L1 inhibitor, or pharmaceutically acceptable salt thereof.

In another aspect, provided herein is a method of treating cancer in a subject in need thereof comprising administering to the subject:
a pharmaceutical composition comprising a KRAS G12D inhibitor, or a pharmaceutically acceptable salt thereof, and at least one pharmaceutically acceptable carrier or excipient; and
a pharmaceutical composition comprising a PD-1 inhibitor or a PD-L1 inhibitor, or pharmaceutically acceptable salt thereof, and at least one pharmaceutically acceptable carrier or excipient.

In an embodiment, the KRAS G12D inhibitor is a compound of Formula I, or a pharmaceutically acceptable salt thereof. In another embodiment, the KRAS G12D inhibitor is selected from a compound listed supra. In another embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

In yet another embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1*”).

In still another embodiment, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1”).

In an embodiment, the KRAS G12D inhibitor is 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1a”).

In an embodiment, the KRAS G12D inhibitor is a compound of Formula IV, or a pharmaceutically acceptable salt thereof. In another embodiment, the KRAS G12D inhibitor is selected from a compound of Formula IV listed supra. In another embodiment, the KRAS G12D inhibitor is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 3”), or a pharmaceutically acceptable salt thereof.

In another embodiment, the KRAS G12D inhibitor is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate, or a pharmaceutically acceptable salt thereof.

In another embodiment, the KRAS G12D inhibitor is methyl (1R,3R,4R,5S)-3-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate, or a pharmaceutically acceptable salt thereof.

In an embodiment, the PD-1 inhibitor is a PD-1 immune checkpoint inhibitor. In another embodiment, the PD-1 inhibitor is an anti-PD-1 antibody. In yet another embodiment, the PD-1 inhibitor is selected from retifanlimab, nivolumab, pembrolizumab, cemiplimab, and dostarlimab. In still another embodiment, the PD-1 inhibitor is retifanlimab.

In an embodiment, the PD-1 inhibitor is a small molecule inhibitor.

In an embodiment, the PD-L1 inhibitor is a small molecule inhibitor. In another embodiment, the PD-L1 inhibitor is Compound 2, or a pharmaceutically acceptable salt thereof.

In another embodiment, the KRAS G12D inhibitor, or a pharmaceutically acceptable salt thereof, is administered in combination with a PD-L1 inhibitor, or pharmaceutically acceptable salt thereof. In yet another embodiment, the PD-L1 inhibitor is an anti-PD-L1 antibody. In still another embodiment, the PD-L1 inhibitor is selected from atezolizumab, avelumab, and durvalumab.

In yet another aspect, provided herein is a method of treating cancer in a subject in need thereof comprising administering to the subject:
a pharmaceutical composition comprising Compound 1, and at least one pharmaceutically acceptable carrier or excipient; and
a pharmaceutical composition comprising retifanlimab, and at least one pharmaceutically acceptable carrier or excipient.

In another aspect, provided herein is a method of treating cancer in a subject in need thereof comprising administering to the subject:
a pharmaceutical composition comprising Compound 1*, and at least one pharmaceutically acceptable carrier or excipient; and
a pharmaceutical composition comprising retifanlimab, and at least one pharmaceutically acceptable carrier or excipient.

In another aspect, provided herein is a method of treating cancer in a subject in need thereof comprising administering to the subject:
a pharmaceutical composition comprising Compound 1*, and at least one pharmaceutically acceptable carrier or excipient; and
a pharmaceutical composition comprising Compound 2, and at least one pharmaceutically acceptable carrier or excipient.

In still another aspect, provided herein is a method of treating cancer in a subject in need thereof comprising administering to the subject:
a pharmaceutical composition comprising Compound 1, and at least one pharmaceutically acceptable carrier or excipient; and
a pharmaceutical composition comprising Compound 2, and at least one pharmaceutically acceptable carrier or excipient.

In still another aspect, provided herein is a method of treating cancer in a subject in need thereof comprising administering to the subject:
a pharmaceutical composition comprising Compound 1*, and at least one pharmaceutically acceptable carrier or excipient; and
a pharmaceutical composition comprising Compound 2, and at least one pharmaceutically acceptable carrier or excipient.

In yet another embodiment, the KRAS G12D inhibitor has an IC₅₀ of about 100 nM or lower. In still another embodiment, the KRAS G12D inhibitor is selective for inhibiting G12D versus wild-type KRAS.

In another embodiment, the KRAS G12D inhibitor is administered to the subject in a pharmaceutical composition comprising the KRAS G12D inhibitor, or a pharmaceutically acceptable salt thereof, and at least one pharmaceutically acceptable carrier or excipient.

In yet another embodiment, the PD-1 inhibitor or PD-L1 inhibitor is administered to the subject in a pharmaceutical composition comprising the PD-1 inhibitor or PD-L1, or a pharmaceutically acceptable salt thereof, and at least one pharmaceutically acceptable carrier or excipient.

In yet another aspect, provided herein is a method of treating cancer in a subject in need thereof comprising administering to the subject a KRAS G12D inhibitor that is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, and a PD-1 inhibitor that is retifanlimab.

In an embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1*”).

In another embodiment, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1”).

In yet another embodiment, the KRAS G12D inhibitor is 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1a”).

In another aspect, provided herein is a method of treating cancer in a subject in need thereof comprising administering to the subject a KRAS G12D inhibitor that is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 3”), or a pharmaceutically acceptable salt thereof, and a PD-1 inhibitor that is retifanlimab.

In another aspect, provided herein is a method of treating cancer in a subject in need thereof comprising administering to the subject a KRAS G12D inhibitor that is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 3”), or a pharmaceutically acceptable salt thereof, and a PD-L1 inhibitor that is Compound 2.

In another embodiment of the methods, the KRAS G12D inhibitor is administered twice daily (BID). In another embodiment, the KRAS G12D inhibitor is administered once daily (QD). In yet another embodiment, the KRAS G12D inhibitor is administered orally (PO).

In an embodiment, the PD-1 inhibitor is administered twice a week (BIW). In another embodiment, the PD-1 inhibitor is administered as an intraperitoneal injection (IP).

In yet another embodiment, the cancer is selected from carcinomas, hematological cancers, sarcomas, and glioblastoma. In still another embodiment, the cancer is a cancer comprising abnormally proliferating cells having a KRAS G12D mutation.

In an embodiment, the method further comprises identifying the presence of abnormally proliferating cells having a KRAS G12D mutation.

In another embodiment, the cancer is a hematological cancer selected from myeloproliferative neoplasms, myelodysplastic syndrome, chronic and juvenile myelomonocytic leukemia, acute myeloid leukemia, acute lymphocytic leukemia, and multiple myeloma.

In another embodiment, the cancer is a carcinoma selected from pancreatic, colorectal, lung, bladder, gastric, esophageal, breast, head and neck, cervical, skin, and thyroid carcinomas. In yet another embodiment, the carcinoma is colorectal carcinoma. In still another embodiment, the carcinoma is lung carcinoma. In an embodiment, the carcinoma is pancreatic carcinoma.

In another embodiment, the cancer is colorectal cancer.

In yet another embodiment, the cancer is non-small cell lung cancer (NSCLC).

In still another embodiment, the cancer is pancreatic ductal adenocarcinoma.

In another aspect, provided herein is a method of treating colorectal cancer in a subject in need thereof comprising administering to the subject a KRAS G12D inhibitor that is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, and a PD-1 inhibitor that is retifanlimab.

In another aspect, provided herein is a method of treating colorectal cancer in a subject in need thereof comprising administering to the subject a KRAS G12D inhibitor that is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, and a PD-1 inhibitor that is Compound 2.

In an embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1*”).

In another embodiment, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1”).

In yet another embodiment, the KRAS G12D inhibitor is 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1a”).

In another aspect, provided herein is a method of treating pancreatic cancer in a subject in need thereof comprising administering to the subject a KRAS G12D inhibitor that is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, and a PD-1 inhibitor that is retifanlimab.

In another aspect, provided herein is a method of treating pancreatic cancer in a subject in need thereof comprising administering to the subject a KRAS G12D inhibitor that is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, and a PD-1 inhibitor that is Compound 2.

In an embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1*”).

In another embodiment, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1”).

In yet another embodiment, the KRAS G12D inhibitor is 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1a”).

In another aspect, provided herein is a method of treating colorectal cancer in a subject in need thereof comprising administering to the subject a KRAS G12D inhibitor that is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 3”), or a pharmaceutically acceptable salt thereof, and a PD-1 inhibitor that is retifanlimab.

In another aspect, provided herein is a method of treating colorectal cancer in a subject in need thereof comprising administering to the subject a KRAS G12D inhibitor that is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 3”), or a pharmaceutically acceptable salt thereof, and a PD-L1 inhibitor that is Compound 2.

In another aspect, provided herein is a method of treating pancreatic cancer in a subject in need thereof comprising administering to the subject a KRAS G12D inhibitor that is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 3”), or a pharmaceutically acceptable salt thereof, and a PD-1 inhibitor that is retifanlimab.

In another aspect, provided herein is a method of treating pancreatic cancer in a subject in need thereof comprising administering to the subject a KRAS G12D inhibitor that is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 3”), or a pharmaceutically acceptable salt thereof, and a PD-L1 inhibitor that is Compound 2.

In an embodiment, the cancer is metastatic.

In an embodiment of the methods, the KRAS inhibitor and PD-1 inhibitor or PD-L1 inhibitor are administered separately.

In another embodiment of the methods, the cancer is a myeloproliferative neoplasm.

In another embodiment of the methods, the cancer is a myelodysplastic syndrome. Myelodysplastic syndromes (MDS) can include hematopoietic stem cell disorders characterized by one or more of the following: ineffective blood cell production, progressive cytopenias, risk of progression to acute leukemia or cellular marrow with impaired morphology and maturation (dysmyelopoiesis). Myelodysplastic syndromes can also include refractory anemia, refractory anemia with ringed sideroblasts, refractory anemia with excess blasts, refractory anemia with excess blasts in transformation and chronic myelomonocytic leukemia.

In yet another embodiment of the methods, the cancer is selected from the group consisting of chronic myeloid leukemia (CML), polycythemia vera (PV), essential thrombocythemia (ET), myelofibrosis (MF), chronic neutrophilic leukemia, chronic eosinophilic leukemia, chronic myelomonocytic leukemia, juvenile myelomonocytic leukemia, hypereosinophilic syndrome, systemic mastocytosis, atypical chronic myelogenous leukemia, acute lymphoblastic leukemia (ALL), and acute myeloid leukemia (AML). In still another embodiment, the cancer is myelofibrosis (MF).

In an embodiment of the methods, the cancer is selected from the group consisting of primary myelofibrosis, post-polycythemia vera myelofibrosis, or post-essential thrombocythemia myelofibrosis.

In another embodiment of the methods, the subject is human.

In yet another embodiment of the methods, the treatment comprises administering the KRAS inhibitor and the PD-1 inhibitor or PD-L1 inhibitor at substantially the same time.

In still another embodiment of the methods, the treatment comprises administering the KRAS inhibitor and the PD-1 inhibitor or PD-L1 inhibitor at different times.

In an embodiment of the methods, the KRAS inhibitor is administered to the subject, followed by administration of the PD-1 inhibitor or PD-L1 inhibitor. In another embodiment, the PD-1 inhibitor or PD-L1 inhibitor is administered to the subject, followed by administration of the KRAS inhibitor.

In another embodiment of the methods, the KRAS inhibitor and/or PD-1 inhibitor or PD-L1 inhibitor are administered at dosages that would not be effective when one or both of the KRAS inhibitor and the PD-1 inhibitor or PD-L1 inhibitor are administered alone, but which amounts are effective in combination.

In an embodiment of the methods, the method involves the administration of a therapeutically effective amount of a combination or composition comprising compounds provided herein, or pharmaceutically acceptable salts thereof, to a subject (including, but not limited to a human or animal) in need of treatment (including a subject identified as in need).

In another embodiment of the methods, the treatment includes co-administering the amount of the KRAS inhibitor and the amount of the PD-1 inhibitor or PD-L1 inhibitor. In an embodiment, the amount of the KRAS inhibitor and the amount of the PD-1 inhibitor or PD-L1 inhibitor are in a single formulation or unit dosage form. In still other embodiments, the amount of the KRAS inhibitor and the amount of the PD-1 inhibitor or PD-L1 inhibitor are in a separate formulations or unit dosage forms.

In the foregoing methods, the treatment can include administering the amount of KRAS inhibitor and the amount of PD-1 inhibitor or PD-L1 inhibitor at substantially the same time or administering the amount of KRAS inhibitor and the amount of PD-1 inhibitor or PD-L1 inhibitor at different times. In some embodiments of the foregoing methods, the amount of KRAS inhibitor and/or the amount of PD-1 inhibitor or PD-L1 inhibitor is administered at dosages that would not be effective when one or both of KRAS inhibitor and PD-1 inhibitor or PD-L1 inhibitor is administered alone, but which amounts are effective in combination.

### Pharmaceutical Combinations

In an aspect, provided herein is a pharmaceutical combination comprising a KRAS G12D inhibitor, or a pharmaceutically acceptable salt thereof, and a PD-1 inhibitor or a PD-L1 inhibitor, or pharmaceutically acceptable salt thereof. In an embodiment, the pharmaceutical combination can include separate pharmaceutical dosage forms or pharmaceutical compositions that are also sold independently of each other. In another embodiment, the pharmaceutical combination is for simultaneous or sequential use for being jointly active. In another embodiment, the pharmaceutical combination can include the components separately or together in a single unit dose.

In an embodiment, the KRAS G12D inhibitor is a compound of Formula I, or a pharmaceutically acceptable salt thereof. In another embodiment, the KRAS G12D inhibitor is selected from a compound listed supra. In another embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

In yet another embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1*”).

In still another embodiment, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1”).

In an embodiment, the KRAS G12D inhibitor is 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1a”).

In an embodiment, the KRAS G12D inhibitor is a compound of Formula IV, or a pharmaceutically acceptable salt thereof. In another embodiment, the KRAS G12D inhibitor is selected from a compound of Formula IV listed supra. In another embodiment, the KRAS G12D inhibitor is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 3”), or a pharmaceutically acceptable salt thereof.

In another embodiment, the KRAS G12D inhibitor is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate, or a pharmaceutically acceptable salt thereof.

In another embodiment, the KRAS G12D inhibitor is methyl (1R,3R,4R,5S)-3-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate, or a pharmaceutically acceptable salt thereof.

In an embodiment, the PD-1 inhibitor is a PD-1 immune checkpoint inhibitor. In another embodiment, the PD-1 inhibitor is an anti-PD-1 antibody. In yet another embodiment, the PD-1 inhibitor is selected from retifanlimab, nivolumab, pembrolizumab, cemiplimab, and dostarlimab. In still another embodiment, the PD-1 inhibitor is retifanlimab.

In an embodiment, the PD-1 inhibitor is a small molecule inhibitor.

In an embodiment, the PD-L1 inhibitor is a small molecule inhibitor. In another embodiment, the PD-L1 inhibitor is Compound 2, or a pharmaceutically acceptable salt thereof.

In another embodiment of the pharmaceutical combinations, the KRAS G12D inhibitor is administered twice daily (BID). In another embodiment, the KRAS G12D inhibitor is administered once daily (QD). In yet another embodiment, the KRAS G12D inhibitor is administered orally (PO).

In an embodiment, the PD-1 inhibitor is administered twice a week (BIW). In another embodiment, the PD-1 inhibitor is administered as an intraperitoneal injection (IP).

The administration of a pharmaceutical combination provided herein may result in a beneficial effect, e.g., a synergistic therapeutic effect, e.g., with regard to alleviating, delaying progression of or inhibiting the symptoms, and may also result in further surprising beneficial effects, e.g., fewer side-effects, an improved quality of life or a decreased morbidity, compared with a monotherapy applying only one of the pharmaceutically active ingredients used in the combination of the invention.

### Pharmaceutical Compositions

In an aspect, provided herein is a pharmaceutical composition comprising
a) a KRAS G12D inhibitor, or a pharmaceutically acceptable salt thereof;
b) a PD-1 inhibitor or a PD-L1 inhibitor, or a pharmaceutically acceptable salt thereof; and
c) at least one pharmaceutically acceptable carrier or excipient.

In an embodiment, the KRAS G12D inhibitor is a compound of Formula I, or a pharmaceutically acceptable salt thereof. In another embodiment, the KRAS G12D inhibitor is selected from a compound listed supra. In another embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

In yet another embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1*”).

In still another embodiment, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1”).

In an embodiment, the KRAS G12D inhibitor is 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1a”).

In an embodiment, the KRAS G12D inhibitor is a compound of Formula IV, or a pharmaceutically acceptable salt thereof. In another embodiment, the KRAS G12D inhibitor is selected from a compound of Formula IV listed supra. In another embodiment, the KRAS G12D inhibitor is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 3”), or a pharmaceutically acceptable salt thereof.

In another embodiment, the KRAS G12D inhibitor is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate, or a pharmaceutically acceptable salt thereof.

In another embodiment, the KRAS G12D inhibitor is methyl (1R,3R,4R,5S)-3-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate, or a pharmaceutically acceptable salt thereof.

In an embodiment, the PD-1 inhibitor is a PD-1 immune checkpoint inhibitor. In another embodiment, the PD-1 inhibitor is an anti-PD-1 antibody. In yet another embodiment, the PD-1 inhibitor is selected from retifanlimab, nivolumab, pembrolizumab, cemiplimab, and dostarlimab. In still another embodiment, the PD-1 inhibitor is retifanlimab.

In an embodiment, the PD-1 inhibitor is a small molecule inhibitor.

In an embodiment, the PD-L1 inhibitor is a small molecule inhibitor. In another embodiment, the PD-L1 inhibitor is Compound 2, or a pharmaceutically acceptable salt thereof.

### Packaged Formulations

Packaged pharmaceutical formulations or pharmaceutical products are included herein. Such packaged formulations include one or more pharmaceutical formulations comprising a combination of a KRAS inhibitor and a PD-1 inhibitor or PD-L1 inhibitor. The combination of compounds in formulated form is contained in a container. The package typically contains instructions for using the formulation to treat an animal (typically a human patient) suffering from cancer.

In certain embodiments the packaged pharmaceutical formulation or pharmaceutical product contains the combination of compounds described herein in a container with instructions for administering the dosage forms on a fixed schedule. In some of these embodiments, the combination of compounds is provided in separate unit dosage forms.

In a particular embodiment, the compounds of the combination can be dosed on the same schedule, whether by administering a single formulation or unit dosage form containing all of the compounds of the combination, or by administering separate formulations or unit dosage forms of the compounds of the combination. However, some of the compounds used in the combination may be administered more frequently than once per day, or with different frequencies that other compounds in the combination. Therefore, in one embodiment the packaged pharmaceutical formation contains a formulation or unit dosage form containing all of the compounds in the combination of compounds, and an additional formulation or unit dosage form that includes one of the compounds in the combination of agents, with no additional active compound, in a container, with instructions for administering the dosage forms on a fixed schedule.

The package formulations provided herein include comprise prescribing information, for example, to a patient or health care provider, or as a label in a packaged pharmaceutical formulation. Prescribing information may include for example efficacy, dosage and administration, contraindication and adverse reaction information pertaining to the pharmaceutical formulation.

In all of the foregoing the combination of compounds of the invention can be administered alone, as mixtures, or with additional active agents.

### Administration/Dosage/Formulations

In another aspect, provided herein is a pharmaceutical composition or pharmaceutical combination comprising the compounds disclosed herein, together with a pharmaceutically acceptable carrier.

Administration of the combination includes administration of the combination in a single formulation or unit dosage form, administration of the individual agents of the combination concurrently but separately, or administration of the individual agents of the combination sequentially by any suitable route. The dosage of the individual agents of the combination may require more frequent administration of one of the agent(s) as compared to the other agent(s) in the combination. Therefore, to permit appropriate dosing, packaged pharmaceutical products may contain one or more dosage forms that contain the combination of agents, and one or more dosage forms that contain one of the combination of agents, but not the other agent(s) of the combination.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In an embodiment, Compound 1 free base equivalent is administered at a dose of about 50 mg to about 2000 mg. In an embodiment, Compound 1 free base equivalent is administered at a dose of about 200 mg to about 1600 mg. In an embodiment, Compound 1 free base equivalent is administered at a dose of about 200 mg to about 1200 mg.

In an embodiment, Compound 1 free base equivalent is administered at a dose of about 25 mg, about 50 mg, about 100 mg, about 200 mg, about 250 mg, about 300 mg, about 350 mg, about 400 mg, about 450 mg, about 500 mg, about 550 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg.

In an embodiment, Compound 1 is administered once, twice, thrice, or four times daily.

In an embodiment, Compound 1* free base equivalent is administered at a dose of about 50 mg to about 2000 mg. In an embodiment, Compound 1* free base equivalent is administered at a dose of about 200 mg to about 1600 mg. In an embodiment, Compound 1* free base equivalent is administered at a dose of about 200 mg to about 1200 mg.

In an embodiment, Compound 1* free base equivalent is administered at a dose of about 100 mg, about 200 mg, about 250 mg, about 300 mg, about 350 mg, about 400 mg, about 450 mg, about 500 mg, about 550 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg.

In an embodiment, Compound 3 free base equivalent is administered at a dose of about 50 mg to about 2000 mg. In an embodiment, Compound 3 free base equivalent is administered at a dose of about 200 mg to about 1600 mg. In an embodiment, Compound 3 free base equivalent is administered at a dose of about 200 mg to about 1200 mg.

In an embodiment, Compound 3 free base equivalent is administered at a dose of about 25 mg, about 50 mg, about 100 mg, about 200 mg, about 250 mg, about 300 mg, about 350 mg, about 400 mg, about 450 mg, about 500 mg, about 550 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg.

In an embodiment, Compound 1* is administered once, twice, thrice, or four times daily.

In an embodiment, retifanlimab is administered at a dose of about 100 mg to about 1000 mg. In an embodiment, retifanlimab is administered at a dose of about 300 mg to about 800 mg. In an embodiment, retifanlimab is administered at a dose of about 400 mg to about 600 mg.

In an embodiment, retifanlimab is administered at a dose of about 100 mg, about 200 mg, about 250 mg, about 300 mg, about 350 mg, about 400 mg, about 450 mg, about 500 mg, about 550 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg.

In an embodiment, retifanlimab is administered intravenously (IV). In an embodiment, retifanlimab is administered once every four weeks (q4w).

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms are dictated by and directly dependent on (a) the unique characteristics of the disclosed compound and the particular therapeutic effect to be achieved, and (b) the limitations inherent in the art of compounding/formulating such a disclosed compound for the treatment of pain, a depressive disorder, or drug addiction in a patient.

In one embodiment, the compounds provided herein are formulated using one or more pharmaceutically acceptable excipients or carriers. In one embodiment, the pharmaceutical compositions provided herein comprise a therapeutically effective amount of a disclosed compound and a pharmaceutically acceptable carrier.

The drug compounds provided herein (for example, a KRAS inhibitor and a PD-1 inhibitor or PD-L1 inhibitor) are present in the combinations, dosage forms, pharmaceutical compositions and pharmaceutical formulations disclosed herein in a ratio in the range of 100:1 to 1:100. For example, the ratio of a PD-1 inhibitor or PD-L1 inhibitor:a KRAS inhibitor can be in the range of 1:100 to 1:1, for example, 1:100, 1:90, 1:80, 1:70, 1:60, 1:50, 1:40, 1:30, 1:20, 1:10, 1:5, 1:2, or 1:1 of PD-1 inhibitor or PD-L1 inhibitor:KRAS inhibitor. In another example, the ratio of a KRAS inhibitor:a PD-1 inhibitor or PD-L1 inhibitor can be in the range of 1:100 to 1:1, for example, 1:100, 1:90, 1:80, 1:70, 1:60, 1:50, 1:40, 1:30, 1:20, 1:10, 1:5, 1:2, or 1:1 of a KRAS inhibitor:a PD-1 inhibitor or PD-L1 inhibitor.

The optimum ratios, individual and combined dosages, and concentrations of the drug compounds that yield efficacy without toxicity are based on the kinetics of the active ingredients' availability to target sites, and are determined using methods known to those of skill in the art.

Routes of administration of any of the compositions discussed herein include oral, nasal, rectal, intravaginal, parenteral, buccal, sublingual or topical. The compounds may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions are not limited to the particular formulations and compositions that are described herein.

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### EXAMPLES

The compounds and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

The KRAS inhibitors provided herein, their syntheses, and their biological activity against KRAS can be found in WO 2023/064857, which is incorporated by reference in its entirety. The KRAS inhibitors provided herein, their syntheses, and their biological activity against KRAS can be found in PCT/US2024/025160, which is incorporated by reference in its entirety. The PD-1 and PD-L1 inhibitors provided herein, their syntheses, and their biological activity against PD-1/PD-L1 can be found in WO 2022/147092, which is incorporated by reference in its entirety.

The following abbreviations may be used herein: AcOH (acetic acid); Ac₂O (acetic anhydride); aq. (aqueous); atm. (atmosphere(s)); Boc (t-butoxycarbonyl); BOP ((benzotriazol-1-yloxy)tris(dimethylamino)phosphonium hexafluorophosphate); br (broad); Cbz (carboxybenzyl); calc. (calculated); d (doublet); dd (doublet of doublets); DBU (1,8-diazabicyclo[5.4.0]undec-7-ene); DCM (dichloromethane); DIAD (N, N′-diisopropyl azidodicarboxylate); DIEA (N,N-diisopropylethylamine); DIPEA (N, N-diisopropylethylamine); DIBAL (diisobutylaluminium hydride); DMF (N,N-dimethylformamide); DMSO (dimethyl sulfoxide); Et (ethyl); EtOAc (ethyl acetate); FCC (flash column chromatography); g (gram(s)); h (hour(s)); HATU (N, N, N′, N′tetramethyl-O-(7-azabenzotriazol-1-yl)uronium hexafluorophosphate); HCl (hydrochloric acid); HPLC (high performance liquid chromatography); Hz (hertz); J (coupling constant); L (liter(s)); LCMS (liquid chromatography-mass spectrometry); LDA (lithium diisopropylamide); m (multiplet); M (molar); mCPBA (3-chloroperoxybenzoic acid); MS (Mass spectrometry); Me (methyl); MeCN (acetonitrile); MeOH (methanol); mg (milligram(s)); min. (minutes(s)); mL (milliliter(s)); mmol (millimole(s)); MTBE (methyl tert-butyl ether); N (normal); NCS (N-chlorosuccinimide); NEt₃ (triethylamine); nM (nanomolar); NMP (N-methylpyrrolidinone); NMR (nuclear magnetic resonance spectroscopy); OTf (trifluoromethanesulfonate); Ph (phenyl); pM (picomolar); PPT(precipitate); RP-HPLC (reverse phase high performance liquid chromatography); r.t. (room temperature), s (singlet); t (triplet or tertiary); TBS (tert-butyldimethylsilyl); tert (tertiary); tt (triplet of triplets); TFA (trifluoroacetic acid); THF (tetrahydrofuran); pg (microgram(s)); μL (microliter(s)); μM (micromolar); wt % (weight percent). Brine is saturated aqueous sodium chloride. In vacuo is under vacuum.

### Example 1: Synthesis Procedures

### Example 1a: 3-(1-((1R,4R,5S)-2-Azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile

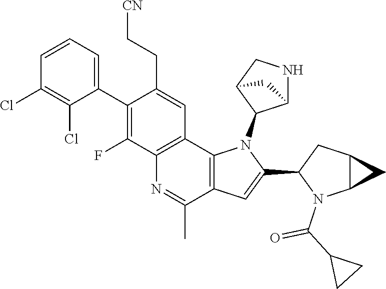 [SMILES: Cc1nc2c(F)c(-c3cccc(Cl)c3Cl)c(CCC#N)cc2c2c1cc([C@H]1C[C@H]3C[C@H]3N1C(=O)C1CC1)n2[C@H]1[C@H]2CN[C@@H]1C2]

### Step 1. Methyl 2-amino-4-bromo-3-fluorobenzoate

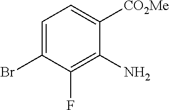 [SMILES: COC(=O)c1ccc(Br)c(F)c1N]

Dimethyl sulfate (823 g, 6.53 mole) was added to a mixture of 2-amino-4-bromo-3-fluorobenzoic acid (1500 g, 6.22 mole) and potassium carbonate (945 g, 6.84 mole) in N,N-dimethylamide or 1,4-dioxane (6 L) at 5-50° C. After the addition, the mixture was stirred at room temperature for 2 hours to complete the reaction. Water (7.5 L) was gradually added to the reaction mixture to precipitate the product. After the water addition, the mixture was stirred at room temperature for 1 hour. The solids were isolated by filtration and the wet cake was washed with water (3×1.5 L). The solids were dried under vacuum at about 50° C. overnight to give desired product (1530 g, 99% yield). LCMS calculated for C₈H₇BrFNO₂: 246.96; Found: 248 (M+H⁺). ¹H NMR (400 MHz, DMSO-d₆) δ 7.49 (dd, J=8.8, 1.7 Hz, 1H), 6.87-6.77 (m, 3H), 3.82 (s, 3H). ¹⁹F NMR (376 MHz, DMSO-d₆) δ −127.24.

### Step 2. Methyl 3-amino-2′,3′-dichloro-2-fluoro-[1,1′-biphenyl]-4-carboxylate

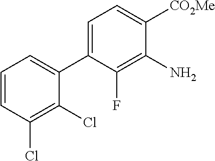 [SMILES: COC(=O)c1ccc(-c2cccc(Cl)c2Cl)c(F)c1N]

Bis(di-tert-butyl(4-dimethylaminophenyl)phosphine)dichloropalladium(II) (Pd-132) (8.12 g, 0.011 mole) was added to a mixture of methyl 2-amino-4-bromo-3-fluorobenzoate (1420 g, 5.72 mole), 2,3-dichlorophenylboronic acid (1226 g, 6.3 mole) and potassium fluoride (732 g, 12.6 mole) in acetonitrile (6 L) and water (1.5 L). The mixture was degassed and refilled with nitrogen and heated to 70° C. for 1 hour to complete the reaction. Water (6 L) was added to the reaction mixture at 50° C. The mixture was cooled to room temperature and stirred for 1 hour. The solids were isolated by filtration and the wet cake was washed with 50% acetonitrile in water (2×2 L) and water (2×2 L). The solids were dried under vacuum at about 50° C. overnight to give desired product (1700 g, 94% yield). LCMS calculated for C₁₄H₉Cl₂FNO₂: 313.01; Found: 314 (M+H⁺). ¹H NMR (400 MHz, DMSO-d₆) δ 7.74 (dd, J=8.0, 1.6 Hz, 1H), 7.64 (dd, J=8.4, 1.4 Hz, 1H), 7.48 (t, J=7.9 Hz, 1H), 7.40 (dd, J=7.9, 1.6 Hz, 1H), 6.70 (s(b), 2H), 6.51 (dd, J=8.3, 6.6 Hz, 1H), 3.86 (s, 3H). ¹⁹F NMR (376 MHz, DMSO-d₆) δ −134.70.

### Step 3. 3-Amino-6-bromo-2′,3′-dichloro-2-fluoro-[1,1′-biphenyl]-4-carboxylic acid

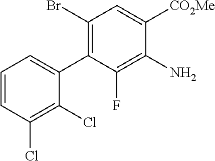 [SMILES: COC(=O)c1cc(Br)c(-c2cccc(Cl)c2Cl)c(F)c1N]

N-Bromosuccinimide (684 g, 3.84 mole) was added to a solution of methyl 3-amino-2′,3′-dichloro-2-fluoro-[1,1′-biphenyl]-4-carboxylate (1150, 3.66 mole) in acetonitrile (5.75 L) at 50-66° C. After the reaction completion, the acetonitrile (3 L) was removed by rotavapor. Water (5.75 L) was added to the concentrated mixture and stirred at room temperature for 2-3 hours. The solids were isolated by filtration and the wet cake was washed with water to give methyl 3-amino-6-bromo-2′,3′-dichloro-2-fluoro-[1,1′-biphenyl]-4-carboxylate. LCMS calculated for C₁₄H₉BrFCl₂NO₂: 390.92; Found: 391 (M+H). ¹H NMR (400 MHz, DMSO-d₆) δ 7.86 (d, J=1.7 Hz, 1H), 7.79 (dd, J=8.1, 1.5 Hz, 1H), 7.52 (t, J=7.9 Hz, 1H), 7.40 (dd, J=7.7, 1.5 Hz, 1H), 6.83 (s(b), 2H), 3.87 (s, 3H). ¹⁹F NMR (376 MHz, DMSO-d₆) δ −128.19.

### Step 4. 3-Amino-6-bromo-2′,3′-dichloro-2-fluoro-[1,1′-biphenyl]-4-carboxylic acid

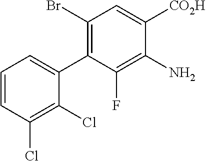 [SMILES: Nc1c(C(=O)O)cc(Br)c(-c2cccc(Cl)c2Cl)c1F]

The wet cake of 3-amino-6-bromo-2′,3′-dichloro-2-fluoro-[1,1′-biphenyl]-4-carboxylic acid was dissolved in THF (3 L) and methanol (1.5 L). Sodium hydroxide (1.5 M) aqueous solution (5 L) was added to the solution and the mixture was stirred at about 50° C. for 2 hours to complete the saponification reaction. Hydrochloric acid (1.5 M) aqueous solution was gradually added to the mixture to adjust the pH to 3-4 and stirred at room temperature for 1 hour. The solids were isolated by filtration and the wet cake was washed with water (3×1.2 L). The solids were dried under vacuum at about 50° C. overnight to give desired product (1354 g, 97.5% yield over two steps). LCMS calculated for C₁₃H₇BrCl₂FNO₂: 376.90; Found: 378 (M+H⁺). ¹H NMR (400 MHz, DMSO-d₆) δ 7.85 (d, J=1.7 Hz, 1H), 7.78 (dd, J=8.1, 1.5 Hz, 1H), 7.52 (t, J=7.9 Hz, 1H), 7.39 (dd, J=7.9, 1.5 Hz, 1H), 6.88. ¹⁹F NMR (376 MHz, DMSO-d₆) δ −128.95.

### Step 5. 6-Bromo-7-(2,3-dichlorophenyl)-8-fluoro-2H-benzo[d][1,3]oxazine-2,4(1H)-dione

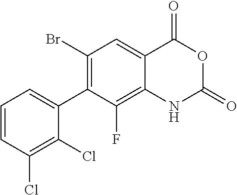 [SMILES: O=c1[nH]c2c(F)c(-c3cccc(Cl)c3Cl)c(Br)cc2c(=O)o1]

Triphosgene (500 g, 1.65 mole) in tetrahydrofuran (THF) (500 mL) was added to the solution of 3-amino-6-bromo-2′,3′-dichloro-2-fluoro-[1,1′-biphenyl]-4-carboxylic acid (1254 g, 3.31 mole) in THF (4 L) at 60° C. and stirred for 1 hour to complete the reaction. The mixture was cooled to 35° C. and n-heptane (10 L) was slowly charged to precipitate the product. The mixture was cooled to room temperature and stirred for 1 hour. The solids were isolated by filtration and washed with n-heptane (2×1 L). The wet cake was dried under vacuum at about 50° C. overnight to give desired product (1385 g, quantitative yield). LCMS calculated for C₁₄H₅BrCl₂FNO₃: 402.88; Found: 404 (M+H⁺). ¹H NMR (400 MHz, DMSO-d₆) δ 12.24 (s, 1H), 8.10 (d, J=1.5 Hz, 1H), 7.85 (dd, J=8.1, 1.5 Hz, 1H), 7.58 (t, J=7.9 Hz, 1H), 7.43 (dd, J=7.7, 1.5 Hz, 1H). ¹⁹F NMR (376 MHz, DMSO-d₆) δ −123.98.

### Step 6. Ethyl 6-bromo-7-(2,3-dichlorophenyl)-8-fluoro-4-hydroxy-2-methylquinoline-3-carboxylate

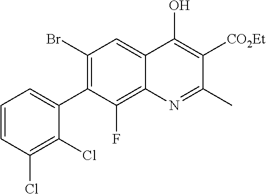 [SMILES: CCOC(=O)c1c(C)nc2c(F)c(-c3cccc(Cl)c3Cl)c(Br)cc2c1O]

A mixture of 6-bromo-7-(2,3-dichlorophenyl)-8-fluoro-2H-benzo[d][1,3]oxazine-2,4(1H)-dione (1078 g, 2.66 mole), ethyl acetoacetate (693 g, 5.32 mole), sodium acetate (393 g, 4.79 mole) and sodium chloride (933 g. 16 mole) in dimethyl sulfoxide (5 L) was heated to 50-60° C. for 5 hours. The temperature was raised to 100° C. and stirred for 1 hour to complete the reaction. The mixture was cooled to about 60° C. and water (10 L) was gradually added to precipitate the product. The mixture was cooled to room temperature and stirred for 1 hour. The solids were isolated by filtration and the wet cake was washed with water (2×2 L). The wet solids were dried under vacuum at about 50° C. overnight to give desired product (1145 g, 91% yield). LCMS calculated for C₁₉H₁₃BrCl₂FNO₂: 470.94; Found: 472 (M+H⁺). ¹H NMR (400 MHz, DMSO-d₆) δ 12.05 (s, 1H), 8.18 (d, J=1.5 Hz, 1H), 7.84 (dd, J=8.0, 1.6 Hz, 1H), 7.58 (t, J=7.9 Hz, 1H), 7.50 (dd, J=7.7, 1.6 Hz, 1H), 4.28 (q, J=7.1 Hz, 2H), 2.46 (s, 3H), 1.29 (t, J=7.1 Hz, 3H). ¹⁹F NMR (376 MHz, DMSO-d₆) δ −124.80.

### Step 6b. Ethyl 6-bromo-7-(2,3-dichlorophenyl)-8-fluoro-4-hydroxy-2-methylquinoline-3-carboxylate

The title compound can alternatively be prepared by the following process. A solution of methyl 3-amino-2′,3′-dichloro-2-fluoro-[1,1′-biphenyl]-4-carboxylate (100 g, 0.254 mole), ethyl acetoacetate (33.1 g, 0.51 mole) and p-toluenesulfonic acid (2,2 g, 0.013 mole) in xylene (1 L) was refluxed for 5 hours to azeotropically remove water. Sodium ethoxide (26 g, 0.381 mole) was added to the mixture and the mixture was refluxed for another 5 hours. The mixture was cooled to room temperature and poured into dilute hydrochloric acid pH=6-7. The organic phase was separated and the aqueous phase was extracted with ethyl acetate. The combined organic phases were concentrated and the product was purified over silica gel column and eluted with ethyl acetate and heptane (0-30%) to give desired product (65 g, 54%). LCMS calculated for C₁₉H₁₃BrCl₂FNO₃: 470.91; Found: 472 (M+H⁺). ¹H NMR (400 MHz, DMSO-d₆) δ 12.05 (s, 1H), 8.18 (d, J=1.5 Hz, 1H), 7.84 (dd, J=8.0, 1.6 Hz, 1H), 7.58 (t, J=7.9 Hz, 1H), 7.50 (dd, J=7.7, 1.6 Hz, 1H), 4.28 (q, J=7.1 Hz, 2H), 2.46 (s, 3H), 1.29 (t, J=7.1 Hz, 3H). ¹⁹F NMR (376 MHz, DMSO-d₆) δ −124.80.

### Step 7. Ethyl 6-(2-cyanovinyl)-7-(2,3-dichlorophenyl)-8-fluoro-4-hydroxy-2-methylquinoline-3-carboxylate

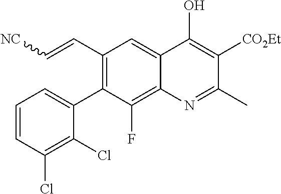 [SMILES: CCOC(=O)c1c(C)nc2c(F)c(-c3cccc(Cl)c3Cl)c(C=CC#N)cc2c1O]

A mixture of ethyl 6-bromo-7-(2,3-dichlorophenyl)-8-fluoro-4-hydroxy-2-methylquinoline-3-carboxylate (246 g, 0.52 mole), acrylonitrile (69 g, 1.3 mole), trimethylamine (156 g, 1.56 mole) and bis(di-tert-butyl)-dimethylaminophenylphosphone dichloride palladium (II) (Pd-132) (14.7 g, 0.02 mole) in N,N-dimethylamide (1.5 L) was heated to 85° C. for about 5 hours to complete the reaction. The mixture was cooled to 50° C. and water (1 L) was gradually added. The mixture was cooled to room temperature and 1 M hydrochloric acid aqueous solution was added to adjust the pH to pH 5-6. The solids were isolated by filtration and the wet cake was washed with water (2×500 mL). The wet solids were dissolved in methanol (1 L) and dichloromethane (9 L). To the solution was added sodium bisulfite (186 g, 1.8 mole) and water (4 L). The mixture was stirred at room temperature for 1 hour and the aqueous phase was separated and discarded. The organic phase was washed with water (2×2 L). Activated charcoal (150 g) was added to the organic solution and the mixture was stirred at room temperature for 1 hour. The mixture was filtered over a diatomaceous earth bed and the bed was rinsed with dichloromethane (2 L). The organic solution was concentrated to about 1 L and heptane (3.5 L) was gradually added to precipitate the product. The solids were isolated by filtration and washed with heptane (2×2 L). The wet solids were dried under vacuum at about 50° C. overnight to give desired product (210 g, 90% yield). LCMS calculated for C₂₂H₁₅Cl₂FNO₃: 444.04; Found: 445 (M+H⁺). ¹H-NMR (400 MHz, DMSO-d₆) (cis and trans mixture): 612.05 (s, 1H), 8.64 (s, OH), 8.39 (s, 1H), 7.86 (td, J=7.7, 1.5 Hz, 1H), 7.63-7.53 (m, 1H), 7.47 (td, J=7.5, 1.6 Hz, 1H), 7.04 (d, J=16.5 Hz, 1H), 6.88 (d, J=11.9 Hz, OH), 6.55 (d, J=16.6 Hz, 1H), 5.91 (d, J=12.0 Hz, OH), 4.29 (q, J=7.1 Hz, 2H), 2.47 (d, J=5.0 Hz, 4H), 1.30 (td, J=7.1, 3.2 Hz, 4H).

### Step 8. Ethyl 6-(2-cyanoethyl)-7-(2,3-dichlorophenyl)-8-fluoro-4-hydroxy-2-methylquinoline-3-carboxylate

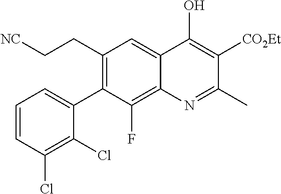 [SMILES: CCOC(=O)c1c(C)nc2c(F)c(-c3cccc(Cl)c3Cl)c(CCC#N)cc2c1O]

A mixture of ethyl 6-(2-cyanovinyl)-7-(2,3-dichlorophenyl)-8-fluoro-4-hydroxyl-2-methylquinoline-3-carboxylate (155 g, 348 mmol), pyridine (450 mL) and 1,4-dioxane (450 mL) was heated to 50-60° C. to give a homogenous solution. To the solution was added sodium borohydride (65.8 g, 1741 mmol) in portions at 50-60° C. The resulting mixture was stirred for 22 hours at 50-60° C. to complete the reduction. After cooling to about 15° C., ethyl acetate (950 mL) was added to the reaction mixture. Concentrated hydrochloric acid was gradually added to the mixture to adjust the aqueous phase pH to 1-2. The organic phase was separated and the aqueous phase was extracted with ethyl acetate (500 mL). The combined ethyl acetate phase was washed with 1 N aqueous hydrochloric acid (500 mL), water (2×500 mL), 10% brine (300 mL) and dried over sodium sulfate (75 g). The solution was concentrated and the residue was purified by silica gel column (0-20% MeOH in DCM) to give desired product (117.8 g, 76%). LCMS calculated for C₂₂H₁₇Cl₂FN₂O₃: 446.06; Found: 447 (M+H⁺). ¹H NMR (400 MHz, DMSO-d₆) δ 11.87 (s, 1H), 8.00 (s, 1H), 7.84 (dd, J=7.9, 1.7 Hz, 1H), 7.71-7.48 (m, 2H), 4.28 (q, J=7.1 Hz, 2H), 2.79 (ddd, J=11.7, 7.4, 3.7 Hz, 1H), 2.73-2.59 (m, 3H), 2.46 (s, 3H), 1.30 (t, J=7.1 Hz, 3H).

### Step 9. Ethyl 4-chloro-6-(2-cyanoethyl)-7-(2,3-dichlorophenyl)-8-fluoro-2-methylquinoline-3-carboxylate

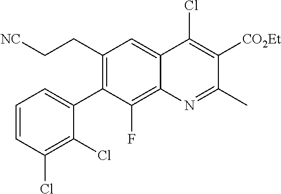 [SMILES: CCOC(=O)c1c(C)nc2c(F)c(-c3cccc(Cl)c3Cl)c(CCC#N)cc2c1Cl]

A mixture of ethyl 6-(2-cyanoethyl)-7-(2,3-dichlorophenyl)-8-fluoro-4-hydroxy-2-methylquinoline-3-carboxylate (60 g, 134 mmol), benzyltriethylammonium chloride (31 g, 135 mmol), N,N-Dimethylaniline (49.1 g, 405 mmol) in acetonitrile (300 mL) was added Phosphorus oxychloride (62 g, 405 mmol) at below 20° C. The mixture was heated to 60° C. for 1 hour to complete the reaction. The mixture was cooled to room temperature and pooled into ice-water (900 mL) at temperature below 20° C. Product precipitated out during the aqueous quench. The mixture was stirred at room temperature for more than 5 hours. The solids were isolated by filtration and the wet cake was washed with 10% acetonitrile in water (2×150 mL). The wet solids were dried under vacuum at about 50° C. overnight to give desired product (57 g, 90% yield). LCMS calculated for C₂₂H₁₆Cl₃FN₂O₂: 464.03; Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H).

### Step 10. Ethyl 4-chloro-6-(2-cyanovinyl)-7-(2,3-dichlorophenyl)-8-fluoro-2-methylquinoline-3-carboxylate

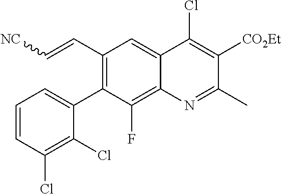 [SMILES: CCOC(=O)c1c(C)nc2c(F)c(-c3cccc(Cl)c3Cl)c(C=CC#N)cc2c1Cl]

A mixture of ethyl 6-(2-cyanovinyl)-7-(2,3-dichlorophenyl)-8-fluoro-4-hydroxy-2-methylquinoline-3-carboxylate (600 g, 1.35 mole), benzyltriethylammonium chloride (307 g, 1.35 mole), N,N-Diethylaniline (603 g, 4.04 mole) in acetonitrile (3 L) was added Phosphorus oxychloride (389.7 g, 4.04 mole) at below 20° C. The mixture was heated to 60° C. for 1 hour to complete the reaction. The mixture was cooled to room temperature and pooled into ice-water (9 L) at temperature below 20° C. Product precipitated out during the aqueous quench. The mixture was stirred at room temperature for more than 5 hours. The solids were isolated by filtration and the wet cake was washed with 10% acetonitrile in water (2×1.5 L). The wet solids were dried under vacuum at about 50° C. overnight to give desired product (563 g, 90% yield). LCMS calculated for C₂₂H₁₄Cl₃FN₂O₂: 462.01; Found: 463 (M+H⁺). ¹H-NMR (400 MHz, DMSO-d₆) (mixture of cis and trans isomers) δ 8.72 (s, 0.3H), 8.51 (s, 1H), 7.87 (ddd, J=7.3, 5.6, 1.5 Hz, 1.3H), 7.64-7.46 (m, 3H), 7.21 (d, J=16.5 Hz, 1H), 7.05 (d, J=11.9 Hz, 0.3H), 6.73 (d, J=16.5 Hz, 1H), 6.08 (d, J=11.9 Hz, 0.3H), 4.53 (qd, J=7.1, 2.0 Hz, 2H), 2.72 (d, J=7.4 Hz, 4H), 1.41 (t, J=7.1 Hz, 4H).

### Step 11. Ethyl 4-chloro-6-(2-cyanoethyl)-7-(2,3-dichlorophenyl)-8-fluoro-2-methylquinoline-3-carboxylate

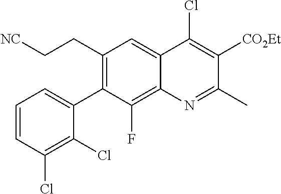 [SMILES: CCOC(=O)c1c(C)nc2c(F)c(-c3cccc(Cl)c3Cl)c(CCC#N)cc2c1Cl]

A mixture of ethyl 4-chloro-6-(2-cyanovinyl)-7-(2,3-dichlorophenyl)-8-fluoro-2-methylquinoline-3-carboxylate (528 g, 1.14 mole) and PMHS (411 g, 6.83 mole) in toluene (1.8 L) were stirred at about 50° C. In another 2-L flask, diacetoxycopper hydrate (4.1 g, 0.02 mole), (9,9-dimethyl-9H-xanthene-4,5-diyl)bis(diphenylphosphane) (13.58 g, 0.023 mol) in toluene (300 ml) and tert-butanol (483 g, 6.52 mole) were stirred for 1-2 hours to a solution. The copper acetate solution was slowly added to the solution of ethyl 4-chloro-6-(2-cyanovinyl)-7-(2,3-dichlorophenyl)-8-fluoro-2-methylquinoline-3-carboxylate and PMHS in toluene at 50-60° C. to complete the reduction. The reaction mixture was concentrated under vacuum distillation to about 2 L. To the 2 L residue was added heptane (8 L) at about 50° C. for 1 hour. The mixture was cooled to room temperature and stirred overnight. The solids were isolated by filtration and the wet cake was washed with heptane (2×1.2 L). The wet cake and silica gel (260 g) in dichloromethane (2.7 L) were stirred for 1 hour. The mixture was filtered over silica gel bed (260 g) and the silica gel bed was rinsed with DCM (4 L) until the eluent was almost colorless. The dichloromethane was removed. Dichloromethane (140 mL) and methyl tert-butyl ether (260 mL) were added to the residue. The solids were isolated by filtration and the wet cake was washed with MTBE (2×1.2 L). The wet solids were dried under vacuum at about 50° C. overnight to give desired product (476 g, 90% yield). LCMS calculated for C₂₂H₁₆Cl₃FN₂O₂: 464.03; Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H).

### Step 12. Ethyl (Rₐ)-4-chloro-6-(2-cyanoethyl)-7-(2,3-dichlorophenyl)-8-fluoro-2-methylquinoline-3-carboxylate

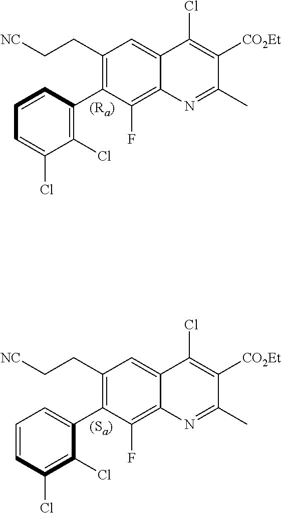

The racemic ethyl 4-chloro-6-(2-cyanoethyl)-7-(2,3-dichlorophenyl)-8-fluoro-2-methylquinoline-3-carboxylate was subject to chiral separation (Chiralpak IB N, MTBE as eluent) to give both ethyl (R)-4-chloro-6-(2-cyanoethyl)-7-(2,3-dichlorophenyl)-8-fluoro-2-methylquinoline-3-carboxylate and ethyl (S)-4-chloro-6-(2-cyanoethyl)-7-(2,3-dichlorophenyl)-8-fluoro-2-methylquinoline-3-carboxylate. LCMS calculated for C₂₂H₁₆Cl₃FN₂O₂: 464.03; Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H).

### Step 13. Ethyl 4-chloro-6-(2-cyanoethyl)-7-(2,3-dichlorophenyl)-8-fluoro-2-methylquinoline-3-carboxylate by racemization

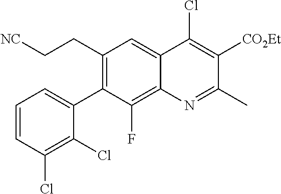 [SMILES: CCOC(=O)c1c(C)nc2c(F)c(-c3cccc(Cl)c3Cl)c(CCC#N)cc2c1Cl]

A mixture of ethyl (S)-4-chloro-6-(2-cyanoethyl)-7-(2,3-dichlorophenyl)-8-fluoro-2-methylquinoline-3-carboxylate (100 g) in sulfolane (200 mL) was heated to 185° C. for 2 hours to give racemic ethyl 4-chloro-6-(2-cyanoethyl)-7-(2,3-dichlorophenyl)-8-fluoro-2-methylquinoline-3-carboxylate. The mixture was cooled to 50° C. and acetonitrile (200 mL) was added. To the solution was added water (700 mL) at 50° C. The mixture was cooled to room temperature and stirred for 4 hours. The solids were isolated by filtration and the wet cake was washed with water (2×200 mL). The wet solids were dried under vacuum at about 50° C. overnight to give desired product (97 g, 97% yield). LCMS calculated for C₂₂H₁₆Cl₃FN₂O₂: 464.03; Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H).

### Step 14. tert-Butyl (1R,4R,5S)-5-(((R)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate

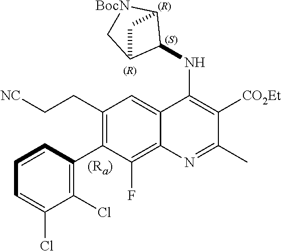

A mixture of ethyl (Rₐ)-4-chloro-6-(2-cyanoethyl)-7-(2,3-dichlorophenyl)-8-fluoro-2-methylquinoline-3-carboxylate (106.3 g, 228 mmol), tert-butyl (1R,4R,5S)-5-amino-2-azabicyclo[2.1.1]hexane-2-carboxylate (58.8 g, 297 mmol), lithium chloride (19 g, 446 mmol), diisopropylethylamine (99.5 g, 670 mmol) in dimethylsulfoxide (400 mL) was heated to 80° C. overnight. The reaction mixture was cooled to room temperature and tert-butyl methyl ether (TBME) (1 L) and water (500 mL) were subsequently added. The organic phase was separated. The organic phase was washed with 0.1 N aqueous hydrochloric acid (500 mL), saturated sodium bicarbonate (500 mL) and water (500 mL). The solvent was removed under reduced pressure to give desired product that was used for next step without further purification. Analytical sample was purified by silica gel column (0-10% MeOH in DCM). LCMS calculated for C₃₂H₃₃Cl₂FN₄O₄: 626.19; Found: 627 (M+H⁺). ¹H-NMR (400 MHz, DMSO-d₆) δ 8.09 (s, 1H), 7.82 (dd, J=8.1, 1.5 Hz, 1H), 7.56 (t, J=7.8 Hz, 1H), 7.38 (dd, J=7.7, 1.5 Hz, 1H), 7.14 (s, 1H), 4.49-4.37 (m, 2H), 4.31 (s, 1H), 3.71 (d, J=4.1 Hz, 1H), 3.65-3.43 (m, 1H), 3.18 (d, J=9.3 Hz, 1H), 3.02 (s, 1H), 2.91-2.74 (m, 2H), 2.70 (dd, J=13.6, 5.9 Hz, 2H), 2.55 (s, 3H), 1.81-1.60 (m, 1H), 1.38 (t, J=7.1 Hz, 3H), 1.34-1.06 (m, 4H), 0.92 (s, 9H).

### Step 14a. tert-Butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate

The title compound can be alternatively prepared by the following method. A mixture of ethyl (R)-4-chloro-6-(2-cyanoethyl)-7-(2,3-dichlorophenyl)-8-fluoro-2-methylquinoline-3-carboxylate (40 g, 85 mmol), lithium carbonate (19 g, 258 mmol), and tert-Butyl (1R,4R,5S)-5-amino-2-azabicyclo[2.1.1]hexane-2-carboxylate oxalate (29.4 g, 98 mmol) in DMSO (120 mL) was heated to 80° C. overnight. The reaction mixture was cooled to r.t. and MTBE (300 mL) and filtered. The solids were rinsed with MTBE (100 mL). The combined filtrate was washed with water (2×320 mL). The organic phase was separated. The solvent was removed under reduced pressure to give the product that was used for next step without further purification. An analytical sample was purified by silica gel column (0-10% MeOH in DCM). LCMS calc. for C₃₂H₃₃Cl₂FN₄O₄: 626.19; Found: 627 (M+H⁺). ¹H-NMR (400 MHz, DMSO-d₆) δ 8.09 (s, 1H), 7.82 (dd, J=8.1, 1.5 Hz, 1H), 7.56 (t, J=7.8 Hz, 1H), 7.38 (dd, J=7.7, 1.5 Hz, 1H), 7.14 (s, 1H), 4.49-4.37 (m, 2H), 4.31 (s, 1H), 3.71 (d, J==4.1 Hz, 1H), 3.65-3.43 (m, 1H), 3.18 (d, J==9.3 Hz, 1H), 3.02 (s, 1H), 2.91-2.74 (m, 2H), 2.70 (dd, J==13.6, 5.9 Hz, 2H), 2.55 (s, 3H), 1.81-1.60 (m, 1H), 1.38 (t, J=7.1 Hz, 3H), 1.34-1.06 (m, 4H), 0.92 (s, 6H).

The alternative atropisomer tert-butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate is prepared by an analogous route by performing an analogous process starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 15. (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid

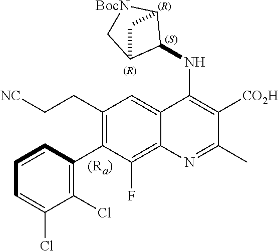

Sodium hydroxide aqueous solution (2 M) (134 mL, 268 mmol) was added to a solution of tert-butyl (1R,4R,5S)-5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (140.0 g, 223 mmol) in acetonitrile (560 ml) and methanol (210 ml) at room temperature. The mixture was heated to 50° C. for 1-1.5 hours. The mixture was cooled to room temperature and acidified with 1 M hydrochloric acid aqueous solution to about pH 5. The acetonitrile and methanol were removed under vacuum. The product was extracted by ethyl acetate (1.7 L). The aqueous phase was separated and extracted with ethyl acetate (420 mL). The combined ethyl acetate phases were concentrated under vacuum to give a residue. Tert-Butyl methyl ether (300 mL) was added to the residue and the mixture slurry was agitated at room temperature for 2 hours. The solids were isolated by filtration and the wet cake was washed with TBME (2×100 mL). The solids were dried under vacuum at about 50° C. to give desired product (135 g, quantitative) that was used for next step without further purification.

### Step 15b. (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid

The tile compound can be alternatively prepared by the following process. Sodium trimethylsinolate (338 g, 95%) was added to a solution of tert-butyl (1R,4R,5S)-5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (1400 g, 2.231 mol) in tetrahydrofuran (14 L) and water (80 mL) at room temperature. The mixture was heated to 50° C. for 1-3 hours to complete the reaction. The mixture was cooled to room temperature and acidified with 1 M hydrochloric acid aqueous solution to about pH 5. The tetrahydrofuran was removed under vacuum. The product was extracted by dichloromethane (6 L). The aqueous phase was separated and extracted with dichloromethane (6 L). The combined organic phases were concentrated under vacuum to give the product in DCM solution (6 L). The concentrated dichloromethane solution was added to tert-butyl methyl ether (7 L) was added to the residue and the mixture slurry was agitated at room temperature for 2 hours. N-Heptane (7 L) was added to the mixture. The dichloromethane was removed under vacuum. The solids were isolated by filtration and the wet cake was washed with n-heptane (2×3 L). The solids were dried under vacuum at about 50° C. to give desired product that was used for next step without further purification.

### Step 16. tert-butyl (1R,4R,5S)-5-(((R)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate

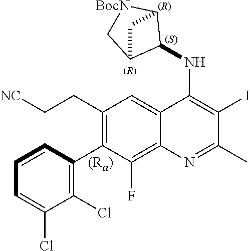

To a mixture of 4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid (132 g, 220 mmol), and sodium phosphate (74.4 g, 440 mmol) in anhydrous acetonitrile (1614 ml) was added N-iodosuccinimide (94 g, 396 mmol) and the mixture was stirred for 1 hour. Water (1.6 L) was added to the mixture and resulting slurry was stirred for 5 hours at room temperature. The solids were isolated by filtration and the wet cake was reslurried in water (2.6 L) at room temperature for 5 hours. The solids were isolated by filtration and the wet cake was washed with water (2×250 mL). The solids were dried under vacuum at about 50° C. to give desired product (120 g, 80% yield). LCMS calculated for C₃₉H₂₈Cl₂FIN₄O₂: 680.06; Found: 681 (M+H⁺). ¹H-NMR (400 MHz, DMSO-d₆) δ 7.94 (s, 1H), 7.82 (dd, J=8.0, 1.6 Hz, 1H), 7.56 (t, J=7.8 Hz, 1H), 7.50 (dd, J=7.7, 1.6 Hz, 1H), 5.49 (s, 1H), 4.28 (s, 2H), 3.09 (s, 1H), 2.96-2.58 (m, 8H), 1.71 (s, 1H), 1.59-0.96 (m, 11H).

### Step 17. tert-Butyl (1R,4R,5S)-5-(((Rₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate

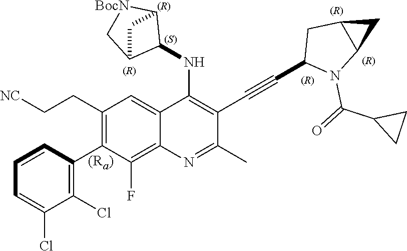

A mixture of cyclopropyl((1R,5R)-3-ethynyl-2-azabicyclo[3.1.0]hexan-2-yl)methanone (47.5 g, 260 mmol), tert-butyl (1R,4R,5S)-5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate (136.5 g, 200 mmol), and tetrabutylammonium acetate (242 g, 801 mmol) in DMF (1100 ml) was subsurface purged with nitrogen gas for 10 minutes. Tris(dibenzylideneacetone)dipalladium(0) (2.75 g, 3 mmol) was added to the mixture. The mixture was subsurface purged with nitrogen gas for another 15 minutes before heating to 70° C. for 1 hour. The reaction mixture was cooled to room temperature and added to half saturated sodium bicarbonate aqueous solution (2200 mL). The solids were isolated by filtration and the wet cake was washed with water (600 mL). The solids were dried under vacuum at about 50° C. and purified by silica gel column eluted with 0-2% methanol in ethyl acetate to give desired product (142 g, 96% yield). ¹H NMR (400 MHz, DMSO-d6) δ 8.03 (d, J=12.4 Hz, 1H), 7.81 (dd, J=8.1, 1.6 Hz, 1H), 7.55 (t, J=7.9 Hz, 1H), 7.36 (d, J=7.3 Hz, 1H), 6.70-6.44 (m, 1H), 5.68-5.13 (m, 1H), 4.54-4.18 (m, 2H), 4.00-3.80 (m, 1H), 3.51 (s, 1H), 3.19 (t, J=9.0 Hz, 1H), 3.07-2.91 (m, 1H), 2.78 (d, J=10.7 Hz, 3H), 2.66 (d, J=9.0 Hz, 3H), 2.57 (d, J=11.7 Hz, 4H), 2.36-2.08 (m, 2H), 1.88 (dd, J=17.9, 10.5 Hz, 2H), 1.35 (d, J=9.7 Hz, 2H), 1.15-0.59 (m, 16H).

### Step 17a. tert-Butyl (1R,4R,5S)-5-(((Rₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate

The title compound can alternatively be prepared by the following method. A mixture of cyclopropyl((1R,5R)-3-ethynyl-2-azabicyclo[3.1.0]hexan-2-yl)methanone (17.7 kg, 101 mol), tert-butyl (1R,4R,5S)-5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate (64.7 kg, 95 mol), copper (1) iodide (0.42 kg 2 mol), tris (4-fluorophenyl)phosphine (0.39 kg, 1 mol) and K₂CO₃ (36.4 kg, 191 mol) in DMSO (488.4 L) was subsurface purged with nitrogen gas for 30 min. Palladium (II) acetate (60 g, 30 mmol) was added to the mixture. The mixture was subsurface purged with nitrogen gas for another 30 min. before heating to 50° C. for more than 10 h. The reaction mixture was cooled to r.t. and EtOAc (906 L) was added, followed by slow addition of water (1267 L) was added. The mixture was stirred at r.t. for 30 min. and filtered over a diatomaceous earth bed. The diatomaceous earth bed was rinsed with EtOAc (33 L). The organic phase was separated from the aqueous phase and the aqueous phase was back extracted with EtOAc (195 L). The combined organic phase was washed with water (195 L). To the EtOAc phase was added water (130 L) and ammonium pyrrolidinedithiocarbamate (3.1 kg, 19 mol). The mixture was agitated at 50° C. for no less than 4 h. The mixture was cooled to r.t. and polish filtered. The aqueous phase was separated and discarded. The organic phase was washed with water (325 L). The organic phase was heated to 50° C. and passed through activated carbon cartridge. The solution is concentrated under vacuum and solvent swapped into toluene to remove residual water to give desired product in 98% solution yield. The toluene solution was solvent swap into NMP for next step indole-cyclization without further purification. ¹H NMR (400 MHz, DMSO-d₆) δ 8.03 (d, J=12.4 Hz, 1H), 7.81 (dd, J=8.1, 1.6 Hz, 1H), 7.55 (t, J=7.9 Hz, 1H), 7.36 (d, J=7.3 Hz, 1H), 6.70-6.44 (m, 1H), 5.68-5.13 (m, 1H), 4.54-4.18 (m, 2H), 4.00-3.80 (m, 1H), 3.51 (s, 1H), 3.19 (t, J=9.0 Hz, 1H), 3.07-2.91 (m, 1H), 2.78 (d, J=10.7 Hz, 3H), 2.66 (d, J=9.0 Hz, 3H), 2.57 (d, J=11.7 Hz, 4H), 2.36-2.08 (m, 2H), 1.88 (dd, J=17.9, 10.5 Hz, 2H), 1.35 (d, J=9.7 Hz, 2H), 1.15-0.59 (m, 16H).

The alternative atropisomer tert-butyl (1R,4R,5S)-5-(((Sₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate is prepared by an analogous route by performing processes analogous to Steps 14-17 starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 18. tert-Butyl (1R,4R,5S)-5-((Rₐ)-8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate

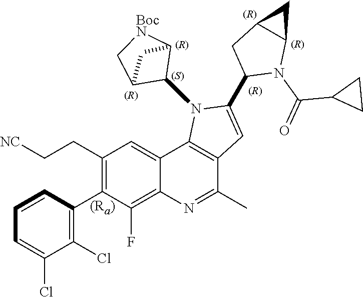

To a mixture of tert-butyl (1R,4R,5S)-5-((6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate (141.0 g, 159 mmol) and cesium carbonate (78 g, 238 mmol) in dimethyl sulfoxide (1 L) or N-methy-2-pyrrolidone was heated to 80-85° C. for 1 hour and half. The reaction was cooled to room temperature and water (2 L) was gradually added. The product was gradually precipitated out of the solution. The resulting slurry was stirred at room temperature for 1 h. The solids were isolated by filtration and the wet cake was washed with water (2×300 mL). The wet solids were dried under vacuum. The solids were purified by flash chromatography with 60-100% ethyl acetate in dichloromethane. The solvents were removed and the solids in heptane (840 mL) were crystallized from ethyl acetate (420 mL) and tert-butyl methyl ether (420 mL) and heptane 9840 mL) to give desired product (122 g, 87% yield). LCMS calculated for C₄₀H₄₀Cl₂FN₅O₃: 727.25; Found: 728 (M+H⁺). ¹H NMR (500 MHz, DMSO-d6) δ 8.12 (s, 1H), 7.81 (dt, J=8.0, 2.1 Hz, 1H), 7.55 (td, J=7.8, 5.0 Hz, 1H), 7.45-7.29 (m, 1H), 6.26 (s, 1H), 5.81-5.49 (m, 1H), 5.34-5.13 (m, 1H), 5.00 (dd, J=14.3, 6.8 Hz, 1H), 4.19-3.97 (m, 1H), 3.63 (dt, J=6.8, 3.1 Hz, 1H), 3.40 (d, J=9.4 Hz, 1H), 3.27-3.09 (m, 1H), 2.95 (dt, J=14.2, 7.6 Hz, 1H), 2.89-2.73 (m, 3H), 2.70 (d, J=2.7 Hz, 4H), 2.34-2.20 (m, 1H), 2.21-1.97 (m, 2H), 1.73 (dp, J=15.0, 4.8 Hz, 1H), 1.66-1.34 (m, 2H), 1.21-1.03 (m, 1H), 1.02-0.79 (m, 4H), 0.78-0.22 (m, 11H).

### Step 19. 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile

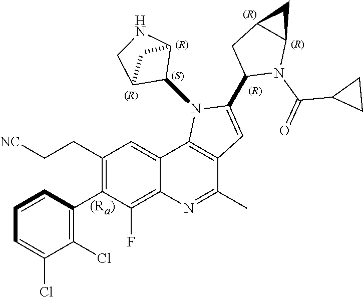

To a solution of tert-Butyl (1R,4R,5S)-5-((Rₐ)-8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate (167.7 g, 230.1 mmol) in dichloromethane (1.35 L) was added trimethylsilyl iodide (69 g, 345 mmol) at room temperature and stirred for 1 hour. Sodium bicarbonate aqueous solution (500 mL) was added to quench the reaction. The organic phase was isolated and washed with water. The solvent was evaporated by rotavapor and the residue was passed over silica gel bed (1-20% methanol in dichloromethane). The solvent was swapped into ethyl acetate and tert-butyl methyl ether to give crystalline product (136 g, 94% yield). LCMS calculated for C₃₅H₃₂Cl₂FN₅O: 627.20; Found: 628 (M+H⁺). ¹H-NMR (400 MHz, DMSO-d₆) δ ¹H NMR (500 MHz, DMSO-d6) δ 8.15 (d, J=13.6 Hz, 1H), 7.89-7.73 (m, 1H), 7.64-7.33 (m, 2H), 6.69-6.14 (m, 1H), 5.76-5.43 (m, 1H), 4.97 (d, J=4.9 Hz, 1H), 4.31 (dd, J=17.0, 6.0 Hz, 1H), 4.18-3.94 (m, 1H), 3.58-3.45 (m, 1H), 2.94 (dt, 2H, J=12.4, 6.1 Hz), 2.89-2.56 (m, 8H), 2.44-2.19 (m, 2H), 2.07 (d, J=12.9 Hz, 1H), 1.96-1.54 (m, 3H), 1.30-1.13 (m, 1H), 1.06-0.20 (m, 6H).

The alternative atropisomer 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile is prepared by an analogous route by performing processes analogous to Steps 14-19 starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 20: 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile monohydrochloride dihydrate (Compound 1)

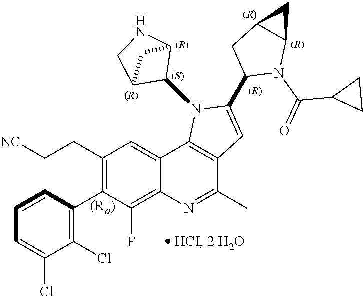

To a solution of dissolved 3-((Rₐ)-1-((1R,4R,5S)-2-azabicyclo[2.1.1]-hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile free base (53.8 g, 85 mmol) in methanol (110 mL), ethyl acetate (50 mL), water (11 mL) and tert-butyl methyl ether (TBME) (110 mL) was added 6N aqueous hydrochloric acid (14.5 mL) at 30-50° C. The mixture was seeded and the solution gradually turned cloudy. TBME (440 mL) was slowly added to the mixture at about 40° C. over 1 hour. The mixture was cooled to about 15° C. and agitated for 2 hours. The solids were isolated by filtration and the wet cake was washed with 5% methanol and 20% ethyl acetate in TBME (2×110 mL). The wet solids were slurried in ethyl acetate (270 mL) and dried under vacuum at about 50° C. to give desired product (53.7 g, 90% yield). LCMS calculated for C₃₅H₃₂Cl₂FN₅O: 627.20; Found: 628 (M+H⁺). ¹H NMR (500 MHz, DMSO-d₆) δ 8.15 (s, 1H), 7.83 (dd, J=8.1, 1.6 Hz, 1H); 7.57 (dd, J=7.9, 7.9,1 H); 7.45 (dd, J=7.7, 1.6 Hz, 1H); 6.44 (s, 1H); 5.65 (s, 1H); 5.51 (d, J=10.6 Hz, 1H); 4.14 (td, J=6.4, 2.6 Hz, 1H); 3.84-3.90 (m, 1H); 3.30-3.37 (m, 1H); 3.43-3.50 (m, 1H); 2.86-2.95 (m, 1H); 2.83-2.92 (m, 1H); 2.79 (s, 3H); 2.70-2.79 (m, 1H); 2.29-2.35 (m, 1H); 2.25-2.32 (m, 1H); 1.97 (dd, J=13.0, 2.6 Hz, 1H); 1.69-1.83 (m, 1H); 1.65 (d, J=9.1 Hz, 1H); 0.91-1.00 (m, 2H); 0.82-0.88 (m, 2H); 0.72-0.80 (m, 1H); 0.63-0.69 (m, 1H). 13C NMR (125 MHz, DMSO-d₆) δ 171.6; 145.8; 132.8; 135.1; 132.8; 131.9; 131.5; 131.4; 129.2; 101.6; 120.7; 57.9; 56.5; 44.5; 42.5; 30.5; 38.3; 32.8; 22.1; 17.5; 17.1; 13.2; 13.0; 7.70; 7.80. 19F NMR (376 MHz, DMSO-d₆) δ −122.1 (s).

The alternative atropisomer 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile monohydrochloride dihydrate is prepared by an analogous route by performing processes analogous to Steps 15-21 starting from 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile instead of 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile.

### Example 1 b: (R)-1-((7-Cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid (Compound 2)

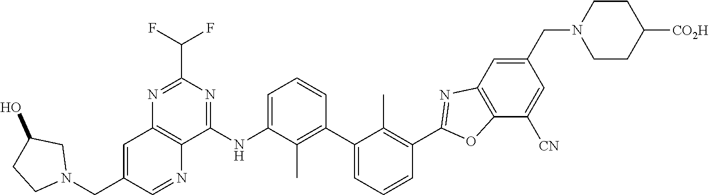 [SMILES: Cc1c(Nc2nc(C(F)F)nc3cc(CN4CC[C@@H](O)C4)cnc23)cccc1-c1cccc(-c2nc3cc(CN4CCC(C(=O)O)CC4)cc(C#N)c3o2)c1C]

### Step 1: 7-Bromo-2-(difluoromethyl)-4H-pyrido[3,2-d][1,3]oxazin-4-one

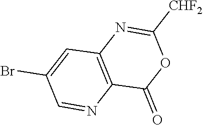 [SMILES: O=c1oc(C(F)F)nc2cc(Br)cnc12]

A mixture of 3-amino-5-bromopicolinic acid (PharmBlock cat #PB0554: 645 mg, 2.97 mmol) and 2,2-difluoroacetic anhydride (4.14 g, 23.8 mmol) was stirred at 60° C. for 3 h. After cooling to r.t., the volatiles were removed by rotavap and high vacuum pump. The residue was used directly for next step. LC-MS calculated for C₈H₄BrF₂N₂O₂ (M+H)⁺: m/z=276.9; found 277.0.

### Step 2: 7-Bromo-2-(difluoromethyl)pyrido[3,2-d]pyrimidin-4-ol

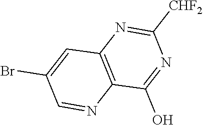 [SMILES: Oc1nc(C(F)F)nc2cc(Br)cnc12]

A mixture of 7-bromo-2-(difluoromethyl)-4H-pyrido[3,2-d][1,3]oxazin-4-one (801 mg, 2.89 mmol) and ammonium hydroxide aq. soln, (8.0 ml, 28%) in a heavy wall glass tube was sealed and stirred at 85° C. for 2 h. After cooling to r.t., the solution was then evaporated and the residue was rediluted with CH₃CN and toluene. The suspension was evaporated again and the residue was used in the next step without further purification. LC-MS calculated for C₈H₅BrF₂N₃O (M+H)⁺: m/z=276.0; found 276.0.

### Step 3: 7-Bromo-N-(3-chloro-2-methylphenyl)-2-(difluoromethyl)pyrido[3,2-d]pyrimidin-4-amine

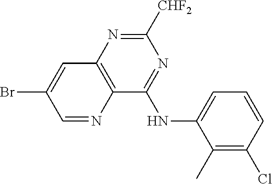 [SMILES: Cc1c(Cl)cccc1Nc1nc(C(F)F)nc2cc(Br)cnc12]

To a mixture of 7-bromo-2-(difluoromethyl)pyrido[3,2-d]pyrimidin-4-ol (crude product from Step 2: 750 mg, 2.72 mmol), benzyltriethylammonium chloride (1238 mg, 5.43 mmol) and N,N-diethylaniline (648 μl, 4.08 mmol) in acetonitrile (13.6 ml) was added phosphoryl chloride (1.52 ml, 16.3 mmol). The mixture was stirred at 75° C. for 2 h. Then the reaction was cooled to r.t. The volatiles were removed under reduced pressure.

To a solution of 3-chloro-2-methylaniline (409 mg, 2.89 mmol) and 7-bromo-4-chloro-2-(difluoromethyl)pyrido[3,2-d]pyrimidine (the residue above) in 2-propanol (14.4 ml) was added methanesulfonic acid (188 μl, 2.89 mmol). The mixture was stirred at 80° C. for 2 h. Then the reaction was cooled to r.t. The mixture was carefully quenched by NaHCO₃ aq solution. The precipitates were filtered, washed by water and dried by air. The solids were used directly for next step. LC-MS calculated for C₁₅H₁₁BrClF₂N₄(M+H)⁺: m/z=399.0; found 399.0.

### Step 4: N-(3-Chloro-2-methylphenyl)-2-(difluoromethyl)-7-vinylpyrido[3,2-d]pyrimidin-4-amine

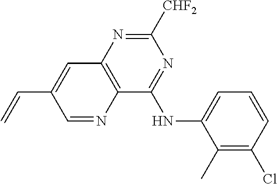 [SMILES: C=Cc1cnc2c(Nc3cccc(Cl)c3C)nc(C(F)F)nc2c1]

A mixture of 7-bromo-N-(3-chloro-2-methylphenyl)-2-(difluoromethyl)pyrido[3,2-d]pyrimidin-4-amine (841 mg, 2.10 mmol), 4,4,5,5-tetramethyl-2-vinyl-1,3,2-dioxaborolane (537 μl, 3.16 mmol), tetrakis(triphenylphosphine) palladium(0) (243 mg, 0.21 mmol) and potassium phosphate (1117 mg, 5.26 mmol) in tert-butanol (7.0 ml) and water (7.0 ml) was purged with N₂ and then stirred at 100° C. for 3 h. The reaction was cooled to room temperature. The reaction mixture was diluted with water and extracted with DCM. The organic layer was dried over MgSO₄, filtered and concentrated to give a crude residue, which was purified by flash chromatography (0-30% EtOAc/DCM). LC-MS calculated for C₁₇H₁₄ClF₂N₄(M+H)⁺: m/z=347.1; found 347.1.

### Step 5: 4-(3-Chloro-2-methylphenylamino)-2-(difluoromethyl)pyrido [3,2-d]pyrimidine-7-carbaldehyde

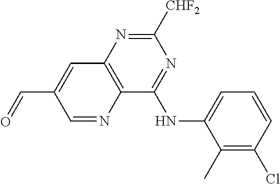 [SMILES: Cc1c(Cl)cccc1Nc1nc(C(F)F)nc2cc(C=O)cnc12]

A vial was charged with N-(3-chloro-2-methylphenyl)-2-(difluoromethyl)-7-vinylpyrido[3,2-d]pyrimidin-4-amine (195 mg, 0.562 mmol), THF (4.5 ml), a stir bar and water (1.1 ml). To this solution was added sodium periodate (601 mg, 2.81 mmol) followed by osmium tetroxide (4% w/w in water, 221 μl, 0.028 mmol). After stirring at r.t. for 1 h, the reaction was quenched with a saturated aqueous solution of sodium thiosulfate. The mixture was then extracted with DCM, and the combined organic layers were washed with water, brine, dried over MgSO₄, filtered, and concentrated in vacuo. The crude residue was used directly in the next step without further purification. LC-MS calculated for C₁₆H₁₂ClF₂N₄O (M+H)+: m/z=349.1; found 349.1.

### Step 6: (R)-1-((4-(3-Chloro-2-methylphenylamino)-2-(difluoromethyl)pyrido[3,2-d]pyrimidin-7-yl)methyl)pyrrolidin-3-ol

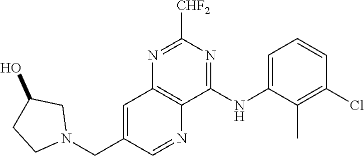 [SMILES: Cc1c(Cl)cccc1Nc1nc(C(F)F)nc2cc(CN3CC[C@@H](O)C3)cnc12]

A mixture of 4-((3-chloro-2-methylphenyl)amino)-2-(difluoromethyl)pyrido[3,2-d]pyrimidine-7-carbaldehyde (101 mg, 0.290 mmol) and (R)-pyrrolidin-3-ol (30.3 mg, 0.348 mmol) in DCM (1931 I) was stirred at r.t. for 30 min. Then sodium triacetoxyborohydride (92 mg, 0.434 mmol) was added. The mixture was further stirred at r.t. for 1 h. The reaction was quenched with NH₄OH aq. solution and extracted by DCM. The organic phase was combined and dried over MgSO₄. After filtration, the DCM solution was concentrated to a residue, which was purified by flash chromatography (0-12% MeOH/DCM). LC-MS calculated for C₂₀H₂₁ClF₂N₅O (M+H)⁺: m/z=420.1; found 420.2.

### Step 7: (R)-2-(3′-(2-(Difluoromethyl)-7-((3-hydroxypyrrolidin-1-yl)methyl)pyrido[3,2-d]pyrimidin-4-ylamino)-2,2′-dimethylbiphenyl-3-yl)-5-formylbenzo[d]oxazole-7-carbonitrile

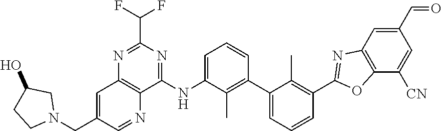 [SMILES: Cc1c(Nc2nc(C(F)F)nc3cc(CN4CC[C@@H](O)C4)cnc23)cccc1-c1cccc(-c2nc3cc(C=O)cc(C#N)c3o2)c1C]

A mixture of (R)-1-((4-(3-chloro-2-methylphenylamino)-2-(difluoromethyl)pyrido[3,2-d]pyrimidin-7-yl)methyl)pyrrolidin-3-ol (34.4 mg, 0.082 mmol), 5-formyl-2-(2-methyl-3-(4,4,5,5-tetramethyl-1,3,2-dioxaborolan-2-yl)phenyl)benzo[d]oxazole-7-carbonitrile (35 mg, 0.090 mmol), chloro(2-dicyclohexylphosphino-2′,4′,6′-triisopropyl-1,1′-biphenyl)[2-(2′-amino-1,1′-biphenyl)]palladium(II) (6.5 mg, 8.2 mol) and potassium phosphate (43.5 mg, 0.205 mmol) in water (140 I) and 1,4-dioxane (690 I) was purged with N₂ and then sealed. The reaction was stirred at 100° C. for 2 h. The reaction was cooled to room temperature. The reaction mixture was diluted with DCM and H₂O. The layers were separated. The aqueous layer was extracted with DCM three times. The organic layer was dried over MgSO₄, filtered and concentrated to give a crude residue, which was used directly in the next step without further purification. LC-MS calculated for C₃₆H₃₀F₂N₇O₃(M+H)⁺: m/z=646.2; found 646.3.

### Step 8: (R)-1-((7-Cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid

A mixture of (R)-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)-5-formylbenzo[d]oxazole-7-carbonitrile (9.5 mg, 0.015 mmol) and tert-butyl piperidine-4-carboxylate (5.45 mg, 0.029 mmol) was stirred at r.t. for 2 h. Then sodium triacetoxyborohydride (9.36 mg, 0.044 mmol) was added. The mixture was stirred at r.t. for 1 h. Then to the mixture was added trifluoroacetic acid (300 I) and stirred for 30 min. The volatiles were evaporated and the residue was diluted with MeOH and then purified by prep-HPLC (pH=2, acetonitrile/water+TFA) to give the desired product as TFA salt. LC-MS calculated for C₄₂H₄₁F₂N₈O₄(M+H)⁺: m/z=759.3; found 759.6. ¹H NMR (500 MHz, DMSO) δ 10.63 (s, 1H), 9.13 (s, 1H), 8.52 (d, J=2.0 Hz, 1H), 8.39 (d, J=1.6 Hz, 1H), 8.19 (dd, 7=7.9, 1.5 Hz, 1H), 8.11 (d, J=2.1 Hz, 1H), 7.64 (dd, J=8.1, 1.3 Hz, 1H), 7.59 (t, J=7.7 Hz, 1H), 7.49 (dd, J=7.5, 1.5 Hz, 1H), 7.41 (t, =7.8 Hz, 1H), 7.16 (dd, =7.6, 1.3 Hz, 1H), 6.74 (t, J=54.5 Hz, 1H), 4.85-4.65 (m, 2H), 4.58-4.40 (m, 3H), 3.74-3.00 (m, 8H), 2.78-2.54 (m, 1H), 2.50 (s, 3H), 2.32-1.91 (m, 5H), 1.95 (s, 3H), 1.79-1.67 (m, 1H).

### Example 1c: Synthesis procedure for Methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (Compound 3)

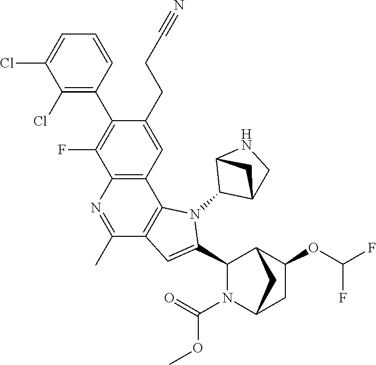 [SMILES: COC(=O)N1[C@@H]2C[C@@H]([C@@H](OC(F)F)C2)[C@@H]1c1cc2c(C)nc3c(F)c(-c4cccc(Cl)c4Cl)c(CCC#N)cc3c2n1[C@H]1[C@H]2CN[C@@H]1C2]

### Step 1. Methyl (1R,3R,4R,5S)-5-((tert-butyldiphenylsilyl)oxy)-3-ethynyl-2-azabicyclo[2.2.1]heptane-2-carboxylate

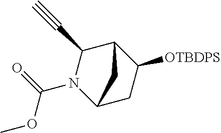

To a solution of Intermediate 5 (1.85 g, 3.89 mmol) in dioxane (10 mL) was added HCl (4 N in dioxane, 10 mL). The reaction was stirred at room temperature for 0.5 h. Upon completion, the volatiles were removed under reduced pressure. The residue was dissolved in DCM (20 mL). Upon stirring, N,N-diisopropylethylamine (2.0 mL, 11.7 mmol) was added followed by methyl chloroformate (0.6 mL, 7.78 mmol). The mixture was stirred for 0.5 h. Once completed, the reaction mixture was diluted with DCM, washed with water, dried over Na₂SO₄, filtered and concentrated. The crude product was purified by flash chromatography (0-50% ethyl acetate/hexanes) to afford the title compound. LC-MS calc. for C₂₆H₃₂NO₃Si (M+H)⁺: m/z=434.2; found 434.2.

### Step 2. Methyl (1R,3R,4R,5S)-3-ethynyl-5-hydroxy-2-azabicyclo[2.2.1]heptane-2-carboxylate

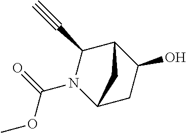

To a solution of methyl (1R,3R,4R,5S)-5-((tert-butyldiphenylsilyl)oxy)-3-ethynyl-2-azabicyclo[2.2.1]heptane-2-carboxylate (1.43 g, 3.30 mmol) in THF (11 mL) was added TBAF (1 N in THF, 4.0 mL, 3.96 mmol). The reaction was stirred at room temperature for 16 h. Upon completion, the volatiles were removed. The crude was purified by flash chromatography (0-10% methanol/DCM) to afford the title compound. LC-MS calc. for C₁₀H₁₄NO₃ (M+H)⁺: m/z=196.1; found 196.1.

### Step 3. Methyl (1R,3R,4R,5S)-5-(difluoromethoxy)-3-ethynyl-2-azabicyclo[2.2.1]heptane-2-carboxylate

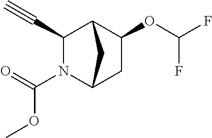 [SMILES: C#C[C@H]1[C@H]2C[C@H](C[C@@H]2OC(F)F)N1C(=O)OC]

To a flask containing methyl (1R,3R,4R,5S)-3-ethynyl-5-hydroxy-2-azabicyclo[2.2.1]heptane-2-carboxylate (0.514 g, 2.63 mmol) and copper(I) iodide (0.100 g, 0.527 mmol) was charged acetonitrile (13 mL). The mixture was stirred at 50° C. before an acetonitrile solution (2 mL) containing 2-(fluorosulfonyl)difluoroacetic acid (0.703 g, 3.95 mmol) was added slowly. The reaction mixture was stirred at 50° C. for 1 h. Upon completion, the mixture was concentrated under reduced pressure. The residue was dissolved in DCM, washed with saturated NaHCO₃ solution and water. The organic layer was dried over Na₂SO₄, filtered and concentrated. The crude product was purified by silica chromatography (0-50% ethyl acetate/hexanes) to afford title compound (0.467 g, 72% yield). LC-MS calc. for C₁₁H₁₄F₂NO₃ (M+H)⁺: m/z=246.1; found 246.1.

### Step 4. tert-Butyl (1R,4R,5S)-5-((Rₐ)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate

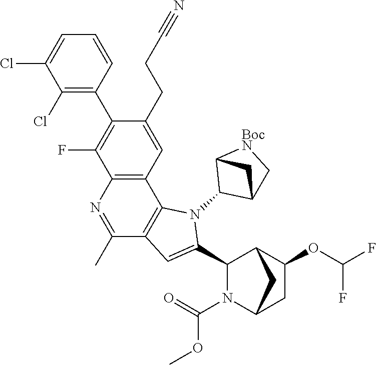 [SMILES: COC(=O)N1[C@@H]2C[C@@H]([C@@H](OC(F)F)C2)[C@@H]1c1cc2c(C)nc3c(F)c(-c4cccc(Cl)c4Cl)c(CCC#N)cc3c2n1[C@H]1[C@@H]2C[C@H]1N(C(=O)OC(C)(C)C)C2]

A mixture of Intermediate 2 (0.500 g, 0.734 mmol), methyl (1R,3R,4R,5S)-5-(difluoromethoxy)-3-ethynyl-2-azabicyclo[2.2.1]heptane-2-carboxylate (0.270 g, 1.10 mmol), copper(I) iodide (0.056 g, 0.294 mmol), tetrakis(triphenylphosphine)palladium(0) (0.170 g, 0.147 mmol) and N,N-diisopropylethylamine (1.3 mL, 7.34 mmol) in DMF (4.6 mL) was sparged with N₂ and heated at 70° C. for 1 h. Then, cesium carbonate (0.717 g, 2.20 mmol) was added to the reaction mixture. The resulting slurry was stirred at 90° C. for another 18 h. Upon completion, the mixture was cooled down to room temperature and poured into water. The solution was extracted with ethyl acetate twice. Then the combined organic layers were washed with brine five times, dried over Na₂SO₄, filtered and concentrated. The crude product was purified by flash chromatography (0-100% EtOAc/hexanes) to afford the title compound. LC-MS calc. for C₄₀H₄₁Cl₂F₃N₅O₅(M+H)⁺: m/z=798.2; found 798.3.

### Step 4. Methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate

To a solution of tert-butyl (1R,4R,5S)-5-((Rₐ)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate (0.350 g, 0.439 mmol) in DCM (7 mL), was added acetonitrile (0.7 mL) and TFA (7 mL). The reaction was stirred at room temperature for 0.5 h. Upon completion, volatiles were removed under reduced pressure and the residue was dissolved in acetonitrile (4 mL) and water (1 mL) and purified by preparative LC-MS (XBridge® C18 column, eluting with a gradient of acetonitrile/water containing 0.1% TFA, at flow rate of 60 mL/min) to afford the title compound. LC-MS calc. for C₃₅H₃₃Cl₂F₃N₅O₃(M+H)⁺: m/z=698.2; found 698.2. ¹H NMR was collected on the TFA salt. ¹H NMR (500 MHz, DMSO-d₆) δ 9.53 (s, 1H), 8.24 (s, 1H), 8.19 (s, 1H), 7.86 (dd, J=8.1, 1.5 Hz, 1H), 7.59 (t, J=7.9 Hz, 1H), 7.47 (td, J=7.8, 1.6 Hz, 1H), 6.97 (s, 1H), 6.83 (t, J=75 Hz, 1H), 5.73 (s, 1H), 4.96 (m, 1H), 4.88 (m, 1H), 4.59 (s, 1H), 4.32 (s, 1H), 3.92 (m, 1H), 3.74 (s, 3H), 3.53 (m, 1H), 3.44 (m, 1H), 3.12-2.99 (m, 1H), 2.97-2.78 (m, 5H), 2.76-2.62 (m, 2H), 2.39-2.32 (m, 1H), 2.32-2.19 (m, 1H), 1.76-1.59 (m, 3H), 1.57-1.47 (m, 1H).

The alternative atropisomer methyl (1R,3R,4R,5S)-3-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate can be prepared by an analogous route by performing processes analogous to the steps above starting from tert-butyl (1R,4R,5S)-5-((Sₐ)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((Rₐ)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate.

### Intermediate 1. Ethyl (R)-4-chloro-6-(2-cyanoethyl)-7-(2,3-dichlorophenyl)-8-fluoro-2-methylquinoline-3-carboxylate

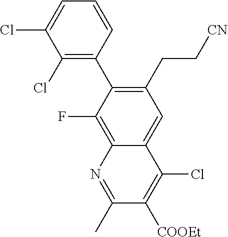 [SMILES: CCOC(=O)c1c(C)nc2c(F)c(-c3cccc(Cl)c3Cl)c(CCC#N)cc2c1Cl]

### Step 1. 3-Amino-2′,3′-dichloro-2-fluoro-[1,1′-biphenyl]-4-carboxylic acid

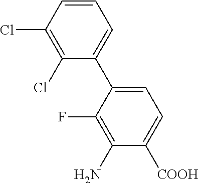 [SMILES: Nc1c(C(=O)O)ccc(-c2cccc(Cl)c2Cl)c1F]

A mixture of 2-amino-4-bromo-3-fluorobenzoic acid (28.0 g, 120 mmol), (2,3-dichlorophenyl)boronic acid (25.1 g, 132 mmol), bis(di-tert-butyl(4-dimethylaminophenyl)phosphine)dichloropalladium(II) (2.12 g, 3.00 mmol) and potassium phosphate (50.8 g, 239 mmol) in 1,4-dioxane (170 mL) and water (30 mL) was sparged with N₂ and heated at 70° C. for 1 h. Once completed, the reaction mixture was cooled down to r.t. and poured into 1 N HCl (200 mL). The mixture was stirred for another 10 min., resulting in precipitation. The solids were collected on a fritted filter, washed with water followed by hexanes and dried under reduced pressure to afford the sub-title compound in near quantitative yield. The crude product was used in next step without further purification. LC-MS calc. for C₁₃H₉Cl₂FNO₂ (M+H)⁺: m/z=300.0; found 300.0.

### Step 2. 3-Amino-6-bromo-2′,3′-dichloro-2-fluoro-[1,1′-biphenyl]-4-carboxylic acid

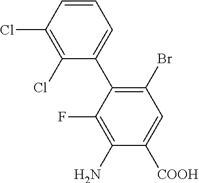 [SMILES: Nc1c(C(=O)O)cc(Br)c(-c2cccc(Cl)c2Cl)c1F]

To a solution of 3-amino-2′,3′-dichloro-2-fluoro-[1,1′-biphenyl]-4-carboxylic acid (35.8 g, 119 mmol) in DMSO (100 mL) was added N-bromosuccinimide (22.3 g, 125 mmol). The resulting mixture was heated at 50° C. for 1 h. Once completed, the reaction mixture was cooled down to r.t. and poured into ice water (400 mL). To the suspension, was added 20 mL saturated Na₂S₂O₃ solution. After stirring for 15 min., the solids were collected on a fritted filter, washed with water followed by hexanes and dried under reduced pressure to afford the sub-title compound (43.0 g, 95% yield). The crude product was used in next step without further purification. LC-MS calc. for C₁₃H₈BrCl₂FNO₂ (M+H)⁺: m/z=377.9, 379.9; found 378.0, 380.0.

### Step 3. 6-Bromo-7-(2,3-dichlorophenyl)-8-fluoro-2H-benzo[d][1,3]oxazine-2,4(1H)-dione

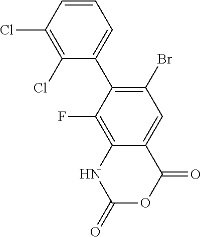 [SMILES: O=c1[nH]c2c(F)c(-c3cccc(Cl)c3Cl)c(Br)cc2c(=O)o1]

To a solution of 3-amino-6-bromo-2′,3′-dichloro-2-fluoro-[1,1′-biphenyl]-4-carboxylic acid (38.6 g, 102 mmol) in THF (300 mL) was added triphosgene (10.6 g, 35.6 mmol) portionwise. After addition, the mixture was heated at 60° C. for 0.5 h. Once completed, the reaction mixture was cooled down to r.t. and poured into heptane (1000 mL). After stirring for 1 h, the solids were collected on a fritted filter, washed with hexanes and dried under reduced pressure to afford the sub-title compound in near quantitative yield. The crude product was used in next step without further purification.

### Step 4. Ethyl 6-bromo-7-(2,3-dichlorophenyl)-8-fluoro-4-hydroxy-2-methylquinoline-3-carboxylate

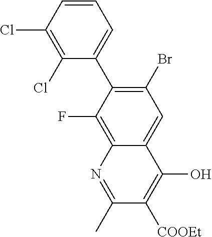 [SMILES: CCOC(=O)c1c(C)nc2c(F)c(-c3cccc(Cl)c3Cl)c(Br)cc2c1O]

To a solution of 6-bromo-7-(2,3-dichlorophenyl)-8-fluoro-2H-benzo[d][1,3]oxazine-2,4(1H)-dione (41.5 g, 102 mmol) in DMSO (200 mL), was added (1-ethoxy-1,3-dioxobutan-2-yl)sodium (18.7 g, 123 mmol) portionwise. After addition, the mixture was heated at 80° C. for 1 h. Once completed, the reaction mixture was cooled down to r.t. and poured into 1 N HCl (400 mL). After stirring for 1 h, the solids were collected on a fritted filter, washed with water followed by hexanes and dried under reduced pressure to afford the sub-title compound (40.0 g, 83% yield). The crude product was used in next step without further purification. LC-MS calc. for C₁₉H₁₄BrCl₂FNO₃ (M+H)⁺: m/z=471.9, 473.9; found 471.9, 474.0.

### Step 5. Ethyl (E)-6-(2-cyanovinyl)-7-(2,3-dichlorophenyl)-8-fluoro-4-hydroxy-2-methylquinoline-3-carboxylate

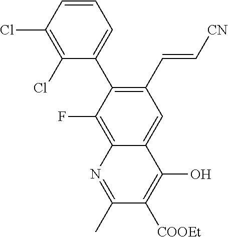 [SMILES: CCOC(=O)c1c(C)nc2c(F)c(-c3cccc(Cl)c3Cl)c(/C=C/C#N)cc2c1O]

To a solution of ethyl 6-bromo-7-(2,3-dichlorophenyl)-8-fluoro-4-hydroxy-2-methylquinoline-3-carboxylate (35.0 g, 74.0 mmol), bis(di-tert-butyl(4-dimethylaminophenyl)phosphine)dichloropalladium(II) (2.62 g, 3.70 mmol) in DMF (100 mL), acrylonitrile (12.3 mL, 185 mmol) and NEt₃ (30.9 mL, 222 mmol) were added. The mixture was sparged with N₂ and heated at 85° C. for 1 h. Once completed, the reaction mixture was cooled down to r.t. and poured into 1 N HCl (500 mL). After stirring for 1 h, the solids were collected on a fritted filter, washed with water followed by hexanes and dried under reduced pressure to afford the sub-title compound (19.2 g, 58% yield). The crude product was used in next step without further purification. LC-MS calc. for C₂₂H₁₆Cl₂FN₂O₃(M+H)⁺: m/z=445.0; found 445.0.

### Step 6. Ethyl (E)-4-chloro-6-(2-cyanovinyl)-7-(2,3-dichlorophenyl)-8-fluoro-2-methylquinoline-3-carboxylate

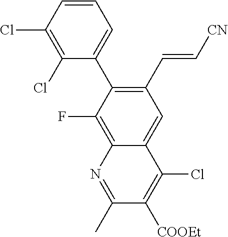 [SMILES: CCOC(=O)c1c(C)nc2c(F)c(-c3cccc(Cl)c3Cl)c(/C=C/C#N)cc2c1Cl]

To a slurry of ethyl (E)-6-(2-cyanovinyl)-7-(2,3-dichlorophenyl)-8-fluoro-4-hydroxy-2-methylquinoline-3-carboxylate (30.0 g, 67.4 mmol) and benzyltriethylammonium chloride (15.4 g, 67.4 mmol) in MeCN (150 mL) at 0° C., was added DIPEA (23.5 mL, 135 mmol). Upon stirring at 0° C., phosphoryl chloride (12.6 mL, 135 mmol) was added dropwise into the mixture. Then the mixture was heated at 60° C. for 1 h. Upon completion, the reaction mixture was cooled to r.t. and slowly poured into ice water (1000 mL). The mixture was extracted with DCM three times, dried over Na₂SO₄, filtered and concentrated. The crude product was further purified by FCC (0-50% EtOAc/hexanes) to afford the sub-title compound (4.5 g, 14% yield). LC-MS calc. for C₂₂H₁₅Cl₃FN₂O₂(M+H)⁺: m/z=463.0; found 463.0.

### Step 7. Ethyl (R)-4-chloro-6-(2-cyanoethyl)-7-(2,3-dichlorophenyl)-8-fluoro-2-methylquinoline-3-carboxylate

A mixture of copper(II) acetate monohydrate (0.19 g, 0.97 mmol) and Xantphos (0.56 g, 0.97 mmol) was stirred in toluene (1 mL) and tert-butanol (9 mL) at 60° C. for 0.5 h to afford a homogeneous solution. In a separate vial, to a mixture of ethyl (E)-4-chloro-6-(2-cyanovinyl)-7-(2,3-dichlorophenyl)-8-fluoro-2-methylquinoline-3-carboxylate (4.5 g, 9.70 mmol) and polymethylhydrosiloxane (3.5 g, 58.2 mmol) in toluene (12 mL) at 60° C., was added the previous copper-containing solution. The mixture was stirred at 60° C. for 0.5 h. Upon completion, the reaction mixture was filtered through diatomaceous earth and concentrated. The crude product was purified using FCC (0-40% EtOAc/DCM) to afford a mixture of two atropisomers (2.0 g, 44% yield). The title compound was separated from its atropisomer using chiral supercritical fluid chromatography (ChiralPak IJ column, eluting with 40% MeOH in CO₂ at a flow rate of 70 mL/min; the title compound eluted after its atropisomer). LC-MS calc. for C₂₂H₁₇Cl₃FN₂O₂(M+H)⁺: m/z=465.0; found 465.0.

### Intermediate 2. tert-Butyl (1R,4R,5S)-5-(((R)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate

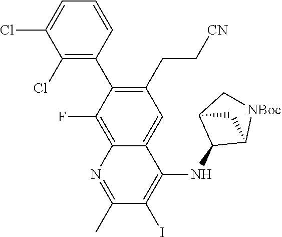 [SMILES: Cc1nc2c(F)c(-c3cccc(Cl)c3Cl)c(CCC#N)cc2c(N[C@H]2[C@@H]3C[C@H]2N(C(=O)OC(C)(C)C)C3)c1I]

### Step 1. tert-Butyl (1R,4R,5S)-5-(((R)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate

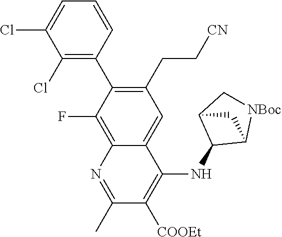 [SMILES: CCOC(=O)c1c(C)nc2c(F)c(-c3cccc(Cl)c3Cl)c(CCC#N)cc2c1N[C@H]1[C@@H]2C[C@H]1N(C(=O)OC(C)(C)C)C2]

To a solution of ethyl (R)-4-chloro-6-(2-cyanoethyl)-7-(2,3-dichlorophenyl)-8-fluoro-2-methylquinoline-3-carboxylate (intermediate 1, 7.2 g, 15.5 mmol) in N-methyl-2-pyrrolidone (21 mL), was added tert-butyl (1R,4R,5S)-5-amino-2-azabicyclo[2.1.1]hexane-2-carboxylate (5.52 g, 27.8 mmol) and DIPEA (8.1 mL, 46.4 mmol). The resulting mixture was heated at 80° C. for 18 h. Once completed, the reaction mixture was cooled down to r.t. and poured into 1 N HCl (300 mL) and ice mixture. After stirring for 0.5 h, the solids were collected on a fritted filter, washed with water followed by hexanes and dried under reduced pressure to afford white solids (8.2 g, 85% yield). The crude product was used in next step without further purification. LC-MS calc. for C₃₂H₃₄Cl₂FN₄O₄(M+H)⁺: m/z=627.2; found 627.1.

### Step 2. (R)-4-(((1R,4R,5S)-2-(tert-Butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid

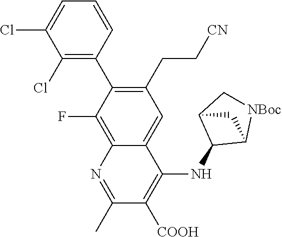 [SMILES: Cc1nc2c(F)c(-c3cccc(Cl)c3Cl)c(CCC#N)cc2c(N[C@H]2[C@@H]3C[C@H]2N(C(=O)OC(C)(C)C)C3)c1C(=O)O]

To a solution of tert-butyl (1R,4R,5S)-5-(((R)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (4.0 g, 6.37 mmol) in MeCN (13 mL), was added 1 N NaOH (16 mL, 15.94 mmol). The mixture was heated at 50° C. for 2 h. Once completed, the reaction mixture was cooled down to r.t. and acidified to pH 5 using 1 N HCl. The organic volatiles were removed under reduced pressure. The residue aqueous phase was extracted with EtOAc three times. The combined organic layers were dried over Na₂SO₄, filtered and concentrated under reduced pressure to give a yellow solid (3.70 g, 97% yield). The crude material was used in the next step without further purification. LC-MS calc. for C₃₀H₃₀Cl₂FN₄O₄(M+H)⁺: m/z=599.2; found 599.1.

### Step 3. tert-Butyl (1R,4R,5S)-5-(((R)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate

To a solution of (R)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid (3.70 g, 6.17 mmol) in MeCN (6.2 mL), was added potassium phosphate (2.62 g, 12.34 mmol) and N-iodosuccinimide (2.50 g, 11.1 mmol). The mixture was stirred at r.t. for 1 h. Once completed, the reaction mixture was poured into saturated Na₂S₂O₃ solution. After stirring for 10 min., the mixture was extracted with EtOAc three times. The combined organic layers were dried over Na₂SO₄, filtered and concentrated under reduced pressure. The crude product was further purified with FCC (0-100% EtOAc/hexanes) to afford the title compound as an off-white solid (1.95 g, 46% yield). LC-MS calc. for C₂₉H₂₉Cl₂FIN₄O₂ (M+H)⁺: m/z=681.1; found 681.0.

### Intermediate 3. Methyl (1R,3R,4S)-2-((S)-1-phenylethyl)-2-azabicyclo[2.2.1]hept-5-ene-3-carboxylate

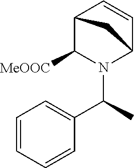 [SMILES: COC(=O)[C@H]1[C@@H]2C=C[C@@H](C2)N1[C@@H](C)c1ccccc1]

### Step 1. Methyl 2-hydroxy-2-methoxyacetate

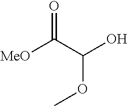

A solution of glyoxylic acid monohydrate (41.4 g, 450 mmol) in anhydrous MeOH (200 mL) was heated to 70° C. overnight. After cooling to r.t., the mixture was stirred with solid NaHCO₃ for 10 min. The resulting mixture was filtered and concentrated under reduced pressure to afford an oily residue. The residue was dissolved in CH₂Cl₂, dried over Na₂SO₄, filtered and concentrated to afford the product (40.0 g, 82% yield). The product was used in next step without further purification.

### Step 2. Methyl (S,E)-2-((1-phenylethyl)imino)acetate

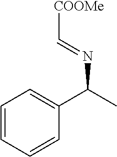

To a solution of methyl 2-hydroxy-2-methoxyacetate (40.0 g, 333 mmol) in toluene (95 mL) was added (S)-1-phenylethan-1-amine (40.4 g, 333 mmol) slowly. The mixture was stirred for 1 h at r.t. and diluted with EtOAc. The organic layer was washed with brine, dried over Na₂SO₄, filtered and concentrated under reduced pressure to a yellow oil. The crude product was used in the next step without further purification.

### Step 3. Methyl (1R,3R,4S)-2-((S)-1-phenylethyl)-2-azabicyclo[2.2.1]hept-5-ene-3-carboxylate

To a solution of methyl (S,E)-2-((1-phenylethyl)imino)acetate (63.7 g, 333 mmol) in 2,2,2-trifluoroethanol (800 mL) at −10° C., was added TFA (25.5 mL, 333 mmol). The reaction mixture was allowed to stir at −10° C. for 1 h before cyclopentadiene (24.2 g, 366 mmol) was added slowly. The mixture was stirred at −10° C. for another 0.5 h and then allowed to warm up to r.t. After removal of volatiles, the residue was diluted with 2 N hydrochloric acid (500 mL) and washed with diethyl ether. The organic layer was extracted with 2 N hydrochloric acid (100 mL). The combined aqueous layer was neutralized with 28% ammonium hydroxide and extracted with EtOAc three times. The combined organic layers was dried over Na₂SO₄, filtered and concentrated. The crude product was purified by FCC (0-10% EtOAc/hexanes) in batches to afford the title compound as a colorless solid. LC-MS calc. for C₁₆H₂₀NO₂ (M+H)*: m/z=258.1; found 258.2. ¹H NMR (500 MHz, CDCl₃) δ 7.32-7.27 (m, 2H), 7.25 (m, 2H), 7.22-7.16 (m, 1H), 6.44 (ddd, J=5.7, 3.1, 1.2 Hz, 1H), 6.29 (dd, J=5.7, 2.0 Hz, 1H), 4.33 (h, J=1.5 Hz, 1H), 3.37 (s, 3H), 3.06 (q, J=6.5 Hz, 1H), 2.93 (dq, J=3.3, 1.6 Hz, 1H), 2.24 (d, J=0.9 Hz, 1H), 2.13 (dt, J=8.4, 1.7 Hz, 1H), 1.48-1.41 (m, 4H).

### Intermediate 4. 2-(tert-butyl) 3-methyl (1R,3R,4R,5S)-5-hydroxy-2-azabicyclo[2.2.1]heptane-2,3-dicarboxylate

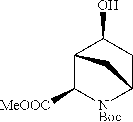 [SMILES: COC(=O)[C@H]1[C@H]2C[C@H](C[C@@H]2O)N1C(=O)OC(C)(C)C]

### Step 1. Methyl (1R,3R,4R,5S)-5-hydroxy-2-((S)-1-phenylethyl)-2-azabicyclo[2.2.1]heptane-3-carboxylate

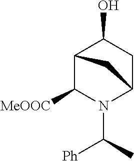 [SMILES: COC(=O)[C@H]1[C@H]2C[C@H](C[C@@H]2O)N1[C@@H](C)c1ccccc1]

To a solution of methyl (1R,3R,4S)-2-((S)-1-phenylethyl)-2-azabicyclo[2.2.1]hept-5-ene-3-carboxylate (intermediate 3, 5.3 g, 20.6 mmol) in THF (70 mL) at 0° C., was added a 0.5 N THF solution of 9-borabicyclo[3.3.1]nonane (51.5 mL, 25.7 mmol). The reaction mixture was allowed to warm up to r.t. and stirred for 18 h. Then the reaction mixture was cooled to 0° C. and a 2 N NaOH solution (36.0 mL, 72.1 mmol) was added followed by hydrogen peroxide (30% aqueous solution, 10.5 mL, 103 mmol). The reaction mixture was allowed to warm up to r.t. and stirred for 1 h. The reaction mixture was diluted with EtOAc, washed with brine, dried over Na₂SO₄, filtered and concentrated. The crude product was purified by FCC (50%-70% EtOAc/hexanes) to afford the sub-title compound (2.0 g, 38% yield). LC-MS calc. for C₁₆H₂₂NO₃ (M+H)⁺: m/z=276.2; found 276.2.

### Step 2. Methyl (1R,3R,4R,5S)-5-hydroxy-2-azabicyclo[2.2.1]heptane-3-carboxylate

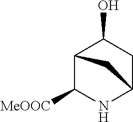 [SMILES: COC(=O)[C@@H]1N[C@@H]2C[C@H]1[C@@H](O)C2]

To a solution of methyl (1R,3R,4R,5S)-5-hydroxy-2-((S)-1-phenylethyl)-2-azabicyclo[2.2.1]heptane-3-carboxylate (2.00 g, 7.26 mmol) in EtOH (35 mL) was added 20% Pd(OH)₂/C (0.58 g). The mixture was stirred under an atmosphere of H₂ for 18 h. The resulting mixture was filtered through diatomaceous earth and concentrated to afford the sub-title compound. The crude material was used for next step without further purification. LC-MS calc. for C₈H₁₄NO₃ (M+H)⁺: m/z=172.1; found 172.1.

### Step 3. 2-(tert-Butyl) 3-methyl (1R,3R,4R,5S)-5-hydroxy-2-azabicyclo[2.2.1]heptane-2,3-dicarboxylate

To methyl (1R,3R,4R,5S)-5-hydroxy-2-azabicyclo[2.2.1]heptane-3-carboxylate (1.24 g, 7.26 mmol) dissolved in THF (10 mL), was added DIPEA (5.10 mL, 29.1 mmol) and Boc₂O (3.96 g, 18.2 mmol). The reaction mixture was stirred at r.t. for 0.5 h and diluted with EtOAc. After washing with 0.01 N HCl and brine, the organic fraction was dried over Na₂SO₄, filtered and concentrated. The crude product was further purified by FCC (0%-100% EtOAc/hexanes) to afford the title compound (1.88 g, 95% yield). LC-MS calc. for C₉H₁₄NO₅ (M-^{t}Bu+2H)⁺: m/z=216.1; found 216.1.

### Intermediate 5. tert-butyl (1R,3R,4R,5S)-5-((tert-butyldiphenylsilyl)oxy)-3-ethynyl-2-azabicyclo[2.2.1]heptane-2-carboxylate

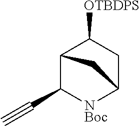

### Step 1. 2-(tert-Butyl) 3-methyl (1R,3R,4R,5S)-5-((tert-butyldiphenylsilyl)oxy)-2-azabicyclo[2.2.1]heptane-2,3-dicarboxylate

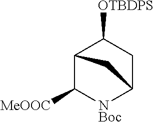

To a solution of 2-(tert-butyl) 3-methyl (1R,3R,4R,5S)-5-hydroxy-2-azabicyclo[2.2.1]heptane-2,3-dicarboxylate (intermediate 4, 1.88 g, 6.92 mmol) in DMF (140 mL), was added tert-butylchlorodiphenylsilane (2.08 g, 7.64 mmol) and imidazole (1.40 g, 20.8 mmol). The reaction mixture was stirred at r.t. for 18 h. The mixture was diluted with EtOAc, washed with brine five times, dried over Na₂SO₄, filtered and concentrated. The crude product was purified by FCC (0%-40% EtOAc/hexanes) to afford the sub-title compound. LC-MS calc. for C₂₅H₃₂NO₅Si (M-^{t}Bu+2H)⁺: m/z=454.2; found 454.2.

### Step 2. tert-Butyl (1R,3R,4R,5S)-5-((tert-butyldiphenylsilyl)oxy)-3-(hydroxymethyl)-2-azabicyclo[2.2.1]heptane-2-carboxylate

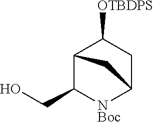

To a solution of 2-(tert-butyl) 3-methyl (1R,3R,4R,5S)-5-((tert-butyldiphenylsilyl)oxy)-2-azabicyclo[2.2.1]heptane-2,3-dicarboxylate (1.53 g, 3.01 mmol) in THF (15 mL), was added a 2 N THF solution of LiBH₄ (3.8 mL, 7.52 mmol). The mixture was stirred at r.t. for 8 h and then quenched by slow addition of a saturated NH₄Cl solution. The mixture was diluted with EtOAc, washed with brine, dried over Na₂SO₄, filtered and concentrated. The crude product was purified by FCC (0-60% EtOAc/hexanes) to afford the sub-title compound (1.39 g, 96% yield). LC-MS calc. for C₂₄H₃₂NO₄Si (M-^{t}Bu+2H)⁺: m/z=426.2; found 426.2.

### Step 3. tert-Butyl (1R,3R,4R,5S)-5-((tert-butyldiphenylsilyl)oxy)-3-formyl-2-azabicyclo[2.2.1]heptane-2-carboxylate

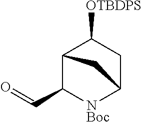

To a solution of oxalyl chloride (0.73 g, 5.76 mmol) in DCM (5.3 mL) cooled to −78° C., was added DMSO (0.61 mL, 8.64 mmol) slowly. After stirring for 10 min., a DCM (1 mL) solution of tert-butyl (1R,3R,4R,5S)-5-((tert-butyldiphenylsilyl)oxy)-3-(hydroxymethyl)-2-azabicyclo[2.2.1]heptane-2-carboxylate (1.39 g, 2.88 mmol) was added. The reaction mixture was stirred at −78° C. for 1 h before DIPEA (1.5 mL) was added. The reaction mixture was allowed to warm up to r.t. and stirred for another 0.5 h. Then the reaction mixture was poured into a DCM (15 mL)/28% ammonium hydroxide (1.5 mL) mixture. After stirring for 10 min., the mixture was diluted with water. The organic phase was separated, dried over Na₂SO₄, filtered and concentrated to give crude product, which was used in the next step without further purification. LC-MS calc. for C₂₄H₃₀NO₄Si (M-^{t}Bu+2H)⁺: m/z=424.2; found 424.3.

### Step 4. tert-Butyl (1R,3R,4R,5S)-5-((tert-butyldiphenylsilyl)oxy)-3-ethynyl-2-azabicyclo[2.2.1]heptane-2-carboxylate

To a solution of tert-butyl (1R,3R,4R,5S)-5-((tert-butyldiphenylsilyl)oxy)-3-formyl-2-azabicyclo[2.2.1]heptane-2-carboxylate (1.38 g, 2.88 mmol) in MeOH (15 mL) was added dimethyl(1-diazo-2-oxopropyl)phosphonate (0.61 g, 3.17 mmol) and K₂CO₃ (1.19 g, 8.64 mmol). After stirring for 18 h, the reaction mixture was filtered through diatomaceous earth. The filtrate was concentrated. The residue was extracted with EtOAc, filtered through diatomaceous earth and concentrated. The crude product was purified by FCC (0-40% EtOAc/hexanes) to afford the title compound (0.20 g, 51% over 2 steps). LC-MS calc. for C₂₅H₃₀NO₃Si (M-^{t}Bu+2H)⁺: m/z=420.2; found 420.2.

### Example 2. Synthesis of cyclopropyl((1R,3R,5R)-3-ethynyl-2-azabicyclo[3.1.0]hexan-2-yl)methanone (step 17a in Example 1)

### Step 1. 1-(tert-Butyl) 2-ethyl (R)-2,3-dihydro-1H-pyrrole-1,2-dicarboxylate

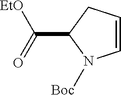 [SMILES: CCOC(=O)[C@H]1CC=CN1C(=O)OC(C)(C)C]

To a solution of 1-(tert-butyl) 2-ethyl (R)-5-oxopyrrolidine-1,2-dicarboxylate (241 g, 0.938 mol) in anhydrous toluene (1.6 L) was added 1 M lithium triethyl borohydride in tetrahydrofuran (1.01 L, 1.01 mol) dropwise at −50-−40° C. over 1 h. After addition, the mixture was stirred for 1 h at about −50° C. DIPEA (726 mL, 4.17 mol) was added to the mixture dropwise over 1 h. 4-Dimethylaminopyridine (1.49 g, 12.2 mmol, 0.013 eq.) was added to the mixture, followed by the dropwise addition of trifluoroacetic anhydride (156.5 mL, 1.126 mol) over 1.5 h. After addition, the mixture was stirred for 1 h at about −50° C., then slowly warmed to r.t. The mixture was stirred for 1 h at r.t. The reaction mixture was cooled to 0° C. and diluted slowly with water (2.41 L), while maintaining the temperature below 10° C. during addition. The organic layer was separated and washed with water (2.41 L) and saturated brine (720 mL). The organic layer was dried over sodium sulfate (120 g). The solution was concentrated under reduced pressure to give desired product (230 g, quant.) as yellow oil. GCMS calc. for C₁₂H₁₉NO₄: 241.1; Found: 214.2 (M⁺). ¹H-NMR (400 MHz, CDCl₃) δ 6.70-6.48 (m, 1H), 4.99-4.86 (m, 1H), 4.70-4.52 (m, 1H), 4.30-4.11 (m, 2H), 3.15-2.98 (m, 1H), 2.73-2.57 (m, 1H), 1.53-1.38 (m, 9H), 1.34-1.21 (m, 4H).

### Step 2. 2-(tert-Butyl) 3-ethyl (1R,3R,5R)-2-azabicyclo[3.1.0]hexane-2,3-dicarboxylate

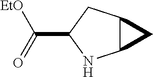

To a solution 1-(tert-butyl) 2-ethyl (R)-2,3-dihydro-1H-pyrrole-1,2-dicarboxylate (230 g, 0.938 mol) in toluene (2.3 L) was added 1.1 M diethylzinc in toluene (1.7 L, 1.87 mol) at −30 to −25° C. over 1 h. Chloroiodomethane (273 mL, 3.752 mol) was added to the mixture dropwise over 2 h at about −30 to −20° C. and the mixture was stirred for 16 h. Half-saturated sodium bicarbonate (2.3 L) was added to the mixture and the mixture was warmed up to r.t. The mixture was filtered over diatomaceous earth to remove white solids and the filter bed was rinsed with toluene (1.5 L). The organic layer was separated from the filtrate and washed with water (2×1.15 L) and saturated brine (1.15 L). The toluene solution was concentrated under reduced pressure to give a 6 to 1 mixture (231 g) of 2-(tert-Butyl) 3-ethyl (1R,3R,5R)-2-azabicyclo[3.1.0]hexane-2,3-dicarboxylate and 2-(tert-butyl) 3-ethyl (1 S,3R,5S)-2-azabicyclo[3.1.0]hexane-2,3-dicarboxylate as yellow oil as determined by GCMS analysis.

Aqueous methyl amine (40%, 344 g) was added to a crude mixture product obtained above (226 g) and the mixture was stirred for 16 h at r.t. Water (340 mL) and methyl tert-butyl ether (340 mL) was added to the mixture. The organic layer was separated and washed with water (340 mL) and saturated brine (230 mL). The solution was concentrated under reduced pressure to give 2-(tert-butyl) 3-ethyl (1R,3R,5R)-2-azabicyclo[3.1.0]hexane-2,3-dicarboxylate (177 g, 73% calc. yield) as yellow oil, which contained 2% 2-(tert-butyl) 3-ethyl (1 S,3R,5S)-2-azabicyclo[3.1.0]hexane-2,3-dicarboxylate as determined by GCMS analysis. GCMS calc. for C₁₃H₂₁NO₄: 255.1; Found: 255.1 (M⁺). ¹H-NMR (400 MHz, CDCl₃) δ 4.56-4.39 (m, 1H), 4.18-4.01 (m, 2H), 3.51-3.36 (m, 1H), 2.60-2.42 (m, 1H), 2.00-1.92 (m, 1H), 1.45-1.32 (m, 9H), 1.23-1.15 (m, 4H), 0.87-0.79 (m, 1H), 0.70-0.56 (m, 1H).

### Step 3. tert-Butyl (1R,3R,5R)-3-(hydroxymethyl)-2-azabicyclo[3.1.0]hexane-2-carboxylate

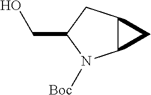

A solution of 2-(tert-butyl) 3-ethyl (1R,3R,5R)-2-azabicyclo[3.1.0]hexane-2,3-dicarboxylate (177 g, 0.694 mol) in tetrahydrofuran (1.56 L) was added to 1 M lithium aluminum hydride solution in tetrahydrofuran (777 mL, 0.777 mol, 1.12 eq.) at about 0-10° C. over 1 h. After addition, the mixture was stirred for 2 h at 3° C. Water (27 mL) was added to the mixture dropwise to quench the reaction. Sodium hydroxide solution (15%, 27 mL) and water (80 mL) were sequentially added to the mixture dropwise. The mixture was stirred at r.t. for 1 h. DCM (2.35 L) was added to the mixture. The suspension was filtered through diatomaceous earth (100 g) bed and rinsed with DCM (300 mL). The filtrate was concentrated under reduced pressure and dried under vacuum oven at 40° C. for 18 h to give tert-butyl (1R,3R,5R)-3-(hydroxymethyl)-2-azabicyclo[3.1.0]hexane-2-carboxylate (133 g, 90% yield) as yellow oil which contained 2% of an isomer as determined by GCMS analysis. GCMS calc. for C₁₁H₁₉NO₃: 213.1; Found: 213.2 (M⁺). ¹H-NMR (400 MHz, CDCl₃) δ 4.83 (brs, 1H), 4.34 (brs, 1H), 2.45 (ddd, 1H), 1.55-1.43 (m, 12H), 0.80 (q, 1H), 0.40 (brs, 1H).

### Step 4. tert-Butyl (1R,3R,5R)-3-formyl-2-azabicyclo[3.1.0]hexane-2-carboxylate

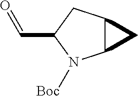

DMSO (42.7 mL, 0.603 mol) was added to oxalyl chloride (26.4 mL, 0.301 mol) in DCM (535 mL) dropwise at −78° C. over 30 min., while maintaining the temperature below −60° C. during addition. After stirring at −78° C. for 30 min. tert-butyl (1R,3R,5R)-3-(hydroxymethyl)-2-azabicyclo[3.1.0]hexane-2-carboxylate (53.5 g, 0.251 mol) in DCM (535 mL) was added to solution dropwise at −78° C. over 40 min. After stirring at −78° C. for 30 min., NEt₃ (104.9 mL, 0.753 mol) was added to solution dropwise at −78° C. over 40 min. After stirring at −78° C. for 1 h, the reaction mixture was warmed to 0° C. and stirred for 30 min. Water (888 mL) was added to the mixture and stirred for 20 min. The aqueous layer was separated and extracted with DCM (2×888 mL). The combined organic layers were sequentially washed with 1 M HCl (888 mL), water (888 mL) and saturated brine (888 mL). The organic layer was concentrated under reduced pressure to give tert-butyl (1R,3R,5R)-3-formyl-2-azabicyclo[3.1.0]hexane-2-carboxylate (44 g, 83% yield) as yellow oil. GCMS calc. for C₁₁H₁₇NO₃: 213.1; Found: 213.2 (M⁺). ¹H-NMR (400 MHz, CDCl₃) δ 9.54-9.31 (m, 1H), 4.64-4.39 (m, 1H), 3.68-3.45 (m, 1H), 2.68-2.33 (m, 1H), 2.24-2.10 (m, 1H), 1.53-1.41 (m, 10H), 0.88-0.71 (m, 1H), 0.39-0.28 (m, 1H).

### Step 5. tert-Butyl (1R,3R,5R)-3-ethynyl-2-azabicyclo[3.1.0]hexane-2-carboxylate

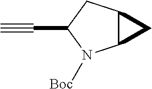

K₂CO₃ (28.8 g, 0.209 mol, 2 eq.) was added to a solution of tert-butyl (1R,3R,5R)-3-formyl-2-azabicyclo[3.1.0]hexane-2-carboxylate (22 g, 0.104 mol) in methanol (352 mL) at 0-5° C. Dimethyl (1-diazo-2-oxopropyl)phosphonate (18.3 mL, 0.110 mol) was added to the mixture dropwise at 0-5° C. for 30 min., while maintaining the temperature at <5° C. during addition. After stirring at 0-5° C. for 15 min., the reaction mixture was warmed up to r.t. and stirred for 2 h. Water (372 mL) and EtOAc (930 mL) was added to the mixture, which was stirred for 15 min. The aqueous layer was separated and extracted with EtOAc (372 mL). The combined organic layers were washed with water (560 mL) and saturated brine (560 mL). The organic solution was concentrated under reduced pressure and purified over silica gel and eluted with a gradient of 0-10% EtOAc in heptane to give a 7 to 1 mixture of tert-butyl (1R,3R,5R)-3-ethynyl-2-azabicyclo[3.1.0]hexane-2-carboxylate and tert-butyl (1R,3S,5R)-3-ethynyl-2-azabicyclo[3.1.0]hexane-2-carboxylate (82 g, 74% calc. yield) as light yellow oil. GCMS calc. for C₁₂H₁₇NO₂: 207.1; Found: 207.0 (M⁺). ¹H-NMR (400 MHz, CDCl₃) δ 4.78-4.54 (m, 1H), 3.60-3.46 (m, 1H), 2.52-2.40 (m, 1H), 2.30-2.22 (m, 1H), 2.18-2.08 (m, 1H), 1.50-1.48 (m, 9H), 1.16-1.05 (m, 1H), 0.91-0.80 (m, 1H), 0.78-0.66 (m, 1H).

### Step 6. Cyclopropyl((1R,3R,5R)-3-ethynyl-2-azabicyclo[3.1.0]hexan-2-yl)methanone

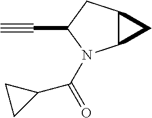

A mixture of tert-butyl (1R,3R,5R)-3-ethynyl-2-azabicyclo[3.1.0]hexane-2-carboxylate and tert-butyl (1R,3S,5R)-3-ethynyl-2-azabicyclo[3.1.0]hexane-2-carboxylate (82 g, 0.39 mol) and 4M HCl in dioxane (297 mL, 1.19 mol, 3 eq.) was stirred at rt for 4 h. The reaction mixture was diluted with THF (1.23 L) and cooled to 0° C. NEt₃ (275.8 mL, 1.98 mol) was added to the reaction at 0° C. dropwise over 1.5 h while maintaining the temperature at <10° C. during addition. Cyclopropanecarbonyl chloride (45,4 g, 0.43 mol) was added to the reaction at 0° C. The reaction was warmed to r.t. and stirred for 3 h. 1 M HCl (410 mL, 5 vol) and DCM (820 mL) was added. The aqueous layer was separated and extracted with DCM (2×820 mL). The combined organic layers were washed with water (820 mL) and saturated brine (820 mL). The organic layer was concentrated under reduced pressure to give a crude residue (60 g). Diatomaceous earth (120 g) was added to the crude residue and the mixture was dried under reduced pressure to give a dried load powder (186 g). The dried load powder was purified on a silica gel column (1.5 kg) and eluted with a gradient of 15 to 40% EtOAc in heptane. The desired fractions were concentrated under reduced pressure and dried under vacuum at 30° C. for 18 h to give the title compound (40.8 g, 59% yield) as brown oil. GCMS calc. for C₁₂H₁₇NO₂: 175.1; Found: 175.0 (M⁺). ¹H-NMR (400 MHz, DMSOd₆) δ 5.14 (dt, 0.45H), 4.81 (dt, 0.55H), 3.82 (t, 0.55H), 3.71 (t, 0.45H), 3.42 (d, 0.45H), 3.15 (d, 0.55H), 2.57 (ddd, 0.45H), 2.44 (ddd, 0.55H), 2.09 (dd, 0.45H), 2.04 (ddd, 0.55H), 1.97 (dd, 0.55H), 1.86-1.69 (m, 1H), 1.62 (dddd, 0.45H), 1.01 (td, 0.55H), 0.90 (td, 0.45H), 0.87-0.68 (m, 5H).

### Example 3. Synthesis of tert-Butyl (1R,4R,5S)-5-amino-2-azabicyclo[2.1.1]hexane-2-carboxylate oxalate (step 14a in Example 1)

### Step 1. (E)-4-Methoxybut-3-en-2-one

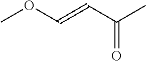

A mixture of 4,4-dimethoxy-2-butanone (350 g, 1.0 eq) and sodium acetate (11 g, 0.05 eq.) was heated to 145-150° C. under nitrogen atmosphere and the resulting methanol is purged during the heating process. When the reaction is complete, the mixture was cooled to 70-80° C. The product was distilled under vacuum to give desired product (130 g, yield 50%). 1H NMR (CD₂Cl₂/CHDOD, 400 MHz): δ 7.60 (d, 1H, J=12.8 Hz) 5.53 (d, 1H, J=12.8 Hz), 3.81 (s, 3H,), 2.17 (s, 3H). 13C NMR (CD₂Cl₂/CD₃OD, 100.6 MHz): δ 27.1, 58.0, 107.0, 165.2, 199.6.

### Step 2. (E)-4-(Allylamino)but-3-en-2-one

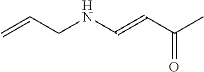

A mixture of (E)-4-methoxybut-3-en-2-one (150 g) and NEt₃ (182 g) in DCM (450 mL) was added was agitated under nitrogen at 10-15° C. Allylamine hydrochloride aqueous solution (60%, 234 g) is slowly added to the mixture at 10-15° C. After the addition, the mixture is agitated for 30 min. When the reaction was completed, water (150 g) was added to the reaction mixture. The organic phase was separated, and the water phase was extracted with DCM (300 mL). The combined organic phases were washed with brine (150 mL) and organic phase was concentrated under vacuum to give crude product as yellow oil (175 g, yield 93%). 1H NMR (500 MHz, CDCl₃): δ 9.75 (bs, 1H); 6.58 (dd, 1H, J=16.8, 2); 5.78-5.86 (m, 1H); 5.19 (d, 1H, J=16.8)); 5,14 (d, 1H, J=10, 1)); 5.00 (d, 1H, J=10, 1); 3.74-3.77 (m, 2H); 2.03, (s, 3H). 13C NMR (125 Hz, CDCl₃): 197.5; 153.2; 165.3; 117.6; 94.9; 51.1; 29.2.

### Step 3. tert-Butyl (E)-allyl(3-oxobut-1-en-1-yl)carbamate

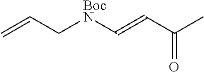 [SMILES: C=CCN(/C=C/C(C)=O)C(=O)OC(C)(C)C]

A mixture of (E)-4-(allylamino)but-3-en-2-one (130 g), trimethylamine (105 g), N,N-dimethylaminopyridine (13 g) in toluene (390 mL) was heated to 50-55° C. (Boc)₂O (259 g) was added in portion while maintained the reaction temperature between 50-55° C. After the reaction mixture was agitated for 2 h at 50-55° C. to complete the reaction. The mixture was cooled to 10-15° C. and 3 M aq. HCl was added to the mixture until the pH 5-6. The organic phase was separated, and the aqueous phase was extracted with toluene (260 mL). The combined organic phases were washed with water (260 mL). Activated charcoal (1 g) was added. The mixture was agitated at 50-55° C. for 1 h before cooling the mixture to 20-30° C. The mixture was filtered over diatomaceous earth bed and the diatomaceous earth bed was rinsed with toluene. The filtrated was concentrated to a residue and the residue was coevaporated with MeCN to give a residue as yellow oil (189 g, 80% yield). 1H NMR (500 MHz, CDCl₃): δ 8.11 (d, 1H, J=15); 5.68-5.73 (m, 1H); 5.49 (d, 1H, J=15); 5.14 (d, 1H, J=18); 5.09 (d, 1H, J=10); 4.13 (t, 2H); 2.20 (s, 3H); 1.50 (s, 9H). 13C NMR (125 MHz, CDCl3): 198.6; 153.0; 143.2; 131.8; 117.8; 109.5; 84.0; 47.0; 28.3; 28.1.

### Step 4. tert-Butyl 5-acetyl-2-azabicyclo[2.1.1]hexane-2-carboxylate

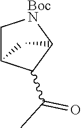

A solution of tert-butyl (E)-allyl(3-oxobut-1-en-1-yl)carbamate (270 g) in MeCN (3240 mL) was subjected to UV-photo reactor. When the reaction was complete, the yellow oil residue (major and minor isomer mixture) was used for next step without further purification. Sample was purified by column to get analytical data. 1H NMR (500 MHz, CDCl₃) δ 4.62-6.78 (bd, 1H); 3.40 (bt, 1H); 3.16 (bs, 1H); 3.06 (bs, 1H); 2.69 (s, 1H); 1.97 (s, 3H); 1.70-1.73 (m, 1H); 1.46 (s, 9H).

### Step 5. tert-Butyl (1R,4R,5S)-5-amino-2-azabicyclo[2.1.1]hexane-2-carboxylate oxalate

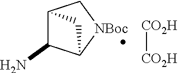 [SMILES: CC(C)(C)OC(=O)N1C[C@H]2C[C@@H]1[C@H]2N ; O=C(O)C(=O)O]

A mixture of tert-butyl 5-acetyl-2-azabicyclo[2.1.1]hexane-2-carboxylate (150 g) in MeCN (1500 mL) was added to sodium hypochlorite (173.5 g) in 30% sodium hydroxide solution at 30-40° C. (1500 mL). The mixture was agitated at 30-40° C. for 30 min. to complete the reaction. The mixture was cooled to 10-15° C. and 6M HCl aq. solution was added to adjust the mixture pH 8-9. The mixture was concentrated under vacuum to remove MeCN at 50-55° C. and methanol (90 mL) was added to the residue. The mixture was cooled to 10-15° C. and 6M HCl was added to adjust the mixture pH 2-3 (solids precipitated out as the pH adjustment) and agitated for additional 2-3 h. The solids were isolated and rinsed with water (300 mL). The wet solids were dried under vacuum at 50-55° C.

Recrystallization: A mixture of the solids in toluene (1500 mL) was heated to 60-70° C. to a solution. (R)-(+)-1-phenylethylamine (80.7 g) was added at 40-70° C. The solution was cooled to 30-35° C. over 90 min. (solids precipitated gradually) and agitated for 1 h. The suspension was cooled to 20-25° C. over 90 min. and agitated for 2 h. The solids were isolated and rinsed with toluene (40 mL). A mixture of the cake and toluene (1200 mL) was heated to 100-105° C. to a solution. The mixture was cooled to 75-85° C. over 90 min. (solids precipitated) and agitated for 1 h. The mixture was cooled to 20-25° C. over 2 h and agitated for 2 h. The solids were isolated and rinsed with toluene (40 mL). The recrystallization process was repeated one more time.

Free base: to a mixture of the wet cake in toluene (225 mL) and water (225 mL) was added 30% aq. NaOH at 10-15° C. to pH 9-10. The mixture was agitated for 30 min. and the organic phase was separated. To the aqueous phase was added 6 M aq. HCl at 10-15° C. to pH 2-3 (solids predicated). The mixture was then cooled to 3-8° C. and agitated for 1 h. The solids were isolated and washed with water (40 mL). The wet cake was dried under vacuum at 50-55° C. to give the desired (1R,4S,5S)-2-(tert-butoxycarbonyl)-2-azabicyclo[2.1.1]hexane-5-carboxylic acid (25 g, 18% yield).

A mixture of the acid (245 g), pyridine (86 g) and ammonium carbonate (111 g) in MeCN (3700 mL) was added (Boc)₂O (310 g) at 15-25° C. The mixture was agitated for 5 h to complete the reaction. The solids were isolated and rinsed with MeCN (250 mL). The filtrate and rinse were combined and concentrated under vacuum at 40-45° C. and azeotroped with heptane. To the residue was added EtOAc (130 mL) and n-heptane (650 mL) at 40-45° C. The mixture was cooled to 10-15° C. (solids precipitated) and agitated for 2 h. The solids were isolated and rinsed with n-heptane (250 mL). The wet cake was dried under vacuum at 50-55° C. to give desired product tert-butyl (1R,4S,5S)-5-carbamoyl-2-azabicyclo[2.1.1]hexane-2-carboxylate quantitatively.

To cooled 15% aq. NaOH (800 mL) at 10-15° C. was added the tert-butyl (1R,4S,5S)-5-carbamoyl-2-azabicyclo[2.1.1]hexane-2-carboxylate (214 g). Sodium hypochlorite (91.2 g) was added at 10-20° C. and the mixture was agitated for 2 h. The mixture was heated to 40-45° C. for 4 h to complete the reaction. The reaction mixture was cooled to 15-20° C. and citric acid was added to adjust pH 5-6. The mixture was basified by addition of sodium hydroxide to pH 14. The basified mixture was extracted with 2-methyltetrahydrofuran (2×1000 mL). The combined organic phase was concentrated under vacuum and the residual was azeotroped with MeCN. The residue was dissolved in (140 mL) and activated charcoal (2 gram) was added. The mixture was agitated at 25-30° C. for 2 h. The mixture was filtered, and the filter bed is rinsed with MeCN (85 mL). The combined filtrate and rinse were added to a solution of oxalic acid (120 g) in MeCN (850 mL) at 40-45° C. The solution was cooled to 3-7° C. and agitated for 1 h. The solids were isolated and rinsed with MeCN (110 mL). The wet cake was dried at 40-50° C. under vacuum to give desired tert-butyl (1R,4R,5S)-5-amino-2-azabicyclo[2.1.1]hexane-2-carboxylate oxalate (248 g, 91% yield) as white solids. HPLC-MS for calc. C₁₀H₁₈N₂O₂: 198.14; Found (M+H): 199.1 ¹H NMR (500 MHz, DMSO-d₆): δ8.44 (s, 3H); 3.34, (m, 1H); 4.24, dt, 1H, J=6.9, 1.7 Hz); 3.20-3.31 (m, 2H); 2.84, (dt, 1H, J=6.5, 3.0); 1.65-1.71 (m, 1H); 1.42 (s, 9H); 1.19 (d, 1H, J=8.1). ¹³C NMR (125 Hz, DMSO-d₆): δ 165.0; 155.8; 79.5; 61.5; 50.6; 44.9; 40.8; 33.8; 28.6.

### Example 4: Antitumor Efficacy and Pharmacodynamic Activity of the Combination of Compound 1 and Retifanlimab or Compound 3 and Retifanlimab in the CT-26 Clone 299 Colorectal Cancer Syngeneic Mouse Model

### CT-26 Clone 299 Syngeneic Efficacy Model

BALB/c-hPD1/hPDL1 mice (GemPharmatech, strain #T004025) express human PDCD1 and CD274 (which encode the PD-1 and PD-L1 proteins, respectively) from the respective murine loci for those genes. Thus, these mice express the human versions of PD-1 and PD-L1 and do not express the murine versions of these genes.

CT-26 Clone 299 cells (generated by Horizon Discovery) are a KRAS G12D expressing murine BALB/c derived colorectal cancer cell line in which both copies of PD-L1 (Cd274) were knocked out and replaced with human CD274 under the control of the endogenous Cd274 promoter.

Note that Compound 1 (monotherapy and combinations) and Compound 3 (monotherapy and combinations) were tested in independent studies. Female BALB/c-hPD1/hPDL1 mice (age 8-10 weeks) were inoculated subcutaneously with 1.0×10⁶ CT-26 Clone 299 cells suspended in phosphate buffered saline. Treatment of tumor-bearing mice started 11 days (combination with Compound 1) or 13 days (combination with Compound 2) or 12 days (combination with Compound 3) after inoculation, when tumor volume reached approximately 120 mm³. CT-26 Clone 299 inoculated mice were randomized by tumor volume into groups of N=10. They were then administered monotherapy with either Compound 1 at (30 mg/kg BID PO or 100 mg/kg QD PO), Compound 3 at (10 mg/kg BID PO or 30 mg/kg QD PO), or retifanlimab at 10 mg/kg BIW IP, both agents in combination at both dose concentrations of Compound 1, both agents in combination at both dose concentrations of Compound 3, or vehicle control PO. Treatment was continuous throughout the study and ended on day 24 post-tumor implant. Mice were weighed and tumor measurements taken once-to-twice a week thru the end of the study on day 60 post-tumor implant. Mice were euthanized when either the group average or individual mouse tumor volume reached 1700 mm³. A partial response was defined as tumor volume ≤50% initial tumor volume for 2 consecutive measurements and a complete response was defined as tumor measuring ≤3 mm×3 mm for 2 consecutive measurements. The tumor volume was calculated in 2 dimensions using the following equation

$$volume = {[ length \times ( width}^{2} ) ] / 2.$$

For survival (Kaplan-Meier) analysis: in the Compound 1 study, events were recorded when tumor volumes reached 1700 mm³ or mice met criteria for humane endpoint (e.g. body weight loss, tumor ulceration, overall condition); in the Compound 3 study, events were recorded when tumor volumes reached 2000 mm³, when mice met criteria for humane endpoint, or upon confirmed recurrence following a complete response (confirmed recurrence defined as a growing tumor ≤100 mm³ following a complete response as defined above).

Tumor growth inhibition (TGI) was calculated using the formula (1−[V_{T}/V_{C}])×100, where V_{T} is the average tumor volume of the treatment group on the last day of treatment and V_{C} is the average tumor volume of the control group on the last day of treatment. Statistical analyses were performed using GraphPad Prism software (v9.3.1; GraphPad Software, Boston, MA). Two-way analysis of variance with Dunnet's multiple comparisons test was used to determine statistical differences between the treatment groups compared to the vehicle group and other dose groups. Kaplan-Meier analysis was used to determine statistical differences in survival between treatment groups.

### CT-26 Clone 299 Pharmacodynamic Model

Female BALB/c-hPD1/hPDL1 mice (GemPharmatech, aged 8-10 weeks) were inoculated subcutaneously with 1.0×10⁶ CT-26 Clone 299 cells suspended in phosphate buffered saline. For the pharmacodynamic portion of the study, treatment of tumor-bearing mice started 17 days after inoculation, when tumor volume reached approximately 680 mm³. The tumor volume was calculated in 2 dimensions using the following equation

$$volume = {[ length \times ( width}^{2} ) ] / 2.$$

CT-26 Clone 299 inoculated mice were randomized by tumor volume into groups of N=4 or 6. They were then administered monotherapy with either Compound 1 at 100 mg/kg QD PO, or Retifanlimab at 10 mg/kg BIW IP, both agents in combination, or vehicle PO. Oral dose administration was continuous for 5 days and 2 doses of Retifanlimab was given. On day 5, plasma and tumor samples were collected 4 hour post oral dose and 24 hours post IP dose following CO₂ asphyxiation. Blood was collected into ethylenediaminetetraacetic acid (450480 Greiner Bio-One) tubes. The plasma was analyzed for drug levels. Two tumor fragments per individual were collected and weighed into Omni Bead Ruptor tubes (19-628 Omni International) and flash frozen. One tumor piece was lysed at a 1:5 ratio with homogenization solution (water:acetonitrile:formic acid, 95:5:0.1, v:v:v) for drug concentration analysis. The second tumor piece was then lysed at a 1:5 ratio with lysis buffer (64KL1 FD, Cisbio) with added protease inhibitors (A32957 and A32965, Thermo Fisher) and Blocking Reagent (64KB1AAC, Cisbio) on a Bead Ruptor Elite homogenizer (19-042E, Omni International, Kennesaw, GA). Tumor lysates were spun at 10,000 revolutions per minute for 10 minutes at 4° C. The protein concentration was determined using the Pierce™ BCA Protein Assay method according to the manufacturer's protocol (23227 Thermo Fisher Scientific). The lysates were diluted with additional lysis buffer to a final concentration of 0.4 μg/μL. Samples were analyzed using the MesoScale Discovery Platform on the Phospho/Total ERK1/2 Whole Cell Lysate Kit (K15107D, MesoScale Discovery, Rockville, MD). The pERK levels were first normalized to total ERK for each sample and then to the average pERK/tERK ratio of the vehicle control group.

### Pharmacokinetic Sample Analysis

Plasma and tumor concentrations of Compound 1 were determined with a calibration curve prepared in plasma. Quality control samples prepared in vehicle tumor homogenate were included to confirm the accuracy of plasma as a surrogate matrix for the tumor homogenate samples. Plasma and tumor homogenate study sample aliquots (25 μL volume) were deproteinized with vigorous mixing with 200 μL of 50 nM Compound 1 in acetonitrile. After centrifugation, 100 μL of the supernatants were transferred to a 96-well plate containing 200 μL of water, mixed well, and analyzed by LC-MS/MS. Chromatography was performed using 5 μL injections of extracts with an ACE C18-AR HPLC column (50×2.1 mm, 3 μm, at 45° C.) under gradient conditions (see Table 1) with a flow rate of 0.75 mL/minute. All tumor samples were above quantitation limit (5000 nM) for Compound 1, additionally, Compound 1 signal saturated on mass spectrometer. Thus, all samples for Compound 1 were re-injected at 2 μL. All tumor samples with concentrations above the upper limit of quantification were reinjected at 0.5 μL (along with a set of QCs) to bring the peak areas within the linear range. Water containing 0.1% formic acid and acetonitrile containing 0.1% formic acid were used for Mobile Phase A and B, respectively. The LC-MS/MS analysis was performed using a Shimadzu Nexera Ultra High-Performance Liquid Chromatography system coupled to the electrospray ionization source (in positive ion MRM mode) of a SCIEX Triple Quad 6500+ mass spectrometer. Peak areas for the MRM transitions 628.228 m/z→517.228 m/z for Compound 1 (retention time=1.18 min), 370.792 m/z→296.652 m/z for methyl (2R)-2-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)pyrrolidine-1-carboxylate (“Compound 3”) (retention time=1.30 min) as Internal Standard for Compound 1 were used to create linear regression calibration equations with 1/x² weighting. SCIEX Analyst® software (v1.7.2) was used to acquire the raw data and to calculate calibration curves and study sample concentrations. The assay range was adjusted for anticipated study concentrations, ranging from 1 to 5000 nM.

TABLE 1
High-Performance Liquid Chromatography
Gradient Elution Scheme

| Time (minutes) | % Mobile Phase B |
|---|---|
| 0.50 | 20 |
| 1.60 | 100 |
| 2.60 | 100 |
| 2.61 | 20 |
| 3.20 | Stop |

Note:
Post-column divert valve: 0.8 to 1.6 minutes flow to mass spectrometry, all else to waste.

### Results

### Antitumor Activity of Compound 1±Retifanlimab in the CT-26 Clone 299 Colorectal Syngeneic Tumor Model

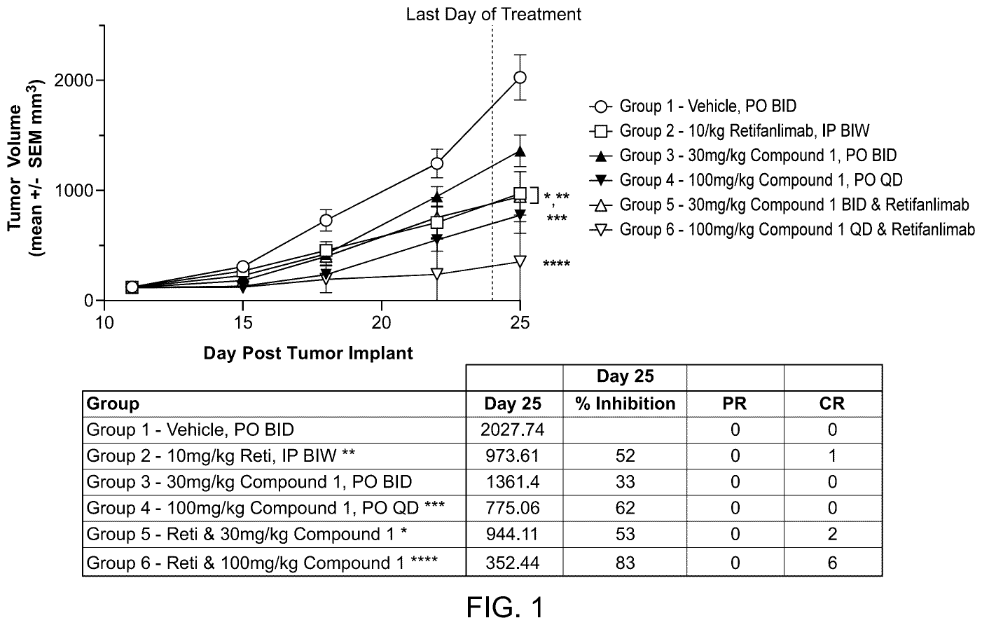

The antitumor activity of the combination of Compound 1 and retifanlimab was evaluated in the CT-26 Clone 299 colorectal model. Mice were administered monotherapy with either Compound 1 at 30 mg/kg BID PO, 100 mg/kg QD PO, or retifanlimab at 10 mg/kg BIW IP, both agents in combination at both dose concentrations of Compound 1, or vehicle PO. When assessed on day 25, following the final treatment day, 30 mg/kg Compound 1 BID PO resulted in a TGI of 33% when compared to vehicle, while 100 mg/kg Compound 1 QD PO resulted in a TGI of 62% when compared to vehicle (p=0.0008). retifanlimab IP BIW group yielded a 52% TGI. The combination of 10 mg/kg of retifanlimab BIW IP with 30 mg/kg of Compound 1 BID PO resulted in 53% TGI (p=0.010) compared to vehicle (see FIG. 1).

In FIG. 1, BALB/c-hPD1/hPDL1 mice bearing subcutaneous CT-26 Clone 299 tumors were treated with monotherapy of either Compound 1 at 30 mg/kg PO BID, 100 mg/kg PO QD, or retifanlimab at 10 mg/kg IP BIW, both agents in combination at both dose concentrations of Compound 1, or vehicle PO. Dose administration began on Day 11 and ended on Day 24. Retifanlimab was given on days 11, 15, 18, 19 and 23 post tumor implant.

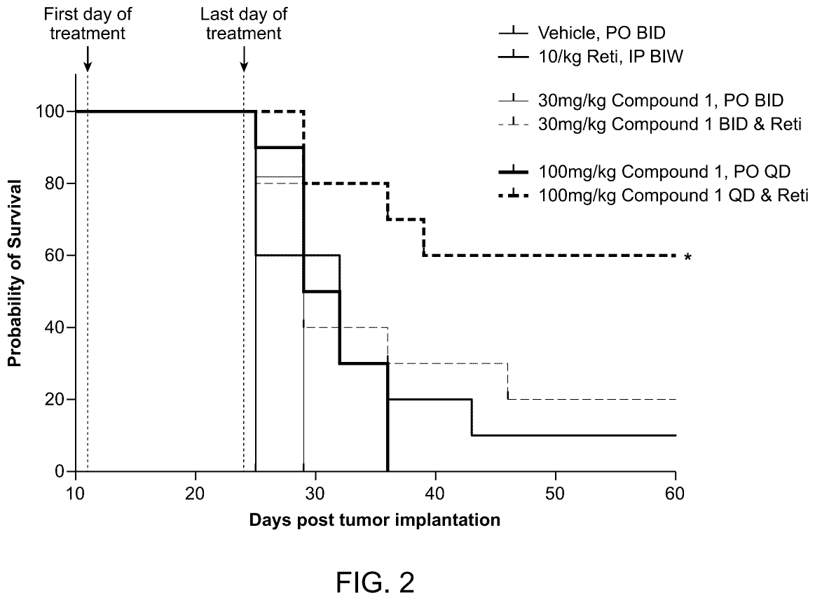

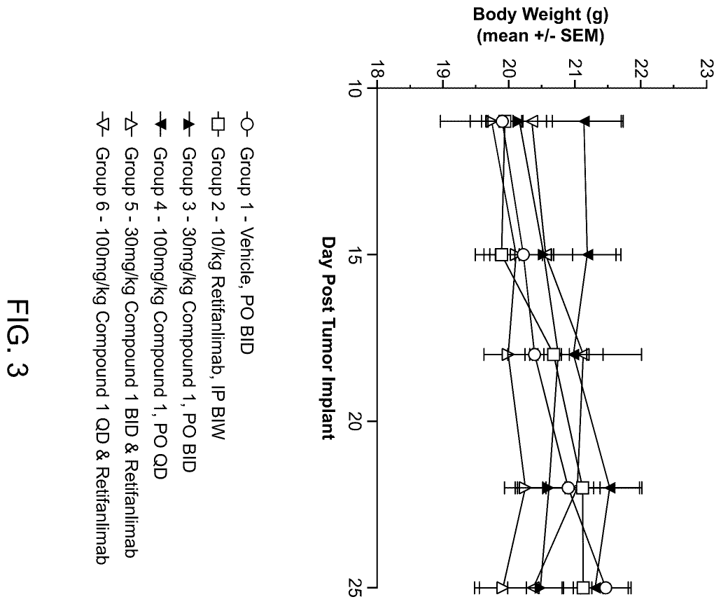

The combination of 10 mg/kg retifanlimab BIW IP with 100 mg/kg Compound 1 QD PO increased the TGI to 83%, when compared to vehicle (p<0.0001). There were 2 complete tumor regressions in the combination group of 10 mg/kg retifanlimab BIW IP with 30 mg/kg Compound 1 BID PO. There were 6 complete tumor regressions in the combination group of 10 mg/kg retifanlimab BIW IP with 100 mg/kg Compound 1 QD PO (see FIG. 2). Dosing ended on day 24 post inoculation with tumor and weight measurements continuing till day 60. Mice were euthanized when either the group average tumor volume of individual tumor volume reached 1700 mm³. Overall, all treatment groups demonstrated delayed tumor growth relative to the vehicle control group (p<0.005 in all groups). Moreover treatment groups that combined retifanlimab plus Compound 1 demonstrated significantly longer tumor growth delay following cessation of treatment on day 24 compared to those treated with Compound 1 alone, and this effect was statistically significant at the 100 mg/kg Compound 1 dose level plus retifanlimab (see FIG. 2). Each dose was tolerated as determined by lack of body weight loss in any treatment group (see FIG. 3).

### Pharmacodynamics and Pharmacokinetics of Compound 1±Retifanlimab in CT-26 Clone 299 Tumor-Bearing Mice

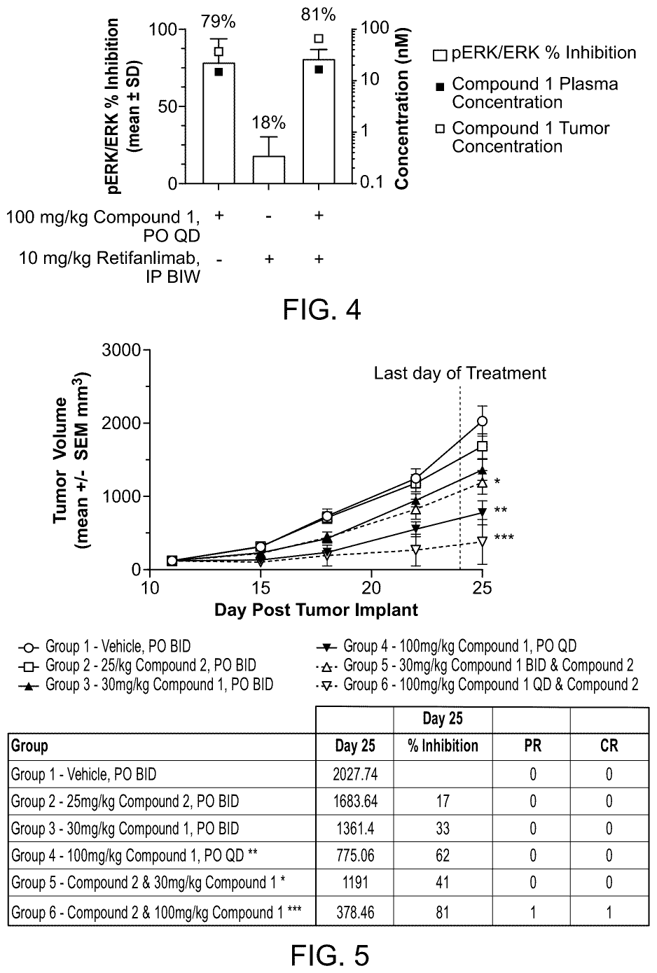

Plasma and tumors were collected 4 hour post oral dose and 24 hours post IP dose following 5 days of treatment with monotherapy of either Compound 1 at 100 mg/kg QD P0, or retifanlimab at 10 mg/kg P BIW, both agents in combination, or vehicle P0. Retifanlimab was collected 24 hours post second dose. Levels of total and phosphorylated ERK were measured from the tumor samples. Compound 1 at 100 mg/kg QD resulted in KRAS inhibition at 79% (see FIG. 4). Retifanlimab at 10 mg/kg BIW P resulted in only 18% pERK inhibition. Combination treatment of Compound 1 and retifanlimab resulted in KRAS inhibition at 81%, indicating a lack of effect on ERK signaling from PD1 inhibition in this model. Also shown in FIG. 4, the drug concentrations for Compound 1 in both the plasma and tumor samples at 4 hours post dose are above an internally defined IC₉₀ for Compound 1.

TABLE 2
Plasma and Tumor Concentration Relative to pERK inhibition

| | Compound 1 | Compound 1 | pERK/ERK |
|---|---|---|---|
| | Plasma | Tumor | Inhibition Relative |
| | Concentration | Concentration | to Vehicle Control |
| Dose and Timepoint | (uM, Mean) | (uM, Mean) | (%, Mean) |
| 100 mg/kg Cmpd1, QD 4 Hr | 14.1 | 29.1 | 85 |
| 100 mg/kg Cmpd1, QD 4 Hr | 19.7 | 55.1 | 89 |
| 100 mg/kg Cmpd1, QD 4 Hr | 17.7 | 39.6 | 84 |
| 100 mg/kg Cmpd1, QD 4 Hr | 8.09 | 23.3 | 56 |
| 100 mg/kg Cmpd1 4 Hr PO & | 17.2 | 60.8 | 72 |
| Retifanlimab 24 Hr IP | | | |
| 100 mg/kg Cmpd1 4 Hr PO & | 18.2 | 90.8 | 81 |
| Retifanlimab 24 Hr IP | | | |
| 100 mg/kg Cmpd1 4 Hr PO & | 15.0 | 56.1 | 89 |
| Retifanlimab 24 Hr IP | | | |
| 100 mg/kg Cmpd1 4 Hr PO & | 18.1 | 68.1 | 80 |
| Retifanlimab 24 Hr IP | | | |
| 100 mg/kg Cmpd1 4 Hr PO & | 18.4 | 48.2 | 86 |
| Retifanlimab 24 Hr IP | | | |
| 100 mg/kg Cmpd1 4 Hr PO & | 13.5 | 72.2 | 79 |
| Retifanlimab 24 Hr IP | | | |

The data demonstrate that dosing Compound 1 in combination with retifanlimab resulted in additional tumor growth inhibition in the CT-26 Clone 299 syngeneic model. Increased efficacy from the combination was due to activity not associated with inhibition of pERK signaling.

### Antitumor Activity of Compound 1±Retifanlimab in the CT-26 Clone 299 Colorectal Syngeneic Tumor Model

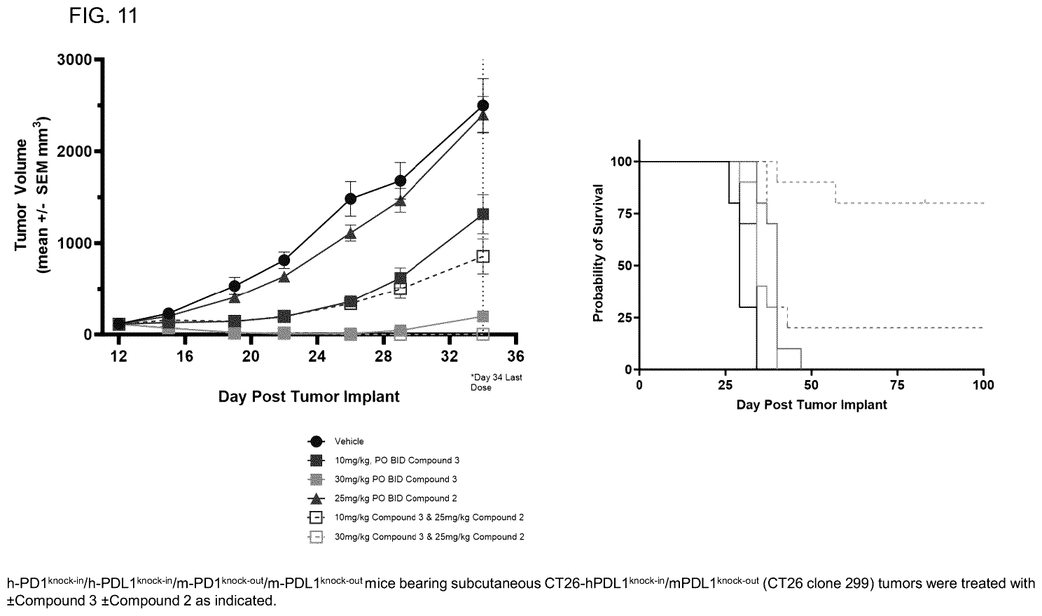

The antitumor activity of the combination of Compound 3 and retifanlimab was evaluated in the CT-26 Clone 299 colorectal model (FIG. 11). Mice were administered monotherapy with either Compound 3 at 10 mg/kg BID or 30 mg/kg QD, or retifanlimab at 10 mg/kg BIW, both agents in combination at both dose concentrations of Compound 3, or vehicle PO. When assessed on day 34, following the final treatment day, 10 mg/kg Compound 3 BID PO resulted in reduced tumor growth compared to vehicle control treated animals, but this did not rise to the level of significance (p=0.1127, TGI 47%). Treatment with 30 mg/kg Compound 3 BID PO resulted in significantly decreased tumor growth compared to vehicle control (p=0.0411, 92% TGI). Treatment with monotherapy retifanlimab was relatively effective in its own right, significantly decreasing tumor growth compared to vehicle control (p=0.0263, 74% TGI). While combination treatment with Compound 3+retifanlimab only further decreased tumor growth compared to monotherapy treatment in the case of the higher dose-level combination of Compound 3 (30 mg/kg BD), this effect was not statistically significant. Assessment of study animals and tumor growth monitoring continued beyond the end of active treatment, to enable assessment of tumor growth delay and the durability and duration of anti-tumor activity induced by these treatment regimens. In this setting, combining Compound 3 at 30 mg/kg BID (but not at 10 mg/kg BID) with retifanlimab offered clear therapeutic benefits in terms of long-term and recurrence-free survival compared to monotherapy treatments. At the lower dose level, survival probability did not differ significantly between animals treated with Compound 3 (10 mg/kg BID)+retifanlimab and retifanlimab monotherapy (p=0.1884). At the higher dose level (Compound 3 30 mg/kg BID) the combination treated animals demonstrated improved though not statistically significant probability of recurrence-free survival compared to retifanlimab monotherapy and significantly greater probability of recurrence-free survival over time compared to Compound 3 monotherapy at 30 mg/kg BID (p=0.0004).

### Example 5: Antitumor Efficacy and Pharmacodynamic Activity of the Combination of Compound 1 and Compound 2 or Compound 3 and Compound 2 in the CT-26 Clone 299 Colorectal Cancer Syngeneic Mouse Model

### CT-26 Clone 299 Syngeneic Efficacy Model

BALB/c-hPD1/hPDL1 mice (GemPharmatech, strain #T004025) express human PDCD1 and CD274 (which encode the PD-1 and PD-L1 proteins, respectively) from the respective murine loci for those genes. Thus, these mice express the human versions of PD-1 and PD-L1 and do not express the murine versions of these genes.

CT-26 Clone 299 cells (generated by Horizon Discovery) are a KRAS G12D expressing murine BALB/c derived colorectal cancer cell line in which both copies of PD-L1 (Cd274) were knocked out and replaced with human CD274 under the control of the endogenous Cd274 promoter.

Note that Compound 1 (monotherapy and combinations) and Compound 3 (monotherapy and combinations) were tested in independent studies. Female BALB/c-hPD1/hPDL1 mice (age 8-10 weeks) were inoculated subcutaneously with 1.0×10⁶ CT-26 Clone 299 cells suspended in phosphate buffered saline. Treatment of tumor-bearing mice started 11 days (combination with Compound 1) or 13 days (combination with Compound 2) or 12 days (combination with Compound 3) after inoculation, when tumor volume reached approximately 120 mm³. CT-26 Clone 299 inoculated mice were randomized by tumor volume into groups of N=10. They were then administered monotherapy with either Compound 1 at (30 mg/kg BID PO or 100 mg/kg QD PO), Compound 3 at (10 mg/kg BID PO or 30 mg/kg QD PO), or Compound 2 at 25 mg/kg BID PO, both agents in combination at both dose concentrations of Compound 1, both agents in combination at both dose concentrations of Compound 3, or vehicle control PO. Treatment was continuous throughout the study and ended on day 24 post-tumor implant. Mice were weighed and tumor measurements taken once-to-twice a week thru the end of the study. A partial was defined as tumor volume ≤50% initial tumor volume for 2 consecutive measurements and a complete response was defined as tumor measuring ≤3 mm×3 mm for 2 consecutive measurements. The tumor volume was calculated in 2 dimensions using the following equation

$$volume = {[ length \times ( width}^{2} ) ] / 2.$$

For survival (Kaplan-Meier) analysis: in the Compound 1 study, events were recorded when tumor volumes reached 1700 mm³ or mice met criteria for humane endpoint (e.g. body weight loss, tumor ulceration, overall condition); in the Compound 3 study, events were recorded when tumor volumes reached 2000 mm³, when mice met criteria for humane endpoint, or upon confirmed recurrence following a complete response (confirmed recurrence defined as a growing tumor ≤100 mm³ following a complete response as defined above).

Tumor growth inhibition (TGI) was calculated using the formula (1−[V_{T}/V_{C}])×100, where V_{T} is the average tumor volume of the treatment group on the last day of treatment and V_{C} is the average tumor volume of the control group on the last day of treatment. Statistical analyses were performed using GraphPad Prism software (v9.3.1; GraphPad Software, Boston, MA). Two-way analysis of variance with Dunnet's multiple comparisons test was used to determine statistical differences between the treatment groups compared to the vehicle group and other dose groups. Kaplan-Meier analysis was used to determine statistical differences in survival between treatment groups.

### Pharmacokinetic Sample Analysis

Plasma and tumor concentrations of Compound 1 and Compound 2 were determined with a calibration curve prepared in plasma. Quality control samples prepared in vehicle tumor homogenate were included to confirm the accuracy of plasma as a surrogate matrix for the tumor homogenate samples. Plasma and tumor homogenate study sample aliquots (25 μL volume) were deproteinized with vigorous mixing with 200 μL of 50 nM Compound 3 & Compound 2-d₅ in acetonitrile. After centrifugation, 100 μL of the supernatants were transferred to a 96-well plate containing 200 μL of water, mixed well, and analyzed by LC-MS/MS. Chromatography was performed using 5 μL injections of extracts with an ACE C18-AR HPLC column (50×2.1 mm, 3 μm, at 45° C.) under gradient conditions (see Table 3) with a flow rate of 0.75 mL/minute. All tumor samples were above quantitation limit (5000 nM) for Compound 1, additionally, Compound 1 signal saturated on mass spectrometer. Thus, all samples for Compound 1 were re-injected at 2 μL. All tumor samples with concentrations above the upper limit of quantification were reinjected at 0.5 μL (along with a set of QCs) to bring the peak areas within the linear range. Water containing 0.1% formic acid and acetonitrile containing 0.1% formic acid were used for Mobile Phase A and B, respectively. The LC-MS/MS analysis was performed using a Shimadzu Nexera Ultra High-Performance Liquid Chromatography system coupled to the electrospray ionization source (in positive ion MRM mode) of a SCIEX Triple Quad 6500+ mass spectrometer. Peak areas for the MRM transitions 628.228 m/z→517.228 m/z for Compound 1 (retention time=1.18 min), 380.159 m/z→530.050 m/z for Compound 2 (retention time=1.18 min), 370.792 m/z→296.652 m/z for Compound 3 (retention time=1.30 min) as Internal Standard for Compound 1, and 764.500 m/z→545.200 m/z for Compound 2-d₅ (retention time=1.18 min) as Internal Standard for Compound 2 were used to create linear regression calibration equations with 1/x² weighting. SCIEX Analyst® software (v1.7.2) was used to acquire the raw data and to calculate calibration curves and study sample concentrations. The assay range was adjusted for anticipated study concentrations, ranging from 1 to 5000 nM.

TABLE 3
High-Performance Liquid Chromatography
Gradient Elution Scheme

| Time (minutes) | % Mobile Phase B |
|---|---|
| 0.5 | 20 |
| 1.60 | 100 |
| 2.60 | 100 |
| 2.61 | 20 |
| 3.20 | Stop |

Note:
Post-column divert valve: 0.8 to 1.6 minutes flow to mass spectrometry, all else to waste.

### Results

### Antitumor Activity of Compound 1±Compound 2 in the CT-26 Clone 299 Colorectal Syngeneic Tumor Model

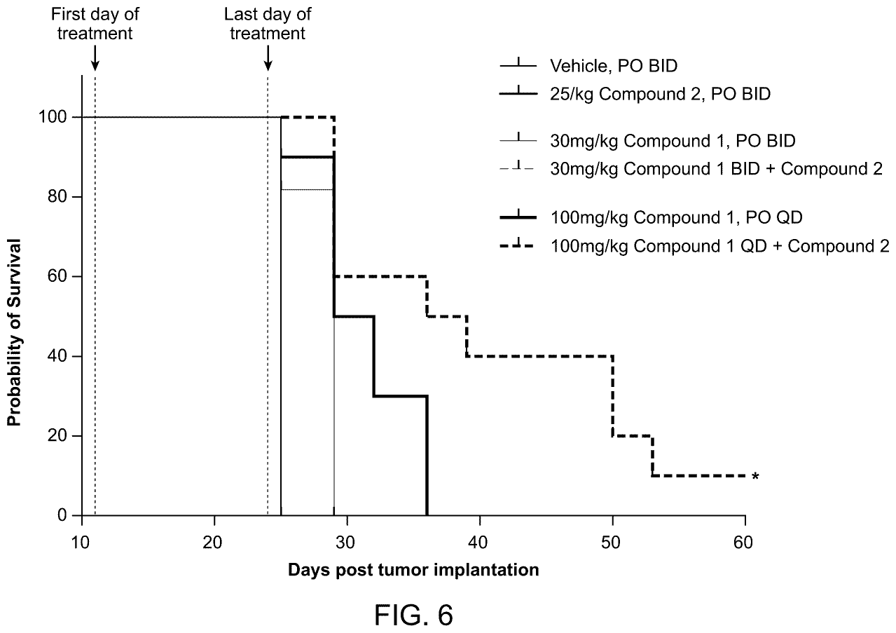

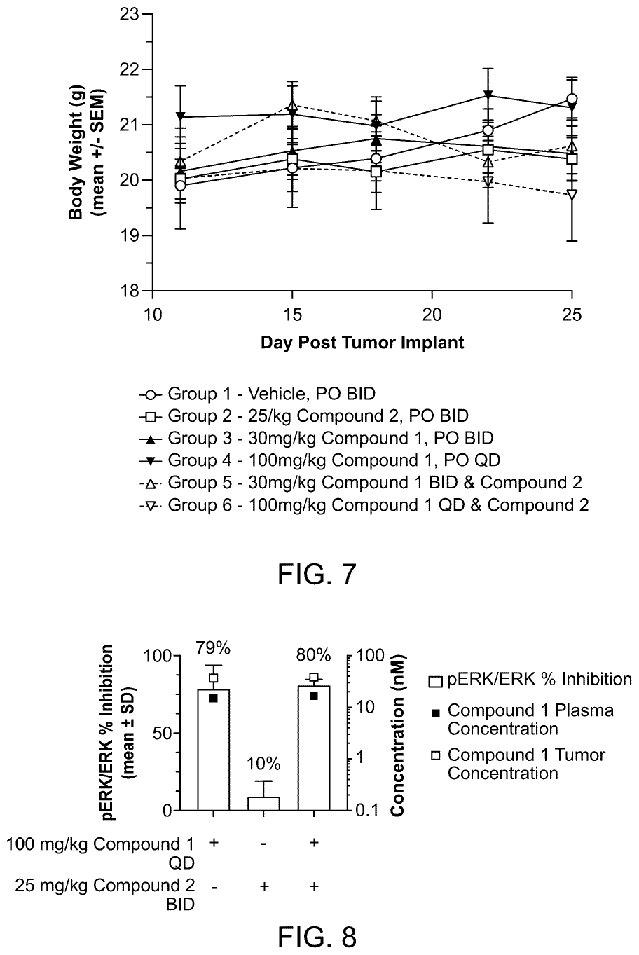

The antitumor activity of the combination of Compound 1 and Compound 2 was evaluated in the CT-26 Clone 299 colorectal model. Mice were administered monotherapy with either Compound 1 at 30 mg/kg BID or 100 mg/kg QD, or Compound 2 at 25 mg/kg BID, both agents in combination at both dose concentrations of Compound 1, or vehicle PO. When assessed on day 25, following the final treatment day, 30 mg/kg Compound 1 BID PO resulted in a TGI of 33% when compared to vehicle while 100 mg/kg Compound 1 QD PO resulted in a TGI of 62% when compared to vehicle (p=0.0008). The Compound 2 BID PO dose yielded a 17% TGI. The combination of 25 mg/kg of Compound 2 BID PO with 30 mg/kg of Compound 1 BID PO showed a TGI of 44% and the combination of 25 mg/kg Compound 2 BID PO with 100 mg/kg Compound 1 QD PO increased the TGI to 81%, when compared to vehicle (p<0.0001) (see FIG. 5). There was 1 partial tumor response and 1 complete tumor regression in the combination group of 25 mg/kg Compound 2 BID PO with 100 mg/kg Compound 1 QD PO Dosing ended on day 24 post inoculation with tumor and weight measurements continuing until day 60. Mice were euthanized when either the group average volume or individual mouse tumor volume reached 1700 mm3. Overall, treatment groups including Compound 2 (i.e., groups 3, 4, 5, and 6) all demonstrated delayed tumor growth relative to the vehicle control group (p<0.0005 in all groups). Moreover, at the 100 mg/kg Compound 1 dose level, combination treatment with Compound 2 demonstrated significantly longer tumor growth delay following cessation of treatment on day 24 compared to treatment with either monotherapy alone (p<0.03)(see FIG. 6). Each dose was tolerated as determined by lack of body weight loss in any treatment group (see FIG. 7).

After treatment cessation, 100% (Compound 1 (100 mg/kg), Compound 2) or 90% (retifanlimab) of monotherapy-treated mice succumbed to tumor growth by day 43. By contrast, combination of Compound 1 and retifanlimab completely eliminated tumors in 60% of treated mice, which remained tumor free >100 days following initial tumor implantation; 10% of mice receiving Compound 1+Compound 2 achieved complete tumor regression (see FIGS. 2 and 6).

Surviving mice rejected tumor rechallenge on day 120, whereas tumor-naive mice challenged in parallel rapidly developed tumors. Similarly, testing the combination of Compound 1+antibodies targeting PD-1 or PD-L1 also improved long-term tumor-free survival in a pancreatic ductal adenocarcinoma (PDAC) mouse tumor model.

### Pharmacodynamics and Pharmacokinetics of Compound 1±Compound 2 in CT-26 Clone 299 Tumor-Bearing Mice

Plasma and tumors were collected 4-hour post dose following 5 days of treatment with monotherapy of either Compound 1 at 100 mg/kg QD, or Compound 2 at 25 mg/kg BID, both agents in combination or, vehicle P0. Levels of total and phosphorylated ERK were measured from the tumor samples. Compound 1 at 100 mg/kg QD resulted in KRAS inhibition at 79% (see FIG. 8). Compound 2 at 25 mg/kg BID resulted in only 10% pERK inhibition. Combination of Compound 1 and Compound 2 resulted in KRAS inhibition at 80%, indicating a lack of effect on ERK signaling from PD-L1 inhibition in this model. Also shown in FIG. 8, the drug concentrations for Compound 1 in both the plasma and tumor samples at 4 hours post dose are above an internally defined IC₉₀ for Compound 1.

TABLE 4
Plasma and Tumor Concentration Relative to pERK inhibition

| | Cmpd 1 | Cmpd 1 | Cmpd 2 | Cmpd 2 | |
|---|---|---|---|---|---|
| | Plasma | Tumor | Plasma | Tumor | pERK/ERK Inhibition |
| Dose and | Conc. (uM, | Conc. (uM, | Conc. (uM, | Conc. (uM, | Relative to Vehicle |
| Timepoint | Mean) | Mean) | Mean) | Mean) | Control (%, Mean) |
| 100 mg/kg Cmpd | 14.1 | 29.1 | | | 85 |
| 1, QD 4 Hr | | | | | |
| 100 mg/kg Cmpd | 19.7 | 55.1 | | | 89 |
| 1, QD 4 Hr | | | | | |
| 100 mg/kg Cmpd | 17.7 | 39.6 | | | 84 |
| 1, QD 4 Hr | | | | | |
| 100 mg/kg Cmpd | 8.09 | 23.3 | | | 56 |
| 1, QD 4 Hr | | | | | |
| 25 mg/kg Cmpd | | | 1.07 | 3.70 | 15 |
| 2, BID 4 Hr | | | | | |
| 25 mg/kg Cmpd | | | 0.461 | 2.55 | 20 |
| 2, BID 4 Hr | | | | | |
| 25 mg/kg Cmpd | | | 0.508 | 1.80 | −2 |
| 2, BID 4 Hr | | | | | |
| 25 mg/kg Cmpd | | | 0.942 | 3.82 | 5 |
| 2, BID 4 Hr | | | | | |
| 100 mg/kg Cmpd | 13.4 | 28.7 | 1.14 | 7.81 | 75 |
| 1 & 25 mg/kg | | | | | |
| Cmpd 2, 4 Hr | | | | | |
| 100 mg/kg Cmpd | 13.1 | 28.8 | 0.764 | 5.12 | 82 |
| 2 & 25 mg/kg | | | | | |
| Cmpd 2, 4 Hr | | | | | |
| 100 mg/kg Cmpd | 14.7 | 58.6 | 0.790 | 8.69 | 80 |
| 1 & 25 mg/kg | | | | | |
| Cmpd 2, 4 Hr | | | | | |
| 100 mg/kg Cmpd | 16.4 | 32.8 | 1.12 | 5.29 | 88 |
| 1 & 25 mg/kg | | | | | |
| Cmpd 2, 4 Hr | | | | | |
| 100 mg/kg Cmpd | 13.7 | 27.0 | 1.53 | 4.94 | 79 |
| 1 & 25 mg/kg | | | | | |
| Cmpd 2, 4 Hr | | | | | |
| 100 mg/kg Cmpd | 12.6 | 62.0 | 1.94 | 13.1 | 76 |
| 1 & 25 mg/kg | | | | | |
| Cmpd 2, 4 Hr | | | | | |

The data demonstrate that dosing Compound 1 in combination with Compound 2 resulted in additional tumor growth inhibition in the CT-26 Clone 299 syngeneic model. Both PD-1 and PD-L1 inhibitors are synergistic with Compound 1 (at 100 mg/kg PO QD) as determined using the “survival curves” described in E. Demidenko, et al., PLoS ONE, 2019, 14(11): e0224137. Increased efficacy from the combination was due to activity not associated with inhibition of pERK signaling.

These results demonstrate that Compound 1, a potent, selective, orally bioavailable KRAS G12D inhibitor that induces major histocompatibility complex (MHC) class I expression on G12D-mutated cancer cells in vitro, sensitizes G12D mutant tumors to immune checkpoint blockade, enhancing antitumor activity and tumor clearance.

### Antitumor Activity of Compound 3±Compound 2 in the CT-26 Clone 299 Colorectal Syngeneic Tumor Model

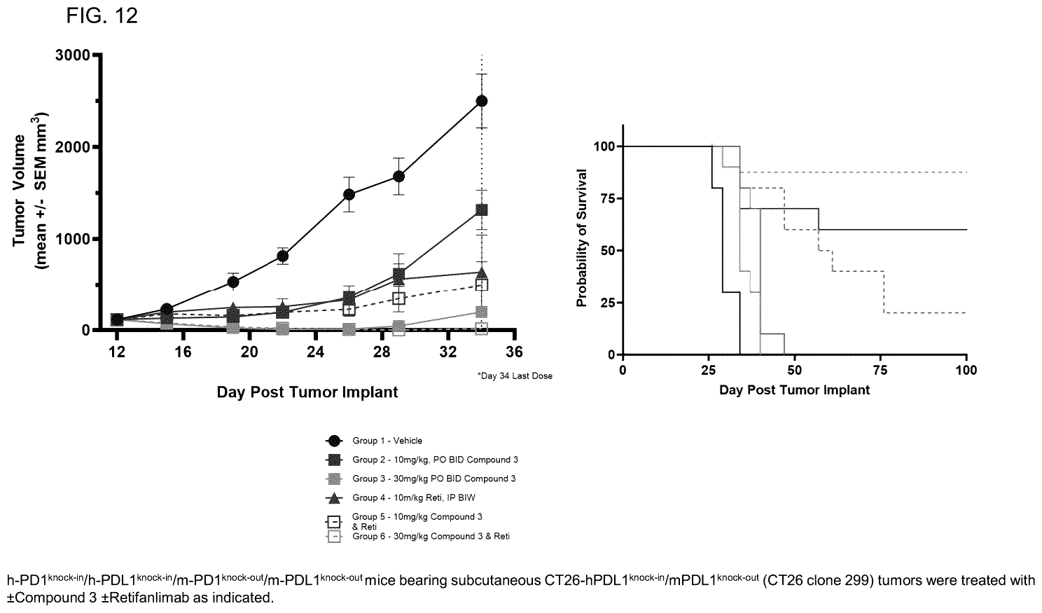

The antitumor activity of the combination of Compound 3 and Compound 2 was evaluated in the CT-26 Clone 299 colorectal model (FIG. 12). Mice were administered monotherapy with either Compound 3 at 10 mg/kg BID or 30 mg/kg QD, or Compound 2 at 25 mg/kg BID, both agents in combination at both dose concentrations of Compound 3, or vehicle PO. When assessed on day 34, following the final treatment day, 10 mg/kg Compound 3 BID PO resulted in reduced tumor growth compared to vehicle control treated animals, but this did not rise to the level of significance (p=0.1127, TGI 47%). Treatment with 30 mg/kg Compound 3 BID PO resulted in significantly decreased tumor growth compared to vehicle control (p=0.0411, 92% TGI). While combination treatment with Compound 3 (at either dose level)+Compound 2 did result in further decreased tumor growth compared to monotherapy treatment with Compound 3 at the same dose level, this effect was not significant through day 34 (10 mg/kg BID combination: p-value=0.5181); 30 mg/kg BID combination: p-value=0.0545). Monotherapy treatment with Compound 2 did not meaningfully alter tumor growth. Assessment of study animals and tumor growth monitoring continued beyond the end of active treatment, to enable assessment of tumor growth delay and the durability and duration of anti-tumor activity induced by these treatment regimens. In this setting, combining Compound 3 with Compound 2 offered clear therapeutic benefits in terms of long-term and recurrence-free survival compared to monotherapy treatments. While animals treated with Compound 3 (10 mg/kg BID)+Compound 2 demonstrated greater probability of survival than did animals treated with Compound 3 (10 mg/kg BID) or Compound 2 monotherapy, this effect was only statistically significant with respect to Compound 2 (p=0.0110); however, at the higher dose level (Compound 2 30 mg/kg BID) the combination treated animals demonstrated significantly greater probability of recurrence-free survival than did either monotherapy treatment with Compound 3 or Compound 2 (p<0.0001 in both cases), with long-term recurrence free survival at 80% through 97 days post tumor implant.

### Example 6: Antitumor Efficacy and Pharmacodynamic Activity of the Combination of Compound 1 and Anti-Mouse-PD-1 Antibody RMP1-14 in the KPCY-013 (2838c3) Pancreatic Cancer Syngeneic Mouse Model

### KPCY-013 (2838c3) Syngeneic Efficacy Model

KPCY-013 cells (also known as 2838c3 cells, described in Li et al, Immunity 2018; 49:178-193, DOI 10.1016/j.immuni.2018.06.006 and obtained under license from the Stanger lab at the University of Pennsylvania) are a KRAS G12D expressing murine pancreatic cancer derived cell line.

Female C57BL/6 mice (The Jackson Laboratory, Bar Harbor, ME; age 11-13 weeks) were inoculated subcutaneously with 2.5×10⁶ KPCY-013 cells suspended in phosphate buffered saline. Treatment of tumor-bearing mice started 9 days after inoculation, when tumor volume reached approximately 110 mm3. KPCY-013 inoculated mice were randomized by tumor volume into groups of N=10. They were then administered monotherapy with either Compound 1 (at 30 mg/kg QD PO or 100 mg/kg QD PO), or anti-mouse PD-1 (clone RMP1-14, at 12.5 mg/kg BIW IP), both agents in combination at both dose concentrations of Compound 1, or vehicle PO. Treatment was continuous throughout the study and ended on day 29 post-tumor implant. Mice were weighed and tumor measurements taken twice a week thru the end of the study on day 85 post-tumor implant. Mice were euthanized when either the group average or individual mouse tumor volume reached 1500 mm³. A partial response being defined as tumor volume ≤50% initial tumor volume for 2 consecutive measurements and a complete response being defined as tumor measuring ≤3 mm×3 mm for 2 consecutive measurements. The tumor volume was calculated in 2 dimensions using the following equation

$$volume = {[ length \times ( width}^{2} ) ] / 2.$$

Tumor growth inhibition (TGI) was calculated using the formula (1−[V_{T}/V_{C}])×100, where V_{T} is the average tumor volume of the treatment group on the last day of treatment and V_{C} is the average tumor volume of the control group on the last day of treatment. Statistical analyses were performed using GraphPad Prism software (v9.3.1; GraphPad Software, Boston, MA). Two-way analysis of variance with Dunnet's multiple comparisons test was used to determine statistical differences between the treatment groups compared to the vehicle group and other dose groups. Kaplan-Meier analysis was used to determine statistical differences in survival between treatment groups.

### Results

### Antitumor Activity of Compound 1±Anti-Mouse-PD-1 Antibody RMP1-14 in the KPCY-013 Pancreatic Cancer Syngeneic Tumor Model

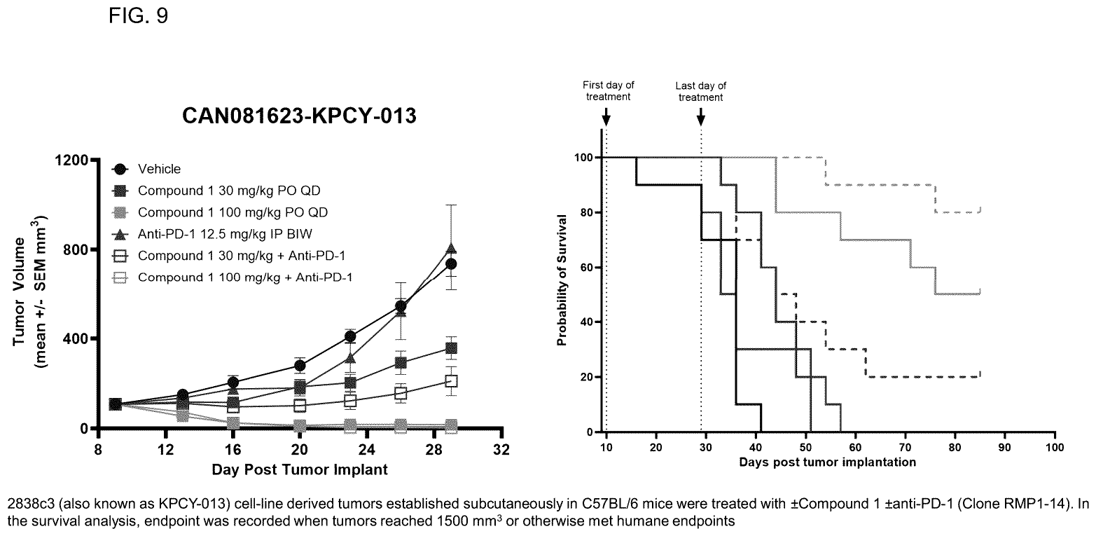

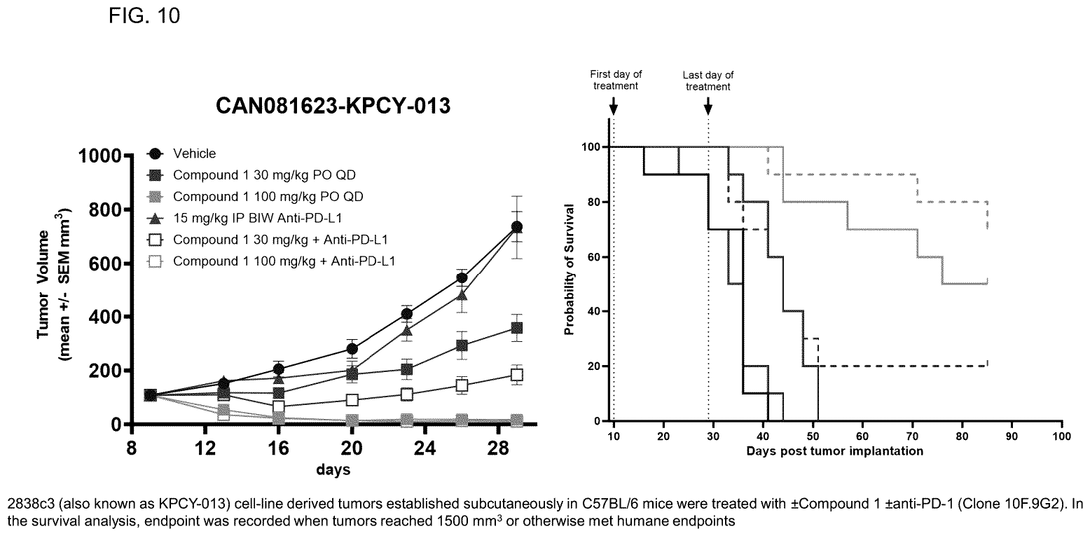

The antitumor activity of the combination of Compound 1 and anti-PD-1 was evaluated in the KPCY-013 (also known as 2838c3) Pancreatic Cancer syngeneic tumor model (FIG. 9). Mice were administered monotherapy with either Compound 1 at 30 mg/kg QD or 100 mg/kg QD, or anti-PD-1 at 12.5 mg/kg BIW, both agents in combination at both dose concentrations of Compound 1, or vehicle control. When assessed on day 29, following the final treatment day, monotherapy treatment with Compound 1 at 30 mg/kg QD or 100 mg/kg QD resulted in significantly decreased tumor growth compared to vehicle control (p s 0.0007), while monotherapy treatment with anti-PD-1 antibody had minimal impact on tumor growth. Combining Compound 1 (30 mg/kg)+anti-PD-1 appeared to further decrease tumor growth, did this effect was not statistically significant. As the higher dose of Compound 1 (100 mg/kg) with or without anti-PD-1 antibody combination decreased tumors to a minimal size by the end of the dosing period, no further decrease in tumor growth could be assessed. Assessment of study animals and tumor growth monitoring continued beyond the end of active treatment, to enable assessment of tumor growth delay and the durability and duration of anti-tumor activity induced by these treatment regimens. In this setting, treatment with Compound 1 at either dose, alone or in combination, resulted in significantly improved survival compared to the vehicle control (p≤0.0031). Combination treatment with Compound 1+anti-PD-1 extended survival over the monitoring period, but did not reach statistical significance.

### Example 7: Antitumor Efficacy and Pharmacodynamic Activity of the Combination of Compound 1 and Compound 2 in the KPCY-013 (2838c3) Pancreatic Cancer Syngeneic Mouse Model

### KPCY-013 (2838c3) Syngeneic Efficacy Model

KPCY-013 cells (also known as 2838c3 cells, described in Li et al, Immunity 2018; 49:178-193, DOI 10.1016/j.immuni.2018.06.006 and obtained under license from the Stanger lab at the University of Pennsylvania) are a KRAS G12D expressing murine pancreatic cancer derived cell line.

Female C57BL/6 mice (The Jackson Laboratory, Bar Harbor, ME; age 11-13 weeks) were inoculated subcutaneously with 2.5×10⁶ KPCY-013 cells suspended in phosphate buffered saline. Treatment of tumor-bearing mice started 9 days after inoculation, when tumor volume reached approximately 110 mm3. KPCY-013 inoculated mice were randomized by tumor volume into groups of N=10. They were then administered monotherapy with either Compound 1 (at 30 mg/kg QD PO or 100 mg/kg QD PO), or anti-mouse PD-L1 (clone 10F.9G2, at 15 mg/kg BIW IP), both agents in combination at both dose concentrations of Compound 1, or vehicle PO. Treatment was continuous throughout the study and ended on day 29 post-tumor implant. Mice were weighed and tumor measurements taken twice a week thru the end of the study on day 85 post-tumor implant. Mice were euthanized when either the group average or individual mouse tumor volume reached 1500 mm³. A partial response being defined as tumor volume ≤50% initial tumor volume for 2 consecutive measurements and a complete response being defined as tumor measuring ≤3 mm×3 mm for 2 consecutive measurements. The tumor volume was calculated in 2 dimensions using the following equation

$$volume = {[ length \times ( width}^{2} ) ] / 2.$$

Tumor growth inhibition (TGI) was calculated using the formula (1−[V_{T}/V_{C}])×100, where V_{T} is the average tumor volume of the treatment group on the last day of treatment and V_{C} is the average tumor volume of the control group on the last day of treatment. Statistical analyses were performed using GraphPad Prism software (v9.3.1; GraphPad Software, Boston, MA). Two-way analysis of variance with Dunnet's multiple comparisons test was used to determine statistical differences between the treatment groups compared to the vehicle group and other dose groups. Kaplan-Meier analysis was used to determine statistical differences in survival between treatment groups.

### Results

The antitumor activity of the combination of Compound 1 and anti-PD-L1 was evaluated in the KPCY-013 (also known as 2838c3) Pancreatic Cancer syngeneic tumor model (FIG. 9). Mice were administered monotherapy with either Compound 1 at 30 mg/kg QD or 100 mg/kg QD, or anti-PD-L1 at 15 mg/kg BIW, both agents in combination at both dose concentrations of Compound 1, or vehicle control. When assessed on day 29, following the final treatment day, monotherapy treatment with Compound 1 at 30 mg/kg QD or 100 mg/kg QD resulted in significantly decreased tumor growth compared to vehicle control (p s 0.0005), while monotherapy treatment with anti-PD-L1 antibody had minimal impact on tumor growth. Combining Compound 1 (30 mg/kg)+anti-PD-L1 appeared to further decrease tumor growth, did this effect was not statistically significant. As the higher dose of Compound 1 (100 mg/kg) with or without anti-PD-1 antibody combination decreased tumors to a minimal size by the end of the dosing period, no further decrease in tumor growth could be assessed. Assessment of study animals and tumor growth monitoring continued beyond the end of active treatment, to enable assessment of tumor growth delay and the durability and duration of anti-tumor activity induced by these treatment regimens. In this setting, treatment with Compound 1 at either dose, alone or in combination, resulted in significantly improved survival compared to the vehicle control (p≤0.0061). Combination treatment with Compound 1+anti-PD-L1 extended survival over the monitoring period, but did not reach statistical significance.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Other embodiments are within the following claims.

1. A method of treating cancer in a subject in need thereof comprising administering to the subject a KRAS G12D inhibitor, or a pharmaceutically acceptable salt thereof, and a PD-1 inhibitor or a PD-L1 inhibitor, or pharmaceutically acceptable salt thereof.

2. The method of claim 1, wherein the PD-1 inhibitor is a PD-1 immune checkpoint inhibitor.

3. The method of claim 1, wherein the PD-1 inhibitor is an anti-PD-1 antibody.

4. The method of claim 1, wherein the PD-1 inhibitor is selected from retifanlimab, nivolumab, pembrolizumab, cemiplimab, and dostarlimab.

5. The method of claim 1, wherein the PD-1 inhibitor is retifanlimab.

6-7. (canceled)

8. The method of claim 1, wherein the PD-L1 inhibitor is an anti-PD-L1 antibody.

9. The method of claim 8, wherein the PD-L1 inhibitor is selected from atezolizumab, avelumab, and durvalumab.

10. The method of claim 1, wherein the PD-L1 inhibitor is a small molecule inhibitor.

11. The method of claim 10, wherein the PD-L1 inhibitor is a compound of the following formula 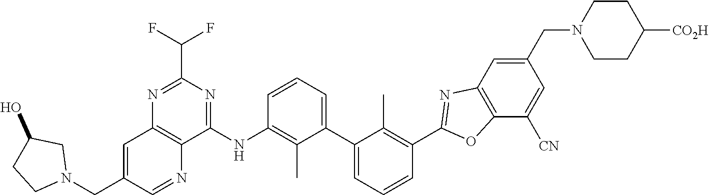 [SMILES: Cc1c(Nc2nc(C(F)F)nc3cc(CN4CC[C@@H](O)C4)cnc23)cccc1-c1cccc(-c2nc3cc(CN4CCC(C(=O)O)CC4)cc(C#N)c3o2)c1C]
or a pharmaceutically acceptable salt thereof.

12. The method of claim 1, wherein the KRAS G12D inhibitor has an IC₅₀ of about 100 nM or lower.

13. The method of claim 1, wherein the KRAS G12D inhibitor is selective for inhibiting G12D versus wild-type KRAS.

14. The method of claim 1, wherein the KRAS G12D inhibitor is a compound of Formula I: 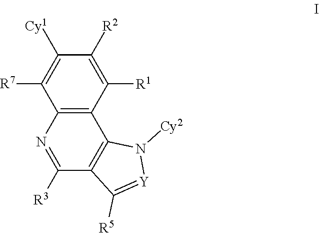
or a pharmaceutically acceptable salt thereof, wherein:
Y is N or CR⁶;
R¹ is selected from H, C₁₋₃ alkyl, C₁₋₃ haloalkyl, cyclopropyl, halo, D, CN, and OR^{a1}; wherein said C₁₋₃ alkyl and cyclopropyl are each optionally substituted with 1 or 2 substituents independently selected from R^{g};
R² is selected from H, C₁₋₃ alkyl, C₁₋₃ haloalkyl, 4-6 membered heterocycloalkyl, phenyl, 5-6 membered heteroaryl, 4-6 membered heterocycloalkyl-C₁₋₃ alkylene, phenyl-C₁₋₃ alkylene, 5-6 membered heteroaryl-C₁₋₃ alkylene, halo, D, CN, and OR^{a2}; wherein said C₁₋₃ alkyl, 4-6 membered heterocycloalkyl, phenyl, 5-6 membered heteroaryl, 4-6 membered heterocycloalkyl-C₁₋₃ alkylene, phenyl-C₁₋₃ alkylene, 5-6 membered heteroaryl-C₁₋₃ alkylene are each optionally substituted with 1 or 2 substituents independently selected from R⁹;
Cy¹ is selected from C₃₋₁₀ cycloalkyl, 4-10 membered heterocycloalkyl, C₆₋₁₀ aryl and 6-10 membered heteroaryl; wherein the 4-10 membered heterocycloalkyl and 6-10 membered heteroaryl each has at least one ring-forming carbon atom and 1, 2, 3, or 4 ring-forming heteroatoms independently selected from N, O, and S; wherein a ring-forming carbon atom of 6-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 6-10 membered heteroaryl are each optionally substituted with 1, 2, 3, or 4 substituents independently selected from R¹⁰;
R³ is selected from H, C₁₋₃ alkyl, C₁₋₃ haloalkyl, C₃₋₆ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, D, CN, OR¹³, C(O)NR^{c3}R^{d3}, NR^{c3}R^{j3}, and NR^{c3}C(O)R^{b3}; wherein said C₁₋₃ alkyl, C₃₋₆ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 substituents independently selected from R³⁰;
R⁵ is selected from H, C₁₋₃ alkyl, C₁₋₃ haloalkyl, cyclopropyl, halo, D, CN, and OR^{a5}; wherein said C₁₋₃ alkyl and cyclopropyl are each optionally substituted with 1 or 2 substituents independently selected from R^{g};
R⁶ is selected from H, C₁₋₃ alkyl, C₁₋₃ haloalkyl, C₃₋₆ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, D, CN, OR^{a6}, and C(O)NR^{c6}R^{d6}; wherein said C₁₋₃ alkyl, C₃₋₆ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1 or 2 substituents independently selected from R⁶⁰;
R⁷ is selected from H, C₁₋₃ alkyl, C₁₋₃ haloalkyl, cyclopropyl, halo, D, CN, and OR^{a7}; wherein said C₁₋₃ alkyl and cyclopropyl are each optionally substituted with 1 or 2 substituents independently selected from R⁹;
Cy² is selected from 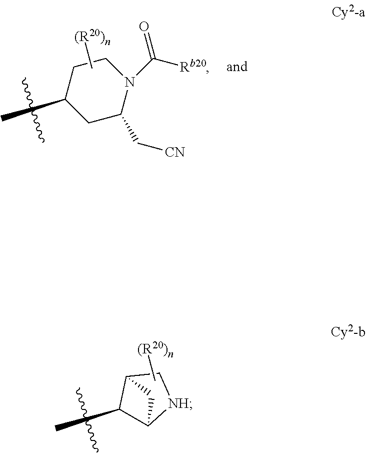
wherein n is 0, 1, or 2;
each R¹⁰ is independently selected from C₁₋₃ alkyl, C₁₋₃ haloalkyl, halo, D, CN, OR^{a10}, C(O)R^{b10}, C(O)NR^{c10}R^{d10}, a(O)OR^{a10}, NR^{c10}R^{d10}, and S(O)₂R^{b10};
each R²⁰ is independently selected from C₁₋₃ alkyl, C₁₋₃ haloalkyl, halo, D, CN, and OR^{a20};
each R³⁰ is independently selected from C₁₋₃ alkyl, C₁₋₃ haloalkyl, C₃₋₆ cycloalkyl, 4-6 membered heterocycloalkyl, phenyl, 5-6 membered heteroaryl, halo, D, CN, OR^{a30}, C(O)R^{b30}, C(O)NR^{c30}R^{d30}C(O)OR^{a30}, NR^{c30}OR^{d30}, and S(O)₂R^{b30}; wherein said C₁₋₃ alkyl, C₃₋₆cycloalkyl, 4-6 membered heterocycloalkyl, phenyl, and 5-6 membered heteroaryl are each optionally substituted with 1 or 2 substituents independently selected from R³¹;
each R³¹ is independently selected from C₁₋₃ alkyl, C₁₋₃ haloalkyl, halo, D, CN, OR^{a31}, C(O)R^{b31}, C(O)NR^{c31}R^{d31}, C(O)OR^{a31}, NR^{c31}R^{d31}, and S(O)₂R^{b31};
each R³³ is independently selected from C₁₋₃ alkyl, C₁₋₃ haloalkyl, C₃₋₆ cycloalkyl, 4-membered heterocycloalkyl, 6-membered heterocycloalkyl, phenyl, 5-6 membered heteroaryl, halo, D, CN, OR^{a30}, C(O)NR^{c30}R^{d30}, and NR^{c30}R^{d30}; wherein said C₁₋₃ alkyl, C₃₋₆cycloalkyl, 4-membered heterocycloalkyl, 6-membered heterocycloalkyl, phenyl, and 5-6 membered heteroaryl are each optionally substituted with 1 or 2 substituents independently selected from R³¹;
each R⁶⁰ is independently selected from C₁₋₃ alkyl, C₁₋₃ haloalkyl, 4-6 membered heterocycloalkyl, 5-6 membered heteroaryl, halo, D, CN, OR^{a60}, C(O)R^{b60}, C(O)NR^{c60}R^{d60}, NR^{c60}C(O)R^{b60}, C(O)OR^{a60}, NR^{c60}C(O)OR^{a60}, NR^{c60}R^{d60}, NR^{c60}S(O)₂R^{b60}, and S(O)₂R^{b60}; wherein said C₁₋₃ alkyl, 4-6 membered heterocycloalkyl, and 5-6 membered heteroaryl are each optionally substituted with 1 or 2 substituents independently selected from R⁶¹;
each R⁶¹ is independently selected from C₁₋₃ alkyl, C₁₋₃ haloalkyl, halo, D, CN, OR^{a61}, and NR^{c61}R^{d61};
R^{a1} is selected from H, C₁₋₃ alkyl, and C₁₋₃ haloalkyl;
each R^{a2} is independently selected from H, C₁₋₃ alkyl, and C₁₋₃haloalkyl;
each R^{b3}, R^{c3} and R^{d3} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R³⁰;
or R^{c3} and R^{d3} attached to the same N atom, together with the N atom to which they are attached, form a 4-, 5-, or 6-membered heterocycloalkyl group optionally substituted with 1, 2, or 3 substituents independently selected from R³⁰;
R^{j3} is selected from C₁₋₃ alkyl, C₁₋₃ haloalkyl, C₃₋₆ cycloalkyl, 4-6 membered heterocycloalkyl, phenyl, and 5-6 membered heteroaryl; wherein said C₁₋₃ alkyl, C₃₋₆cycloalkyl, 4-6 membered heterocycloalkyl, phenyl, and 5-6 membered heteroaryl are each optionally substituted with 1, 2, or 3 substituents independently selected from R³⁰;
or R^{c3} and R^{j3} attached to the same N atom, together with the N atom to which they are attached, form a 4-, 5-, or 6-membered heterocycloalkyl group optionally substituted with 1, 2, or 3 substituents independently selected from R³⁰;
R^{f3} is selected from C₁₋₃ haloalkyl, C₃₋₆ cycloalkyl, 4-6 membered heterocycloalkyl, phenyl and 5-6 membered heteroaryl; wherein said C₁₋₃ haloalkyl, C₃₋₆ cycloalkyl, 4-6 membered heterocycloalkyl, phenyl, and 5-6 membered heteroaryl are each optionally substituted with 1, 2, or 3 substituents independently selected from R³⁰; or
R^{f3} is selected from 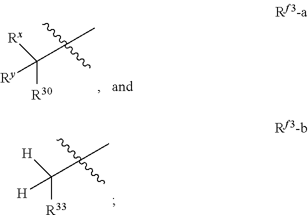
wherein R^{x} is H or C₁₋₂ alkyl and R^{y} is C₁₋₂ alkyl;
or R^{x} and R^{y}, together with the C atom to which they are attached, form a 3-, or 4-membered cycloalkyl group;
R^{a5} is selected from H, C₁₋₃ alkyl, and C₁₋₃ haloalkyl;
each R^{a6}, R^{c6} and R^{d6} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said C₁₋₃ alkyl, C₃₋₆ cycloalkyl, 4-6 membered heterocycloalkyl, phenyl and 5-6 membered heteroaryl are each optionally substituted with 1 or 2 substituents independently selected from R⁶⁰;
R^{a7} is selected from H, C₁₋₃ alkyl, and C₁₋₃ haloalkyl;
each R^{a10}, R^{b10}, R^{c10} and R^{d10} is independently selected from H, C₁₋₃ alkyl, and C₁₋₃ haloalkyl;
each R^{a20} is independently selected from H, C₁₋₃ alkyl, and C₁₋₃ haloalkyl;
R^{b20} is selected from NH₂, C₁₋₃ alkyl, and C₁₋₃ haloalkyl;
each R^{a30}, R^{b30}, R^{c30} and R^{d30} is independently selected from H, C₁₋₃ alkyl, and C₁₋₃ haloalkyl;
each R^{a31}, R^{b31}, R^{c31} and R^{d31} is independently selected from H, C₁₋₃ alkyl, and C₁₋₃ haloalkyl;
each R^{a60}, R^{b60}, R^{c60} and R^{d60} is independently selected from H, C₁₋₃ alkyl, C₁₋₃ haloalkyl, C₃₋₆ cycloalkyl, 4-6 membered heterocycloalkyl, and 5-6 membered heteroaryl; wherein said C₁₋₃ alkyl, C₃₋₆ cycloalkyl, 4-6 membered heterocycloalkyl, and 5-6 membered heteroaryl are each optionally substituted with 1 or 2 substituents independently selected from R⁶¹;
or any R^{c60} and R^{d60} attached to the same N atom, together with the N atom to which they are attached, form a 4-, 5-, or 6-membered heterocycloalkyl group optionally substituted with 1 or 2 substituents independently selected from R⁶¹;
each R^{a61}, R^{c61}, and R^{d61}, is independently selected from H, C₁₋₃ alkyl, and C₁₋₃ haloalkyl; and
each R⁹ is independently selected from D, OH, CN, halo, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino.

15-16. (canceled)

17. The method of claim 14, wherein
Y is CR⁶;
R¹ is H;
R² is —CH₂CH₂CN;
Cy¹ is phenyl; wherein the phenyl is optionally substituted with 1 or 2 substituents independently selected from R¹⁰;
R³ is selected from H, C₁₋₃ alkyl, phenyl, and 5-6 membered heteroaryl; wherein said C₁₋₃ alkyl, phenyl, and 5-6 membered heteroaryl are each optionally substituted with 1, 2 or 3 substituents independently selected from R³⁰;
R⁵ is selected from H and halo;
R⁶ is selected from 4-8 membered heterocycloalkyl; wherein said 4-8 membered heterocycloalkyl is optionally substituted with 1 or 2 substituents independently selected from R⁶⁰; or
R⁶ is selected from C₁₋₃ alkyl; wherein said C₁₋₃ alkyl is substituted with 1 or 2 substituents independently selected from R⁶⁰;
R⁷ is halo;
Cy² is 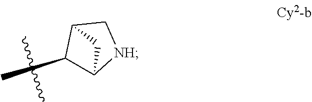
each R¹⁰ is independently selected from C₁₋₃ alkyl and halo;
each R³⁰ is independently selected from C₁₋₃ alkyl, halo, D, OH, and C(O)NR^{c30}R^{d30}, wherein said C₁₋₃ alkyl is optionally substituted with 1 substituent independently selected from R³¹;
each R³¹ is OR^{a31};
each R⁶⁰ is independently selected from C₁₋₃ alkyl, C₁₋₃ haloalkoxy, 4-6 membered heterocycloalkyl, 5-6 membered heteroaryl, halo, C(O)R^{b60}, C(O)NR^{c60}R^{d60}, NR^{c60}C(O)R^{b60}, C(O)OR^{a60}, NR^{c60}C(O)OR^{a60}, and NR^{c60}S(O)₂R^{b60}; wherein said C₁₋₃ alkyl, 4-6 membered heterocycloalkyl, and 5-6 membered heteroaryl are each optionally substituted with 1 or 2 substituents independently selected from R⁶¹;
each R⁶¹ is independently selected from C₁₋₃ alkyl, and halo;
each R^{c30} and R^{d30} is independently selected from H and C₁₋₃ alkyl;
each R^{a31} is independently selected from H and C₁₋₃ alkyl; and
each R^{a60}, R^{b60}, R^{c60} and R^{d60} is independently selected from H, C₁₋₃ alkyl, C₁₋₃ haloalkyl, C₃₋₆ cycloalkyl, 4-6 membered heterocycloalkyl, and 5-6 membered heteroaryl; wherein said C₁₋₃ alkyl, C₃₋₆ cycloalkyl, 4-6 membered heterocycloalkyl, and 5-6 membered heteroaryl are each optionally substituted with 1 or 2 substituents independently selected from R⁶¹;
or any R^{c60} and R^{d60} attached to the same N atom, together with the N atom to which they are attached, form a 4-, 5-, or 6-membered heterocycloalkyl group optionally substituted with 1 or 2 substituents independently selected from R⁶¹.

18. The method of claim 14, wherein
Y is CR⁶;
R¹ is H;
R² is —CH₂CH₂CN;
Cy¹ is phenyl; wherein the phenyl is optionally substituted with 1 or 2 substituents independently selected from R¹⁰;
R³ is selected from H, methyl, ethyl, phenyl, 1,2,4-triazolyl, pyrazyl, and pyridyl; wherein said methyl, phenyl, 1,2,4-triazolyl, pyrazyl, and pyridyl are each optionally substituted with 1, 2 or 3 substituents independently selected from R³⁰;
R⁵ is selected from H and chloro;
R⁶ is selected from pyrrolidinyl, 2-azabicyclo[3.1.0]hexanyl, 2-azabicyclo[2.2.1]heptanyl, and 5-oxo-1,2,3,5-tetrahydroindolizin-3-yl; wherein said pyrrolidinyl, 2-azabicyclo[3.1.0]hexanyl, 2-azabicyclo[2.2.1]heptanyl, and 5-oxo-1,2,3,5-tetrahydroindolizin-3-yl are optionally substituted with 1 or 2 substituents independently selected from R⁶⁰;
R⁷ is fluoro;
Cy² is 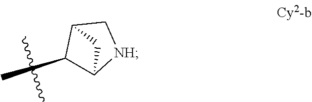
each R¹⁰ is independently selected from methyl, fluoro, and chloro;
each R³⁰ is independently selected from methyl, fluoro, OH, D, and C(O)NR^{c30}R^{d30};
wherein said methyl is optionally substituted with 1 substituent that is R³¹;
each R³¹ is OR^{a31};
each R⁶⁰ is independently selected from methyl, fluoro, C₁₋₂ haloalkoxy, 3-oxomorpholinyl, 2-oxopyrazin-1(2H)-yl), C(O)R^{b60}, C(O)NR^{c60}R^{d60}, NR^{c60}C(O)R^{b60}, C(O)OR^{a6}, NR^{c60}C(O)OR^{a60}, and NR^{c60}S(O)₂R^{b60}; wherein said 3-oxomorpholinyl, and 2-oxopyrazin-1(2H)-yl) are each optionally substituted with 1 or 2 substituents independently selected from R⁶¹;
each R⁶¹ is independently selected from methyl and fluoro;
each R^{c30} and R^{d30} is independently selected from H and methyl;
each R^{a31} is independently selected from H and methyl; and
each R^{a60}, R^{b60}, R^{c60} and R^{d60} is independently selected from H, C₁₋₂ alkyl, C₁ haloalkyl, cyclopropyl, tetrahydrofuranyl, and thiazolyl; wherein said C₁₋₂ alkyl, cyclopropyl, tetrahydrofuranyl, and thiazolyl are each optionally substituted with 1 or 2 substituents independently selected from R⁶¹;
or any R^{c60} and R^{d60} attached to the same N atom, together with the N atom to which they are attached, form an azetidinyl group optionally substituted with 1 or 2 substituents independently selected from R⁶¹.

19. (canceled)

20. The method of claim 14, wherein the compound of Formula I is a compound of Formula III: 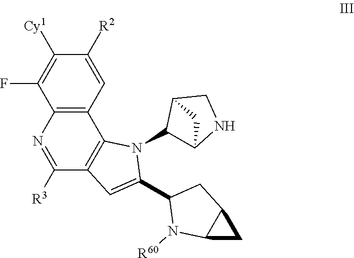
or a pharmaceutically acceptable salt thereof.

21. The method of claim 1, wherein the KRAS G12D inhibitor is selected from
3-(1-((1R,4R,5S)-2-azabicyclo[2.1.1]hexan-5-yl)-7-(7-chloro-3-hydroxynaphthalen-1-yl)-6-fluoro-2-methyl-4-(1H-1,2,4-triazol-1-yl)-1H-pyrrolo[3,2-c]quinolin-8-yl)propanenitrile;
3-(1-((1R,4R,5S)-2-azabicyclo[2.1.1]hexan-5-yl)-7-(5,7-difluoro-1H-indol-3-yl)-6-fluoro-2-methyl-4-((S)-1-((S)-1-methylpyrrolidin-2-yl)ethoxy)-1H-pyrrolo[3,2-c]quinolin-8-yl)propanenitrile;
3-(1-((1R,4R,5S)-2-azabicyclo[2.1.1]hexan-5-yl)-6-fluoro-7-(6-fluoro-5-methyl-1H-indol-3-yl)-2-methyl-4-((S)-1-((S)-1-methylpyrrolidin-2-yl)ethoxy)-1H-pyrrolo[3,2-c]quinolin-8-yl)propanenitrile;
3-(2-(3-(azetidin-1-yl)-3-oxopropyl)-1-((1R,4R,5S)-2-azabicyclo[2.1.1]hexan-5-yl)-7-(2,3-dichlorophenyl)-6-fluoro-4-((S)-1-((S)-1-methylpyrrolidin-2-yl)ethoxy)-1H-pyrrolo[3,2-c]quinolin-8-yl)propanenitrile;
3-((1-((1R,4R,5S)-2-azabicyclo[2.1.1]hexan-5-yl)-6-fluoro-7-(3-hydroxynaphthalen-1-yl)-8-methyl-4-((S)-1-((S)-1-methylpyrrolidin-2-yl)ethoxy)-1H-pyrrolo[3,2-c]quinolin-2-yl)methyl)oxazolidin-2-one;
8-(1-((1R,4R,5S)-2-azabicyclo[2.1.1]hexan-5-yl)-6-fluoro-2,8-dimethyl-4-((S)-1-((S)-1-methylpyrrolidin-2-yl)ethoxy)-1H-pyrrolo[3,2-c]quinolin-7-yl)-1-naphthonitrile;
1-((2S,4S)-1-acetyl-2-(cyanomethyl)piperidin-4-yl)-7-(8-cyanonaphthalen-1-yl)-6-fluoro-4-((S)-1-((S)-1-methylpyrrolidin-2-yl)ethoxy)-1H-pyrazolo[4,3-c]quinoline-8-carbonitrile;
8-(1-((1R,4R,5S)-2-azabicyclo[2.1.1]hexan-5-yl)-6-fluoro-8-methyl-4-((S)-1-((S)-1-methylpyrrolidin-2-yl)ethoxy)-2-((3-oxomorpholino)methyl)-1H-pyrrolo[3,2-c]quinolin-7-yl)-1-naphthonitrile;
3-(7-(benzo[b]thiophen-3-yl)-1-((1R,4R,5S)-2-azabicyclo[2.1.1]hexan-5-yl)-6-fluoro-4-((S)-1-((S)-1-methylpyrrolidin-2-yl)ethoxy)-2-((2-oxopyrrolidin-1-yl)methyl)-1H-pyrrolo[3,2-c]quinolin-8-yl)propanenitrile;
3-(1-((1R,4R,5S)-2-azabicyclo[2.1.1]hexan-5-yl)-4-(((S)-1-(dimethylamino)propan-2-yl)oxy)-6-fluoro-7-(7-fluoronaphthalen-1-yl)-2-((2-oxopyrrolidin-1-yl)methyl)-1H-pyrrolo[3,2-c]quinolin-8-yl)propanenitrile;
8-(1-((1R,4R,5S)-2-azabicyclo[2.1.1]hexan-5-yl)-8-(2-cyanoethyl)-6-fluoro-2-methyl-4-((S)-1-((S)-1-methylpyrrolidin-2-yl)ethoxy)-1H-pyrrolo[3,2-c]quinolin-7-yl)-1-naphthonitrile;
3-(1-((1R,4R,5S)-2-azabicyclo[2.1.1]hexan-5-yl)-7-(2,3-dichloro-5-hydroxyphenyl)-6-fluoro-2-methyl-4-((S)-1-((S)-1-methylpyrrolidin-2-yl)ethoxy)-1H-pyrrolo[3,2-c]quinolin-8-yl)propanenitrile;
3-(1-((1R,4R,5S)-2-azabicyclo[2.1.1]hexan-5-yl)-8-(2-cyanoethyl)-6-fluoro-4-((3-fluoro-1-methylazetidin-3-yl)methoxy)-7-(3-hydroxynaphthalen-1-yl)-1H-pyrrolo[3,2-c]quinolin-2-yl)-N,N-dimethylpropanamide;
3-(1-((1R,4R,5S)-2-azabicyclo[2.1.1]hexan-5-yl)-6-fluoro-7-(3-hydroxynaphthalen-1-yl)-2-methyl-4-(5-methylpyrazin-2-yl)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoronaphthalen-1-yl)-4-methyl-2-((4-methyl-2-oxopiperazin-1-yl)methyl)-1H-pyrrolo[3,2-c]quinolin-8-yl)propanenitrile;
3-(1-((1R,4R,5S)-2-azabicyclo[2.1.1]hexan-5-yl)-7-(2,3-dichloro-5-hydroxyphenyl)-4-ethoxy-6-fluoro-2-((4-isopropyl-2-oxopiperazin-1-yl)methyl)-1H-pyrrolo[3,2-c]quinolin-8-yl)propanenitrile;
3-(1-((1R,4R,5S)-2-azabicyclo[2.1.1]hexan-5-yl)-4-(3-(dimethylamino)-3-methylazetidin-1-yl)-6-fluoro-7-(7-fluoronaphthalen-1-yl)-2-((3-oxomorpholino)methyl)-1H-pyrrolo[3,2-c]quinolin-8-yl)propanenitrile;
3-(1-((1R,4R,5S)-2-azabicyclo[2.1.1]hexan-5-yl)-4-ethoxy-6-fluoro-7-(3-hydroxynaphthalen-1-yl)-2-(1-(3-oxomorpholino)ethyl)-1H-pyrrolo[3,2-c]quinolin-8-yl)propanenitrile;
3-(1-((endo)-2-azabicyclo[2.1.1]hexan-5-yl)-6-fluoro-7-(3-hydroxynaphthalen-1-yl)-4-((S)-1-((S)-1-methylpyrrolidin-2-yl)ethoxy)-2-(pyridin-3-yl)-1H-pyrrolo[3,2-c]quinolin-8-yl)propanenitrile;
3-(2-(3-(azetidin-1-yl)-3-oxopropyl)-1-((1R,4R,5S)-2-azabicyclo[2.1.1]hexan-5-yl)-7-(7,8-difluoronaphthalen-1-yl)-6-fluoro-4-((S)-1-((S)-1-methylpyrrolidin-2-yl)ethoxy)-1H-pyrrolo[3,2-c]quinolin-8-yl)propanenitrile;
3-(2-(3-(azetidin-1-yl)-3-oxopropyl)-1-((1R,4R,5S)-2-azabicyclo[2.1.1]hexan-5-yl)-7-(6,7-difluoronaphthalen-1-yl)-6-fluoro-4-((S)-1-((S)-1-methylpyrrolidin-2-yl)ethoxy)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoro-3-hydroxynaphthalen-1-yl)-2-methyl-4-((S)-1-((S)-1-methylpyrrolidin-2-yl)ethoxy)-1H-pyrrolo[3,2-c]quinolin-8-yl)propanenitrile;
1-(1-((2S,4S)-1-acetyl-2-(cyanomethyl)piperidin-4-yl)-8-chloro-6-fluoro-4-((S)-1-((S)-1-methylpyrrolidin-2-yl)ethoxy)-1H-pyrazolo[4,3-c]quinolin-7-yl)isoquinoline-8-carbonitrile;
8-(1-((2S,4S)-1-acetyl-2-(cyanomethyl)piperidin-4-yl)-8-chloro-6-fluoro-4-((S)-1-((S)-1-methylpyrrolidin-2-yl)ethoxy)-1H-pyrrolo[3,2-c]quinolin-7-yl)-1-naphthonitrile;
8-(1-((2S,4S)-1-acetyl-2-(cyanomethyl)piperidin-4-yl)-8-chloro-6-fluoro-4-((S)-1-((S)-1-methylpyrrolidin-2-yl)ethoxy)-1H-pyrazolo[4,3-c]quinolin-7-yl)-1-naphthonitrile;
3-(1-((1R,4R,5S)-2-azabicyclo[2.1.1]hexan-5-yl)-6-fluoro-7-(7-fluoro-3-hydroxynaphthalen-1-yl)-2-methyl-4-(1H-1,2,4-triazol-1-yl)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoronaphthalen-1-yl)-2-methyl-4-((S)-1-((S)-1-methylpyrrolidin-2-yl)ethoxy)-1H-pyrrolo[3,2-c]quinolin-8-yl)propanenitrile;
(2R)-2-(1-((1R,4R,5S)-2-azabicyclo[2.1.1]hexan-5-yl)-8-(2-cyanoethyl)-7-(2,3-dichlorophenyl)-6-fluoro-4-(1H-1,2,4-triazol-1-yl)-1H-pyrrolo[3,2-c]quinolin-2-yl)-N,N-dimethylpyrrolidine-1-carboxamide;
methyl (2R)-2-(1-((1R,4R,5S)-2-azabicyclo[2.1.1]hexan-5-yl)-7-(2-chloro-3-methylphenyl)-8-(2-cyanoethyl)-6-fluoro-4-(1H-1,2,4-triazol-1-yl)-1H-pyrrolo[3,2-c]quinolin-2-yl)pyrrolidine-1-carboxylate;
methyl (1S,3R,5S)-3-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)-2-azabicyclo[3.1.0]hexane-2-carboxylate;
3-(1-((1R,4R,5S)-2-azabicyclo[2.1.1]hexan-5-yl)-7-(2,3-dichlorophenyl)-6-fluoro-4-methyl-2-(5-oxo-1,2,3,5-tetrahydroindolizin-3-yl)-1H-pyrrolo[3,2-c]quinolin-8-yl)propanenitrile;
methyl (2R)-2-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)pyrrolidine-1-carboxylate;
methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(methylcarbamoyl)pyridin-3-yl)-1H-pyrrolo[3,2-c]quinolin-2-yl)pyrrolidine-1-carboxylate;
3-(1-((1R,4R,5S)-2-azabicyclo[2.1.1]hexan-5-yl)-7-(2-chloro-3-fluorophenyl)-2-((R)-1-(cyclopropanecarbonyl)pyrrolidin-2-yl)-6-fluoro-4-methyl-1H-pyrrolo[3,2-c]quinolin-8-yl)propanenitrile;
8-(2-((R)-1-acetylpyrrolidin-2-yl)-1-((1R,4R,5S)-2-azabicyclo[2.1.1]hexan-5-yl)-6-fluoro-8-methyl-4-(2-methylpyridin-4-yl)-1H-pyrrolo[3,2-c]quinolin-7-yl)-1,2,3,4-tetrahydronaphthalene-1-carbonitrile;
5-(1-((1R,4R,5S)-2-azabicyclo[2.1.1]hexan-5-yl)-7-(3-chloro-2-methylphenyl)-8-(2-cyanoethyl)-6-fluoro-2-((R)-1-(2-oxopyrazin-1(2H)-yl)ethyl)-1H-pyrrolo[3,2-c]quinolin-4-yl)-N-methylpicolinamide;
3-(1-((1R,4R,5S)-2-azabicyclo[2.1.1]hexan-5-yl)-6-fluoro-7-(7-fluoronaphthalen-1-yl)-4-(6-(2-hydroxypropan-2-yl)pyridin-3-yl)-2-((R)-1-(2-oxopyrazin-1(2H)-yl)ethyl)-1H-pyrrolo[3,2-c]quinolin-8-yl)propanenitrile;
3-(1-((1R,4R,5S)-2-azabicyclo[2.1.1]hexan-5-yl)-7-(3-chloro-2-methylphenyl)-6-fluoro-4-(5-methylpyrazin-2-yl)-2-((R)-1-(2-oxopyrazin-1(2H)-yl)ethyl)-1H-pyrrolo[3,2-c]quinolin-8-yl)propanenitrile;
methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(5-fluoro-6-(methylcarbamoyl)pyridin-3-yl)-1H-pyrrolo[3,2-c]quinolin-2-yl)pyrrolidine-1-carboxylate;
methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;
ethyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;
3-(1-((1R,4R,5S)-2-azabicyclo[2.1.1]hexan-5-yl)-7-(2,3-dichlorophenyl)-2-((R)-1-(3,3-difluoroazetidine-1-carbonyl)pyrrolidin-2-yl)-6-fluoro-4-(methyl-d3)-1H-pyrrolo[3,2-c]quinolin-8-yl)propanenitrile;
3-(1-((1R,4R,5S)-2-azabicyclo[2.1.1]hexan-5-yl)-7-(2,3-dichlorophenyl)-2-((R)-1-(3,3-difluoroazetidine-1-carbonyl)pyrrolidin-2-yl)-6-fluoro-1H-pyrrolo[3,2-c]quinolin-8-yl)propanenitrile;
3-(1-((1R,4R,5S)-2-azabicyclo[2.1.1]hexan-5-yl)-7-(3-chloro-2-methylphenyl)-6-fluoro-4-(5-methylpyrazin-2-yl)-2-((R)-1-(3-oxomorpholino)ethyl)-1H-pyrrolo[3,2-c]quinolin-8-yl)propanenitrile;
5-(1-((1R,4R,5S)-2-azabicyclo[2.1.1]hexan-5-yl)-8-(2-cyanoethyl)-6-fluoro-7-(7-fluoronaphthalen-1-yl)-2-((R)-1-(3-oxomorpholino)ethyl)-1H-pyrrolo[3,2-c]quinolin-4-yl)-N-methylpicolinamide;
3-(1-((1R,4R,5S)-2-azabicyclo[2.1.1]hexan-5-yl)-6-fluoro-7-(7-fluoronaphthalen-1-yl)-4-(6-(2-hydroxypropan-2-yl)pyridin-3-yl)-2-((R)-1-(3-oxomorpholino)ethyl)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoronaphthalen-1-yl)-4-(5-methylpyrazin-2-yl)-2-((R)-1-(3-oxomorpholino)ethyl)-1H-pyrrolo[3,2-c]quinolin-8-yl)propanenitrile;
methyl (1R,3R,5R)-3-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)-2-azabicyclo[3.1.0]hexane-2-carboxylate;
3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-7-(2,3-dichlorophenyl)-6-fluoro-4-(6-(2-hydroxypropan-2-yl)pyridin-3-yl)-2-((R)-1-(2-oxopyrazin-1(2H)-yl)ethyl)-1H-pyrrolo[3,2-c]quinolin-8-yl)propanenitrile;
methyl (2R,4S)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)-4-fluoropyrrolidine-1-carboxylate;
methyl (2R,5R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)-5-methylpyrrolidine-1-carboxylate;
methyl (2R)-2-(1-((1R,4R,5S)-2-azabicyclo[2.1.1]hexan-5-yl)-3-chloro-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;
4-(1-((1R,4R,5S)-2-azabicyclo[2.1.1]hexan-5-yl)-8-(2-cyanoethyl)-7-(2,3-dichlorophenyl)-6-fluoro-2-((R)-1-(2-oxopyrazin-1(2H)-yl)ethyl)-1H-pyrrolo[3,2-c]quinolin-4-yl)-2-fluoro-N-methylbenzamide;
methyl ((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)carbamate;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-2,2-difluoroacetamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-2,2-difluoroacetamide;
(2S)—N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)tetrahydrofuran-2-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)cyclopropanesulfonamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)thiazole-4-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-N-methylcyclopropanecarboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-methylcyclopropane-1-carboxamide;
3-(1-((1R,4R,5S)-2-azabicyclo[2.1.1]hexan-5-yl)-7-(2,3-dichlorophenyl)-6-fluoro-4-(1-hydroxyethyl)-2-((1R,3R,5R)-2-(1-methylcyclopropane-1-carbonyl)-2-azabicyclo[3.1.0]hexan-3-yl)-1H-pyrrolo[3,2-c]quinolin-8-yl)propanenitrile;
3-(1-((1R,4R,5S)-2-azabicyclo[2.1.1]hexan-5-yl)-7-(2,3-dichlorophenyl)-6-fluoro-2-((1R,3R,5R)-2-(1-fluorocyclopropane-1-carbonyl)-2-azabicyclo[3.1.0]hexan-3-yl)-4-(1-hydroxyethyl)-1H-pyrrolo[3,2-c]quinolin-8-yl)propanenitrile;
3-(1-((1R,4R,5S)-2-azabicyclo[2.1.1]hexan-5-yl)-7-(2,3-dichlorophenyl)-6-fluoro-2-((1R,3R,5R)-2-(1-fluorocyclopropane-1-carbonyl)-2-azabicyclo[3.1.0]hexan-3-yl)-4-methyl-1H-pyrrolo[3,2-c]quinolin-8-yl)propanenitrile;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-fluorocyclopropane-1-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-fluorocyclobutane-1-carboxamide;
3-(1-((1R,4R,5S)-2-azabicyclo[2.1.1]hexan-5-yl)-7-(3-chloro-2-methylphenyl)-2-(1-(2,6-dimethyl-3-oxo-2,3-dihydropyridazin-4-yl)ethyl)-6-fluoro-4-methyl-1H-pyrrolo[3,2-c]quinolin-8-yl)propanenitrile;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)pyrimidine-4-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)pyridazine-3-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-3,3-difluoroazetidine-1-carboxamide;
3-(1-((1R,4R,5S)-2-azabicyclo[2.1.1]hexan-5-yl)-7-(2,3-dichlorophenyl)-6-fluoro-4-methyl-2-((R)-1-((1-methyl-1H-pyrazol-4-yl)amino)ethyl)-1H-pyrrolo[3,2-c]quinolin-8-yl)propanenitrile;
5-(1-((1R,4R,5S)-2-azabicyclo[2.1.1]hexan-5-yl)-8-(2-cyanoethyl)-7-(2,3-dichlorophenyl)-6-fluoro-2-((R)-1-(1-fluorocyclopropane-1-carbonyl)pyrrolidin-2-yl)-1H-pyrrolo[3,2-c]quinolin-4-yl)-N,N-dimethylpicolinamide; and
methyl (2R)-2-(1-((1R,4R,5S)-2-azabicyclo[2.1.1]hexan-5-yl)-8-(2-cyanoethyl)-7-(2,3-dichlorophenyl)-4-(4-((dimethylamino)methyl)-2,3-difluorophenyl)-6-fluoro-1H-pyrrolo[3,2-c]quinolin-2-yl)pyrrolidine-1-carboxylate;
and pharmaceutically acceptable salts thereof.

22. The method of claim 1, wherein the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

23. (canceled)

24. The method of claim 1, wherein the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate.

25. (canceled)

26. The method of claim 1, wherein the KRAS G12D inhibitor is a compound of Formula IV 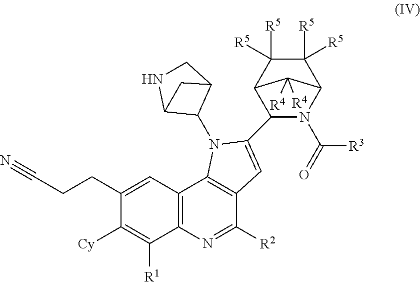
or a pharmaceutically acceptable salt thereof, wherein:
Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
R¹ is halogen;
R² is selected from H, D, C₁₋₃ alkyl, C₂₋₃ alkenyl, C₂₋₃ alkynyl, C₁₋₃ haloalkyl, C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃alkylene, 5-6 membered heteroaryl-C₁₋₃alkylene, halo, CN, OR^{a2}, C(O)R^{b2}, C(O)NR^{c2}R^{d2}, NR^{c2}R^{e2}, and NR^{c2}C(O)R^{b2}; wherein the C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene forming R² are each optionally substituted with 1, 2, or 3 substituents independently selected from R^{2A}; wherein the ring-forming atoms of the 4-6 membered heterocycloalkyl, 5-6 membered heteroaryl, C₃₋₆ cycloalkyl-C₁₋₃ alkylene, and 4-6 membered heterocycloalkyl-C₁₋₃alkylene forming R² consist of at least one carbon atom and 1, 2, 3, or 4 heteroatoms selected from O, N, and S; wherein a ring-forming carbon atom of the 4-6 membered heterocycloalkyl, 5-6 membered heteroaryl, C₃₋₆ cycloalkyl-C₁₋₃ alkylene, and 4-6 membered heterocycloalkyl-C₁₋₃alkylene forming R² is optionally substituted by oxo to form a carbonyl group; and wherein the C₁₋₃ alkyl, C₂₋₃ alkenyl, and C₂₋₃ alkynyl forming R² are each optionally substituted with 1, 2, or 3 substituents independently selected from R^{2B};
each R^{a2} is independently selected from C₁₋₆ alkyl, C₂₋₆ alkenyl, C₂₋₆ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a2} are each optionally substituted with 1, 2, or 3 substituents independently selected from R^{2A}; wherein the ring-forming atoms of the 4-6 membered heterocycloalkyl and 5-6 membered heteroaryl forming R^{a2} consist of at least one carbon atom and 1, 2, 3, or 4 heteroatoms selected from O, N, and S; wherein a ring-forming carbon atom of the 4-6 membered heterocycloalkyl and 5-6 membered heteroaryl forming R^{a2} is optionally substituted by oxo to form a carbonyl group; and wherein the C₁₋₆ alkyl, C₂₋₆ alkenyl, and C₂₋₆ alkynyl forming R^{a2} are each optionally substituted with 1, 2, or 3 substituents independently selected from R^{2B};
each R^{b2}, R^{c2}, and R^{d2} is independently selected from H, C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{b2}, R^{c2}, and R^{d2} are each optionally substituted with 1, 2, or 3 substituents independently selected from R^{2A}; the ring-forming atoms of the 4-6 membered heterocycloalkyl and 5-6 membered heteroaryl forming R^{b2}, R^{c2}, and R^{d2} consist of at least one carbon atom and 1, 2, 3, or 4 heteroatoms selected from O, N, and S; wherein a ring-forming carbon atom of the 4-6 membered heterocycloalkyl and 5-6 membered heteroaryl forming R^{b2}R^{c2}, and R^{d2} is optionally substituted by oxo to form a carbonyl group; and wherein the C₁₋₃ alkyl, C₂₋₃ alkenyl, and C₂₋₃ alkynyl forming R^{b2}, R^{c2}, and R^{d2} are each optionally substituted with 1, 2, or 3 substituents independently selected from R^{2B}; or
any R^{c2} and R^{d2} attached to the same N atom, together with the N atom to which they are attached, form a 4-, 5-, or 6-membered heterocycloalkyl group optionally substituted with 1, 2, or 3 substituents independently selected from R^{2B};
each R^{e2} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl and 5-6 membered heteroaryl; wherein the C₃₋₆ cycloalkyl, 4-6 membered heterocycloalkyl, phenyl and 5-6 membered heteroaryl forming R^{e2} are each optionally substituted with 1, 2, or 3 substituents independently selected from R^{2A}; wherein the ring-forming atoms of the 4-6 membered heterocycloalkyl and 5-6 membered heteroaryl forming R^{e2} consist of at least one carbon atom and 1, 2, 3, or 4 heteroatoms selected from O, N, and S; wherein a ring-forming carbon atom of the 4-6 membered heterocycloalkyl and 5-6 membered heteroaryl forming R^{e2} is optionally substituted by oxo to form a carbonyl group; and wherein the C₁₋₃ alkyl, C₂₋₃ alkenyl, and C₂₋₃ alkynyl, forming R^{e2} are each optionally substituted with 1, 2, or 3 substituents independently selected from R^{2B}; or
R^{c2} and R^{e2} attached to the same N atom, together with the N atom to which they are attached, form a 4-, 5-, or 6-membered heterocycloalkyl group optionally substituted with 1, 2, or 3 substituents independently selected from R^{2B};
each R^{2A} is independently selected from C₁₋₃ alkyl, C₂₋₃ alkenyl, C₂₋₃ alkynyl, C₁₋₃ haloalkyl, and R^{2B}, wherein the C₁₋₃ alkyl, C₂₋₃ alkenyl, and C₂₋₃ alkynyl, forming R^{2A} are each optionally substituted with 1, 2 or 3 substituents independently selected from R^{2B};
each R^{2B} is independently selected from C₃₋₆cycloalkyl, 4-10 membered heterocycloalkyl, phenyl, 5-6 membered heteroaryl, halo, D, CN, OR^{a2}B, C(O)R^{b2B}, C(O)NR^{c2B}R^{d2B}, C(O)OR^{a2B}, NR^{c2B}R^{d2B}, and S(O)₂R^{b2B}; wherein the C₁₋₃ alkyl, C₃₋₆cycloalkyl, 4-6 membered heterocycloalkyl, phenyl, and 5-6 membered heteroaryl are each optionally substituted with 1, 2, or 3 substituents independently selected from R^{2C};
each R^{2C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, 5-6 membered heteroaryl, halo, D, CN, OR^{a2C}, C(O)R^{b2C}, C(O)NR^{c2}CR^{d2C}(O)OR^{a2C}, NR^{c2C}R^{d2C}, and S(O)₂R^{b2C};
each R^{a2B}, R^{b2B}, R^{c2B} and R^{d2B} is independently selected from H, C₁₋₃ alkyl, and C₁₋₃ haloalkyl;
each R^{a2C}, R^{b2C}, R^{c2C} and R^{d2C} is independently selected from H, C₁₋₃ alkyl, and C₁₋₃ haloalkyl;
R³ is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, OR^{3A}, and NR^{3B}R^{3C}; wherein the C₃₋₁₀ cycloalkyl, 4-10 membered heterocycloalkyl, C₆₋₁₀ aryl, and 5-10 membered heteroaryl C₁₋₃ alkyl forming R³ are each optionally substituted with 1, 2, or 3 substituents independently selected from R^{3D}; wherein the ring-forming atoms of the 4-10 membered heterocycloalkyl and 5-10 membered heteroaryl forming R³ consist of at least one carbon atom and 1, 2, 3, or 4 heteroatoms selected from O, N, and S; wherein a ring-forming carbon atom of the 4-10 membered heterocycloalkyl or 5-10 membered heteroaryl forming R³ is optionally substituted by oxo to form a carbonyl group; and wherein the C₁₋₃ alkyl, C₂₋₃ alkenyl, and C₂₋₃ alkynyl forming R³ are each optionally substituted with 1, 2, or 3 substituents independently selected from R^{3E};
R^{3A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, and 5-10 membered heteroaryl; wherein the C₃₋₁₀ cycloalkyl, 4-10 membered heterocycloalkyl, C₆₋₁₀ aryl, and 5-10 membered heteroaryl C₁₋₃ alkyl forming R^{3A} are each optionally substituted with 1, 2, or 3 substituents independently selected from R^{3D}; wherein the ring-forming atoms of the 4-10 membered heterocycloalkyl and 5-10 membered heteroaryl forming R^{3A} consist of at least one carbon atom and 1, 2, 3, or 4 heteroatoms selected from 0 N, and S; wherein a ring-forming carbon atom of the 4-10 membered heterocycloalkyl or 5-10 membered heteroaryl forming R^{3A} is optionally substituted by oxo to form a carbonyl group; and wherein the C₁₋₃ alkyl, C₂₋₃ alkenyl, and C₂₋₃ alkynyl forming R^{3A} are each optionally substituted with 1, 2, or 3 substituents independently selected from R^{3E};
R^{3B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, and 5-10 membered heteroaryl; wherein the C₃₋₁₀ cycloalkyl, 4-10 membered heterocycloalkyl, C₆₋₁₀ aryl, and 5-10 membered heteroaryl forming R^{3B} are each optionally substituted with 1, 2, or 3 substituents independently selected from R^{3D} wherein the ring-forming atoms of the 4-10 membered heterocycloalkyl and 5-10 membered heteroaryl forming R^{3B} consist of at least one carbon atom and 1, 2, 3, or 4 heteroatoms selected from O, N, and S; wherein a ring-forming carbon atom of the 4-10 membered heterocycloalkyl and 5-10 membered heteroaryl forming R^{3B} is optionally substituted by oxo to form a carbonyl group; and wherein the C₁₋₃ alkyl, C₂₋₃ alkenyl, and C₂₋₃ alkynyl forming R^{3B} are each optionally substituted with 1, 2, or 3 substituents independently selected from R^{3E};
R^{3B} and R^{3C}, together with the N atom to which they are both attached, optionally form a 4-, 5-, 6-, or 7-membered heterocycloalkyl group that is optionally substituted with 1, 2, or 3 substituents independently selected from independently selected from R^{3D};
R^{3C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{3C} are each optionally substituted with 1, 2, or 3 substituents independently selected from R^{3E};
each R^{3D} is independently selected from C₁₋₃ alkyl, C₁₋₃ haloalkyl, C₂₋₃ alkenyl, C₂₋₃ alkynyl, and R^{3E}; wherein each of the C₁₋₃ alkyl, C₂₋₃ alkenyl, and C₂₋₃ alkynyl forming R^{3D} is optionally substituted with 1, 2, or 3 substituents independently selected from R^{3E};
each R^{3E} is independently selected from D, halo, CN, OR^{a3}, SR^{a3}, C(O)R^{b3}C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3} NR^{c3}C(O)OR^{a3}, C(═NR^{e3})NR^{c3}R^{d3}, NR^{c3}C(═NR^{e3})NR^{c3}R^{d3} S(O)R^{b3}, S(O)NR^{c3}R^{d3}, S(O)₂R^{b3} NR^{c3}S(O)₂R^{b3}, and S(O)₂NR^{c3}R^{d3};
R^{a3}, R^{b3}, R^{c3}, and R^{d3} are each independently selected from H, C₁₋₃ alkyl, C₂₋₃ alkenyl, C₂₋₃ alkynyl, C₆₋₁₀ aryl, C₃₋₇ cycloalkyl, 5-10 membered heteroaryl, 4-10 membered heterocycloalkyl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a3}, R^{b3}, R^{c3}, and R^{d3} are each optionally substituted with 1, 2, 3, 4, or 5 substituents independently selected from C₁₋₃ alkyl, C₁₋₃ haloalkyl, C₂₋₃ alkenyl, C₂₋₃ alkynyl, halo, CN, OR^{a3A}, SR^{a3A}, C(O)R^{b3A}, C(O)NR^{c3A}R^{d3A}, C(O)OR^{a3A}, OC(O)R^{b3A}, OC(O)NR^{c3A}R^{d3A}, NR^{c3A}R^{d3A}, NR^{c3A}C(O)R^{b3A}, NR^{c3A}C(O)NR^{c3A}R^{d3A}, NR^{c3A}C(O)OR^{a3A}, C(═NR^{e3A})NR^{c3A}R^{d3A}, NR^{c3A}C(═NR^{e3A})NR^{c3A}R^{d3A}, S(O)R^{b3A}, S(O)NR^{c3A}R^{d3A}, S(O)₂R^{b3A}, NR^{c3A}S(O)₂R^{b3A} and S(O)₂NR^{c3A}R^{d3A}; wherein the ring-forming atoms of the 4-10 membered heterocycloalkyl, 5-10 membered heteroaryl, 5-10 membered heteroaryl-C₁₋₃alkyl, and 4-10 membered heterocycloalkyl-C₁₋₃ alkyl forming R^{a3}, R^{b3}, R^{c3}, and R^{d3} consist of at least one carbon atom and 1, 2, 3, or 4 heteroatoms selected from O, N, and S; wherein a ring-forming carbon atom of the 4-10 membered heterocycloalkyl, 5-10 membered heteroaryl, 5-10 membered heteroaryl-C₁₋₃ alkyl, and 4-10 membered heterocycloalkyl-C₁₋₃ alkyl forming R^{a3}, R^{b3}, R^{c3}, and R^{d3} is optionally substituted by oxo to form a carbonyl group; or
R^{c3} and R^{d3} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{a3A}, SR^{a3A}, C(O)R^{b3A}, C(O)NR^{c3A}R^{d3A}, C(O)OR^{a3A}, OC(O)R^{b3A}, OC(O)NR^{c3A}R^{d3A}, NR^{c3A}R^{d3A}, NR^{c3A}C(O)R^{b3A}, NR^{c3A}C(O)NR^{c3A}R^{d3A}, NR^{c3A}C(O)OR^{a3A}, C(═NR^{e3A})NR^{c3A}R^{d3A}, NR^{c3A}C(═NR^{e3A})NR^{c3A}R^{d3A}, S(O)R^{b3A}, S(O)NR^{c3A}R^{d3A}, S(O)₂R^{b3A}, NR^{c3A}S(O)₂R^{b3A}, and S(O)₂NR^{c3A}R^{d3A};
R^{a3A}, R^{b3A}, R^{c3A} and R^{d3A} are each independently selected from H, C₁₋₃ alkyl, C₁₋₃ haloalkyl, C₂₋₃ alkenyl, C₂₋₃ alkynyl, aryl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₁₋₆ alkyl, C₁₋₆ haloalkyl, C₂₋₆ alkenyl, C₂₋₆ alkynyl C₆₋₁₀ aryl-C₁₋₃alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a3A}, R^{b3A}, R^{c3A} and R^{d3A} are each optionally substituted with 1, 2, or 3 substituents independently selected from OH, CN, amino, NH(C₁₋₆ alkyl), N(C₁₋₆ alkyl)₂, halo, C₁₋₆ alkyl, C₁₋₆ alkoxy, C₁₋₆ haloalkyl, and C₁₋₆ haloalkoxy; wherein the ring-forming atoms of the 4-10 membered heterocycloalkyl, 5-10 membered heteroaryl, 5-10 membered heteroaryl-C₁₋₃alkyl, and 4-10 membered heterocycloalkyl-C₁₋₃alkyl forming R^{a3A}, R^{b3A}, R^{c3A}, and R^{d3A} consist of at least one carbon atom, and 1, 2, 3, or 4 heteroatoms selected from O, N, and S; and wherein a ring-forming carbon atom of the 4-10 membered heterocycloalkyl, 5-10 membered heteroaryl, 5-10 membered heteroaryl-C₁₋₃alkyl, and 4-10 membered heterocycloalkyl-C₁₋₃alkyl forming R^{a3A}, R^{b3A}, R^{c3A} and R^{d3A} is optionally substituted by oxo to form a carbonyl group; or
R^{c3A} and R^{d3A} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₆ alkyl, C₁₋₆ alkoxy, C₁₋₆ haloalkyl, and C₁₋₆ haloalkoxy;R^{e3}, and R^{e3A} are each, independently, H, CN or NO₂;
each R⁴ is independently selected from H, D, C₁₋₃ alkyl, C₁₋₃ haloalkyl, C₂₋₃ alkenyl, C₂₋₃ alkynyl, halo, and OR^{a4};
each R^{a4} is independently selected from H, C₁₋₃ alkyl, and C₁₋₃ haloalkyl;
one R⁵ is R^{5A}; and each other R⁵ is independently selected from H, D, halo, C₁₋₃ alkyl, OR^{a5}, C₁₋₃ haloalkyl, C₂₋₃ alkenyl, and C₂₋₃ alkynyl; or, optionally, two other R⁵ attached to the same carbon atom, together with the carbon atom to which they are both attached, form a spiro C₃₋₆ cycloalkyl ring that is optionally substituted with 1, 2, 3, or 4 substituents each selected from D, C₁₋₃ alkyl, and halo; or, optionally, two other R⁵ attached to adjacent carbon atoms, together with the carbon atoms to which they are each attached, form a fused C₃₋₆ cycloalkyl ring that is optionally substituted with 1, 2, 3, or 4 substituents each selected from D, C₁₋₃ alkyl, and halo;
R^{5A} is H, D, C₁₋₃ alkyl, C₁₋₃ haloalkyl, C₂₋₃ alkenyl, C₂₋₃ alkynyl, halo, OR^{a5A}, CN, or Cy²; wherein the C₁₋₃ alkyl forming R^{5A} is optionally substituted with 1, 2, 3 or 4 substituents each selected from R^{5B} and also optionally substituted with Cy^{Z}, or, optionally, R^{5A} and R⁵ attached to the same carbon atom, together with the carbon atom to which they are both attached, form a spiro C₃₋₆ cycloalkyl ring that is optionally substituted with 1, 2, 3, or 4 substituents each selected from D, C₁₋₃ alkyl, and halo; or, optionally, R^{5A} and R⁵ attached to adjacent carbon atoms, together with the carbon atoms to which they are each attached, form a fused C₃₋₆ cycloalkyl ring that is optionally substituted with 1, 2, 3, or 4 substituents each selected from D, C₁₋₃ alkyl, and halo;
each R^{5B} is independently selected from 0 and halo;
each R^{a5} is independently selected from H, C₁₋₃ alkyl, and C₁₋₃ haloalkyl;
R^{a5A} is selected from H, C₁₋₃ alkyl, C₁₋₃ haloalkyl, and Cy², wherein the C₁₋₃ alkyl forming R^{a5A} is optionally substituted with 1, 2, 3 or 4 substituents each selected from R^{5B} and also optionally substituted with Cy²;
Cy² is selected from C₃₋₇ cycloalkyl, 4-10 membered heterocycloalkyl, C₆₋₁₀ aryl, and 5-10 membered heteroaryl; wherein the C₃₋₇ cycloalkyl, 4-10 membered heterocycloalkyl, C₆₋₁₀ aryl, 5-10 membered heteroaryl, C₆₋₁₀ aryl, and 5-10 membered heteroaryl forming Cy² is optionally substituted with 1, 2, 3, or 4 substituents independently selected from R^{Cy2}; wherein the ring-forming atoms of the 4-10 membered heterocycloalkyl and 5-10 membered heteroaryl forming Cy² consist of at least one carbon atom and 1, 2, 3, or 4 heteroatoms independently selected from N, O, and S; and wherein a ring-forming carbon atom of the 4-10 membered heterocycloalkyl and 5-10 membered heteroaryl forming Cy² is optionally substituted by oxo to form a carbonyl group;
each R^{Cy2} is independently selected from D, C₁₋₃ alkyl, C₁₋₃ haloalkyl, C₂₋₃ alkenyl, C₂₋₃ alkynyl, C₃₋₆ cycloalkyl, 4-10 membered heterocycloalkyl, C₆₋₁₀ aryl, 5-10 membered heteroaryl, halo, CN, OR^{aCy21}, SR^{aCy21}, C(O)R^{bCy21}, C(O)NR^{cCy21}R^{dCy21}, C(O)OR^{aCy21}, OC(O)R^{bCy21} OC(O)NR^{cCy21}R^{dCy21}, NR^{cCy21}, R^{dCy21}, NR^{cCy21}, C(O)R^{bCy21}, NR^{cCy21}, C(O)NR^{cCy21}, R^{dCy21} NR^{cCy21}C(O)OR^{aCy21}, C(═NR^{eCy21})NR^{cCy21}R^{dCy21}, NR^{cCy21}C(═NR^{eCy21})NR^{cCy21}R^{dCy21}, S(O)R^{bCy21} S(O)NR^{cCy21}R^{dCy21}, S(O)₂R^{bCy21}, NR^{cCy21}S(O)₂R^{bCy21}, and S(O)₂NR^{cCy21}R^{dCy21} wherein the C₃₋₆ cycloalkyl, 4-10 membered heterocycloalkyl, C₆₋₁₀ aryl, and 5-10 membered heteroaryl forming R^{Cy2} are each optionally substituted by 1, 2, 3, or 4 substituents independently selected from R^{Cy2A}; wherein the ring-forming atoms of the 4-10 membered heterocycloalkyl and 5-10 membered heteroaryl forming R^{Cy2} consist of at least one carbon atom and 1, 2, 3, or 4 heteroatoms independently selected from N, O, and S; wherein a ring-forming carbon atom of the 4-10 membered heterocycloalkyl and 5-10 membered heteroaryl forming R^{Cy2} is optionally substituted by oxo to form a carbonyl group; and wherein the C₁₋₃ alkyl, C₂₋₃ alkenyl, and C₂₋₃ alkynyl forming R^{Cy2} are each optionally substituted by 1, 2, or 3 substituents independently selected from R^{Cy2B};
each R^{Cy2A} is independently selected from C₁₋₃ alkyl, C₁₋₃ haloalkyl, C₂₋₃ alkenyl, C₂₋₃ alkynyl, and R^{Cy2B}; wherein the C₁₋₃ alkyl, C₂₋₃ alkenyl, and C₂₋₃ alkynyl forming R^{Cy2A} are each optionally substituted by 1, 2, or 3 substituents independently selected from R^{Cy2B},
each R^{Cy2B} is independently selected from D, halo, CN, OR^{aCy21}, SR^{aCy21}, C(O)R^{bCy21}, C(O)NR^{cCy21}R^{dCy21}, C(O)OR^{aCy21}, OC(O)R^{bCy21}, OC(O)NR^{cCy21}R^{dCy21}, NR^{cCy21}R^{dCy21} NR^{cCy21}C(O)R^{bCy21}, NR^{cCy21}C(O)NR^{cCy21}R^{dCy21}, NR^{cCy21}C(O)OR^{aCy21}, C(═NR^{eCy21})NR^{cCy21}R^{dCy21} NR^{cCy21}C(═NR^{eCy21})NR^{cCy21}R^{dCy21}, S(O)R^{bCy21}, S(O)NR^{cCy21}R^{dCy21}, S(O)₂R^{bCy21} NR^{cCy21}S(O)₂R^{bCy21}, and S(O)₂NR^{cCy21}R^{dCy21};
R^{aCy21}, R^{bCy21}, R^{cCy21}, and R^{dCy21} are each independently selected from H, C₁₋₃ alkyl, C₁₋₃ haloalkyl, C₂₋₃ alkenyl, C₂₋₃ alkynyl, C₆₋₁₀ aryl, C₃₋₇ cycloalkyl, 5-10 membered heteroaryl, 4-10 membered heterocycloalkyl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃alkyl forming R^{aCy21}, R^{bCy21}, R^{cCy21}, and R^{dCy21} are each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{aCy22}, SR^{aCy22}, C(O)R^{bCy22}, C(O)NR^{cCy22}R^{dCy22}, C(O)OR^{aCy22}, OC(O)R^{bCy22}, OC(O)NR^{cCy22}R^{dCy22}, NR^{cCy22}R^{dCy22}, NR^{cCy22}C(O)R^{bCy22}, NR^{cCy22}C(O)NR^{cCy22}R^{dCy22}, NR^{cCy22}C(O)OR^{aCy22}, C(═NR^{cCy22})NR^{cCy22}R^{dCy22}, NR^{cCy22}C(═NR^{cCy22})NR^{cCy22}R^{dCy22}, S(O)R^{bCy22}, S(O)NR^{cCy22}R^{dCy22}, S(O)₂R^{bCy22}, NR^{cCy22}S(O)₂R^{bCy22}, and S(O)₂NR^{cCy22}R^{dCy22}; wherein the ring-forming atoms each of the 5-10 membered heteroaryl, 4-10 membered heterocycloalkyl, 5-10 membered heteroaryl-C₁₋₃ alkyl, and 4-10 membered heterocycloalkyl-C₁₋₃ alkyl forming R^{aCy21}, R^{bCy21}, R^{cCy21}, and R^{dCy21} consist of at least one carbon atom and 1, 2, 3, or 4 heteroatoms independently selected from N, O, and S; and wherein a ring-forming carbon atom of the 5-10 membered heteroaryl, 4-10 membered heterocycloalkyl, 5-10 membered heteroaryl-C₁₋₃ alkyl, and 4-10 membered heterocycloalkyl-C₁₋₃alkyl forming R^{aCy21}, R^{bCy21}, R^{cCy21}, and R^{dCy21} is optionally substituted by oxo to form a carbonyl group;
or R^{cCy21} and R^{dCy21} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{aCy22}, SR^{aCy22}, C(O)R^{bCy22}, C(O)NR^{cCy22}R^{dCy22}, C(O)OR^{aCy22}, OC(O)R^{bCy22}, OC(O)NR^{cCy22}R^{dCy22}, NR^{cCy22}R^{dCy22}, NR^{cCy22}C(O)R^{bCy22}, NR^{cCy22}C(O)NR^{cCy22}R^{dCy22}, NR^{cCy22}C(O)OR^{aCy22}, C(═NR^{cCy22})NR^{cCy22}R^{dCy22}, NR^{cCy22}C(═NR^{cCy22})NR^{cCy22}R^{dCy22}, S(O)R^{bCy22}, S(O)NR^{cCy22}R^{dCy22}, S(O)₂R^{bCy22}, NR^{cCy22}S(O)₂R^{bCy22}, and S(O)₂NR^{cCy22}R^{dCy22};
R^{aCy22}, R^{bCy22}, R^{cCy22}, and R^{dCy22} are each independently selected from H, C₁₋₃ alkyl, C₁₋₃ haloalkyl, C₂₋₃ alkenyl, C₂₋₃ alkynyl, aryl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₁₋₃ alkyl, C₂₋₃ alkenyl, C₂₋₃ alkynyl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{aCy22}, R^{bCy22}, R^{cCy22}, and R^{dCy22} are each optionally substituted with 1, 2, or 3 substituents independently selected from OH, CN, amino, NH(C₁₋₃ alkyl), N(C₁₋₃ alkyl)₂, halo, C₁₋₃ alkyl, C₁₋₃ alkoxy, C₁₋₃ haloalkyl, and C₁₋₃ haloalkoxy; wherein the ring-forming atoms each of the 5-10 membered heteroaryl, 4-10 membered heterocycloalkyl, 5-10 membered heteroaryl-C₁₋₃ alkyl, and 4-10 membered heterocycloalkyl-C₁₋₃ alkyl forming R^{aCy22}, R^{bCy22}, R^{cCy22}, and R^{dCy22} consist of at least one carbon atom and 1, 2, 3, or 4 heteroatoms independently selected from N, O, and S; and wherein a ring-forming carbon atom of the 5-10 membered heteroaryl, 4-10 membered heterocycloalkyl, 5-10 membered heteroaryl-C₁₋₃ alkyl, and 4-10 membered heterocycloalkyl-C₁₋₃ alkyl forming R^{aCy22}, R^{bCy22}, R^{cCy22}, and R^{dCy22} is optionally substituted by oxo to form a carbonyl group; or
R^{cCy22} and R^{dCy22} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₃ alkyl, C₁₋₃ alkoxy, C₁₋₃ haloalkyl, and C₁₋₃ haloalkoxy; and
R^{cCy21} and R^{cCy22} are each, independently, H, CN or NO₂.

27. The method of claim 26, wherein
Cy¹ is phenyl optionally substituted with 1 or 2 substituents each selected from D, C₁₋₃ alkyl, C₁₋₃ haloalkyl, halo, OH, and C₁₋₃ alkoxy;
R¹ is halo;
R² is C₁₋₃ alkyl optionally substituted with OH;
R³ is C₃₋₁₀ cycloalkyl optionally substituted with halo;
each R⁴ is H;
one R⁵ is R^{5A}; and each other R⁵ is independently selected from H, D, halo, C₁₋₃ alkyl, OC₁₋₃ alkyl, C₁₋₃ haloalkyl; or, optionally, two other R⁵ attached to adjacent carbon atoms, together with the carbon atoms to which they are each attached, form a fused C₃₋₆ cycloalkyl ring that is optionally substituted with 1 or 2 substituents each selected from D, C₁₋₃ alkyl, and halo; and
R^{5A} is H, halo, or OR^{a5A};
R^{a5A} is selected from C₁₋₃ alkyl, C₁₋₃ haloalkyl, and Cy², wherein the C₁₋₃ alkyl forming R^{a5A} is optionally substituted with 1, 2, or 3 D, and also optionally substituted with Cy²; and
Cy² is selected from C₆₋₁₀ aryl and 5-10 membered heteroaryl.

28-29. (canceled)

30. The method of claim 26, wherein the compound of Formula IV is a compound of Formula IV-B 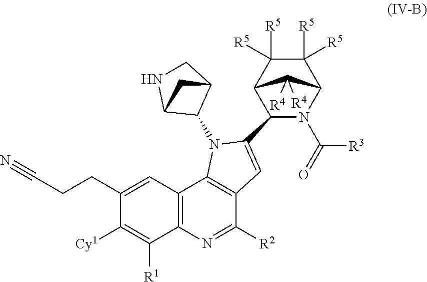
or a pharmaceutically acceptable salt thereof.

31. The method of claim 26, to wherein the compound of Formula IV is selected from:
3-(1-(2-azabicyclo[2.1.1]hexan-5-yl)-2-(2-(cyclopropanecarbonyl)-5-methoxy-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-fluoro-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-6-azatricyclo[3.2.1.02,4]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-(methoxy-d3)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-(pyridin-3-yloxy)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(2-(5-(benzyloxy)-2-(cyclopropanecarbonyl)-2-azabicyclo[2.2.1]heptan-3-yl)-1-(2-azabicyclo[2.1.1]hexan-5-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-(difluoromethoxy)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-7-(2,3-dichlorophenyl)-6-fluoro-2-(5-fluoro-2-(1-fluorocyclopropane-1-carbonyl)-2-azabicyclo[2.2.1]heptan-3-yl)-4-((R)-1-hydroxyethyl)-1H-pyrrolo[3,2-c]quinolin-8-yl)propanenitrile;
3-(1-(2-azabicyclo[2.1.1]hexan-5-yl)-2-(2-(cyclopropanecarbonyl)-5-(difluoromethyl)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
5-(1-(2-azabicyclo[2.1.1]hexan-5-yl)-8-(2-cyanoethyl)-2-(2-(cyclopropanecarbonyl)-2-azabicyclo[2.2.1]heptan-3-yl)-7-(2,3-dichlorophenyl)-6-fluoro-1H-pyrrolo[3,2-c]quinolin-4-yl)-N,N-dimethylpicolinamide;
3-(1-(2-azabicyclo[2.1.1]hexan-5-yl)-2-(2-(cyclopropanecarbonyl)-2-azabicyclo[2.2.1]heptan-3-yl)-7-(2,3-dichlorophenyl)-6-fluoro-4-(6-(2-hydroxypropan-2-yl)pyridin-3-yl)-1H-pyrrolo[3,2-c]quinolin-8-yl)propanenitrile;
4-(1-(2-azabicyclo[2.1.1]hexan-5-yl)-8-(2-cyanoethyl)-2-(2-(cyclopropanecarbonyl)-2-azabicyclo[2.2.1]heptan-3-yl)-7-(2,3-dichlorophenyl)-6-fluoro-1H-pyrrolo[3,2-c]quinolin-4-yl)-2-fluoro-N-methylbenzamide;
3-(1-(2-azabicyclo[2.1.1]hexan-5-yl)-2-(2-(cyclopropanecarbonyl)-5-methyl-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-hydroxy-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-(pyridin-2-yloxy)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-(pyridin-4-yloxy)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-7-(2,3-dichlorophenyl)-2-(5-(difluoromethoxy)-2-(1-fluorocyclopropane-1-carbonyl)-2-azabicyclo[2.2.1]heptan-3-yl)-6-fluoro-4-methyl-1H-pyrrolo[3,2-c]quinolin-8-yl)propanenitrile;
3-(1-(2-azabicyclo[2.1.1]hexan-5-yl)-7-(2,3-dichlorophenyl)-6-fluoro-2-(5-fluoro-2-(1-fluorocyclopropane-1-carbonyl)-2-azabicyclo[2.2.1]heptan-3-yl)-4-(1-hydroxyethyl)-1H-pyrrolo[3,2-c]quinolin-8-yl)propanenitrile;
3-(1-(2-azabicyclo[2.1.1]hexan-5-yl)-2-(5-chloro-2-(1-fluorocyclopropane-1-carbonyl)-2-azabicyclo[2.2.1]heptan-3-yl)-7-(2,3-dichlorophenyl)-6-fluoro-4-(1-hydroxyethyl)-1H-pyrrolo[3,2-c]quinolin-8-yl)propanenitrile;
3-(1-(2-azabicyclo[2.1.1]hexan-5-yl)-2-(2-(cyclopropanecarbonyl)-5-(trifluoromethoxy)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-7-(2,3-dichlorophenyl)-2-(5-(difluoromethoxy)-2-(1-fluorocyclopropane-1-carbonyl)-2-azabicyclo[2.2.1]heptan-3-yl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-cyclopropoxy-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-7-(2,3-dichlorophenyl)-2-(5-(difluoromethoxy)-2-(1-fluorocyclopropane-1-carbonyl)-2-azabicyclo[2.2.1]heptan-3-yl)-6-fluoro-4-(1-hydroxyethyl)-1H-pyrrolo[3,2-c]quinolin-8-yl)propanenitrile;
3-(1-(2-azabicyclo[2.1.1]hexan-5-yl)-2-(5-cyclopropoxy-2-(1-fluorocyclopropane-1-carbonyl)-2-azabicyclo[2.2.1]heptan-3-yl)-7-(2,3-dichlorophenyl)-6-fluoro-4-(1-hydroxyethyl)-1H-pyrrolo[3,2-c]quinolin-8-yl)propanenitrile;
methyl 3-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(trifluoromethoxy)-2-azabicyclo[2.2.1]heptane-2-carboxylate;
3-(1-(2-azabicyclo[2.1.1]hexan-5-yl)-7-(2,3-dichlorophenyl)-6-fluoro-2-(2-(1-fluorocyclopropane-1-carbonyl)-5-(trifluoromethoxy)-2-azabicyclo[2.2.1]heptan-3-yl)-4-methyl-1H-pyrrolo[3,2-c]quinolin-8-yl)propanenitrile;
methyl 3-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethyl)-2-azabicyclo[2.2.1]heptane-2-carboxylate; and
3-(1-(2-azabicyclo[2.1.1]hexan-5-yl)-7-(2,3-dichlorophenyl)-2-(5-(difluoromethyl)-2-(1-fluorocyclopropane-1-carbonyl)-2-azabicyclo[2.2.1]heptan-3-yl)-6-fluoro-4-methyl-1H-pyrrolo[3,2-c]quinolin-8-yl)propanenitrile;
and pharmaceutically acceptable salts thereof.

32. The method of claim 26, wherein the KRAS G12D inhibitor is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate, or a pharmaceutically acceptable salt thereof.

33. (canceled)

34. The method of claim 1, wherein the PD-1 inhibitor or PD-L1 inhibitor is administered to the subject in a pharmaceutical composition comprising the PD-1 inhibitor or PD-L1, or a pharmaceutically acceptable salt thereof, and at least one pharmaceutically acceptable carrier or excipient.

35. (canceled)

36. A method of treating cancer in a subject in need thereof comprising administering to the subject a KRAS G12D inhibitor that is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, and a PD-1 inhibitor that is retifanlimab.

37. (canceled)

38. The method of claim 36, wherein the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate.

39. (canceled)

40. The method of claim 1, wherein the KRAS G12D inhibitor is administered twice daily (BID) or once daily (QD), and the KRAS G12D inhibitor is administered orally (PO).

41-42. (canceled)

43. The method of claim 1, wherein the PD-1 inhibitor is administered twice a week (BIW) and the PD-1 inhibitor is administered as an intraperitoneal injection (IP).

44. (canceled)

45. The method of claim 1, wherein the cancer is selected from carcinomas, hematological cancers, sarcomas, and glioblastoma.

46. The method of claim 1, wherein the cancer is a cancer comprising abnormally proliferating cells having a KRAS G12D mutation.

47. (canceled)

48. The method of claim 45, wherein the cancer is a hematological cancer selected from myeloproliferative neoplasms, myelodysplastic syndrome, chronic and juvenile myelomonocytic leukemia, acute myeloid leukemia, acute lymphocytic leukemia, and multiple myeloma.

49. The method of claim 45, wherein the cancer is a carcinoma selected from pancreatic, colorectal, lung, bladder, gastric, esophageal, breast, head and neck, cervical, skin, and thyroid carcinomas.

50. The method of claim 49, wherein the carcinoma is colorectal carcinoma, lung carcinoma, or pancreatic carcinoma.

51-52. (canceled)

53. The method of claim 1, wherein the cancer is colorectal cancer, non-small cell lung cancer (NSCLC), or pancreatic ductal adenocarcinoma.

54-55. (canceled)

56. The method of claim 1, wherein the cancer is metastatic.

57-66. (canceled)

67. A pharmaceutical combination comprising a KRAS G12D inhibitor, or a pharmaceutically acceptable salt thereof, and a PD-1 inhibitor or a PD-L1 inhibitor, or pharmaceutically acceptable salt thereof.

68. The pharmaceutical combination of claim 67, wherein the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

69. (canceled)

70. The pharmaceutical combination of claim 67, wherein the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate.

71. (canceled)

72. The pharmaceutical combination of claim 67, wherein the KRAS G12D inhibitor is the KRAS G12D inhibitor is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate, or a pharmaceutically acceptable salt thereof.

73. The pharmaceutical combination of claim 67, wherein the PD-1 inhibitor is retifanlimab.

74. The pharmaceutical combination of claim 67, wherein the PD-L1 inhibitor is 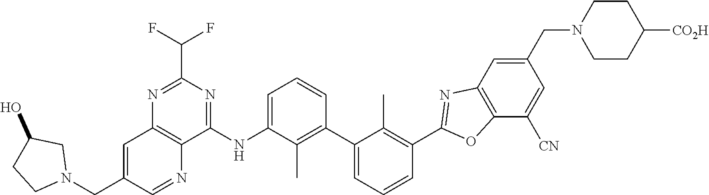 [SMILES: Cc1c(Nc2nc(C(F)F)nc3cc(CN4CC[C@@H](O)C4)cnc23)cccc1-c1cccc(-c2nc3cc(CN4CCC(C(=O)O)CC4)cc(C#N)c3o2)c1C]
or a pharmaceutically acceptable salt thereof.
